(19) United States
(12) Patent Application Publication
(10) Pub. No.: US 2023/0115350 A1
(43) Pub. Date: Apr. 13, 2023

(54) TEAD INHIBITORS AND USES THEREOF

(71) Applicant: Cedilla Therapeutics, Inc., Cambridge, MA (US)

(72) Inventors: Glen Robert Rennie, Somerville, MA (US); C. Eric Schwartz, Marblehead, MA (US); Louise Kirman, Swampscott, MA (US); Dale Porter, Cambridge, MA (US); Ling Song, Cambridge, MA (US)

(21) Appl. No.: 17/816,232

(22) Filed: Jul. 29, 2022

Related U.S. Application Data
(60) Provisional application No. 63/227,159, filed on Jul. 29, 2021.

(51) Int. Cl. C07D 265/36; C07C 235/38; C07D 311/60; C07D 319/20; C07D 213/56; C07C 311/13; C07D 309/06; C07D 333/56; C07D 333/38; C07D 277/24; C07D 317/40; C07D 317/66; C07D 471/04; C07D 257/04; C07D 215/06

(52) U.S. Cl. CPC C07D 265/36; C07C 235/38; C07D 311/60; C07D 319/20; C07D 213/56; C07C 311/13; C07D 309/06; C07D 333/56; C07D 333/38; C07D 277/24; C07D 317/40; C07D 317/66; C07D 471/04; C07D 257/04; C07D 215/06

(57) ABSTRACT

The present disclosure provides compounds, pharmaceutically acceptable compositions thereof, and methods of using the same.

### TECHNICAL FIELD OF INVENTION

The present disclosure relates to compounds and methods useful for inhibition of Transcriptional Enhancer Associate Domain (TEAD) transcription factors. The disclosure also provides pharmaceutically acceptable compositions comprising compounds of the present disclosure and methods of using said compositions in the treatment of various diseases, disorders, and conditions as described herein.

### BACKGROUND OF THE INVENTION

Yes-associated protein (YAP) and transcriptional co-activator with PDZ-binding motif (TAZ) are transcriptional co-activators of the Hippo signaling pathway and regulate cell proliferation, migration, and apoptosis. Inhibition of the Hippo signaling pathway promotes YAP/TAZ translocation to the nucleus, where YAP/TAZ interact with TEAD transcription factors to co-activate the expression of target genes and promote cell proliferation. Hyperactivation of YAP and TAZ and/or mutations in one or more members of the Hippo signaling pathway have been implicated in various diseases, disorders, and conditions.

### SUMMARY OF THE INVENTION

In some embodiments, the present disclosure provides the recognition that there remains a need to find inhibitors of the Hippo signaling pathway useful as therapeutic agents. It has now been found that compounds of the present disclosure, and pharmaceutically acceptable salts and compositions thereof, are effective as inhibitors of TEAD transcription factors (e.g., TEAD1, TEAD2, TEAD3, and/or TEAD4). In some embodiments, such compounds have general Formula I:

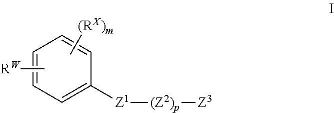

or a pharmaceutically acceptable salt thereof, wherein:
R^{w} is a warhead group;
each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
Z³ is an optionally substituted C₁₋₆ aliphatic or

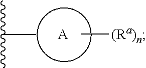

Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur;
each R^{a} is independently oxo, halogen, —CN, —NR₂, —OR, —SR, —C(O)R, —C(O)OR, NO₂, or an optionally substituted C₁₋₆ aliphatic group;
Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or
   when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or
   R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur;
each Z² is independently —CR^{z}—;
each R^{z} is independently selected from hydrogen or optionally substituted C₁₋₆ aliphatic;
m is 0, 1, 2, 3, or 4;
n is 0, 1, 2, 3, 4, or 5; and
p is 0, 1, 2, or 3.

In some embodiments, the present disclosure provides a pharmaceutical composition comprising a compound described herein, and a pharmaceutically acceptable carrier.

Compounds described herein, and pharmaceutically acceptable compositions thereof, are useful for treating a variety of diseases, disorders, or conditions associated with the Hippo signaling pathway. Such diseases, disorders, or conditions include those described herein.

Compounds provided herein are also useful for the study of the Hippo signaling pathway in, e.g., biological and pathological phenomena, and the comparative evaluation of new TEAD transcription factor inhibitors.

### BRIEF DESCRIPTION OF THE DRAWINGS

FIG. 1 depicts mass modification of TEAD1 by compound 58.

FIG. 2 depicts mass modification of TEAD1 by compound 80.

FIG. 3 depicts mass modification of TEAD1 by compound 89.

FIG. 4 depicts mass modification of TEAD1 by compound 73.

FIG. 5 depicts mass modification of TEAD1 by compound 53.

FIG. 6 depicts mass modification of TEAD1 by compound 59.

### DETAILED DESCRIPTION OF CERTAIN EMBODIMENTS

### 1. General Description of Certain Embodiments of the Invention

In certain embodiments, the present disclosure provides inhibitors of TEAD transcription factors. In some embodiments, such compounds include those of the formulae described herein, or a pharmaceutically acceptable salt thereof, wherein each variable is as defined and described herein.

In one aspect, the present disclosure provides a compound of Formula I:

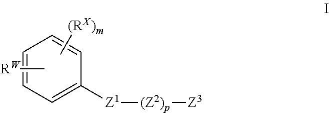

or a pharmaceutically acceptable salt thereof, wherein:
R^{w} is a warhead group;
each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
Z³ is an optionally substituted C₁₋₆ aliphatic or

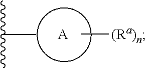

Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur;
each R^{a} is independently oxo, halogen, —CN, —NR₂, —OR, —SR, —C(O)R, —C(O)OR, NO₂, or an optionally substituted C₁₋₆ aliphatic group;
Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or
   when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or
   R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur;
each Z² is independently —CR^{z}—;
each R^{z} is independently selected from hydrogen or optionally substituted C₁₋₆ aliphatic;
m is 0, 1, 2, 3, or 4;
n is 0, 1, 2, 3, 4, or 5; and
p is 0, 1, 2, or 3.

### 2. Compounds and Definitions

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “halogen” means F, Cl, Br, or I.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl”, as used herein, does not differ significantly from the common meaning of the term in the art, and refers to a cyclic aromatic radical having from five to twelve ring atoms of which one ring atom is selected from S, O and N; zero, one, two, three, four, or five ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

The term “heteroaryl” as used herein, refers to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, tetrahydroquinolinyl, and tetrahydroisoquinolinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

Additionally, it will be appreciated that, when two groups cyclize to form an optionally substituted heteroaryl ring having at least one nitrogen atom, the nitrogen atom in the ring can be, as valency permits, N or N—R^{†}, as defined infra.

As used herein, the terms “heterocycle”, “heterocyclyl”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, tetrahydroquinolinyl, or tetrahydroisoquinolinyl where the radical or point of attachment is on the heterocyclyl ring. A heterocyclyl group may be mono- or bicyclic.

Additionally, it will be appreciated that, when two groups cyclize to form an optionally substituted heterocyclic ring having at least one nitrogen atom, the nitrogen atom in the ring can be, as valency permits, N or N—R^{†}, as defined infra.

As described herein, compounds may contain “optionally substituted” moieties. In general, the term “substituted”, whether preceded by the term “optionally” or not, means that one or more hydrogens of the designated moiety of compounds are replaced with a suitable substituent. “Substituted” applies to one or more hydrogens that are either explicit or implicit from the structure (e.g.,

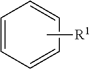

refers to at least

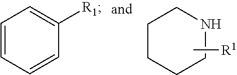

and refers to at least

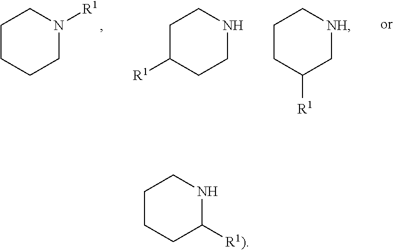

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6 membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{†}, -(haloR^{†}), —OH, —OR^{†}, —O(haloR^{†}), —CN, —C(O)OH, —C(O)OR^{†}, —NH₂, —NHR^{†}, —NR^{†}₂, or —NO₂, wherein each R^{†} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

As used herein, the term “irreversible” or “irreversible inhibitor” refers to an inhibitor (i.e. a compound) that is able to be covalently bonded to a target protein kinase in a substantially non-reversible manner. That is, whereas a reversible inhibitor is able to bind to (but is generally unable to form a covalent bond) the target protein kinase, and therefore can become dissociated from the target protein kinase, an irreversible inhibitor will remain substantially bound to the target protein kinase once covalent bond formation has occurred. Irreversible inhibitors usually display time dependency, whereby the degree of inhibition increases with the time with which the inhibitor is in contact with the enzyme. Methods for identifying if a compound is acting as an irreversible inhibitor are known to one of ordinary skill in the art. Such methods include, but are not limited to, enzyme kinetic analysis of the inhibition profile of the compound with the protein kinase target, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

It will be appreciated that certain reactive functional groups can act as “warheads.” As used herein, the term “warhead” or “warhead group” refers to a functional group present on a compound of the present invention wherein that functional group is capable of covalently binding to a side chain of an amino acid residue (such as cysteine, lysine, histidine, or other residues capable of being covalently modified) present in the binding pocket of the target protein (e.g., a TEAD transcription factor such as TEAD1, TEAD2, TEAD3, or TEAD4), thereby irreversibly inhibiting the protein. In some embodiments, the R^{w} group, as defined and described herein, provides such warhead groups for covalently, and irreversibly, inhibiting the protein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

As used herein, a “disease or disorder associated with TEAD” or, alternatively, “a TEAD-mediated disease or disorder” means any disease or other deleterious condition in which TEAD, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds described herein that may be combined with the carrier materials to produce a composition in a single dosage form will vary depending upon the host treated, the particular mode of administration, etc.

The expression “unit dosage form” as used herein refers to a physically discrete unit of a provided compound and/or compositions thereof appropriate for the subject to be treated. It will be understood, however, that the total daily usage of the active agent (i.e., compounds and compositions described herein) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of a TEAD-mediated disease or disorder.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

### 3. Description of Exemplary Embodiments

In some embodiments, the present disclosure provides a compound of Formula I:

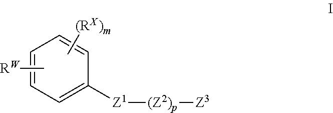

or a pharmaceutically acceptable salt thereof, wherein:
R^{w} is a warhead group;
each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
Z³ is an optionally substituted C₁₋₆ aliphatic or

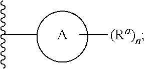

Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur;
each R^{a} is independently oxo, halogen, —CN, —NR₂, —OR, —SR, —C(O)R, —C(O)OR, NO₂, or an optionally substituted C₁₋₆ aliphatic group;
Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or
   when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or
   R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur;
each Z² is independently —CR^{z}—;
each R^{z} is independently selected from hydrogen or optionally substituted C₁₋₆ aliphatic;
m is 0, 1, 2, 3, or 4;
n is 0, 1, 2, 3, 4, or 5; and
p is 0, 1, 2, or 3.

As defined generally above, each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur. In some embodiments, R^{x} is halogen, —CN, —NR₂, —OR, or an optionally substituted group selected from C₁₋₆ aliphatic or a 3- to 7-membered saturated or partially unsaturated carbocyclic ring. In some embodiments, R^{x} is halogen, —CN, —OR, or an optionally substituted group selected from C₁₋₆ aliphatic or a 3- to 7-membered saturated or partially unsaturated carbocyclic ring. In some embodiments, R^{x} is halogen, —CN, —NR₂, or an optionally substituted group selected from C₁₋₆ aliphatic or a 3- to 7-membered saturated or partially unsaturated carbocyclic ring. In some embodiments, R^{x} is halogen, —CN, or an optionally substituted group selected from C₁₋₆ aliphatic or a 3- to 7-membered saturated or partially unsaturated carbocyclic ring.

In some embodiments, R^{x} is halogen. In some embodiments, R^{x} is fluoro or chloro. In some embodiments, R^{x} is fluoro. In some embodiments, R^{x} is chloro. In some embodiments, R^{x} is —CN. In some embodiments, R^{x} is —OR. In some embodiments, R^{x} is —OCH₃.

In some embodiments, R^{x} is an optionally substituted C₁₋₆ aliphatic. In some embodiments, R^{x} is C₁₋₆ aliphatic optionally substituted with halogen, —(CH₂)₀₋₄R^{∘}, or —(CH₂)₀₋₄C(O)NR^{∘}₂. In some embodiments, R^{x} is C₁₋₆ aliphatic optionally substituted with halogen. In some embodiments, R^{x} is C₁₋₆ aliphatic optionally substituted with fluoro. In some embodiments, R^{x} is C₁₋₃ aliphatic optionally substituted with fluoro. In some embodiments, R^{x} is —CF₃. In some embodiments, R^{x} is C₁₋₆ aliphatic optionally substituted with —(CH₂)₀₋₄R^{∘}. In some embodiments, R^{x} is C₁₋₆ aliphatic optionally substituted with —R^{∘}. In some embodiments, R^{x} is C₁₋₆ aliphatic optionally substituted with —R^{∘}, wherein —R^{∘} is a 3- to 6-membered saturated, partially unsaturated, or aryl ring having 0-4 heteroatoms independently selected from nitrogen, oxygen, or sulfur. In some embodiments, R^{x} is C₁₋₆ aliphatic optionally substituted with —R^{∘}, wherein —R^{∘} is a 3- to 6-membered saturated, partially unsaturated, or aryl ring having 0-1 nitrogen atom. In some embodiments, R^{x} is C₁₋₆ aliphatic optionally substituted with cyclopropyl or pyridinyl. In some embodiments, R^{x} is C₁₋₃ aliphatic optionally substituted with cyclopropyl or pyridinyl. In some embodiments, R^{x} is

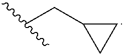 [SMILES: *CC1CC1]

In some embodiments, R^{x} is

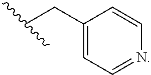 [SMILES: *Cc1ccncc1]

In some embodiments, R^{x} is C₁₋₆ aliphatic optionally substituted with —(CH₂)₀₋₄C(O)NR^{∘}₂. In some embodiments, R^{x} is C₁₋₆ aliphatic optionally substituted with —C(O)NR^{∘}₂. In some embodiments, R^{x} is C₁₋₃ aliphatic optionally substituted with —C(O)NR^{∘}₂. In some embodiments, R^{x} is

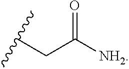 [SMILES: *CC(N)=O]

In some embodiments, R^{x} is an optionally substituted 3- to 7-membered saturated or partially unsaturated carbocyclic ring. In some embodiments, R^{x} is an optionally substituted 3- to 6-membered saturated or partially unsaturated carbocyclic ring. In some embodiments, R^{x} is optionally substituted cyclopropyl. In some embodiments, R^{x} is optionally substituted cyclohexyl. In some embodiments, R^{x} is cyclopropyl. In some embodiments, R^{x} is cyclohexyl.

In some embodiments R^{x} is selected from the group consisting of: —CN, —F, —Cl, —CH₃, —CF₃, —OMe,

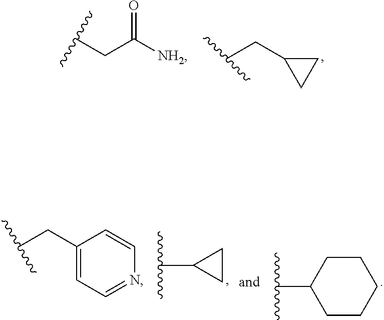

In some embodiments R^{x} is selected from the group consisting of: —CN, —F, —Cl, —CH₃, —CF₃,

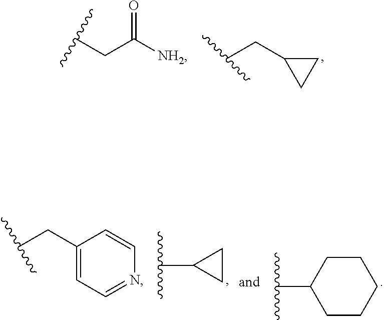

As defined generally above, each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R is hydrogen or an optionally substituted C₁₋₆ aliphatic group. In some embodiments, R is hydrogen. In some embodiments, R is an optionally substituted C₁₋₆ aliphatic group. In some embodiments, R is methyl. In some embodiments, R is ethyl. In some embodiments, R is propyl. In some embodiments, R is n-propyl. In some embodiments, R is isopropyl. In some embodiments, R is butyl. In some embodiments, R is n-butyl. In some embodiments, R is sec-butyl. In some embodiments, R is isobutyl. In some embodiments, R is tert-butyl. In some embodiments, R is pentyl. In some embodiments, R is hexyl.

As defined generally above, Z³ is an optionally substituted C₁₋₆ aliphatic or

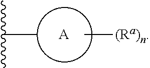

In some embodiments, Z³ is

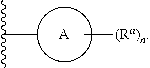

In some embodiments, Z³ is an optionally substituted C₁₋₆ aliphatic. In some embodiments, Z³ is C₁₋₆ aliphatic optionally substituted with halogen. In some embodiments, Z³ is C₁₋₆ aliphatic optionally substituted with fluoro. In some embodiments, Z³ is C₃₋₆ aliphatic optionally substituted with halogen. In some embodiments, Z³ is C₂₋₆ aliphatic optionally substituted with fluoro. In some embodiments, Z³ is C₃₋₆ aliphatic optionally substituted with fluoro. In some embodiments, Z³ is selected from the group consisting of:

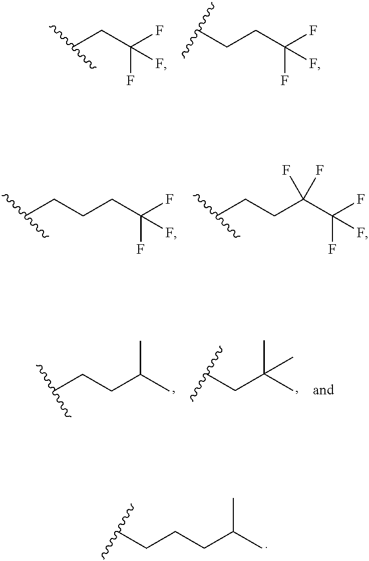

As defined generally above, Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur. In some embodiments, Ring A is selected from a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur.

In some embodiments, Ring A is a 3- to 7-membered saturated or partially unsaturated carbocyclic ring. In some embodiments, Ring A is a 3- to 6-membered saturated or partially unsaturated carbocyclic ring. In some embodiments, Ring A is cyclopropyl or cyclohexyl. In some embodiments, Ring A is phenyl.

In some embodiments, Ring A is a 4- to 7-membered saturated or partially unsaturated heterocyclic ring having 1-3 heteroatoms independently selected from nitrogen, oxygen, and sulfur. In some embodiments, Ring A is a 5- to 6-membered saturated or partially unsaturated heterocyclic ring having 1-3 heteroatoms independently selected from nitrogen, oxygen, and sulfur. In some embodiments, Ring A is a 5- to 6-membered saturated or partially unsaturated heterocyclic ring having 1-2 heteroatoms independently selected from nitrogen and oxygen. In some embodiments, Ring A is tetrahydropyranyl or dioxolyl.

In some embodiments, Ring A is a 5- to 6-membered heteroaryl ring having 1-4 heteroatoms independently selected from nitrogen, oxygen, and sulfur. In some embodiments, Ring A is a 5- to 6-membered heteroaryl ring having 1-2 heteroatoms independently selected from nitrogen, oxygen, and sulfur. In some embodiments, Ring A is a 5-membered heteroaryl ring having 1-2 heteroatoms independently selected from nitrogen, oxygen, and sulfur. Ring A is a 5-membered heteroaryl ring having 1-2 heteroatoms independently selected from nitrogen and sulfur. In some embodiments, Ring A is a thiophenyl or thiazolyl. In some embodiments, Ring A is a 6-membered heteroaryl ring having 1-2 nitrogen atoms. In some embodiments, Ring A is pyridinyl or pyrimidinyl.

In some embodiments, Ring A is an 8- to 10-membered bicyclic heteroaryl ring having 1-4 heteroatoms independently selected from nitrogen, oxygen, and sulfur. In some embodiments, Ring A is a 10-membered bicyclic heteroaryl ring having 1-4 heteroatoms independently selected from nitrogen, oxygen, and sulfur. In some embodiments, Ring A is a 9-membered bicyclic heteroaryl ring having 1 sulfur heteroatom. In some embodiments, Ring A is benzothiophenyl. In some embodiments, Ring A is a 7-11 membered spirofused ring having 0-4 heteroatoms independently selected from nitrogen, oxygen, and sulfur.

In some embodiments, Ring A is cyclopropyl, cyclohexyl, phenyl, tetrahydropyranyl, dioxolyl, thiophenyl, thiazolyl, pyridinyl, pyrimidinyl, or benzothiophenyl.

In some embodiments, Ring A is selected from the group consisting of:

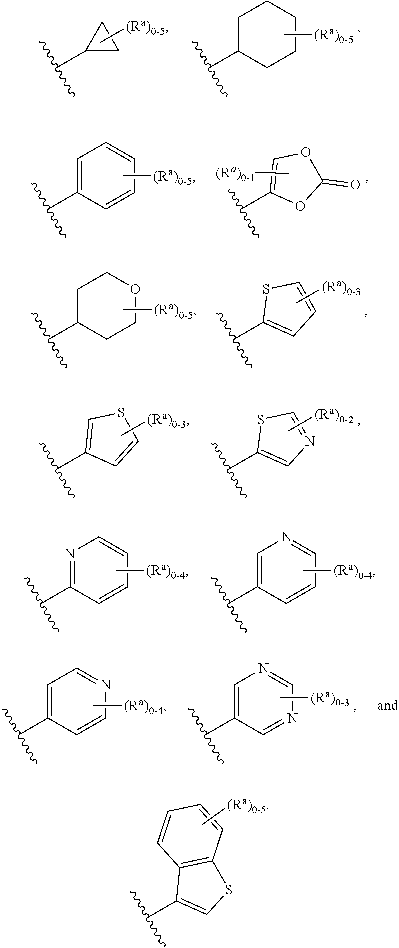

In some embodiments, Ring A is selected from the group consisting of:

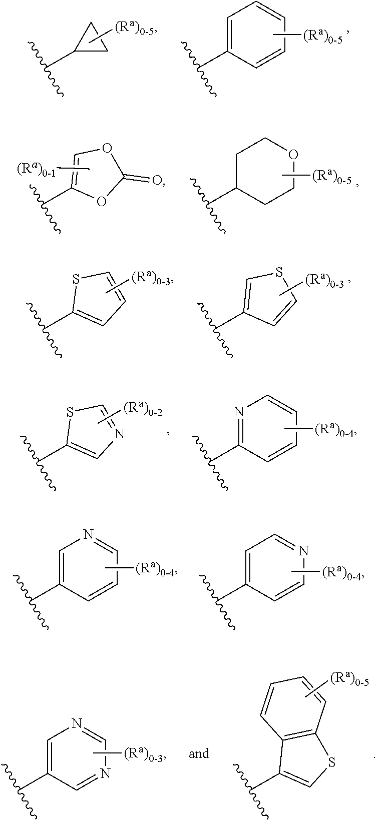

As defined generally above, each R^{a} is independently oxo, halogen, —CN, —NR₂, —OR, —SR, —C(O)R, —C(O)OR, NO₂, or an optionally substituted C₁₋₆ aliphatic group. In some embodiments, R^{a} is oxo, halogen, —CN, —NR, —OR, —C(O)OR, or an optionally substituted C₁₋₆ aliphatic group. In some embodiments, each R^{a} is independently halogen, —CN, —NR₂, —OR, —SR, —C(O)R, —C(O)OR, NO₂, or an optionally substituted C₁₋₆ aliphatic group. In some embodiments, R^{a} is oxo. In some embodiments, R^{a} is halogen. In some embodiments, R^{a} is fluoro, chloro, or bromo. In some embodiments, R^{a} is fluoro. In some embodiments, R^{a} is chloro. In some embodiments, R^{a} is bromo. In some embodiments, R^{a} is —CN. In some embodiments, R^{a} is —NR₂. In some embodiments, R^{a} is —NH₂. In some embodiments, R^{a} is —OR. In some embodiments, R^{a} is —OH. In some embodiments, R^{a} is —OCH₃. In some embodiments, R^{a} is —SR. In some embodiments, R^{a} is —C(O)OR. In some embodiments, R^{a} is —C(O)OCH₃. In some embodiments, R^{a} is —C(O)OCH₂CH₃. In some embodiments, R^{a} is optionally substituted C₁₋₆ aliphatic. In some embodiments, R^{a} is C₁₋₆ aliphatic optionally substituted with halogen. In some embodiments, R^{a} is C₁₋₆ aliphatic optionally substituted with fluoro. In some embodiments, R^{a} is C₁₋₆ aliphatic optionally substituted with oxo. In some embodiments, R^{a} is —CF₃. In some embodiments, R^{a} is optionally substituted C₁₋₄ aliphatic. In some embodiments, R^{a} is C₁₋₆ aliphatic. In some embodiments, R^{a} is C₁₋₄ aliphatic. In some embodiments, R^{a} is methyl, ethyl, ethynyl, n-propyl, isopropyl, n-butyl, s-butyl, or t-butyl. In some embodiments, R^{a} is methyl, ethyl, ethynyl, or t-butyl. In some embodiments, R^{a} is selected from the group consisting of oxo, fluoro, chloro, bromo, —CN, —OCH₃, —C(O)OCH₃, and —C(O)OCH₂CH₃.

In some embodiments, R^{a} is selected from the group consisting of oxo, fluoro, chloro, bromo, —CN, methyl, ethyl, ethynyl, —CF₃, —OCH₃, —C(O)OCH₃, and —C(O)OCH₂CH₃.

As defined generally above, Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur.

In some embodiments, Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—. In some embodiments, Z¹ is selected from an optionally substituted bivalent straight C₂₋₄ hydrocarbon chain wherein one or two carbon atoms of Z¹ are optionally and independently replaced by a group selected from —O—, —N(R)—, or —C(O)—. In some embodiments, Z¹ is selected from an optionally substituted bivalent straight C₂₋₃ hydrocarbon chain wherein one or two carbon atoms of Z¹ are optionally and independently replaced by a group selected from —O—, —N(R)—, or —C(O)—. In some embodiments, Z¹ is selected from an optionally substituted bivalent straight C₂ hydrocarbon chain wherein one or two carbon atoms of Z¹ are optionally and independently replaced by a group selected from —O—, —N(R)—, or —C(O)—.

In some embodiments, Z¹ is selected from an optionally substituted bivalent straight C₂ hydrocarbon chain wherein one or two carbon atoms of Z¹ are optionally and independently replaced by a group selected from —N(R)— or —C(O)—. In some embodiments, Z¹ is selected from an optionally substituted bivalent straight C₂ hydrocarbon chain wherein one carbon atom of Z¹ is replaced by —N(R)—, and another carbon atom of Z¹ is replaced by —C(O)—. In some embodiments, Z¹ is

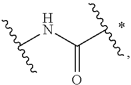 [SMILES: *NC(*)=O]

wherein * represents the point of attachment to Z². In some embodiments, Z¹ is

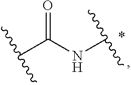 [SMILES: *NC(*)=O]

wherein * represents the point of attachment to Z².

In some embodiments, Z¹ is selected from an optionally substituted bivalent straight C₂ hydrocarbon chain wherein one or two carbon atoms of Z¹ are optionally and independently replaced by a group selected from —O— or —N(R)—. In some embodiments, Z¹ is selected from an optionally substituted bivalent straight C₂ hydrocarbon chain wherein one carbon atom of Z¹ is optionally replaced by a group selected from —O— or —N(R)—. In some embodiments, Z¹ is selected from an optionally substituted bivalent straight C₂ hydrocarbon chain wherein one carbon atom of Z¹ is replaced by a group selected from —O— or —N(R)—. In some embodiments, Z¹ is selected from an optionally substituted bivalent straight C₂ hydrocarbon chain wherein one carbon atom of Z¹ is optionally replaced by —O—. In some embodiments, Z¹ is

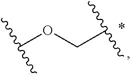 [SMILES: *CO*]

wherein * represents the point of attachment to Z². In some embodiments, Z¹ is

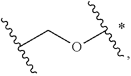 [SMILES: *CO*]

wherein * represents the point of attachment to Z². In some embodiments, Z¹ is

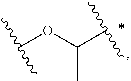 [SMILES: *OC(*)C]

wherein * represents the point of attachment to Z². In some embodiments, Z¹ is selected from an optionally substituted bivalent straight C₂ hydrocarbon chain wherein one carbon atom of Z¹ is optionally replaced by —N(R)—. In some embodiments, Z¹ is

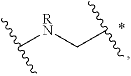

wherein * represents the point of attachment to Z², and R is an optionally substituted C₁₋₆ aliphatic. In some embodiments, Z¹ is

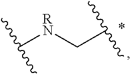

wherein * represents the point of attachment to Z², and R is C₁₋₆ aliphatic optionally substituted with —(CH₂)₀₋₄R^{∘}; wherein R^{∘} is a 3- to 6-membered saturated, partially unsaturated, or aryl ring having 0-4 heteroatoms independently selected from nitrogen, oxygen, or sulfur, and R^{∘} may be substituted with halogen. In some embodiments, Z¹ is

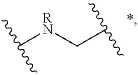

wherein * represents the point of attachment to Z², and R is C₁₋₆ aliphatic optionally substituted with —R^{∘}; wherein R^{∘} is a 3- to 6-membered saturated, partially unsaturated, or aryl ring having 0-1 nitrogen atom, and R^{∘} may be substituted with halogen. In some embodiments, Z¹ is

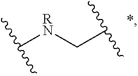

wherein * represents the point of attachment to Z², and R is C₁₋₆ aliphatic optionally substituted with —R^{∘}; wherein R^{∘} is cyclopropyol, phenyl, or pyridinyl, and R^{∘} may be substituted with fluoro. In some embodiments, Z¹ is

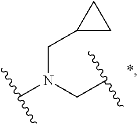 [SMILES: *CN(*)CC1CC1]

wherein * represents the point of attachment to Z². In some embodiments, Z¹ is

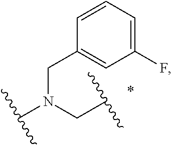 [SMILES: *CN(*)Cc1cccc(F)c1]

wherein * represents the point of attachment to Z². In some embodiments, Z¹ is

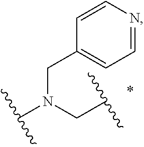 [SMILES: *CN(*)Cc1ccncc1]

wherein * represents the point of attachment to Z².

In some embodiments, Z¹ is selected from an optionally substituted bivalent straight C₂ hydrocarbon chain. In some embodiments, Z¹ is

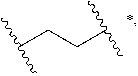 [SMILES: *CC*]

wherein * represents the point of attachment to Z². In some embodiments, Z¹ is

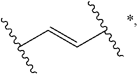 [SMILES: */C=C/*]

wherein * represents the point of attachment to Z².

In some embodiments, Z¹ is selected from the group consisting of:

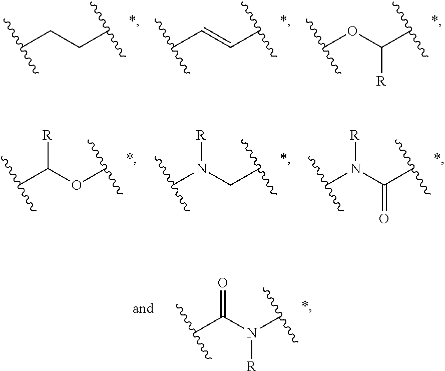

wherein * represents the point of attachment to Z².

In some embodiments, Z¹ is selected from the group consisting of:

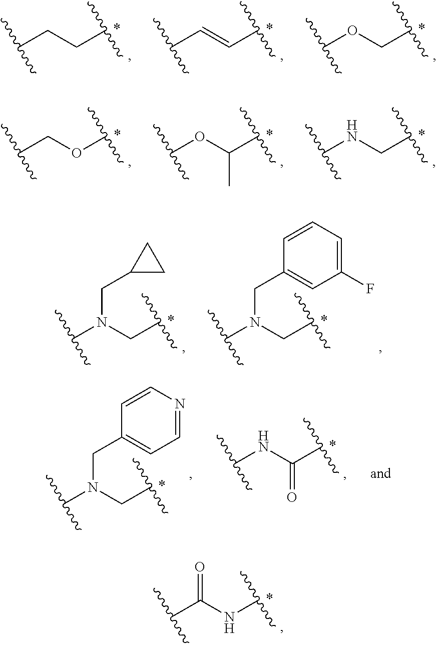

wherein * represents the point of attachment to Z².

In some embodiments, Z¹ is selected from the group consisting of:

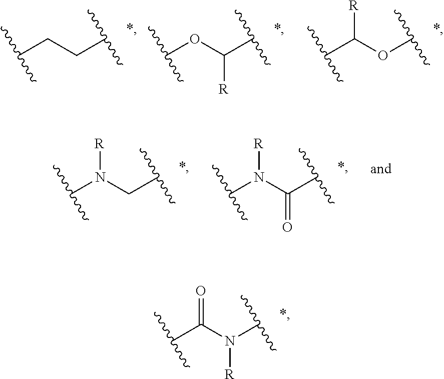

wherein * represents the point of attachment to Z².

In some embodiments, Z¹ is selected from the group consisting of:

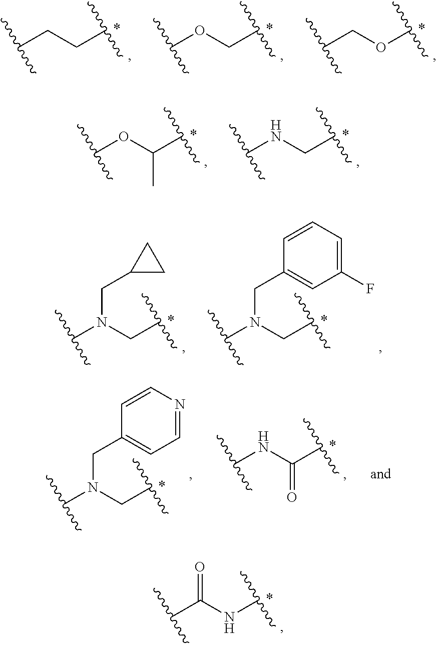

wherein * represents the point of attachment to Z².

In some embodiments, Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur. It will be understood that, when R^{x} and Z¹ come together to form Ring B as defined above and described herein, each of R^{x} and Z¹ is first independently defined as described above and herein and then taken together to form Ring B. For example, when R^{x} is attached to an atom adjacent to the atom where Z¹ is attached, R^{x} is —OH, and Z¹ is

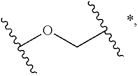 [SMILES: *CO*]

R^{x} and Z¹ may come together to form

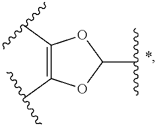 [SMILES: *C1=C(*)OC(*)O1]

wherein * represents the point of attachment to Z². In some embodiments, Ring B is selected from an optionally substituted 5- to 6-membered partially unsaturated or aryl ring having 0-2 heteroatoms independently selected from nitrogen, oxygen, and sulfur.

In some embodiments, Ring B is selected from an optionally substituted 5- to 6-membered partially unsaturated or aryl ring having 1-2 heteroatoms independently selected from nitrogen and oxygen.

In some embodiments, Ring B is selected from an optionally substituted 5-membered partially unsaturated or aryl ring having 1-2 heteroatoms independently selected from nitrogen and oxygen. In some embodiments, Ring B is selected from an optionally substituted 5-membered partially unsaturated or aryl ring having 2 heteroatoms independently selected from nitrogen and oxygen. In some embodiments, Ring B is selected from

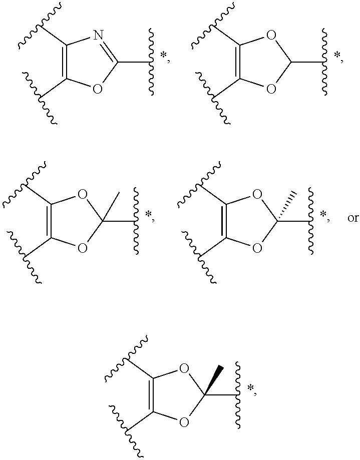

wherein * represents the point of attachment to Z².

In some embodiments, Ring B is selected from an optionally substituted 6-membered partially unsaturated or aryl ring having 1-2 heteroatoms independently selected from nitrogen and oxygen. In some embodiments, Ring B is selected from an optionally substituted 5-membered partially unsaturated ring having 2 heteroatoms independently selected from nitrogen and oxygen. In some embodiments, Ring B is selected from

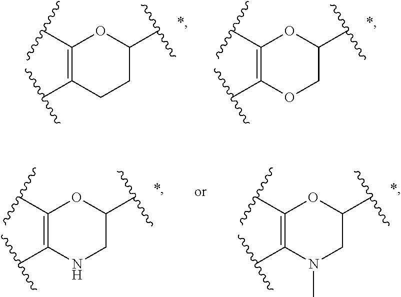

wherein * represents the point of attachment to Z².

In some embodiments, Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or R^{a} and Z¹, together with their intervening atoms, may form Ring C, wherein Ring C is selected from an optionally substituted 5- to 6-membered saturated, partially unsaturated, or aryl ring having 0-2 heteroatoms independently selected from nitrogen, oxygen, and sulfur. It will be understood that, when R^{a} and Z¹ come together to form Ring C as defined above and described herein, each of R^{a} and Z¹ is first independently defined as described above and herein and then taken together to form Ring C. For example, when R^{a} is —OMe and Z¹ is

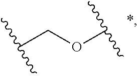 [SMILES: *CO*]

R^{a} and Z¹ may come together to form

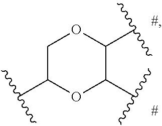 [SMILES: *C1COC(*)C(*)O1]

wherein each # represents a point of attachment to Ring A. In some such embodiments, Ring C is selected from an optionally substituted 5- to 6-membered saturated, partially unsaturated, or aryl ring having 0-2 heteroatoms independently selected from nitrogen, oxygen, and sulfur.

In some embodiments, Ring C is selected from an optionally substituted 6-membered saturated, partially unsaturated, or aryl ring having 0-2 heteroatoms independently selected from nitrogen, oxygen, and sulfur. In some embodiments, Ring C is selected from an optionally substituted 6-membered saturated, partially unsaturated, or aryl ring. In some embodiments, Ring C is selected from an optionally substituted 6-membered saturated, partially unsaturated, or aryl ring having 1 heteroatom independently from nitrogen, oxygen, and sulfur. In some embodiments, Ring C is selected from an optionally substituted 6-membered saturated, partially unsaturated, or aryl ring having 1 oxygen heteroatom. In some embodiments, Ring C is selected from an optionally substituted 6-membered saturated, partially unsaturated, or aryl ring having 2 heteroatoms independently selected from nitrogen, oxygen, and sulfur. In some embodiments, Ring C is selected from an optionally substituted 6-membered saturated, partially unsaturated, or aryl ring having 2 heteroatoms independently selected from nitrogen and oxygen.

In some embodiments, Ring C is selected from a 6-membered saturated, partially unsaturated, or aryl ring having 0-2 heteroatoms independently selected from nitrogen, oxygen, and sulfur. In some embodiments, Ring C is selected from a 6-membered saturated, partially unsaturated, or aryl ring. In some embodiments, Ring C is selected from a 6-membered saturated, partially unsaturated, or aryl ring having 1 heteroatom independently from nitrogen, oxygen, and sulfur. In some embodiments, Ring C is selected from a 6-membered saturated, partially unsaturated, or aryl ring having 1 oxygen heteroatom. In some embodiments, Ring C is selected from a 6-membered saturated, partially unsaturated, or aryl ring having 2 heteroatoms independently selected from nitrogen, oxygen, and sulfur. In some embodiments, Ring C is selected from a 6-membered saturated, partially unsaturated, or aryl ring having 2 heteroatoms independently selected from nitrogen and oxygen. In some embodiments, Ring C is selected from

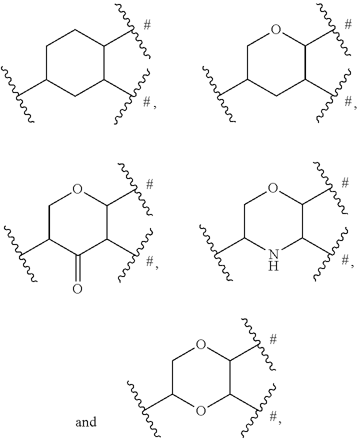 [SMILES: *C1CCC(*)C(*)C1 ; *C1COC(*)C(*)C1 ; *C1COC(*)C(*)C1=O ; *C1COC(*)C(*)N1 ; *C1COC(*)C(*)O1]

wherein each # represents a point of attachment to Ring A.

As defined generally above, each R^{z} is independently selected from hydrogen or optionally substituted C₁₋₆ aliphatic. In some embodiments, R^{z} is hydrogen. In some embodiments, R^{z} is optionally substituted C₁₋₆ aliphatic. In some embodiments, R^{z} is methyl. In some embodiments, R^{z} is ethyl. In some embodiments, R^{z} is propyl. In some embodiments, R^{z} is n-propyl. In some embodiments, R^{z} is isopropyl. In some embodiments, R^{z} is butyl. In some embodiments, R^{z} is n-butyl. In some embodiments, R^{z} is sec-butyl. In some embodiments, R^{z} is isobutyl. In some embodiments, R^{z} is tert-butyl. In some embodiments, R^{z} is pentyl. In some embodiments, R^{z} is hexyl.

As defined generally above, m is 0, 1, 2, 3, or 4. In some embodiments, m is 0, 1, 2, or 3. In some embodiments, m is 0, 1, or 2. In some embodiments, m is 0 or 1. In some embodiments, m is 1, 2, 3, or 4. In some embodiments, m is 1, 2, or 3. In some embodiments, m is 1 or 2. In some embodiments, m is 0. In some embodiments, m is 1. In some embodiments, m is 2. In some embodiments, m is 3. In some embodiments, m is 4.

As defined generally above, n is 0, 1, 2, 3, 4, or 5. In some embodiments, n is 0, 1, 2, 3, or 4. In some embodiments, n is 0, 1, 2, or 3. In some embodiments, n is 0, 1, or 2. In some embodiments, n is 0 or 1. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 0. In some embodiments, n is 1. In some embodiments, n is 2. In some embodiments, n is 3. In some embodiments, n is 4. In some embodiments, n is 5.

As defined generally above, p is 0, 1, 2, or 3. In some embodiments, p is 0, 1, or 2. In some embodiments, p is 0 or 1. In some embodiments, p is 1 or 2. In some embodiments, p is 0. In some embodiments, p is 1. In some embodiments, p is 2. In some embodiments, p is 3.

As defined generally above, R^{w} is a warhead group. In some embodiments, R^{w} is a warhead group -L¹-X¹—Y¹, wherein:
L¹ is selected from a covalent bond or a straight or branched C₁-C₆ aliphatic, wherein one or two carbon atoms of L¹ are optionally and independently replaced by a group selected from —O— or —N(R)—;
X¹ is:

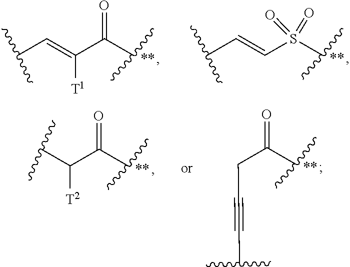

** represents the point of attachment to L¹;
T¹ is hydrogen, —CN, halogen, or optionally substituted C₁₋₆ aliphatic;
T² is halogen or —CN; and
Y¹ is hydrogen or optionally substituted C₁₋₆ aliphatic.

As defined generally above, L¹ is selected from a covalent bond or a straight or branched C₁-C₆ aliphatic, wherein one or two carbon atoms of L¹ are optionally and independently replaced by a group selected from —O— or —N(R)—. In some embodiments, L¹ is a covalent bond. In some embodiments, L¹ is a straight or branched C₁-C₆ aliphatic, wherein one or two carbon atoms of L¹ are optionally and independently replaced by a group selected from —O— or —N(R)—. In some embodiments, L¹ is a straight or branched C₁-C₃ aliphatic, wherein one or two carbon atoms of L¹ are optionally and independently replaced by a group selected from —O— or —N(R)—. In some embodiments, L¹ is a straight or branched C₁-C₃ aliphatic, wherein one carbon atom of L¹ is optionally and independently replaced by a group selected from —O— or —N(R)—. In some embodiments, L¹ is C₁ aliphatic, wherein one carbon atom of L¹ is optionally and independently replaced by a group selected from —O— or —N(R)—. In some embodiments, L¹ is —N(R)—. In some embodiments, L¹ is —N(H)—. In some embodiments, L¹ is —N(Me)-. In some embodiments, L¹ is —O—. In some embodiments, L¹ is a covalent bond, —N(H)—, or —O—.

As defined generally above, X¹ is:

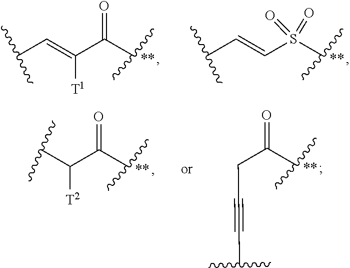

wherein ** represents the point of attachment to L¹; and T¹ and T² are as defined above and described herein.

In some embodiments, X¹ is:

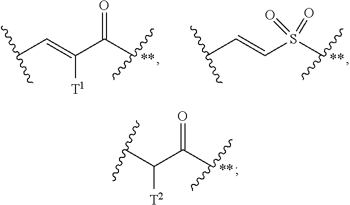

wherein ** represents the point of attachment to L¹; and
T¹ and T² are as defined above and described herein

In some embodiments, X¹ is:

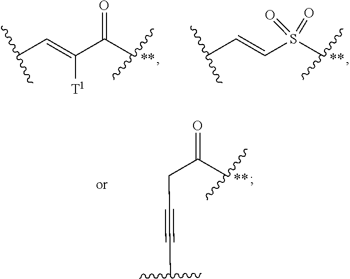

wherein ** represents the point of attachment to L¹; and
T¹ is as defined above and described herein.

In some embodiments, X¹ is

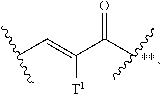

wherein ** represents the point of attachment to L¹. In some embodiments, X¹ is

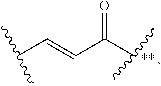 [SMILES: */C=C/C(*)=O]

wherein ** represents the point of attachment to L¹. In some embodiments, X¹ is

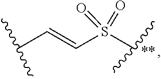 [SMILES: */C=C/S(*)(=O)=O]

wherein ** represents the point of attachment to L¹. In some embodiments, X¹ is

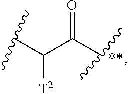

wherein ** represents the point of attachment to L¹. In some embodiments, X¹ is

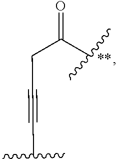 [SMILES: *C#CCC(*)=O]

wherein ** represents the point of attachment to L¹.

As defined generally above, T¹ is hydrogen, —CN, halogen, or optionally substituted C₁₋₆ aliphatic. In some embodiments, T¹ is hydrogen. In some embodiments, T¹ is —CN. In some embodiments, T¹ is halogen. In some embodiments, T¹ is fluoro. In some embodiments, T¹ is optionally substituted C₁₋₆ aliphatic. In some embodiments, T¹ is optionally substituted C₁₋₃ aliphatic. In some embodiments, T¹ is methyl.

As defined generally above, T² is halogen or —CN. In some embodiments, T² is —CN. In some embodiments, T² is halogen. In some embodiments, T² is fluoro or chloro. In some embodiments, T² is fluoro. In some embodiments, T² is chloro.

As defined generally above, Y¹ is hydrogen or optionally substituted C₁₋₆ aliphatic. In some embodiments, Y¹ is hydrogen. In some embodiments, Y¹ is optionally substituted C₁₋₆ aliphatic. In some embodiments, Y¹ is optionally substituted C₁₋₃ aliphatic. In some embodiments, Y¹ is C₁₋₃ aliphatic optionally substituted with —(CH₂)₀₋₄N(R^{∘})₂, wherein each R^{∘} is independently selected from hydrogen or C₁₋₆ aliphatic. In some embodiments, Y¹ is C₁₋₃ aliphatic optionally substituted with —N(R^{∘})₂, wherein each R^{∘} is independently selected from hydrogen or C₁₋₆ aliphatic. In some embodiments, Y¹ is C₁ aliphatic optionally substituted with —N(R^{∘})₂, wherein each R^{∘} is independently selected from hydrogen or C₁₋₆ aliphatic. In some embodiments, Y¹ is hydrogen or —CH₂N(CH₃).

In some embodiments, R^{w} is selected from the group consisting of:

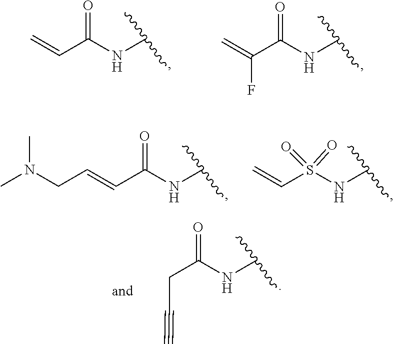

In some embodiments, R^{w} is

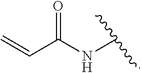 [SMILES: *NC(=O)C=C]

In some embodiments, R^{w} is a warhead group -L-Y, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N— or —C(═N₂)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein:
   Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, —SO₂—, —N(R)C(O)—, —C(O)N(R)—, —N(R)SO₂—, or —SO₂N(R)—; and
   Z is hydrogen or C₁₋₆ aliphatic optionally substituted with oxo, halogen, NO₂, or CN.

In some embodiments, L is a covalent bond.

In some embodiments, L is a bivalent C₁₋₈ saturated or unsaturated, straight or branched, hydrocarbon chain. In some embodiments, L is —CH₂—.

In some embodiments, L is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L is a bivalent C₂₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is a bivalent C₂₋₈ unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In some embodiments, L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—.

In some embodiments, L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—.

As described above, in certain embodiments, L is a bivalent C₂₋₈ straight or branched, hydrocarbon chain wherein L has at least one double bond. One of ordinary skill in the art will recognize that such a double bond may exist within the hydrocarbon chain backbone or is “exo” to the backbone chain and thus forming an alkylidene group. By way of example, such an L group having an alkylidene branched chain includes —CH₂C(═CH₂)CH₂—. Thus, in some embodiments, L is a bivalent C₂₋₈ straight or branched, hydrocarbon chain wherein L has at least one alkylidenyl double bond. Exemplary L groups include —NHC(O)C(═CH₂)CH₂—.

In some embodiments, L is a bivalent C₂₋₈ straight or branched, hydrocarbon chain wherein L has at least one double bond and at least one methylene unit of L is replaced by —C(O)—. In some embodiments, L is —C(O)CH═CH(CH₃)—, —C(O)CH═CHCH₂N(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂N(CH₃)—, —CH₂CH₂C(O)CH═CH(CH₃)—, or —CH(CH₃)OC(O)CH═CH—.

In some embodiments, L is a bivalent C₂₋₈ straight or branched, hydrocarbon chain wherein L has at least one double bond and at least one methylene unit of L is replaced by —OC(O)—.

In some embodiments, L is a bivalent C₂₋₈ straight or branched, hydrocarbon chain wherein L has at least one double bond and at least one methylene unit of L is replaced by —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—. In some embodiments, L is —CH₂OC(O)CH═CHCH₂—, —CH₂—OC(O)CH═CH—, or —CH(CH═CH₂)OC(O)CH═CH—.

In some embodiments, L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)C(═CH₂)CH₂—, —CH₂NRC(O)—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN.

In some embodiments, L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)C(═CH₂)CH₂—, —CH₂NHC(O)—, —CH₂CH₂NHC(O)—, or —CH₂NHC(O)cyclopropylene-.

In some embodiments, L is a bivalent C₂₋₈ straight or branched, hydrocarbon chain wherein L has at least one triple bond. In some embodiments, L is a bivalent C₂₋₈ straight or branched, hydrocarbon chain wherein L has at least one triple bond and one or two additional methylene units of L are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L has at least one triple bond and at least one methylene unit of L is replaced by —N(R)—, —N(R)C(O)—, —C(O)—, —C(O)O—, or —OC(O)—, or —O—.

Exemplary L groups include —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂OC(═O)C≡C—.

In certain embodiments, L is a bivalent C₂₋₈ straight or branched, hydrocarbon chain wherein one methylene unit of L is replaced by cyclopropylene and one or two additional methylene units of L are independently replaced by —C(O)—, —NRC(O)—, —C(O)NR—, —N(R)SO₂—, or —SO₂N(R)—. Exemplary L groups include —NHC(O)-cyclopropylene-SO₂— and —NHC(O)— cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In some embodiments, Y is hydrogen.

In some embodiments, Y is C₁₋₆ aliphatic optionally substituted with oxo, halogen, NO₂, or CN. In some embodiments, Y is C₂₋₆ alkenyl optionally substituted with oxo, halogen, NO₂, or CN. In some embodiments, Y is C₂₋₆ alkynyl optionally substituted with oxo, halogen, NO₂, or CN. In some embodiments, Y is C₂₋₆ alkenyl. In some embodiments, Y is C₂₋₄ alkynyl.

In some embodiments, Y is C₁₋₆ alkyl substituted with oxo, halogen, NO₂, or CN. Such Y groups include —CH₂F, —CH₂Cl, —CH₂CN, and —CH₂NO₂.

In some embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In some embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In some embodiments, Y is

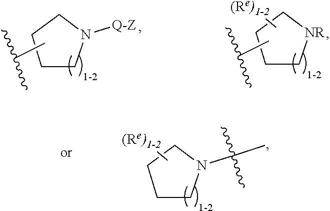

wherein each R, Q, Z, and R^{e} is as defined above and described herein.

In some embodiments, Y is a saturated 3-6 membered carbocyclic ring, wherein said ring is substituted with 1-4 R^{e} groups, wherein each R^{e} is as defined above and described herein. In some embodiments, Y is cyclopropyl, cyclobutyl, cyclopentyl, or cyclohexyl, wherein each ring is substituted with 1-4 R^{e} groups, wherein each R^{e} is as defined above and described herein. In some embodiments, Y is

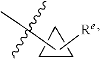

wherein R^{e} is as defined above and described herein. In some embodiments, Y is cyclopropyl optionally substituted with halogen, CN or NO₂.

In some embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined above and described herein. In some embodiments, Y is

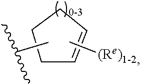

wherein each R^{e} is as defined above and described herein.

In some embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In some embodiments, Y is selected from:

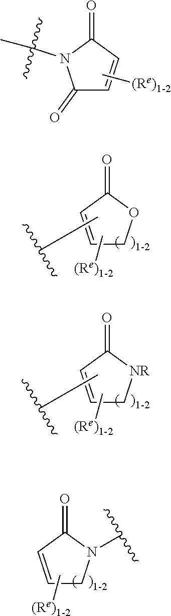

wherein each R and R^{e} is as defined above and described herein.

In some embodiments, Y is a 6-membered aromatic ring having 0-2 nitrogens wherein said ring is substituted with 1-4 R^{e} groups, wherein each R^{e} group is as defined above and described herein. In some embodiments, Y is phenyl, pyridyl, or pyrimidinyl, wherein each ring is substituted with 1-4 R^{e} groups, wherein each R^{e} is as defined above and described herein.

In some embodiments, Y is selected from:

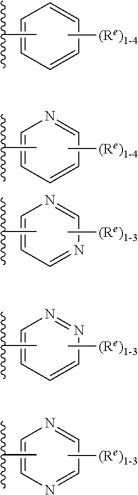

wherein each R^{e} is as defined above and described herein.

In some embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In some embodiments, Y is selected from:

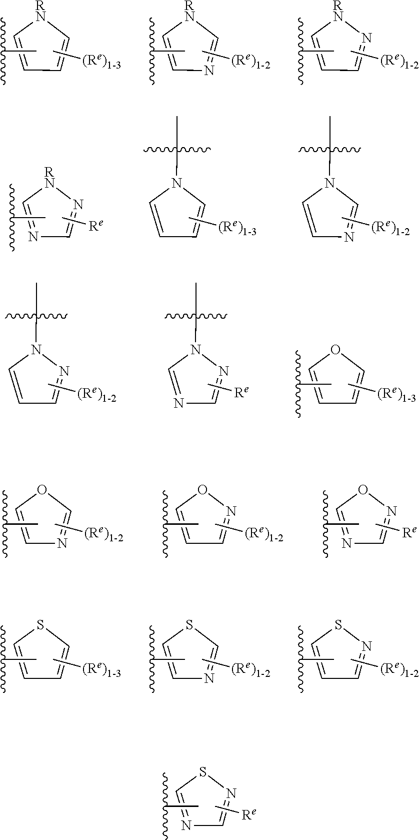

wherein each R and R^{e} is as defined above and described herein.

In some embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. In some embodiments, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In some embodiments, R^{e} is C₁₋₆ aliphatic optionally substituted with oxo, halogen, NO₂, or CN. In some embodiments, R^{e} is oxo, NO₂, halogen, or CN.

In some embodiments, R^{e} is -Q-Z, wherein Q is a covalent bond and Z is hydrogen (i.e., R^{e} is hydrogen). In some embodiments, R^{e} is -Q-Z, wherein Q is a bivalent C₁₋₆ saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In some embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In some embodiments, the Z moiety of the R^{e} group is hydrogen. In some embodiments, -Q-Z is —NHC(O)CH═CH₂ or —C(O)CH═CH₂.

In some embodiments, each R^{e} is independently selected from oxo, NO₂, CN, fluoro, chloro, —NHC(O)CH═CH₂, —C(O)CH═CH₂, —CH₂CH═CH₂, —C—CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In some embodiments, R^{e} is a suitable leaving group, i.e., a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetoxy, methanesulfonyloxy (mesyloxy), tosyloxy, triflyloxy, nitro-phenylsulfonyloxy (nosyloxy), and bromo-phenylsulfonyloxy (brosyloxy).

In some embodiments, the following embodiments and combinations of -L-Y apply:
(a) L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(b) L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(c) L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(d) L is a bivalent C₂₋₈ straight or branched, hydrocarbon chain wherein L has at least one double bond and at least one methylene unit of L is replaced by —C(O)—; and Y is hydrogen or C₁₋₆ aliphatic optionally substituted with oxo, halogen, NO₂, or CN; or
(e) L is a bivalent C₂₋₈ straight or branched, hydrocarbon chain wherein L has at least one double bond and at least one methylene unit of L is replaced by —OC(O)—; and Y is hydrogen or C₁₋₆ aliphatic optionally substituted with oxo, halogen, NO₂, or CN; or
(f) L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)C(═CH₂)CH₂—, —CH₂NRC(O)—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or
(g) L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)C(═CH₂)CH₂—, —CH₂NHC(O)—, —CH₂CH₂NHC(O)—, or —CH₂NHC(O)cyclopropylene-; and Y is hydrogen or C₁₋₆ aliphatic optionally substituted with oxo, halogen, NO₂, or CN; or
(h) L is a bivalent C₂₋₈ straight or branched, hydrocarbon chain wherein L has at least one alkylidenyl double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(i) L is a bivalent C₂₋₈ straight or branched, hydrocarbon chain wherein L has at least one triple bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or
(j) L is —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂OC(═O)C≡C—; and Y is hydrogen or C₁₋₆ aliphatic optionally substituted with oxo, halogen, NO₂, or CN; or
(k) L is a bivalent C₂₋₈ straight or branched, hydrocarbon chain wherein one methylene unit of L is replaced by cyclopropylene and one or two additional methylene units of L are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or
(l) L is a covalent bond and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

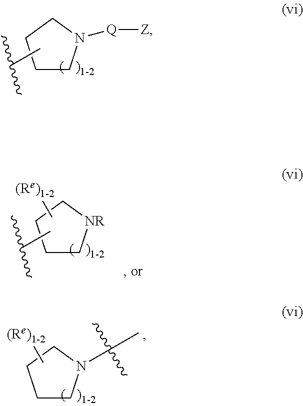

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

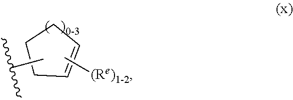

wherein each R^{e} is as defined above and described herein; or
   (xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

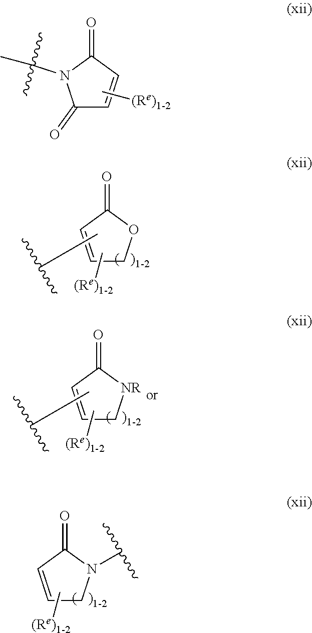

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

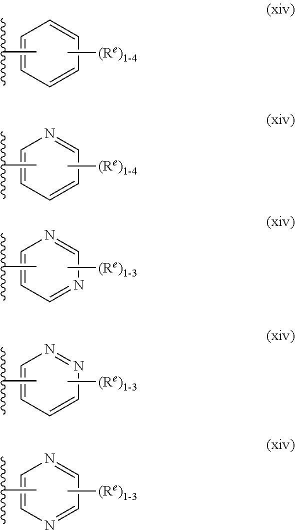

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

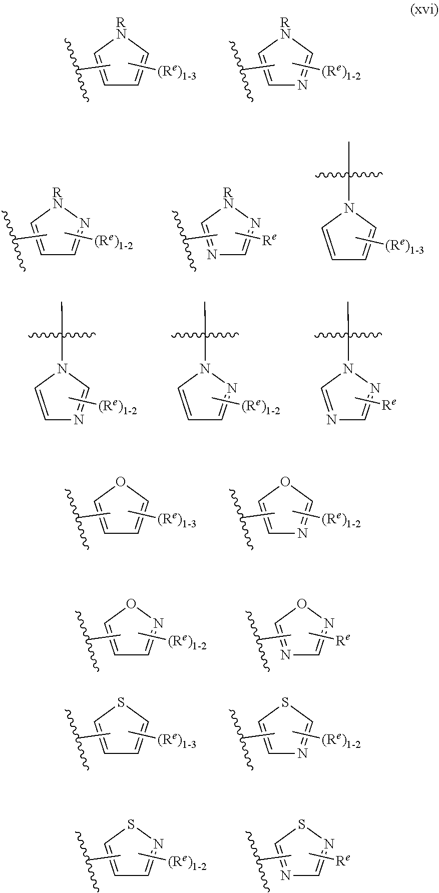

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein; or
(m) L is —C(O)— and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

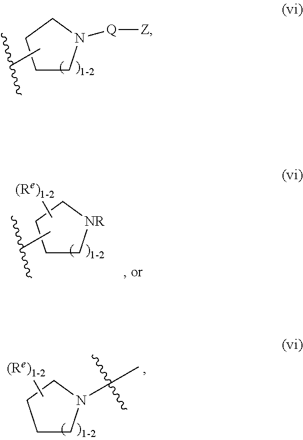

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

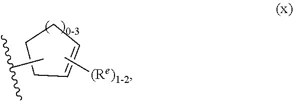

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

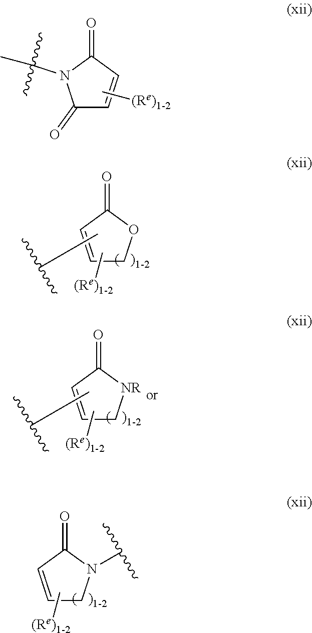

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

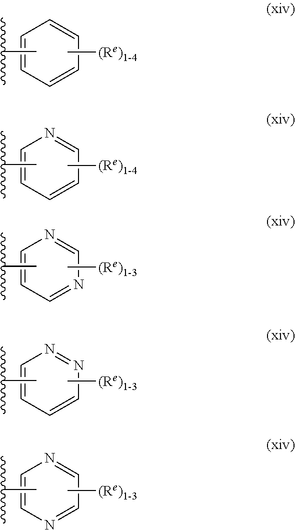

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

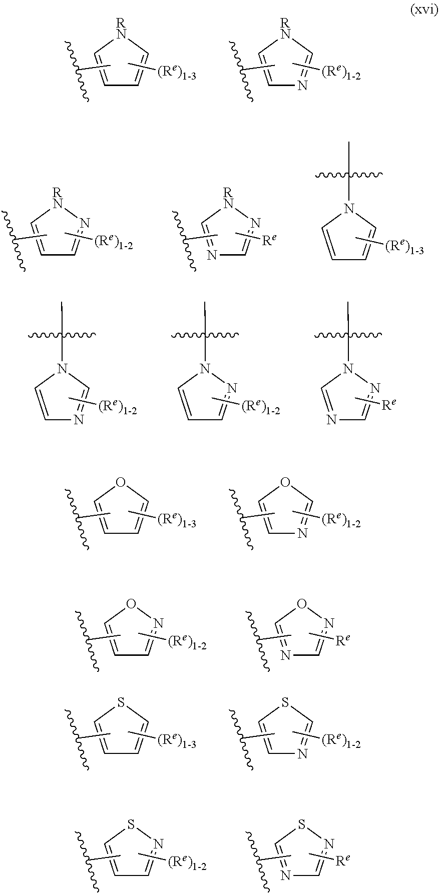

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein; or
(n) L is —N(R)C(O)— and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

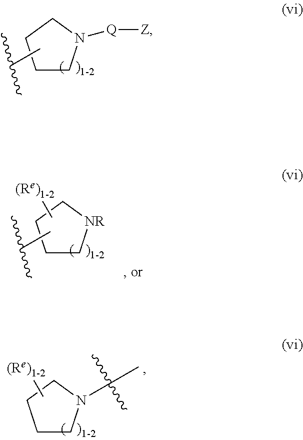

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

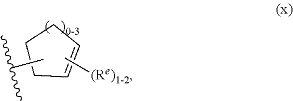

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

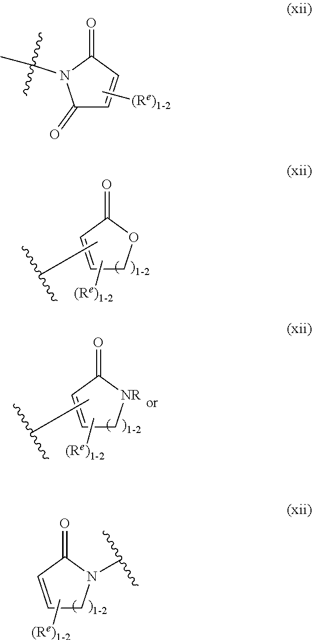

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

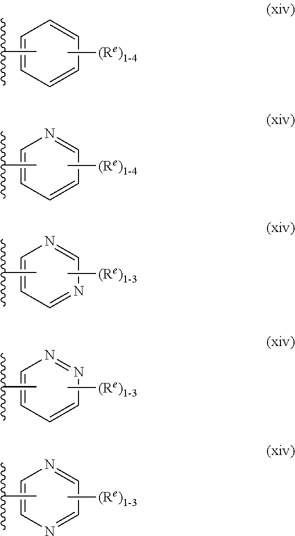

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

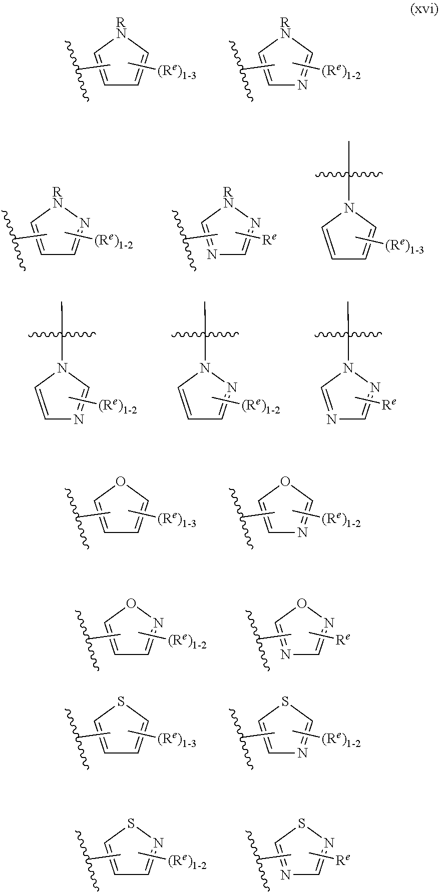

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein; or
(o) L is a bivalent C₁₋₈ saturated or unsaturated, straight or branched, hydrocarbon chain; and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

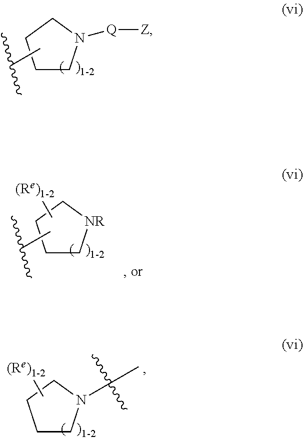

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

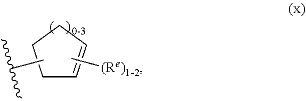

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

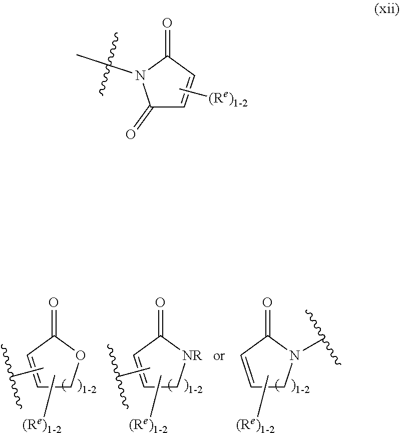

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

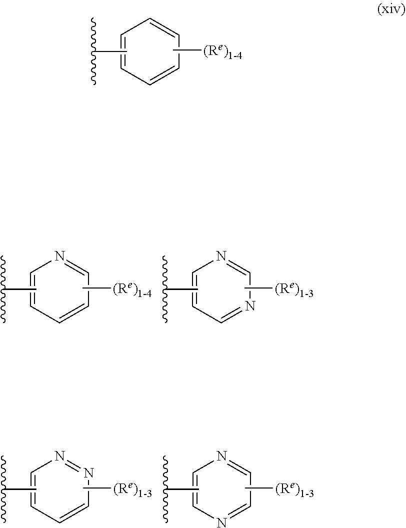

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

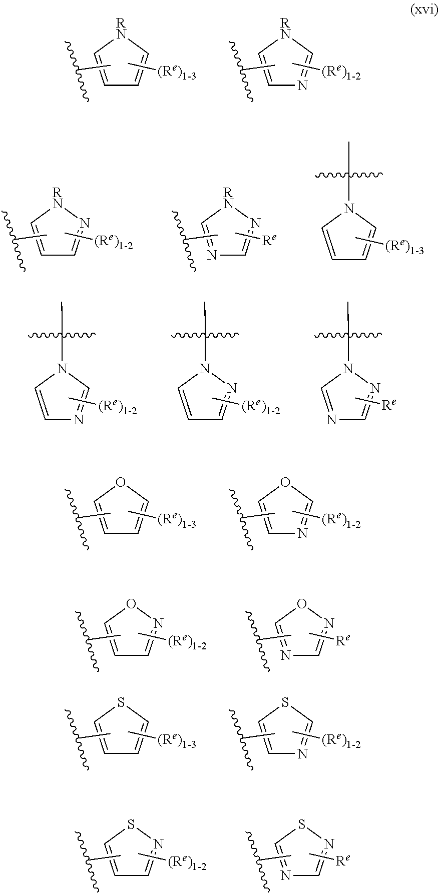

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein; or
(p) L is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, or —SO₂NH—; and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

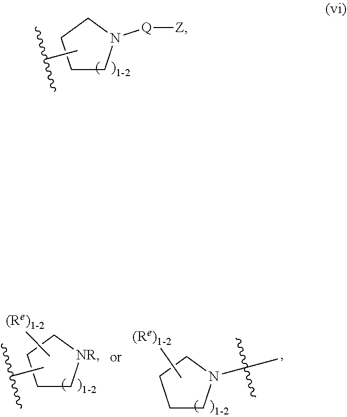

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

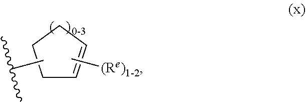

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

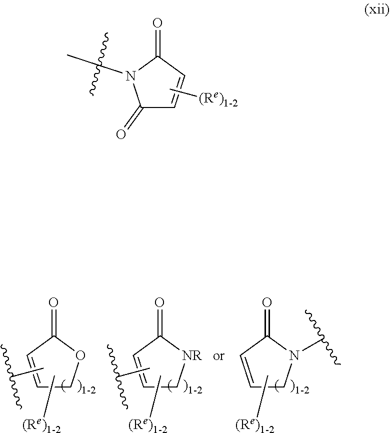

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

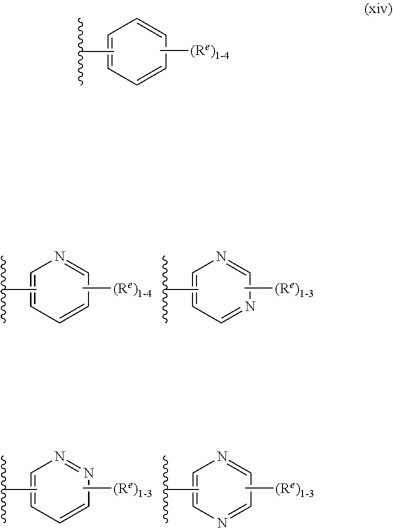

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

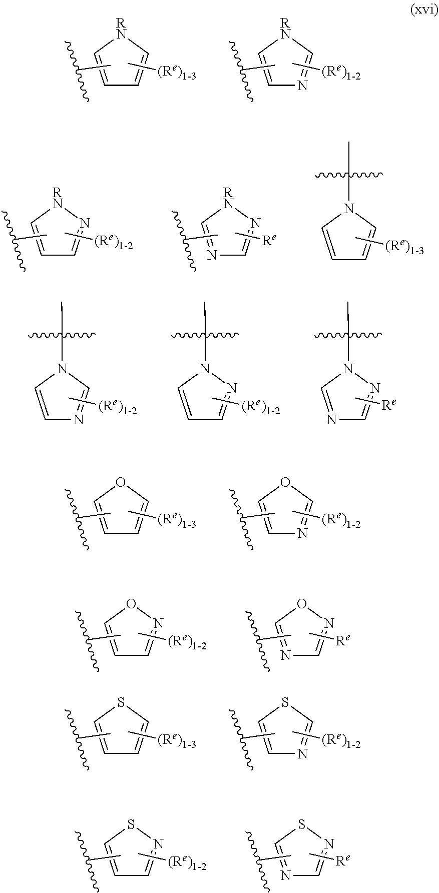

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein; or
(q) L is a bivalent C₂₋₈ straight or branched, hydrocarbon chain wherein two or three methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(r) L-Y is a “pro-warhead” that is converted in vitro or in vivo to an irreversible warhead. For example, when L is —NHC(O)CH₂—, and Y is —CH₂-halo, the “pro-warhead” is converted to an irreversible warhead according to the following:

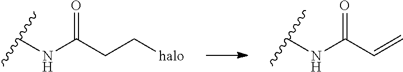

In certain embodiments, the Y group of any of the formulae herein is selected from those set forth in Table 1, wherein each wavy line indicates the point of attachment to the rest of the molecule.

In certain embodiments, R^{w} is -L-Y, wherein:
L is a covalent bond or a bivalent C₁₋₈ saturated or unsaturated, straight or branched, hydrocarbon chain, wherein one, two, or three methylene units of L are optionally and independently replaced by —N(R)—, —N(R)C(O)—, —N(R)SO₂—, —O—, —C(O)—, or —SO₂—; and
Y is hydrogen or C₁₋₆ aliphatic optionally substituted with oxo, halogen, N(R)₂, NO₂, or CN.

In certain embodiments, the Y group of R^{w} group, -L-Y, is selected from those set forth in Table 1, below, wherein each wavy line indicates the point of attachment to the rest of the molecule.

TABLE 1
Exemplary Y groups:

| | |
|---|---|
| 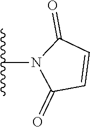 [SMILES: *N1C(=O)C=CC1=O] | a |
| 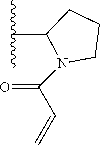 [SMILES: *C1CCCN1C(=O)C=C] | b |
| 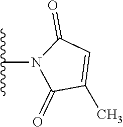 [SMILES: *N1C(=O)C=C(C)C1=O] | c |
| 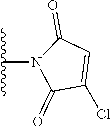 [SMILES: *N1C(=O)C=C(Cl)C1=O] | d |
| 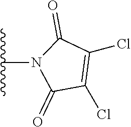 [SMILES: *N1C(=O)C(Cl)=C(Cl)C1=O] | e |
| 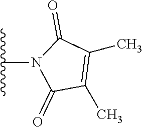 [SMILES: *N1C(=O)C(C)=C(C)C1=O] | f |
| 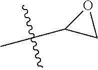 [SMILES: *C1CO1] | g |
| 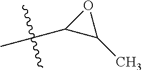 [SMILES: *C1OC1C] | h |
| 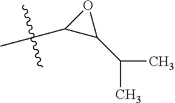 [SMILES: *C1OC1C(C)C] | i |
| 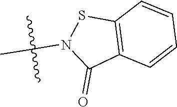 [SMILES: *n1sc2ccccc2c1=O] | j |
| 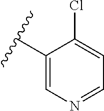 [SMILES: *c1cnccc1Cl] | k |
| 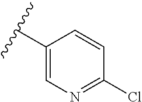 [SMILES: *c1ccc(Cl)nc1] | l |
| 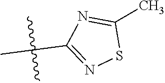 [SMILES: *c1nsc(C)n1] | m |
| 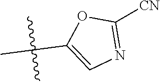 [SMILES: *c1cnc(C#N)o1] | n |
| 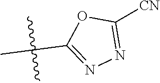 [SMILES: *c1nnc(C#N)o1] | o |
| 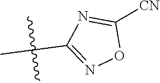 [SMILES: *c1noc(C#N)n1] | p |
| 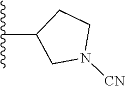 [SMILES: *C1CCN(C#N)C1] | q |
| 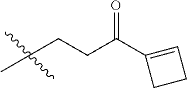 [SMILES: *CCC(=O)C1=CCC1] | r |
| 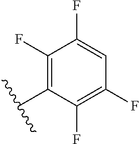 [SMILES: *c1c(F)c(F)cc(F)c1F] | s |
| 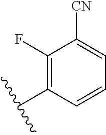 [SMILES: *c1cccc(C#N)c1F] | t |
| 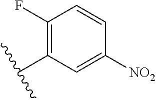 [SMILES: *c1cc([N+](=O)[O-])ccc1F] | u |
| 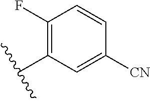 [SMILES: *c1cc(C#N)ccc1F] | v |
| 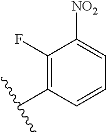 [SMILES: *c1cccc([N+](=O)[O-])c1F] | w |
| 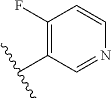 [SMILES: *c1cnccc1F] | x |
| 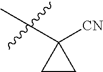 [SMILES: *C1(C#N)CC1] | y |
| 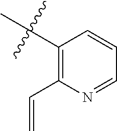 [SMILES: *c1cccnc1C=C] | z |
| 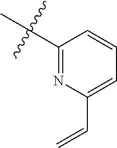 [SMILES: *c1cccc(C=C)n1] | aa |
| 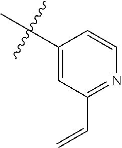 [SMILES: *c1ccnc(C=C)c1] | bb |
| 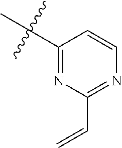 [SMILES: *c1ccnc(C=C)n1] | cc |
| 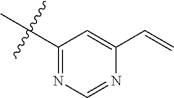 [SMILES: *c1cc(C=C)ncn1] | dd |
| 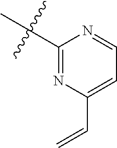 [SMILES: *c1nccc(C=C)n1] | ee |
| 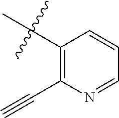 [SMILES: *c1cccnc1C#C] | ff |
| 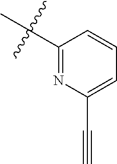 [SMILES: *c1cccc(C#C)n1] | gg |
| 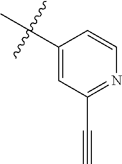 [SMILES: *c1ccnc(C#C)c1] | hh |
| 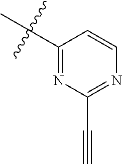 [SMILES: *c1ccnc(C#C)n1] | ii |
| 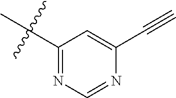 [SMILES: *c1cc(C#C)ncn1] | jj |
| 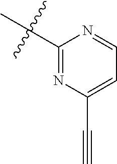 [SMILES: *c1nccc(C#C)n1] | kk |
| 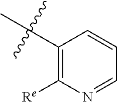 | ll |
| 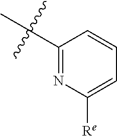 | mm |
| 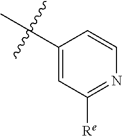 | nn |
| 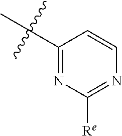 | oo |
| 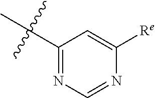 | pp |
| 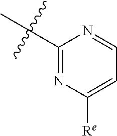 | qq |
| 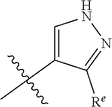 | rr |
| 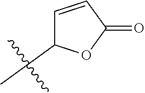 [SMILES: *C1C=CC(=O)O1] | ss |
| 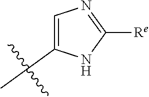 | tt |
| 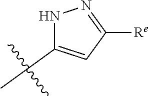 | uu |
| 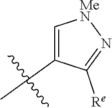 | vv |
| 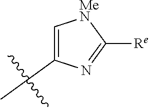 | ww |
| 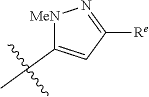 | xx |
| 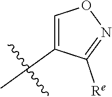 | yy |
| 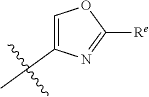 | zz |
| 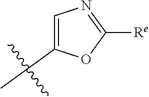 | aaa |
| 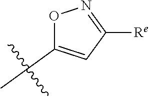 | bbb |
| 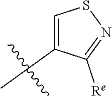 | ccc |
| 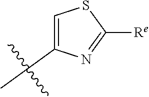 | ddd |
| 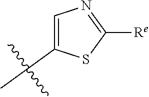 | eee |
| 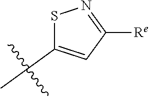 | fff |
| 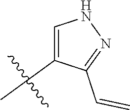 [SMILES: *c1c[nH]nc1C=C] | ggg |
| 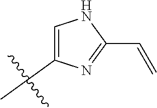 [SMILES: *c1c[nH]c(C=C)n1] | hhh |
| 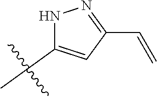 [SMILES: *c1cc(C=C)n[nH]1] | iii |
| 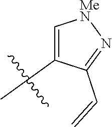 [SMILES: *c1cn(C)nc1C=C] | jjj |
| 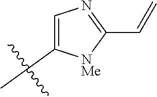 [SMILES: *c1cnc(C=C)n1C] | kkk |
| 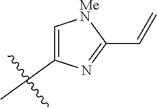 [SMILES: *c1cn(C)c(C=C)n1] | lll |
| 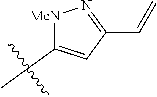 [SMILES: *c1cc(C=C)nn1C] | mmm |
| 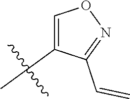 [SMILES: *c1conc1C=C] | nnn |
| 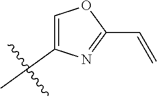 [SMILES: *c1coc(C=C)n1] | ooo |
| 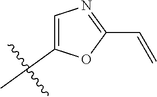 [SMILES: *c1cnc(C=C)o1] | ppp |
| 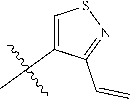 [SMILES: *c1csnc1C=C] | qqq |
| 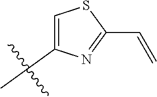 [SMILES: *c1csc(C=C)n1] | rrr |
| 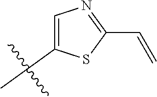 [SMILES: *c1cnc(C=C)s1] | sss |
| 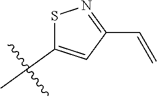 [SMILES: *c1cc(C=C)ns1] | ttt |
| 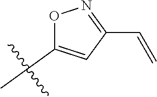 [SMILES: *c1cc(C=C)no1] | uuu |
| 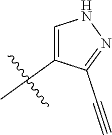 [SMILES: *c1c[nH]nc1C#C] | vvv |
| 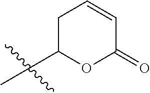 [SMILES: *C1CC=CC(=O)O1] | qqq |
| 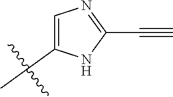 [SMILES: *c1cnc(C#C)[nH]1] | www |
| 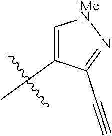 [SMILES: *c1cn(C)nc1C#C] | xxx |
| 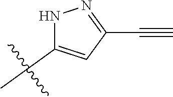 [SMILES: *c1cc(C#C)n[nH]1] | yyy |
| 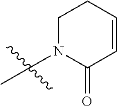 [SMILES: *N1CCC=CC1=O] | zzz |
| 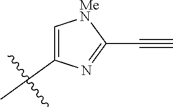 [SMILES: *c1cn(C)c(C#C)n1] | aaaa |
| 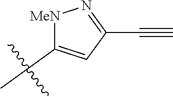 [SMILES: *c1cc(C#C)nn1C] | bbbb |
| 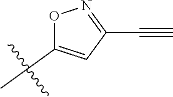 [SMILES: *c1cc(C#C)no1] | cccc |
| 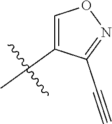 [SMILES: *c1conc1C#C] | dddd |
| 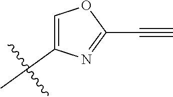 [SMILES: *c1coc(C#C)n1] | eeee |
| 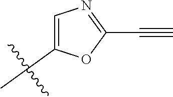 [SMILES: *c1cnc(C#C)o1] | ffff |
| 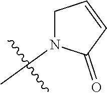 [SMILES: *N1CC=CC1=O] | gggg |
| 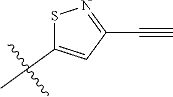 [SMILES: *c1cc(C#C)ns1] | hhhh |
| 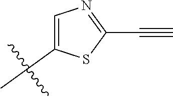 [SMILES: *c1cnc(C#C)s1] | iiii |
| 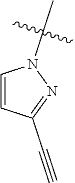 [SMILES: *n1ccc(C#C)n1] | jjjj |
| 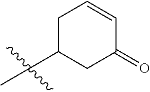 [SMILES: *C1CC=CC(=O)C1] | kkkk |
| 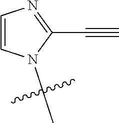 [SMILES: *n1ccnc1C#C] | llll |
| 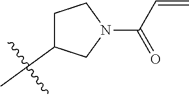 [SMILES: *C1CCN(C(=O)C=C)C1] | mmmm |
| 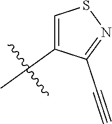 [SMILES: *c1csnc1C#C] | nnnn |
| 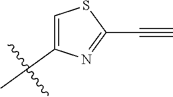 [SMILES: *c1csc(C#C)n1] | oooo |
| 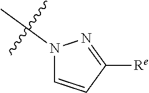 | pppp |
| 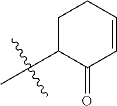 [SMILES: *C1CCC=CC1=O] | qqqq |
| 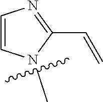 | rrrr |
| 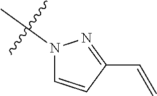 [SMILES: *n1ccc(C=C)n1] | ssss |
| 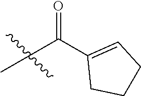 [SMILES: *C(=O)C1=CCCC1] | tttt |
| 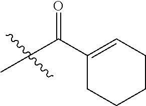 [SMILES: *C(=O)C1=CCCCC1] | uuuu |
| 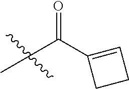 [SMILES: *C(=O)C1=CCC1] | vvvv |
| 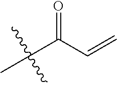 [SMILES: *C(=O)C=C] | wwww |
| 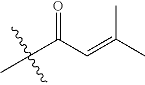 [SMILES: *C(=O)C=C(C)C] | xxxx |
| 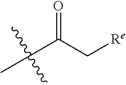 | yyyy |
| 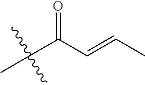 [SMILES: *C(=O)/C=C/C] | zzzz |
| 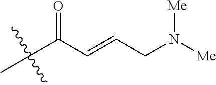 [SMILES: *C(=O)/C=C/CN(C)C] | aaaaa |
| 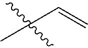 [SMILES: *C=C] | bbbbb |
| 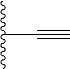 [SMILES: *C#C] | ccccc |

wherein each R^{e} is independently a suitable leaving group, NO₂, CN, or oxo.

In some embodiments, R^{w} is —C(O)CH₂CH₂C(O)CH═C(CH₃)₂, —C(O)CH₂CH₂C(O)CH═CH(cyclopropyl), —C(O)CH₂CH₂C(O)CH═CHCH₃, —C(O)CH₂CH₂C(O)CH═CHCH₂CH₃, or —C(O)CH₂CH₂C(O)C(═CH₂)CH₃. In some embodiments, R^{w} is —C(O)CH₂NHC(O)CH═CH₂, —C(O)CH₂NHC(O)CH₂CH₂C(O)CH═CHCH₃, or —C(O)CH₂NHC(O)CH₂CH₂C(O)C(═CH₂)CH₃. In some embodiments, R^{w} is —S(O)₂CH₂CH₂NHC(O)CH₂CH₂C(O)CH═C(CH₃)₂, —S(O)₂CH₂CH₂NHC(O)CH₂CH₂C(O)CH═CHCH₃, or —S(O)₂CH₂CH₂NHC(O)CH₂CH₂C(O)CH═CH₂. In some embodiments, R^{w} is —C(O)(CH₂)₃NHC(O)CH₂CH₂C(O)CH═CHCH₃ or —C(O)(CH₂)₃NHC(O)CH₂CH₂C(O)CH═CH₂.

In some embodiments, R^{w} is —C—CH, —C≡CCH₂NH(isopropyl), —NHC(O)C≡CCH₂CH₃, —CH₂—C—C≡CH₃, —C≡CCH₂OH, —CH₂C(O)C≡CH, —C(O)C≡CH, or —CH₂OC(═O)C—CH. In some embodiments, R^{w} is selected from —NHC(O)CH═CH₂, —NHC(O)CH═CHCH₂N(CH₃)₂, or —CH₂NHC(O)CH═CH₂.

In some embodiments, R^{w} is selected from those set forth in Table 2, below, wherein each wavy line indicates the point of attachment to the rest of the molecule.

TABLE 2
Exemplary R^{w} Groups

| | |
|---|---|
| 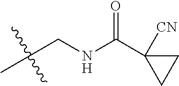 [SMILES: *CNC(=O)C1(C#N)CC1] | a |
| 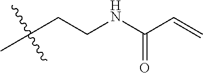 [SMILES: *CCNC(=O)C=C] | b |
| 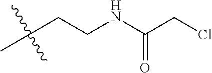 [SMILES: *CCNC(=O)CCl] | c |
| 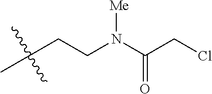 [SMILES: *CCN(C)C(=O)CCl] | d |
| 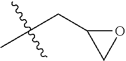 [SMILES: *CC1CO1] | e |
| 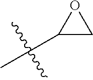 [SMILES: *C1CO1] | f |
| 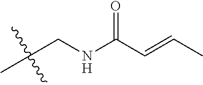 [SMILES: *CNC(=O)/C=C/C] | g |
| 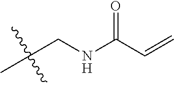 [SMILES: *CNC(=O)C=C] | h |
| 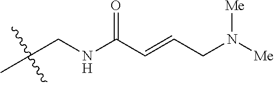 [SMILES: *CNC(=O)/C=C/CN(C)C] | i |
| 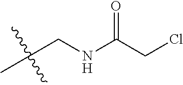 [SMILES: *CNC(=O)CCl] | j |
| 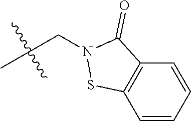 [SMILES: *Cn1sc2ccccc2c1=O] | k |
| 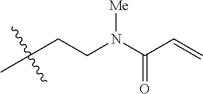 [SMILES: *CCN(C)C(=O)C=C] | l |
| 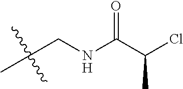 [SMILES: *CNC(=O)[C@H](C)Cl] | m |
| 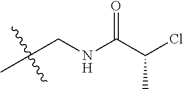 [SMILES: *CNC(=O)[C@@H](C)Cl] | n |
| 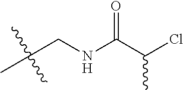 [SMILES: *CNC(=O)C(C)Cl] | o |
| 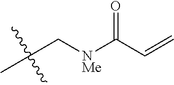 [SMILES: *CN(C)C(=O)C=C] | p |
| 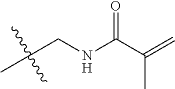 [SMILES: *CNC(=O)C(=C)C] | q |
| 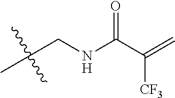 [SMILES: *CNC(=O)C(=C)C(F)(F)F] | r |
| 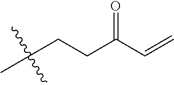 [SMILES: *CCC(=O)C=C] | s |
| 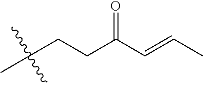 [SMILES: *CCC(=O)/C=C/C] | t |
| 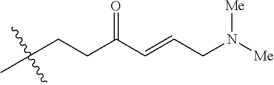 [SMILES: *CCC(=O)/C=C/CN(C)C] | u |
| 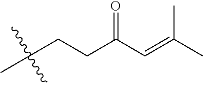 [SMILES: *CCC(=O)C=C(C)C] | v |
| 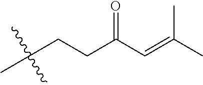 [SMILES: *CCC(=O)C=C(C)C] | w |
| 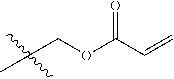 [SMILES: *COC(=O)C=C] | x |
| 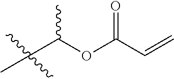 [SMILES: *C(C)OC(=O)C=C] | y |
| 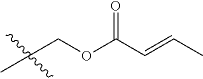 [SMILES: *COC(=O)/C=C/C] | z |
| 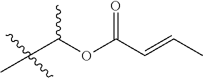 [SMILES: *C(C)OC(=O)/C=C/C] | aa |
| 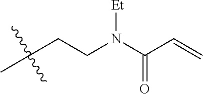 [SMILES: *CCN(CC)C(=O)C=C] | bb |
| 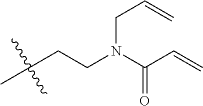 [SMILES: *CCN(CC=C)C(=O)C=C] | cc |
| 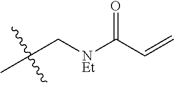 | dd |
| 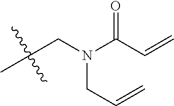 [SMILES: *CN(CC=C)C(=O)C=C] | ee |
| 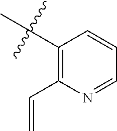 [SMILES: *c1cccnc1C=C] | ff |
| 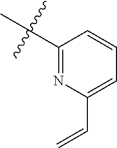 [SMILES: *c1cccc(C=C)n1] | gg |
| 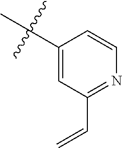 [SMILES: *c1ccnc(C=C)c1] | hh |
| 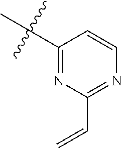 [SMILES: *c1ccnc(C=C)n1] | ii |
| 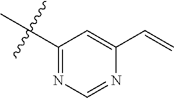 [SMILES: *c1cc(C=C)ncn1] | jj |
| 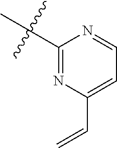 [SMILES: *c1nccc(C=C)n1] | kk |
| 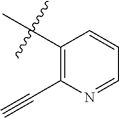 [SMILES: *c1cccnc1C#C] | ll |
| 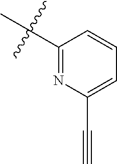 [SMILES: *c1cccc(C#C)n1] | mm |
| 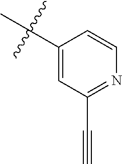 [SMILES: *c1ccnc(C#C)c1] | nn |
| 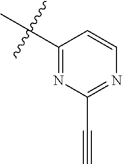 [SMILES: *c1ccnc(C#C)n1] | oo |
| 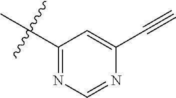 [SMILES: *c1cc(C#C)ncn1] | pp |
| 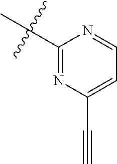 [SMILES: *c1nccc(C#C)n1] | qq |
| 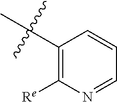 | rr |
| 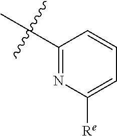 | ss |
| 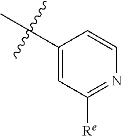 | tt |
| 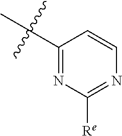 | uu |
| 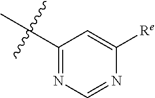 | vv |
| 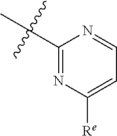 | ww |
| 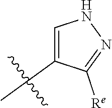 | xx |
| 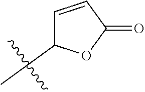 [SMILES: *C1C=CC(=O)O1] | yy |
| 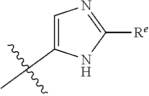 | zz |
| 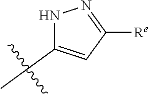 | aaa |
| 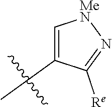 | bbb |
| 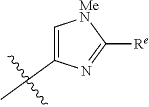 | ccc |
| 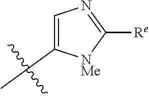 | ddd |
| 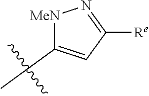 | eee |
| 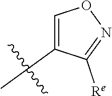 | fff |
| 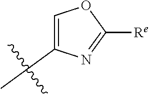 | ggg |
| 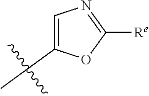 | hhh |
| 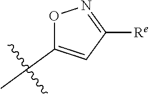 | iii |
| 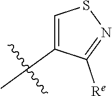 | jjj |
| 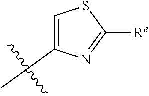 | kkk |
| 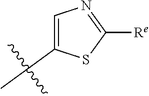 | lll |
| 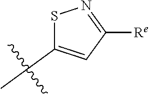 | mmm |
| 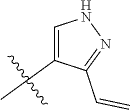 [SMILES: *c1c[nH]nc1C=C] | nnn |
| 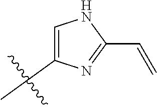 [SMILES: *c1c[nH]c(C=C)n1] | ooo |
| 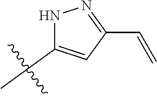 [SMILES: *c1cc(C=C)n[nH]1] | ppp |
| 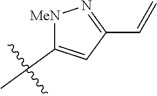 [SMILES: *c1cc(C=C)nn1C] | |
| 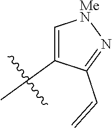 [SMILES: *c1cn(C)nc1C=C] | qqq |
| 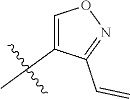 [SMILES: *c1conc1C=C] | rrr |
| 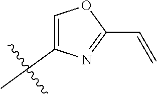 [SMILES: *c1coc(C=C)n1] | sss |
| 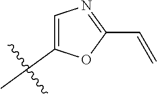 [SMILES: *c1cnc(C=C)o1] | ttt |
| 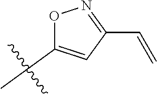 [SMILES: *c1cc(C=C)no1] | uuu |
| 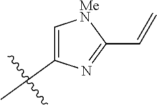 [SMILES: *c1cn(C)c(C=C)n1] | vvv |
| 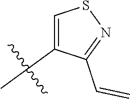 [SMILES: *c1csnc1C=C] | www |
| 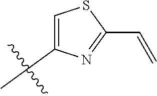 [SMILES: *c1csc(C=C)n1] | xxx |
| 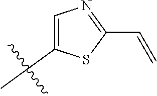 [SMILES: *c1cnc(C=C)s1] | yyy |
| 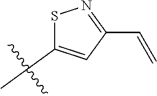 [SMILES: *c1cc(C=C)ns1] | zzz |
| 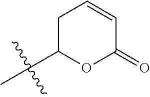 [SMILES: *C1CC=CC(=O)O1] | aaaa |
| 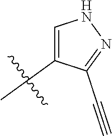 [SMILES: *c1c[nH]nc1C#C] | bbbb |
| 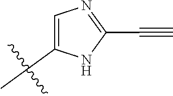 [SMILES: *c1cnc(C#C)[nH]1] | cccc |
| 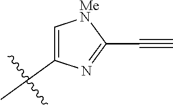 [SMILES: *c1cn(C)c(C#C)n1] | dddd |
| 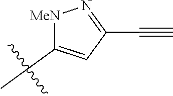 [SMILES: *c1cc(C#C)nn1C] | eeee |
| 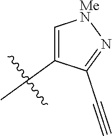 [SMILES: *c1cn(C)nc1C#C] | ffff |
| 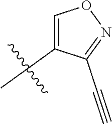 [SMILES: *c1conc1C#C] | gggg |
| 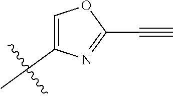 [SMILES: *c1coc(C#C)n1] | hhhh |
| 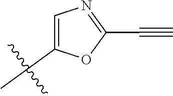 [SMILES: *c1cnc(C#C)o1] | iiii |
| 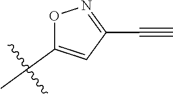 [SMILES: *c1cc(C#C)no1] | jjjj |
| 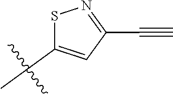 [SMILES: *c1cc(C#C)ns1] | kkkk |
| 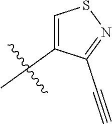 [SMILES: *c1csnc1C#C] | llll |
| 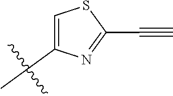 [SMILES: *c1csc(C#C)n1] | mmmm |
| 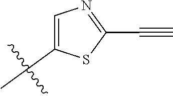 [SMILES: *c1cnc(C#C)s1] | nnnn |
| 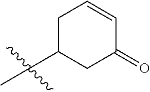 [SMILES: *C1CC=CC(=O)C1] | oooo |
| 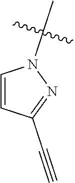 [SMILES: *n1ccc(C#C)n1] | pppp |
| 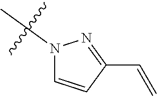 [SMILES: *n1ccc(C=C)n1] | qqqq |
| 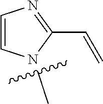 | rrrr |
| 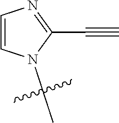 [SMILES: *n1ccnc1C#C] | ssss |
| 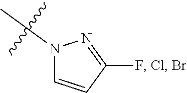 | tttt |
| 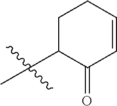 [SMILES: *C1CCC=CC1=O] | uuuu |
| 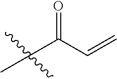 [SMILES: *C(=O)C=C] | vvvv |
| 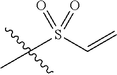 [SMILES: *S(=O)(=O)C=C] | wwww |
| 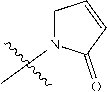 [SMILES: *N1CC=CC1=O] | xxxx |
| 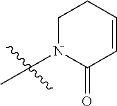 [SMILES: *N1CCC=CC1=O] | yyyy |
| 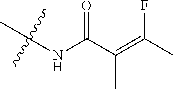 [SMILES: *NC(=O)/C(C)=C(/C)F] | zzzz |
| 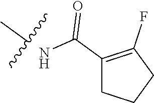 | aaaaa |
| 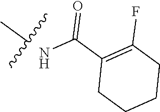 | bbbbb |
| 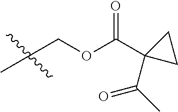 [SMILES: *COC(=O)C1(C(C)=O)CC1] | ccccc |
| 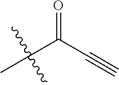 [SMILES: *C(=O)C#C] | ddddd |
| 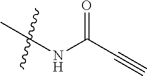 [SMILES: *NC(=O)C#C] | eeeee |
| 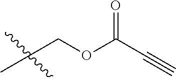 [SMILES: *COC(=O)C#C] | fffff |
| 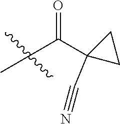 [SMILES: *C(=O)C1(C#N)CC1] | ggggg |
| 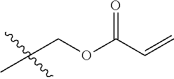 [SMILES: *COC(=O)C=C] | hhhhh |
| 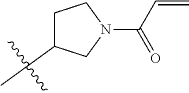 [SMILES: *C1CCN(C(=O)C=C)C1] | iiiii |
| 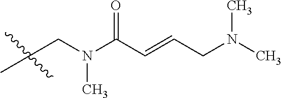 [SMILES: *CN(C)C(=O)/C=C/CN(C)C] | jjjjj |
| 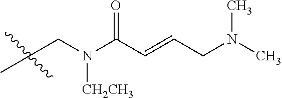 [SMILES: *CN(CC)C(=O)/C=C/CN(C)C] | kkkkk |
| 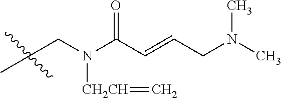 | lllll |
| 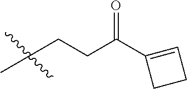 [SMILES: *CCC(=O)C1=CCC1] | mmmmm |
| 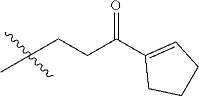 [SMILES: *CCC(=O)C1=CCCC1] | nnnnn |
| 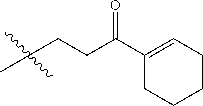 [SMILES: *CCC(=O)C1=CCCCC1] | ooooo |
| 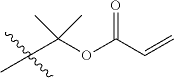 [SMILES: *C(C)(C)OC(=O)C=C] | ppppp |
| 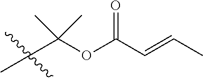 [SMILES: *C(C)(C)OC(=O)/C=C/C] | qqqqq |
| 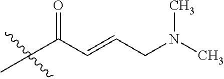 [SMILES: *C(=O)/C=C/CN(C)C] | rrrrr |
| 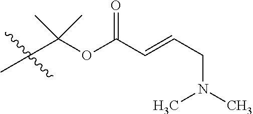 [SMILES: *C(C)(C)OC(=O)/C=C/CN(C)C] | sssss |
| 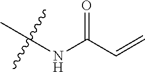 [SMILES: *NC(=O)C=C] | ttttt |
| 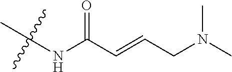 [SMILES: *NC(=O)/C=C/CN(C)C] | uuuuu |
| 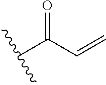 [SMILES: *C(=O)C=C] | vvvvv |
| 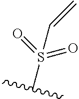 [SMILES: *S(=O)(=O)C=C] | wwwww |
| 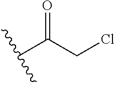 [SMILES: *C(=O)CCl] | xxxxx |
| 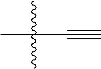 [SMILES: *C#C] | yyyyy |
| 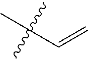 [SMILES: *C=C] | zzzzz |
| 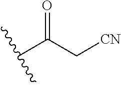 [SMILES: *C(=O)CC#N] | aaaaaa |
| 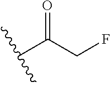 [SMILES: *C(=O)CF] | bbbbbb |
| 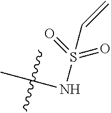 [SMILES: *NS(=O)(=O)C=C] | cccccc |
| 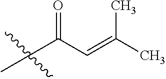 [SMILES: *C(=O)C=C(C)C] | dddddd |
| 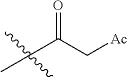 [SMILES: *C(=O)CC(C)=O] | eeeeee |
| 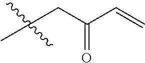 [SMILES: *CC(=O)C=C] | ffffff |
| 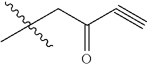 [SMILES: *CC(=O)C#C] | gggggg |
| 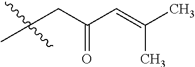 [SMILES: *CC(=O)C=C(C)C] | hhhhhh |
| 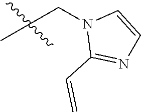 [SMILES: *Cn1ccnc1C=C] | iiiiii |
| 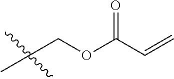 [SMILES: *COC(=O)C=C] | jjjjjj |
| 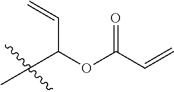 [SMILES: *C(C=C)OC(=O)C=C] | kkkkkk |
| 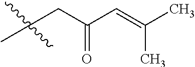 [SMILES: *CC(=O)C=C(C)C] | llllll |
| 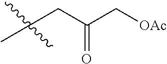 [SMILES: *CC(=O)COC(C)=O] | mmmmmm |
| 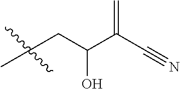 [SMILES: *CC(O)C(=C)C#N] | nnnnnn |
| 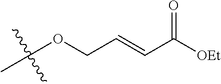 [SMILES: *OC/C=C/C(=O)OCC] | oooooo |
| 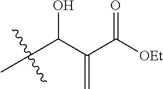 [SMILES: *C(O)C(=C)C(=O)OCC] | pppppp |
| 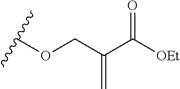 [SMILES: *OCC(=C)C(=O)OCC] | qqqqqq |
| 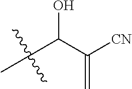 [SMILES: *C(O)C(=C)C#N] | rrrrrr |
| 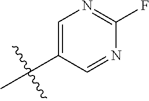 [SMILES: *c1cnc(F)nc1] | ssssss |
| 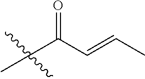 [SMILES: *C(=O)/C=C/C] | tttttt |
| 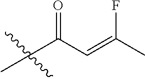 [SMILES: *C(=O)/C=C(/C)F] | uuuuuu |
| 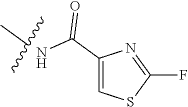 | vvvvvv |
| 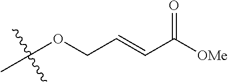 [SMILES: *OC/C=C/C(=O)OC] | wwwwww or |
| 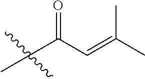 [SMILES: *C(=O)C=C(C)C] | xxxxxx |

wherein each R^{e} is independently a suitable leaving group, NO₂, CN, or oxo.

In some embodiments, Y is an isooxazolinyl or derivative capable of covalently binding to a side chain of an amino acid (e.g., to cysteine or serine). In some embodiments, Y is an isoxazolinyl or derivative described in PCT Publication WO 2010/135360, the entire contents of which are incorporated herein by reference. It will be appreciated that, when Y is an isooxazolinyl or derivative described in PCT Publication WO 2010/135360, the isooxazolinyl is bound to L of the warhead group at any reasonable position as allowed by valency of the isooxazolinyl compound or derivative. In some embodiments, Y is:

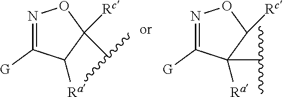

wherein G, R^{a′}, and R^{c′} are:

| G | R^{a’} | R^{c’} |
|---|---|---|
| —Br | —H | —H |
| —Cl | —H | —H |
| 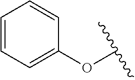 [SMILES: *Oc1ccccc1] | —H | —H |
| 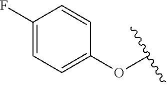 [SMILES: *Oc1ccc(F)cc1] | —H | —H |
| 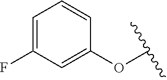 [SMILES: *Oc1cccc(F)c1] | —H | —H |
| 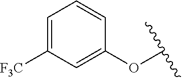 [SMILES: *Oc1cccc(C(F)(F)F)c1] | —H | —H |
| 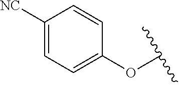 [SMILES: *Oc1ccc(C#N)cc1] | —H | —H |
| 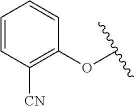 [SMILES: *Oc1ccccc1C#N] | —H | —H |
| 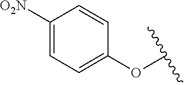 [SMILES: *Oc1ccc([N+](=O)[O-])cc1] | —H | —H |
| 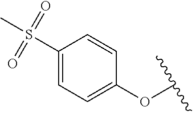 [SMILES: *Oc1ccc(S(C)(=O)=O)cc1] | —H | —H |
| 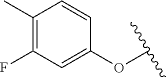 [SMILES: *Oc1ccc(C)c(F)c1] | —H | —H |
| 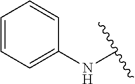 [SMILES: *Nc1ccccc1] | —H | —H |
| 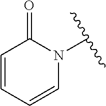 [SMILES: *n1ccccc1=O] | —H | —H |
| 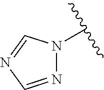 [SMILES: *n1cncn1] | —H | —H |
| 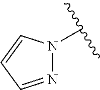 [SMILES: *n1cccn1] | —H | —H |
| 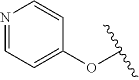 [SMILES: *Oc1ccncc1] | —H | —H |
| 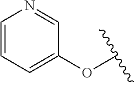 [SMILES: *Oc1cccnc1] | —H | —H |
| 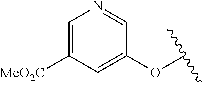 [SMILES: *Oc1cncc(C(=O)OC)c1] | —H | —H |
| 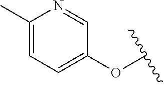 [SMILES: *Oc1ccc(C)nc1] | —H | —H |
| 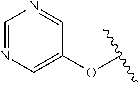 [SMILES: *Oc1cncnc1] | —H | —H |
| 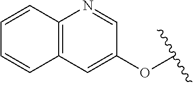 [SMILES: *Oc1cnc2ccccc2c1] | —H | —H |
| 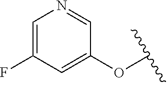 [SMILES: *Oc1cncc(F)c1] | —H | —H |
| 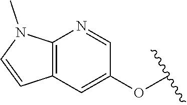 [SMILES: *Oc1cnc2c(ccn2C)c1] | —H | —H |
| —OMe | —H | —H |
| 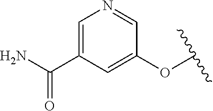 [SMILES: *Oc1cncc(C(N)=O)c1] | —H | —H |
| 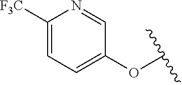 [SMILES: *Oc1ccc(C(F)(F)F)nc1] | —H | —H |
| 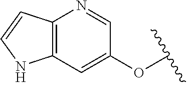 [SMILES: *Oc1cnc2cc[nH]c2c1] | —H | —H |
| 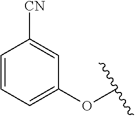 [SMILES: *Oc1cccc(C#N)c1] | —H | —H |
| 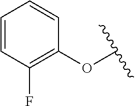 [SMILES: *Oc1ccccc1F] | —H | —H |
| 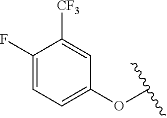 [SMILES: *Oc1ccc(F)c(C(F)(F)F)c1] | —H | —H |
| 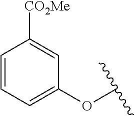 [SMILES: *Oc1cccc(C(=O)OC)c1] | —H | —H |
| 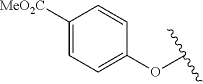 [SMILES: *Oc1ccc(C(=O)OC)cc1] | —H | —H |
| 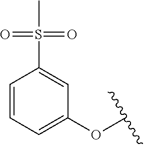 [SMILES: *Oc1cccc(S(C)(=O)=O)c1] | —H | —H |
| 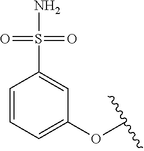 [SMILES: *Oc1cccc(S(N)(=O)=O)c1] | —H | —H |
| 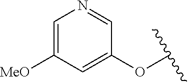 [SMILES: *Oc1cncc(OC)c1] | —H | —H |
| 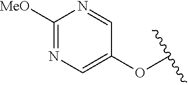 [SMILES: *Oc1cnc(OC)nc1] | —H | —H |
| 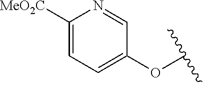 [SMILES: *Oc1ccc(C(=O)OC)nc1] | —H | —H |
| 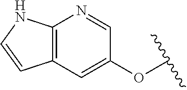 [SMILES: *Oc1cnc2[nH]ccc2c1] | —H | —H |
| 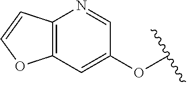 [SMILES: *Oc1cnc2ccoc2c1] | —H | —H |
| 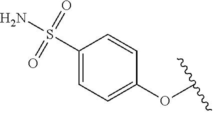 [SMILES: *Oc1ccc(S(N)(=O)=O)cc1] | —H | —H |
| 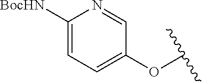 [SMILES: *Oc1ccc(NC(=O)OC(C)(C)C)nc1] | —H | —H |
| 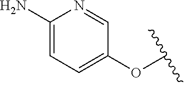 [SMILES: *Oc1ccc(N)nc1] | —H | —H |
| 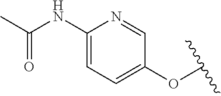 [SMILES: *Oc1ccc(NC(C)=O)nc1] | —H | —H |
| 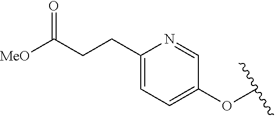 [SMILES: *Oc1ccc(CCC(=O)OC)nc1] | —H | —H |
| 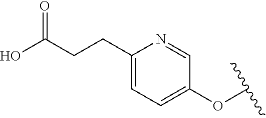 [SMILES: *Oc1ccc(CCC(=O)O)nc1] | —H | —H |
| 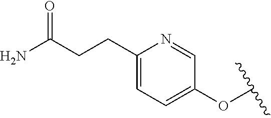 [SMILES: *Oc1ccc(CCC(N)=O)nc1] | —H | —H |
| 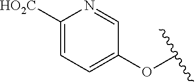 [SMILES: *Oc1ccc(C(=O)O)nc1] | —H | —H |
| 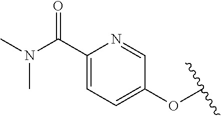 [SMILES: *Oc1ccc(C(=O)N(C)C)nc1] | —H | —H |
| 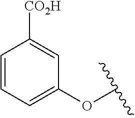 [SMILES: *Oc1cccc(C(=O)O)c1] | —H | —H |
| 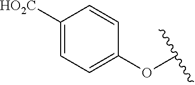 [SMILES: *Oc1ccc(C(=O)O)cc1] | —H | —H |
| 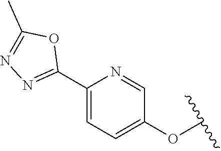 [SMILES: *Oc1ccc(-c2nnc(C)o2)nc1] | —H | —H |
| 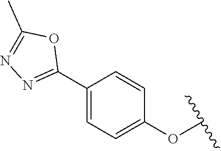 [SMILES: *Oc1ccc(-c2nnc(C)o2)cc1] | —H | —H |
| 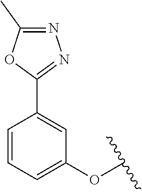 [SMILES: *Oc1cccc(-c2nnc(C)o2)c1] | —H | —H |
| 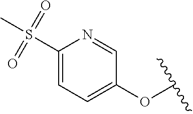 [SMILES: *Oc1ccc(S(C)(=O)=O)nc1] | —H | —H |
| 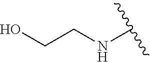 [SMILES: *NCCO] | —H | —H |
| 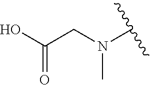 [SMILES: *N(C)CC(=O)O] | —H | —H |
| 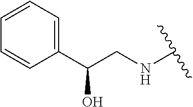 [SMILES: *NC[C@@H](O)c1ccccc1] | —H | —H |
| 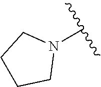 [SMILES: *N1CCCC1] | —H | —H |
| 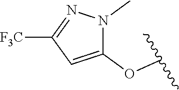 [SMILES: *Oc1cc(C(F)(F)F)nn1C] | —H | —H |
| 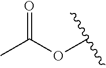 [SMILES: *OC(C)=O] | —H | —H |
| —Br | —CH₃ | —H |
| —Br | —CH₃ | —H |
| 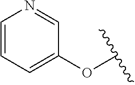 [SMILES: *Oc1cccnc1] | —CH₃ | —H |
| —Br | —H | —CH₃ |
| 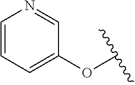 [SMILES: *Oc1cccnc1] | —H | —CH₃ |
| —Br | —H | —CF₃ |
| 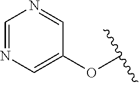 [SMILES: *Oc1cncnc1] | —H | —CF₃ |
| —Br | —H | —CH₂H₃ |

Without wishing to be bound by any particular theory, it is believed that such warhead groups are particularly suitable for covalently binding to a side chain of a key amino acid residue (e.g., a cysteine residue) in a TEAD transcription factor (e.g., TEAD1, TEAD2, TEAD3, or TEAD4). Thus, in some embodiments, R^{w} as described above and herein is capable of covalently binding to a side chain of an amino acid residue (e.g., cysteine) of a TEAD transcription factor (e.g., TEAD1, TEAD2, TEAD3, or TEAD4), thereby irreversibly inhibiting the enzyme.

In some embodiments, a provided compound is not:

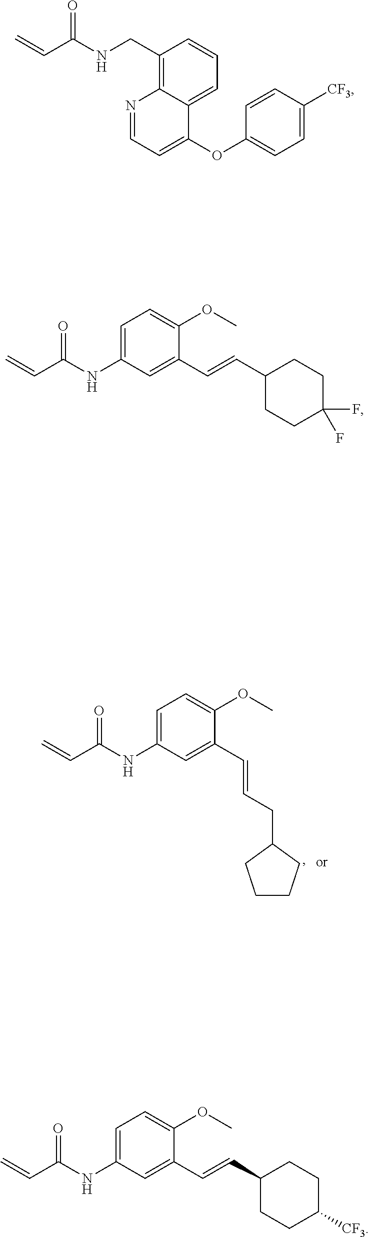 [SMILES: C=CC(=O)NCc1cccc2c(Oc3ccc(C(F)(F)F)cc3)ccnc12 ; C=CC(=O)Nc1ccc(OC)c(/C=C/C2CCC(F)(F)CC2)c1 ; C=CC(=O)Nc1ccc(OC)c(/C=C/CC2CCCC2)c1 ; C=CC(=O)Nc1ccc(OC)c(/C=C/[C@H]2CC[C@H](C(F)(F)F)CC2)c1]

In some embodiments, the present disclosure provides a compound of Formulae I-a, I-b, or I-c:

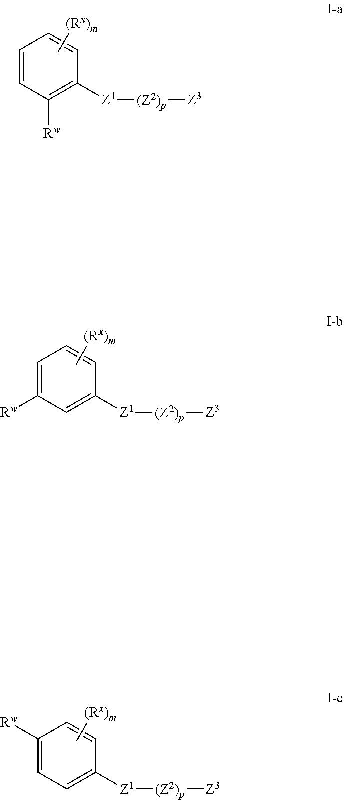

or a pharmaceutically acceptable salt thereof, wherein each of R^{w}, R^{x}, Z¹, Z², Z³, m, and p is as defined above and described herein.

In some embodiments, the present disclosure provides a compound of Formulae I-d, I-e, or I-f:

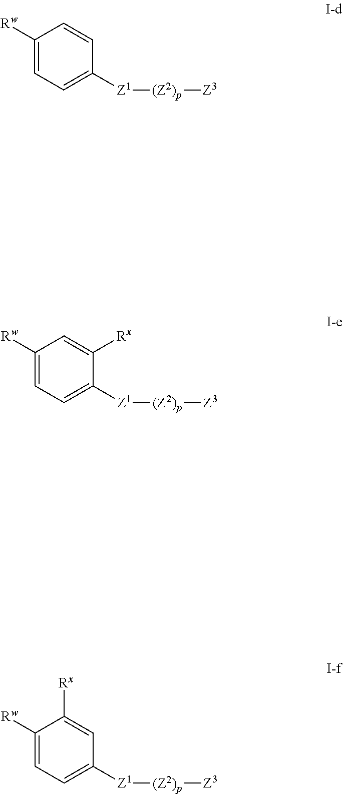

or a pharmaceutically acceptable salt thereof, wherein each of R^{w}, R^{x}, Z¹, Z², Z³, and p is as defined above and described herein.

In some embodiments, the present disclosure provides a compound of Formulae I-1, I-a1, I-b1, I-c1, I-d1, I-e1, or I-f1:

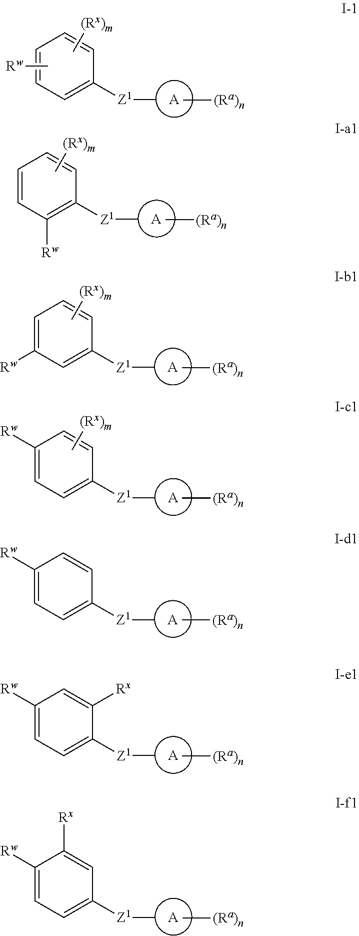

or a pharmaceutically acceptable salt thereof, wherein each of R^{w}, R^{x}, Z¹, Ring A, R^{a}, m, and n is as defined above and described herein.

In some embodiments, the present disclosure provides a compound of Formulae 1-2, I-a2, I-b2, I-c2, I-d2, I-e2, or I-f2:

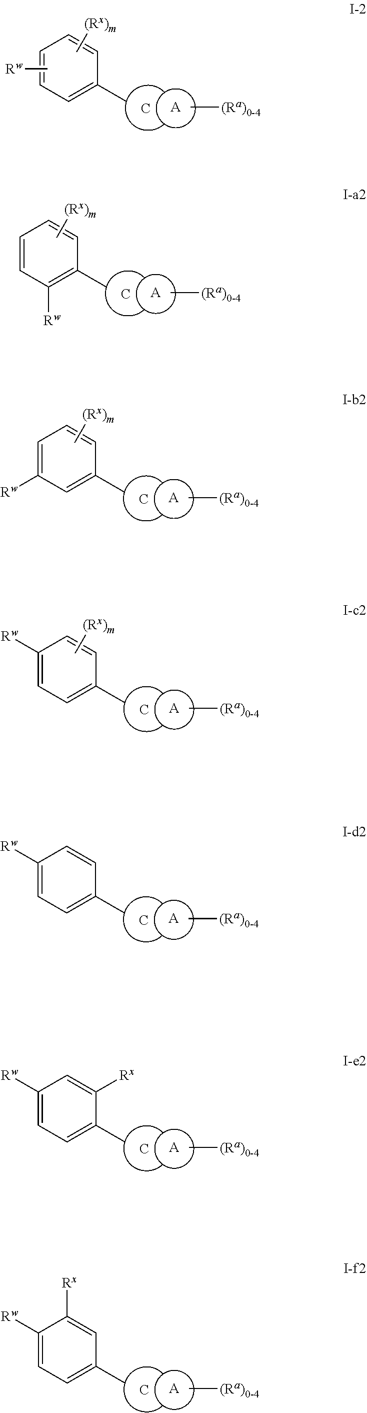

or a pharmaceutically acceptable salt thereof, wherein each of R^{w}, R^{x}, Ring A, Ring C, R^{a}, m, and n is as defined above and described herein.

In some embodiments, the present disclosure provides a compound of Formulae II, II-a, or II-b:

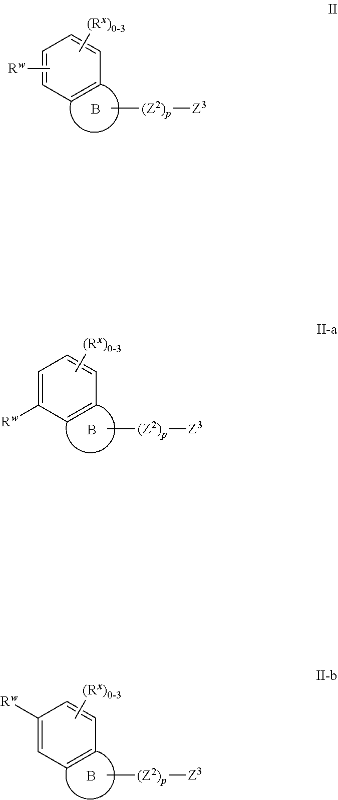

or a pharmaceutically acceptable salt thereof, wherein each of R^{w}, R^{x}, Ring B, Z², Z³, and p is as defined above and described herein.

In some embodiments, the present disclosure provides a compound of Formulae III, III-a, III-b, III-c, or III-d:

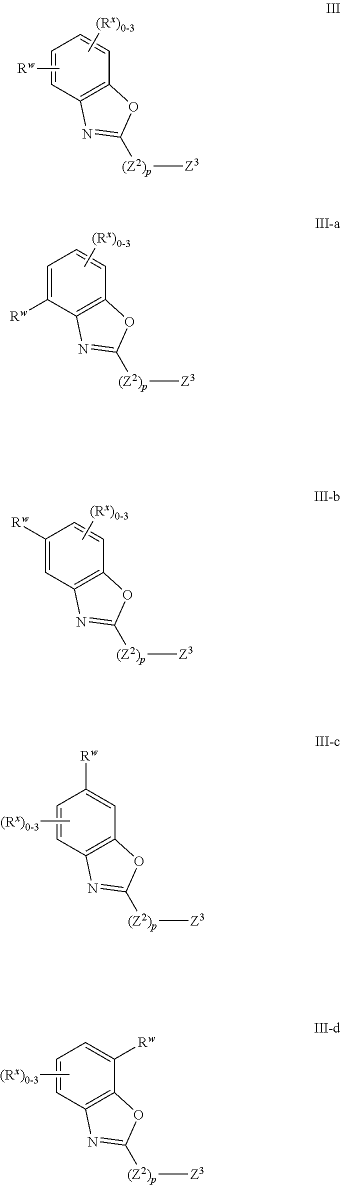

or a pharmaceutically acceptable salt thereof, wherein each of R^{w}, R^{x}, m, Z², Z³, and p is as defined above and described herein.

In some embodiments, the present disclosure provides a compound of Formulae IV, IV-a, or IV-b:

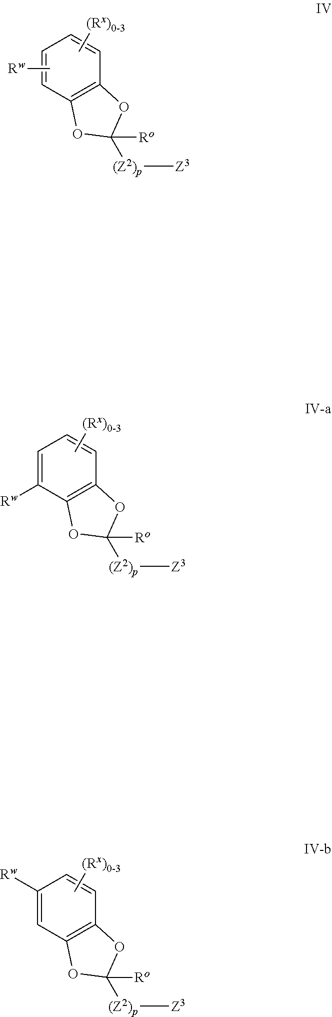

or a pharmaceutically acceptable salt thereof, wherein
R^{∘} is hydrogen or C₁₋₆ aliphatic; and
each of R^{w}, R^{x}, Z², Z³, and p is as defined above and described herein.

In some embodiments, the present disclosure provides a compound of Formulae V, V-a, V-b, V-c, or V-d:

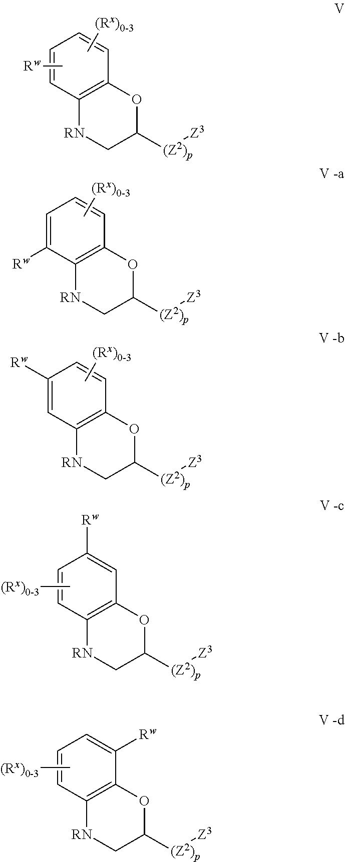

or a pharmaceutically acceptable salt thereof, wherein each of R^{w}, R^{x}, R, Z², Z³, and p is as defined above and described herein.

In some embodiments, the present disclosure provides a compound of Formulae VI, VI-a, VI-b, VI-c, or VI-d:

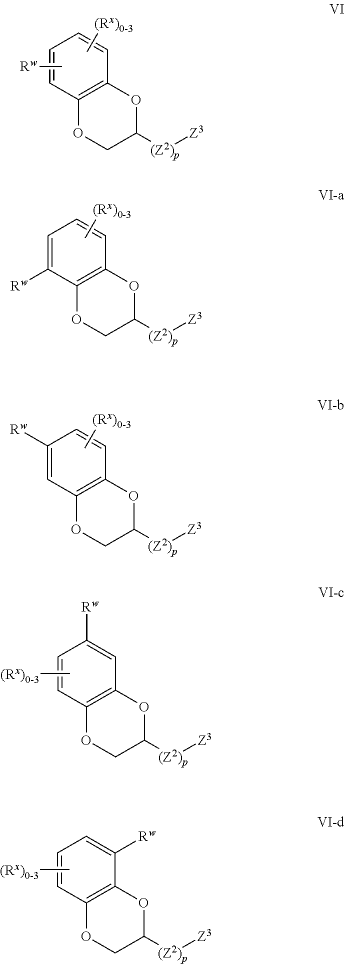

or a pharmaceutically acceptable salt thereof, wherein each of R^{w}, R^{x}, Z², Z³, and p is as defined above and described herein.

In some embodiments, the present disclosure provides a compound of Formulae VII, VII-a, VII-b, VII-c, or VII-d:

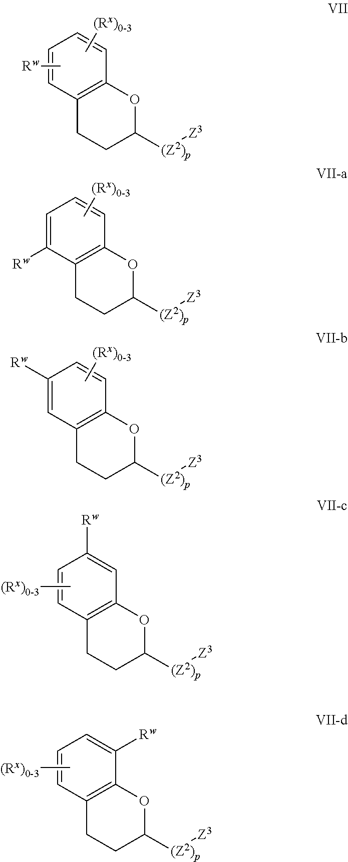

or a pharmaceutically acceptable salt thereof, wherein each of R^{w}, R^{x}, Z², Z³, and p is as defined above and described herein.

It will be understood that, unless otherwise specified or prohibited by the foregoing definitions of Formulae I-a through VII-d, embodiments of variables as defined above for formula I and described in classes and subclasses herein also apply to compounds of Formulae I-a through VII-d, mutatis mutandis, both singly and in combination.

In some embodiments, a provided compound is selected from the group consisting of:

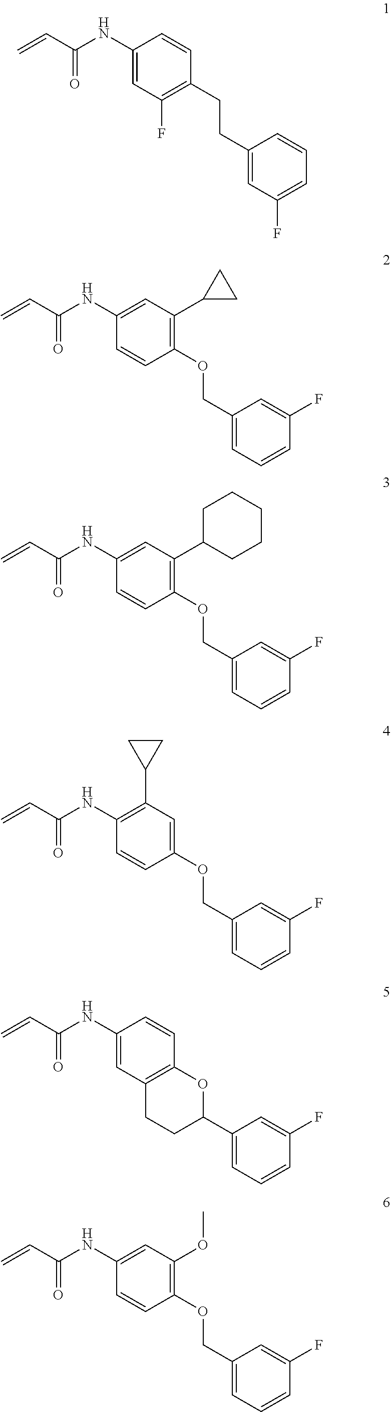

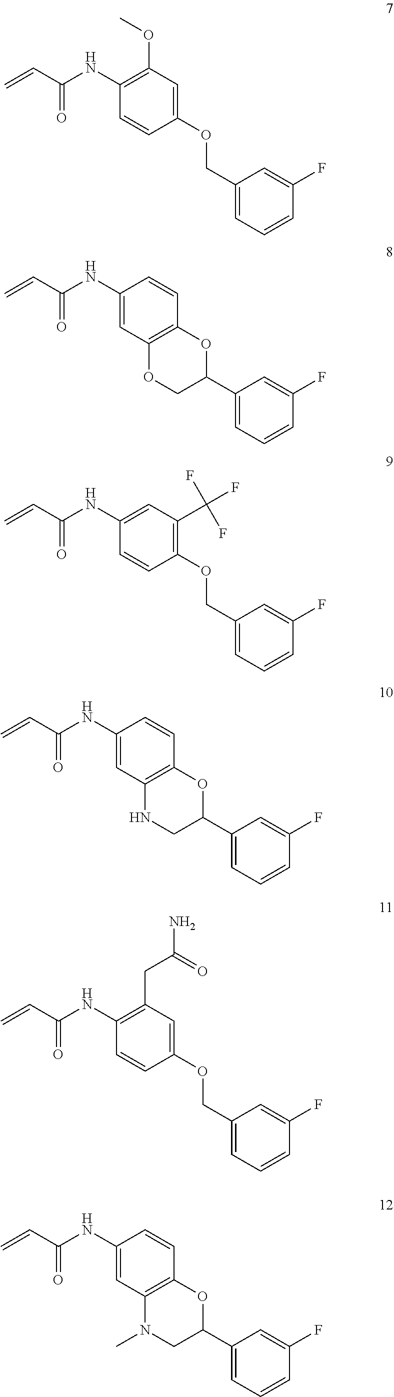 [SMILES: C=CC(=O)Nc1ccc(OCc2cccc(F)c2)cc1OC ; C=CC(=O)Nc1ccc2c(c1)OCC(c1cccc(F)c1)O2 ; C=CC(=O)Nc1ccc(OCc2cccc(F)c2)c(C(F)(F)F)c1 ; C=CC(=O)Nc1ccc2c(c1)NCC(c1cccc(F)c1)O2 ; C=CC(=O)Nc1ccc(OCc2cccc(F)c2)cc1CC(N)=O ; C=CC(=O)Nc1ccc2c(c1)N(C)CC(c1cccc(F)c1)O2]

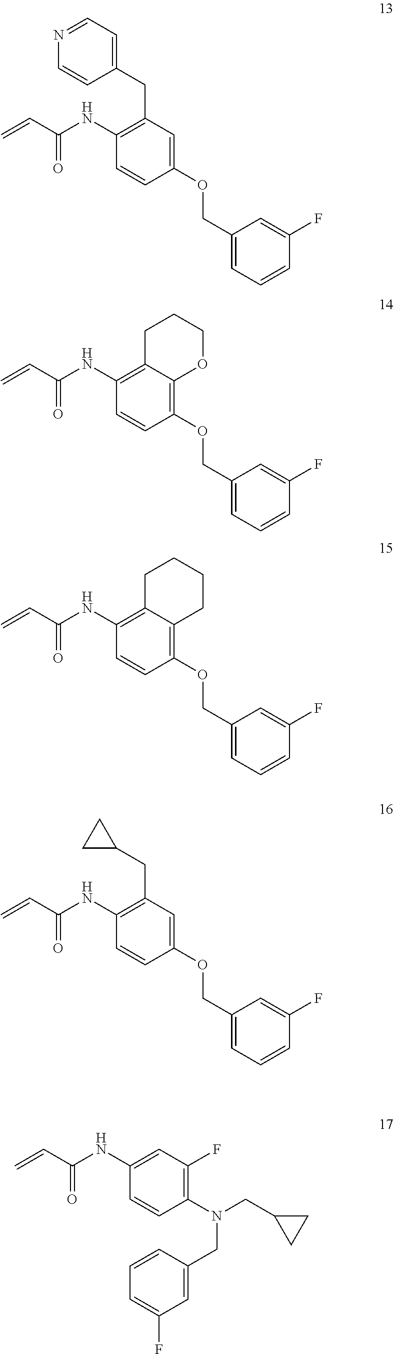 [SMILES: C=CC(=O)Nc1ccc(OCc2cccc(F)c2)cc1Cc1ccncc1 ; C=CC(=O)Nc1ccc(OCc2cccc(F)c2)c2c1CCCO2 ; C=CC(=O)Nc1ccc(OCc2cccc(F)c2)c2c1CCCC2 ; C=CC(=O)Nc1ccc(OCc2cccc(F)c2)cc1CC1CC1 ; C=CC(=O)Nc1ccc(N(Cc2cccc(F)c2)CC2CC2)c(F)c1]

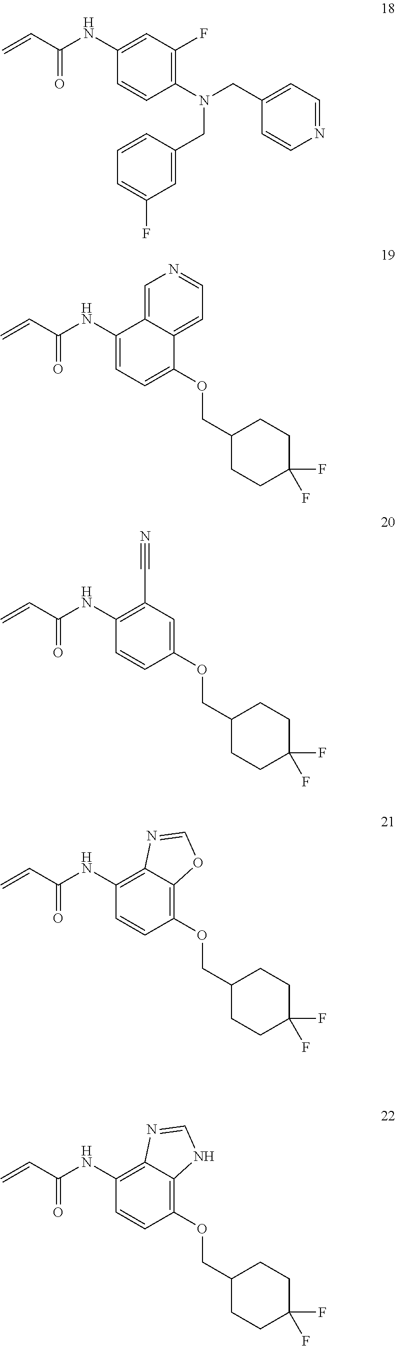

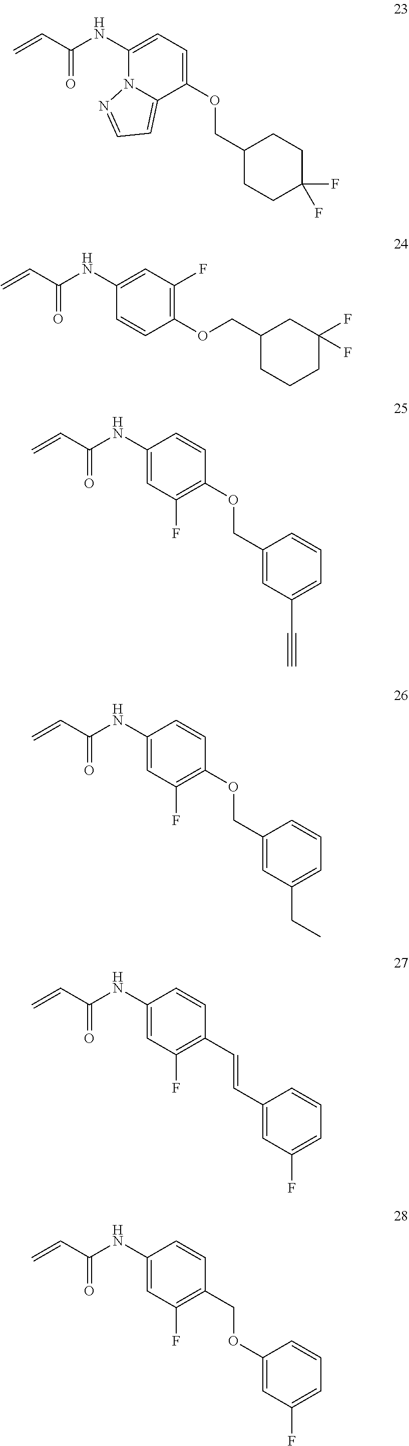

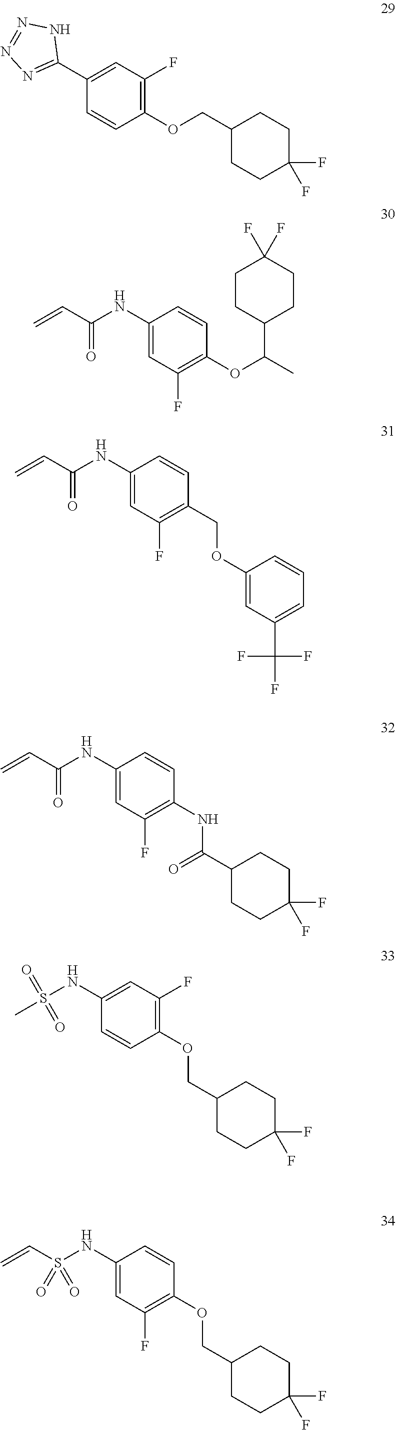

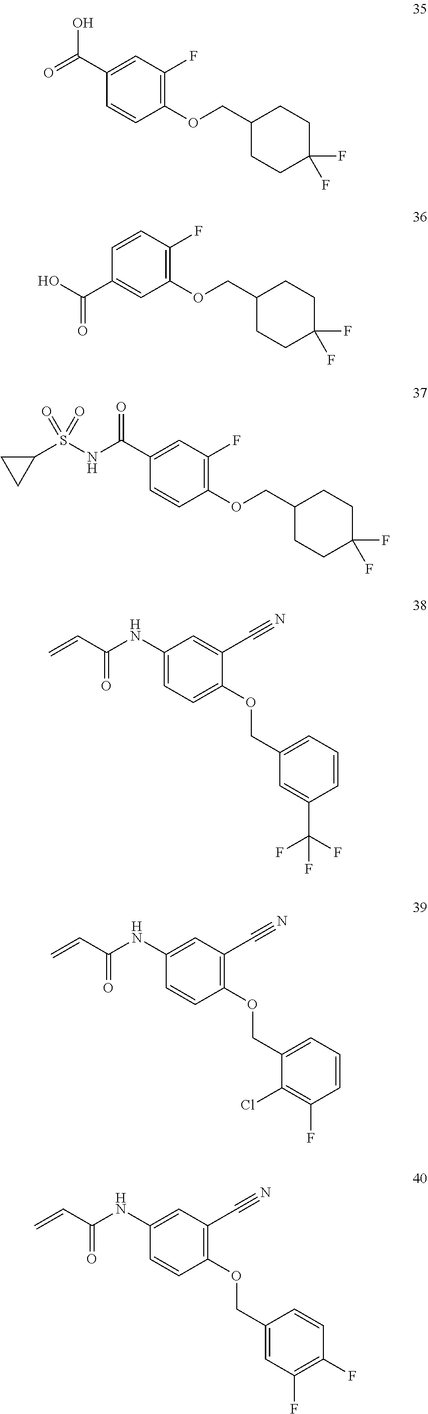 [SMILES: O=C(O)c1ccc(OCC2CCC(F)(F)CC2)c(F)c1 ; O=C(O)c1ccc(F)c(OCC2CCC(F)(F)CC2)c1 ; O=C(NS(=O)(=O)C1CC1)c1ccc(OCC2CCC(F)(F)CC2)c(F)c1 ; C=CC(=O)Nc1ccc(OCc2cccc(C(F)(F)F)c2)c(C#N)c1 ; C=CC(=O)Nc1ccc(OCc2cccc(F)c2Cl)c(C#N)c1 ; C=CC(=O)Nc1ccc(OCc2ccc(F)c(F)c2)c(C#N)c1]

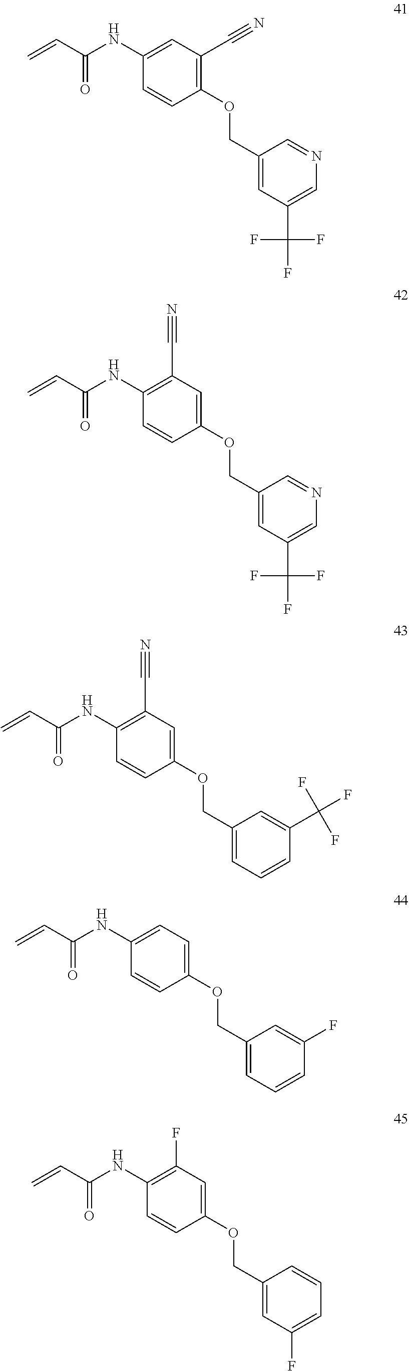 [SMILES: C=CC(=O)Nc1ccc(OCc2cncc(C(F)(F)F)c2)c(C#N)c1 ; C=CC(=O)Nc1ccc(OCc2cncc(C(F)(F)F)c2)cc1C#N ; C=CC(=O)Nc1ccc(OCc2cccc(C(F)(F)F)c2)cc1C#N ; C=CC(=O)Nc1ccc(OCc2cccc(F)c2)cc1 ; C=CC(=O)Nc1ccc(OCc2cccc(F)c2)cc1F]

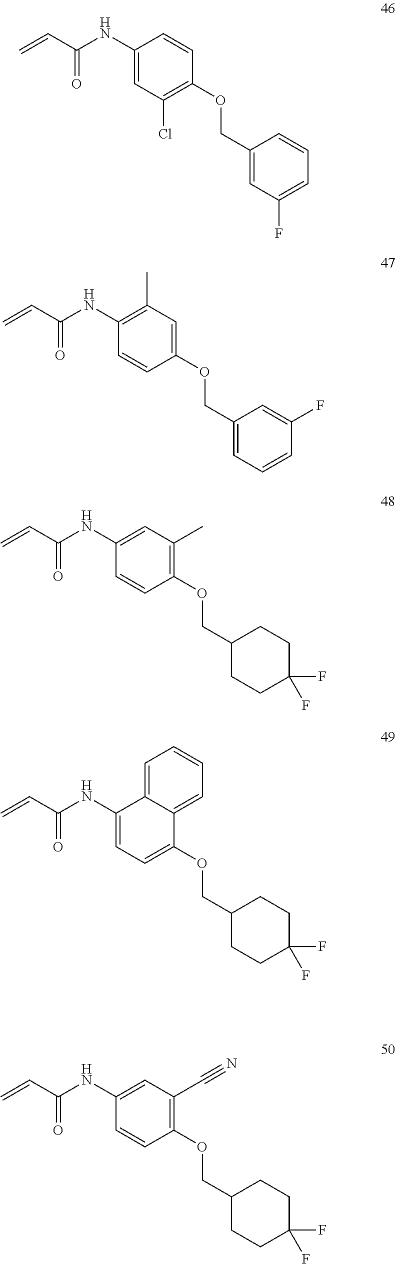 [SMILES: C=CC(=O)Nc1ccc(OCc2cccc(F)c2)c(Cl)c1 ; C=CC(=O)Nc1ccc(OCc2cccc(F)c2)cc1C ; C=CC(=O)Nc1ccc(OCC2CCC(F)(F)CC2)c(C)c1 ; C=CC(=O)Nc1ccc(OCC2CCC(F)(F)CC2)c2ccccc12 ; C=CC(=O)Nc1ccc(OCC2CCC(F)(F)CC2)c(C#N)c1]

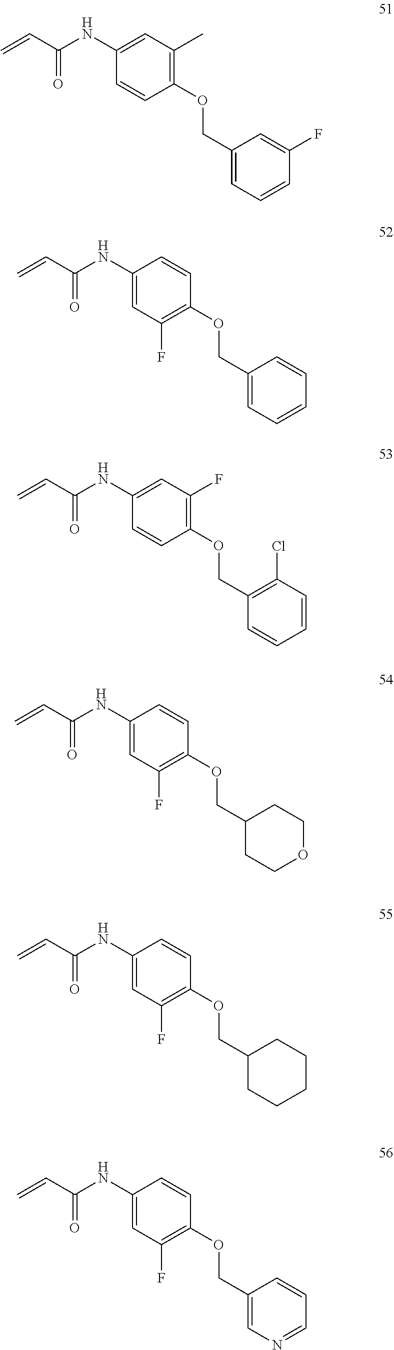 [SMILES: C=CC(=O)Nc1ccc(OCc2cccc(F)c2)c(C)c1 ; C=CC(=O)Nc1ccc(OCc2ccccc2)c(F)c1 ; C=CC(=O)Nc1ccc(OCc2ccccc2Cl)c(F)c1 ; C=CC(=O)Nc1ccc(OCC2CCOCC2)c(F)c1 ; C=CC(=O)Nc1ccc(OCC2CCCCC2)c(F)c1 ; C=CC(=O)Nc1ccc(OCc2cccnc2)c(F)c1]

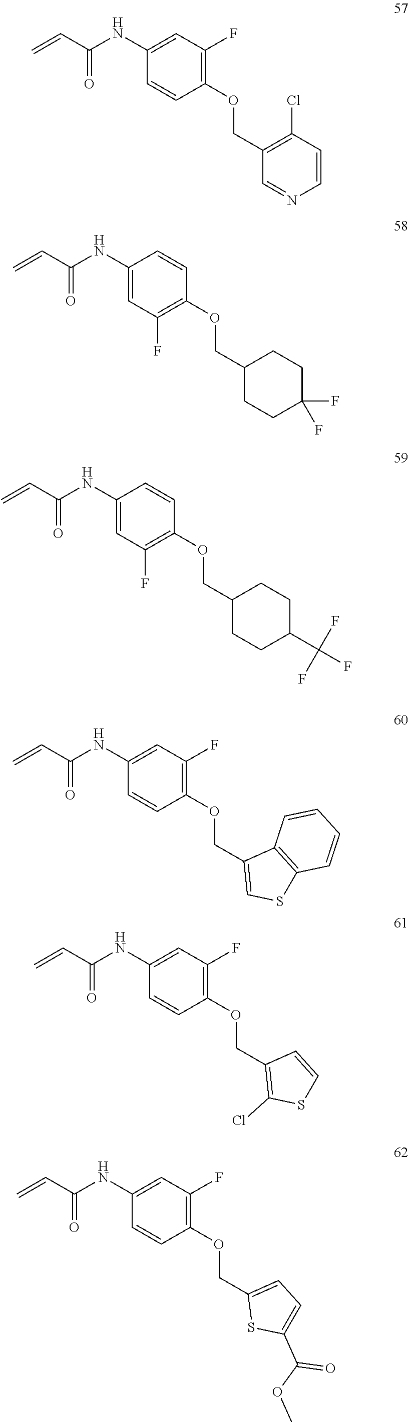 [SMILES: C=CC(=O)Nc1ccc(OCc2cnccc2Cl)c(F)c1 ; C=CC(=O)Nc1ccc(OCC2CCC(F)(F)CC2)c(F)c1 ; C=CC(=O)Nc1ccc(OCC2CCC(C(F)(F)F)CC2)c(F)c1 ; C=CC(=O)Nc1ccc(OCc2csc3ccccc23)c(F)c1 ; C=CC(=O)Nc1ccc(OCc2ccsc2Cl)c(F)c1 ; C=CC(=O)Nc1ccc(OCc2ccc(C(=O)OC)s2)c(F)c1]

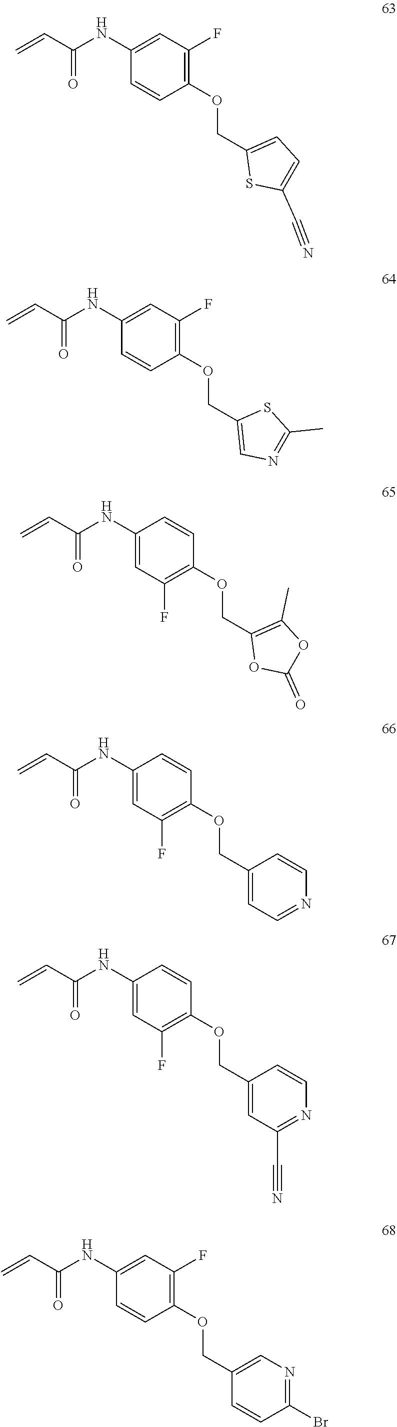

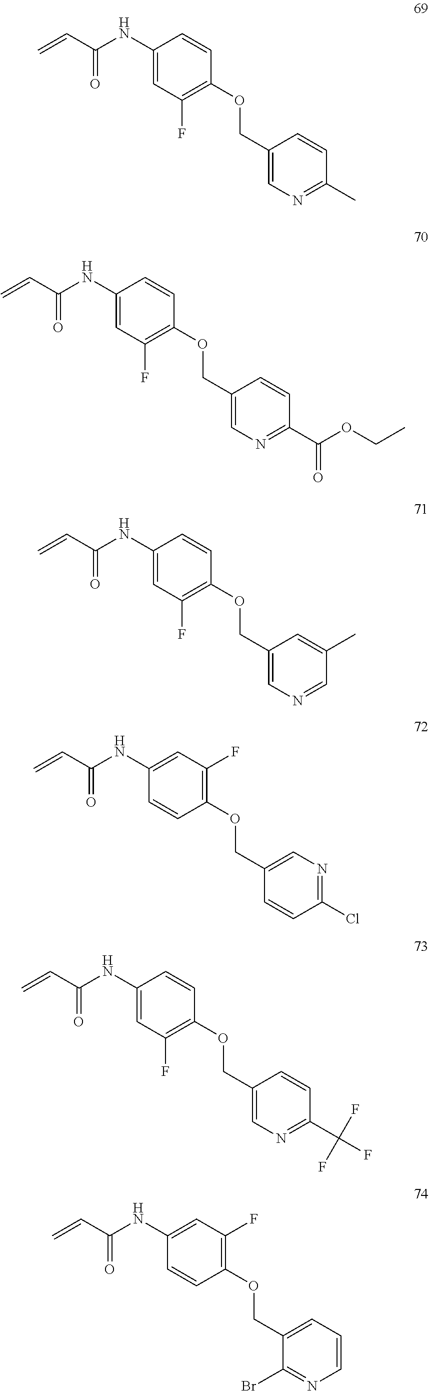

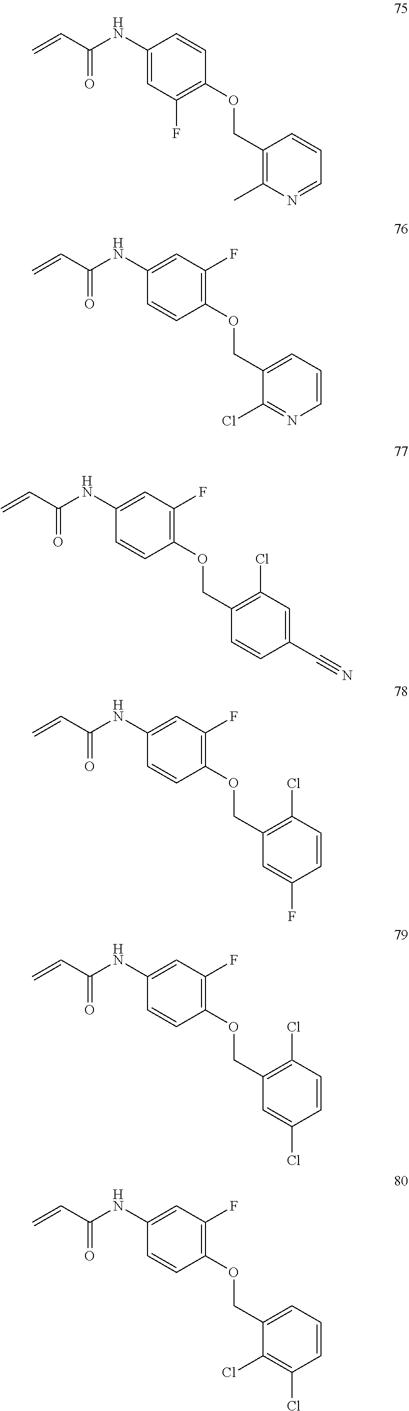 [SMILES: C=CC(=O)Nc1ccc(OCc2cccnc2C)c(F)c1 ; C=CC(=O)Nc1ccc(OCc2cccnc2Cl)c(F)c1 ; C=CC(=O)Nc1ccc(OCc2ccc(C#N)cc2Cl)c(F)c1 ; C=CC(=O)Nc1ccc(OCc2cc(F)ccc2Cl)c(F)c1 ; C=CC(=O)Nc1ccc(OCc2cc(Cl)ccc2Cl)c(F)c1 ; C=CC(=O)Nc1ccc(OCc2cccc(Cl)c2Cl)c(F)c1]

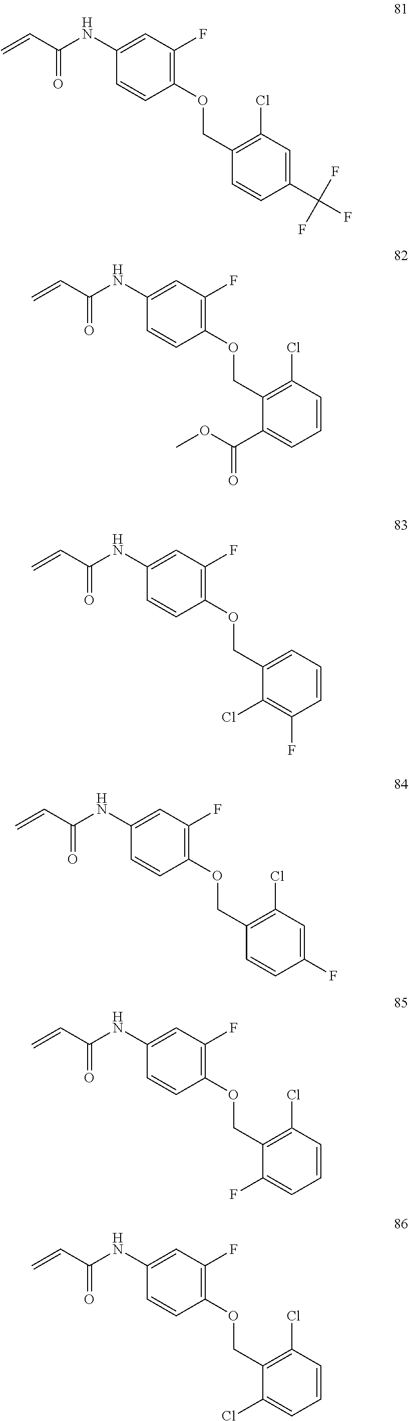 [SMILES: C=CC(=O)Nc1ccc(OCc2ccc(C(F)(F)F)cc2Cl)c(F)c1 ; C=CC(=O)Nc1ccc(OCc2c(Cl)cccc2C(=O)OC)c(F)c1 ; C=CC(=O)Nc1ccc(OCc2cccc(F)c2Cl)c(F)c1 ; C=CC(=O)Nc1ccc(OCc2ccc(F)cc2Cl)c(F)c1 ; C=CC(=O)Nc1ccc(OCc2c(F)cccc2Cl)c(F)c1 ; C=CC(=O)Nc1ccc(OCc2c(Cl)cccc2Cl)c(F)c1]

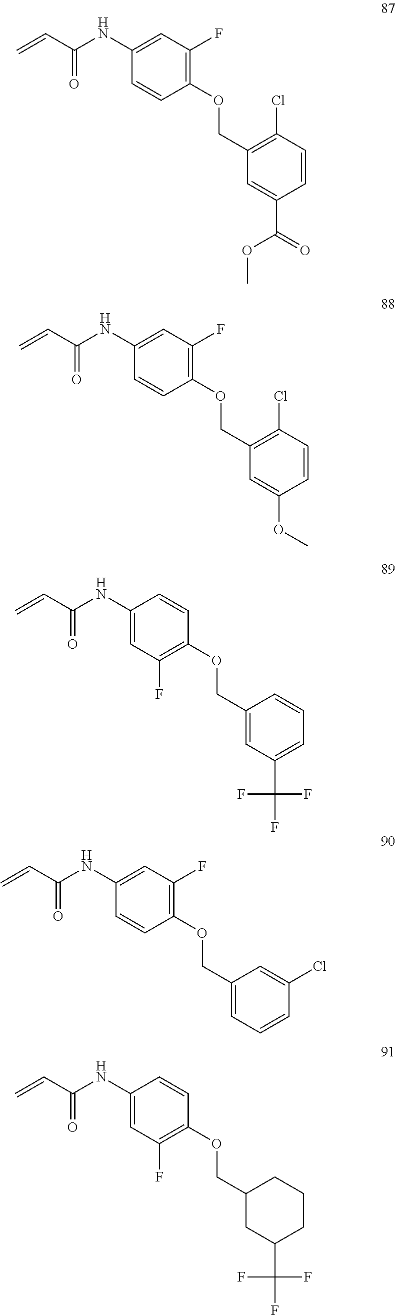

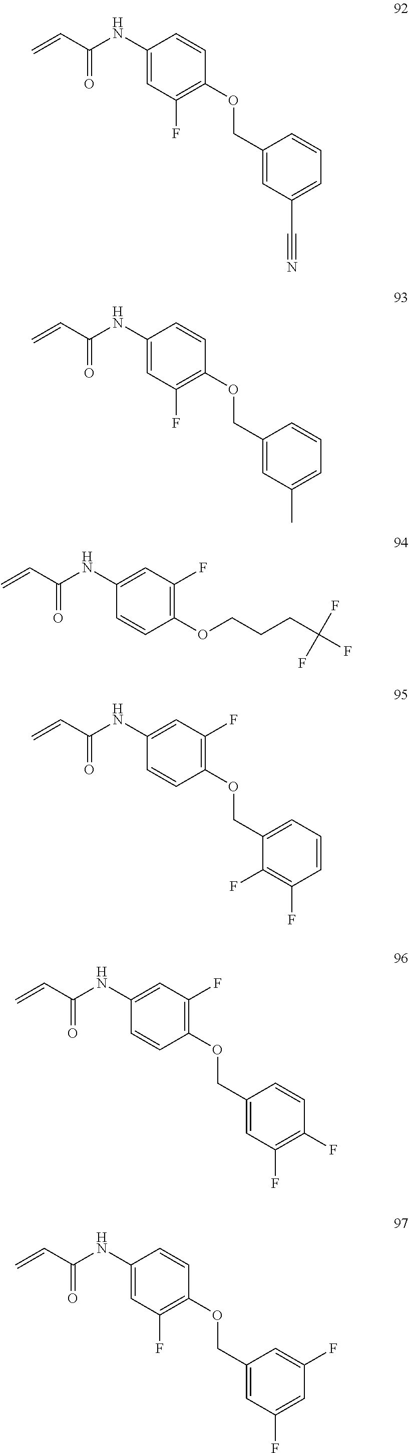 [SMILES: C=CC(=O)Nc1ccc(OCc2cccc(C#N)c2)c(F)c1 ; C=CC(=O)Nc1ccc(OCc2cccc(C)c2)c(F)c1 ; C=CC(=O)Nc1ccc(OCCCC(F)(F)F)c(F)c1 ; C=CC(=O)Nc1ccc(OCc2cccc(F)c2F)c(F)c1 ; C=CC(=O)Nc1ccc(OCc2ccc(F)c(F)c2)c(F)c1 ; C=CC(=O)Nc1ccc(OCc2cc(F)cc(F)c2)c(F)c1]

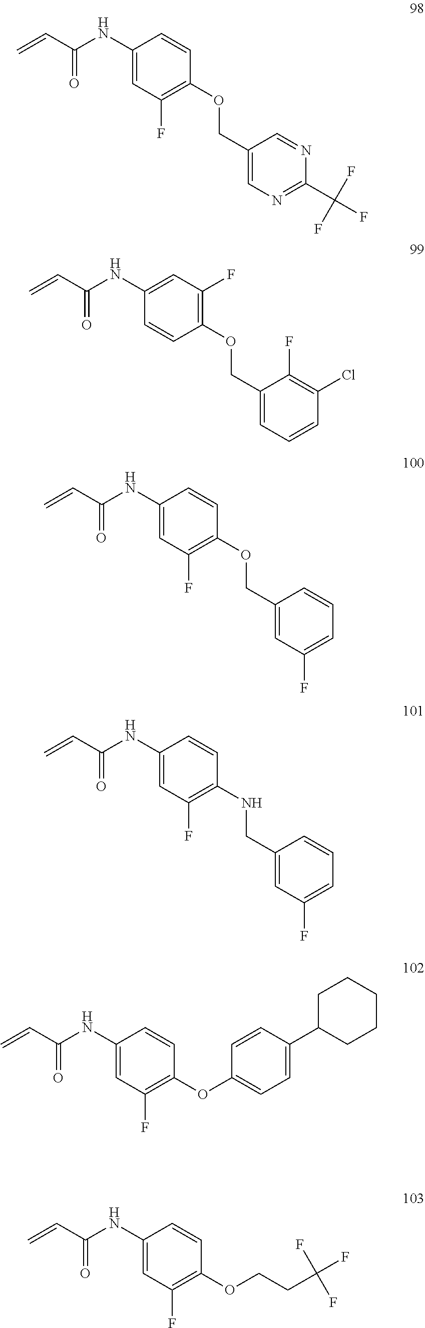 [SMILES: C=CC(=O)Nc1ccc(OCc2cnc(C(F)(F)F)nc2)c(F)c1 ; C=CC(=O)Nc1ccc(OCc2cccc(Cl)c2F)c(F)c1 ; C=CC(=O)Nc1ccc(OCc2cccc(F)c2)c(F)c1 ; C=CC(=O)Nc1ccc(NCc2cccc(F)c2)c(F)c1 ; C=CC(=O)Nc1ccc(Oc2ccc(C3CCCCC3)cc2)c(F)c1 ; C=CC(=O)Nc1ccc(OCCC(F)(F)F)c(F)c1]

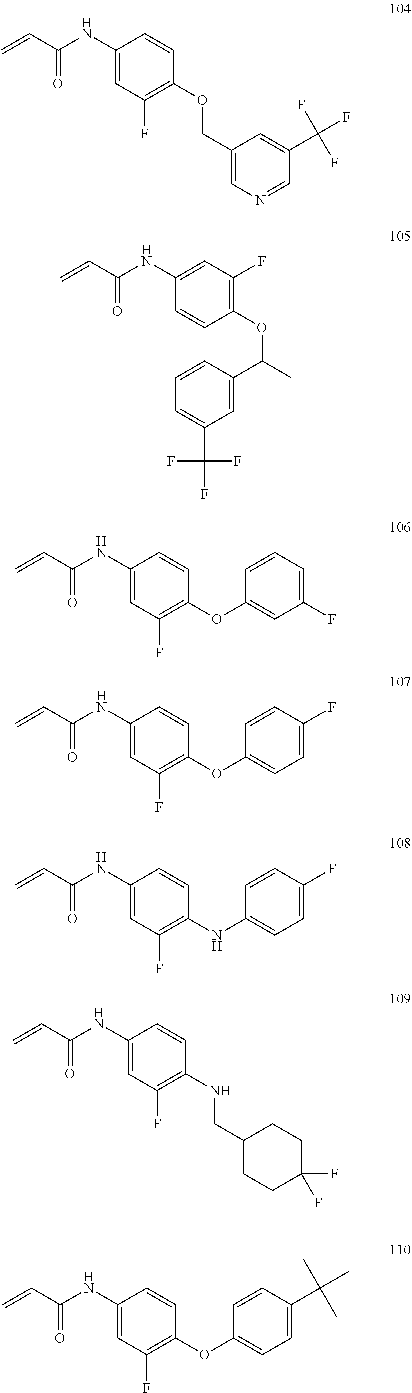

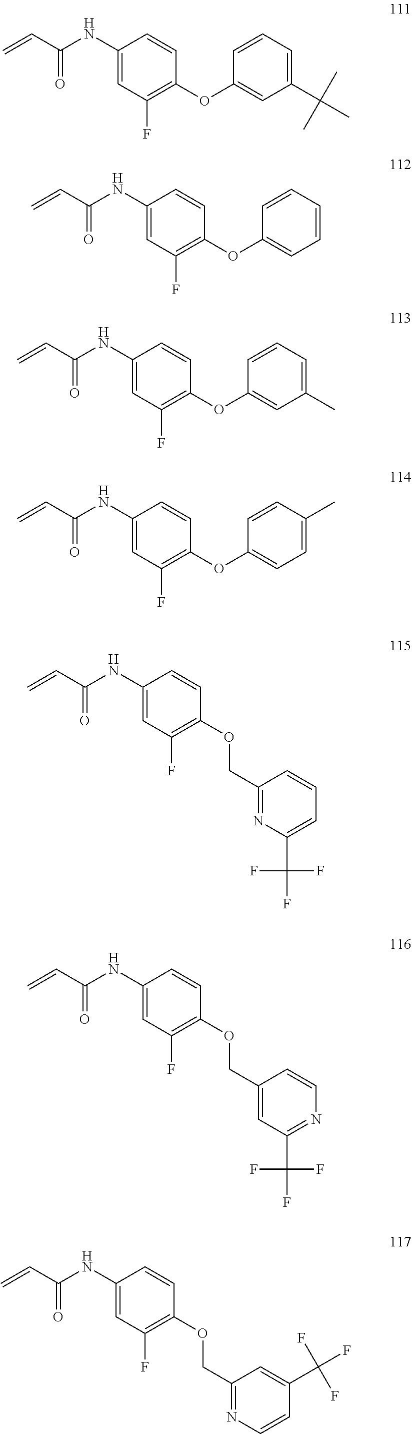

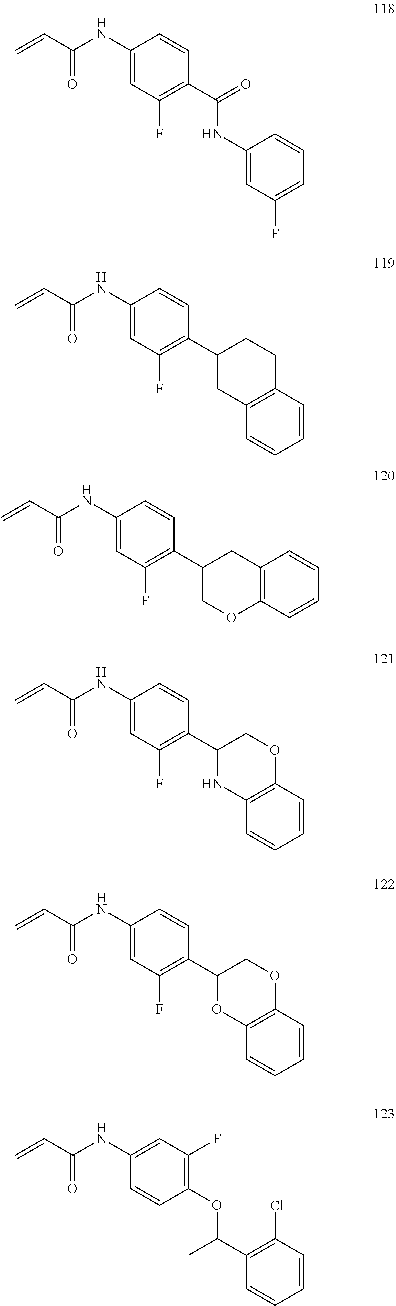 [SMILES: C=CC(=O)Nc1ccc(C(=O)Nc2cccc(F)c2)c(F)c1 ; C=CC(=O)Nc1ccc(C2CCc3ccccc3C2)c(F)c1 ; C=CC(=O)Nc1ccc(C2COc3ccccc3C2)c(F)c1 ; C=CC(=O)Nc1ccc(C2COc3ccccc3N2)c(F)c1 ; C=CC(=O)Nc1ccc(C2COc3ccccc3O2)c(F)c1 ; C=CC(=O)Nc1ccc(OC(C)c2ccccc2Cl)c(F)c1]

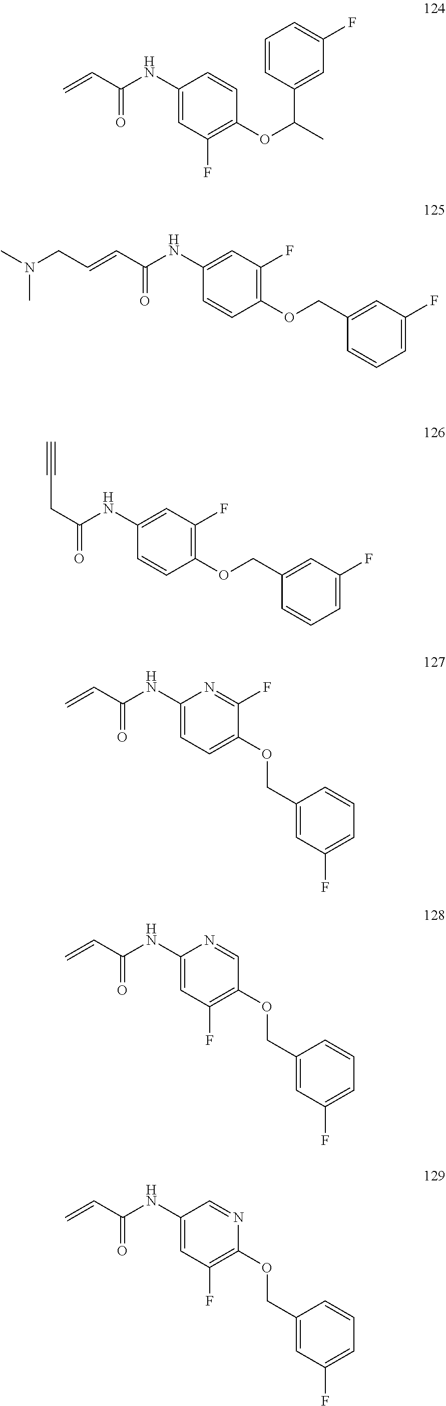 [SMILES: C=CC(=O)Nc1ccc(OC(C)c2cccc(F)c2)c(F)c1 ; CN(C)C/C=C/C(=O)Nc1ccc(OCc2cccc(F)c2)c(F)c1 ; C#CCC(=O)Nc1ccc(OCc2cccc(F)c2)c(F)c1 ; C=CC(=O)Nc1ccc(OCc2cccc(F)c2)c(F)n1 ; C=CC(=O)Nc1cc(F)c(OCc2cccc(F)c2)cn1 ; C=CC(=O)Nc1cnc(OCc2cccc(F)c2)c(F)c1]

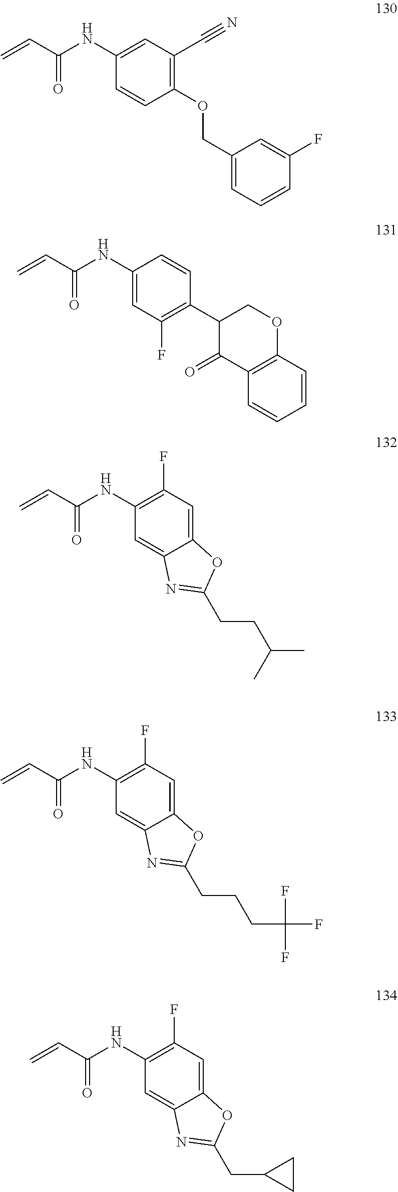

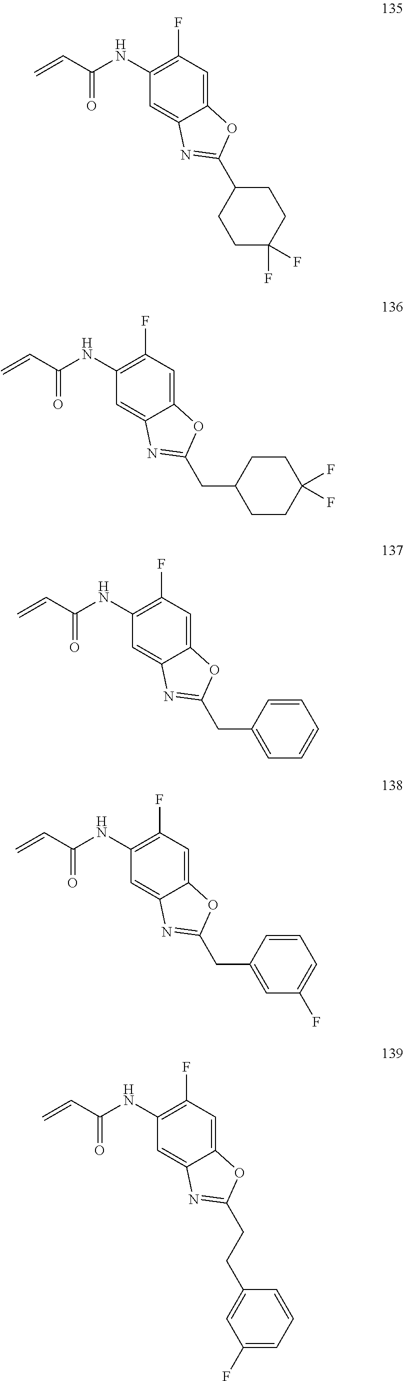 [SMILES: C=CC(=O)Nc1cc2nc(C3CCC(F)(F)CC3)oc2cc1F ; C=CC(=O)Nc1cc2nc(CC3CCC(F)(F)CC3)oc2cc1F ; C=CC(=O)Nc1cc2nc(Cc3ccccc3)oc2cc1F ; C=CC(=O)Nc1cc2nc(Cc3cccc(F)c3)oc2cc1F ; C=CC(=O)Nc1cc2nc(CCc3cccc(F)c3)oc2cc1F]

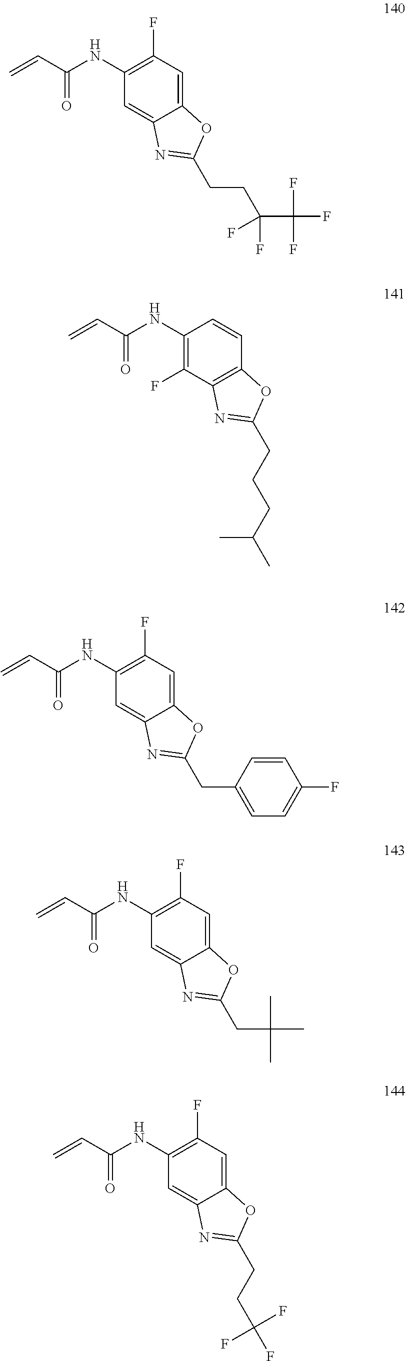

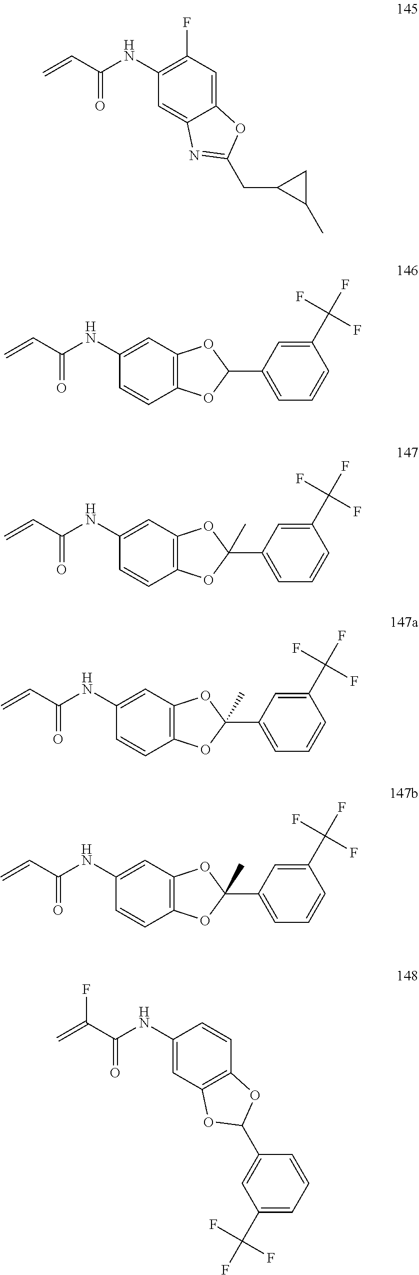

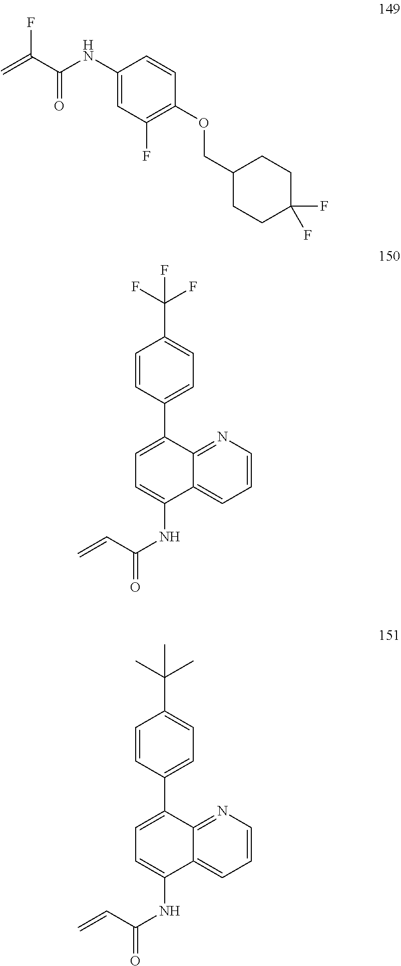 [SMILES: C=C(F)C(=O)Nc1ccc(OCC2CCC(F)(F)CC2)c(F)c1 ; C=CC(=O)Nc1ccc(-c2ccc(C(F)(F)F)cc2)c2ncccc12 ; C=CC(=O)Nc1ccc(-c2ccc(C(C)(C)C)cc2)c2ncccc12]

or a pharmaceutically acceptable salt thereof.

In some embodiments, a provided compound is selected from the group consisting of:

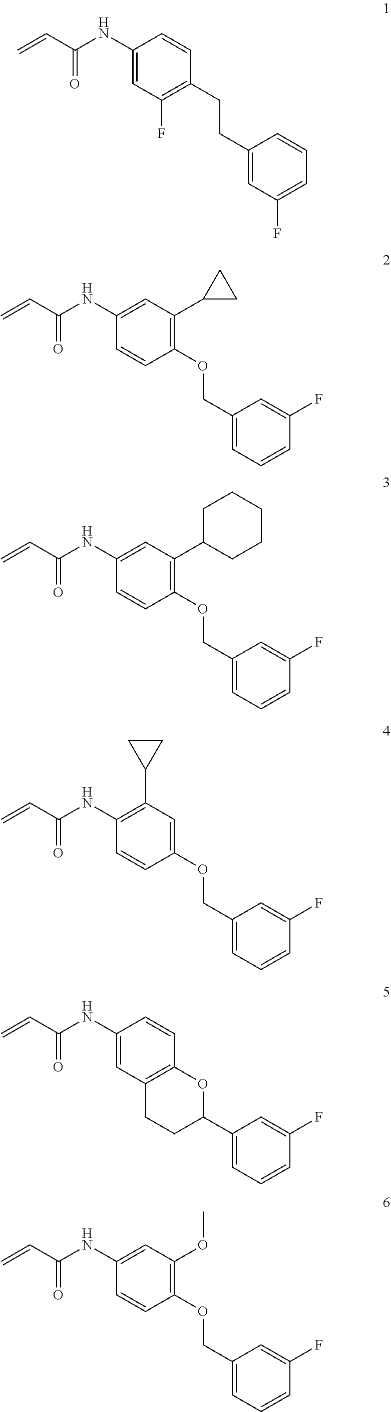

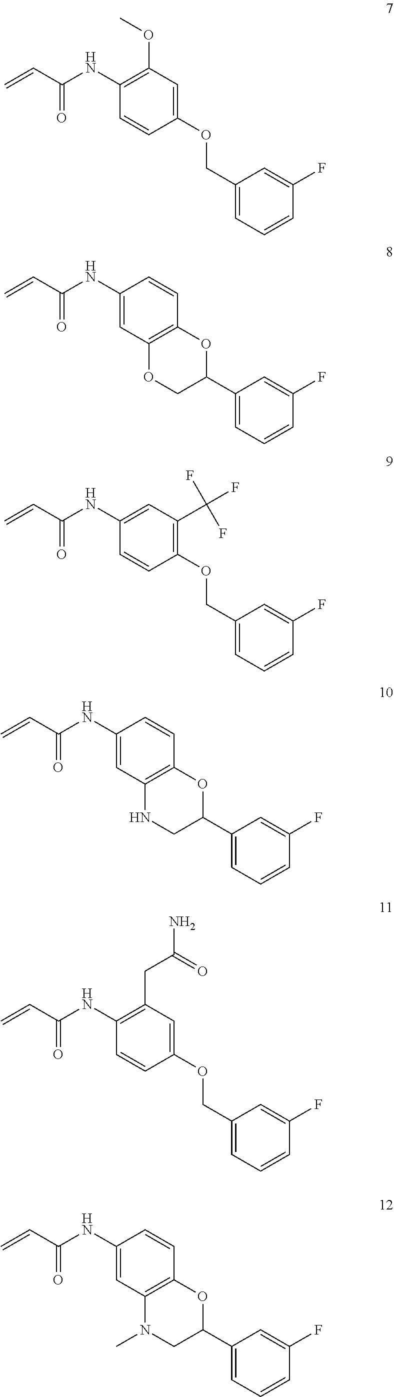 [SMILES: C=CC(=O)Nc1ccc(OCc2cccc(F)c2)cc1OC ; C=CC(=O)Nc1ccc2c(c1)OCC(c1cccc(F)c1)O2 ; C=CC(=O)Nc1ccc(OCc2cccc(F)c2)c(C(F)(F)F)c1 ; C=CC(=O)Nc1ccc2c(c1)NCC(c1cccc(F)c1)O2 ; C=CC(=O)Nc1ccc(OCc2cccc(F)c2)cc1CC(N)=O ; C=CC(=O)Nc1ccc2c(c1)N(C)CC(c1cccc(F)c1)O2]

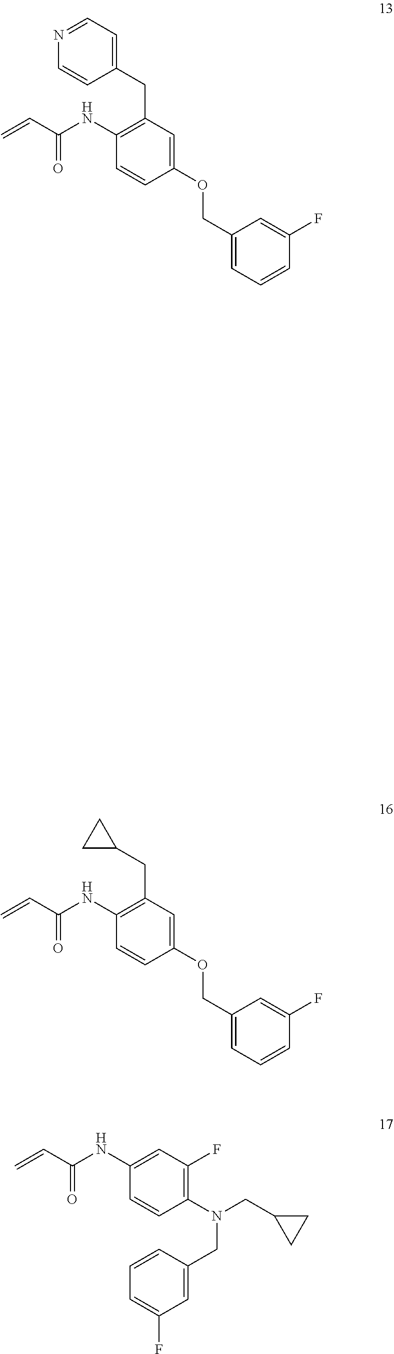 [SMILES: C=CC(=O)Nc1ccc(OCc2cccc(F)c2)cc1Cc1ccncc1 ; C=CC(=O)Nc1ccc(OCc2cccc(F)c2)cc1CC1CC1 ; C=CC(=O)Nc1ccc(N(Cc2cccc(F)c2)CC2CC2)c(F)c1]

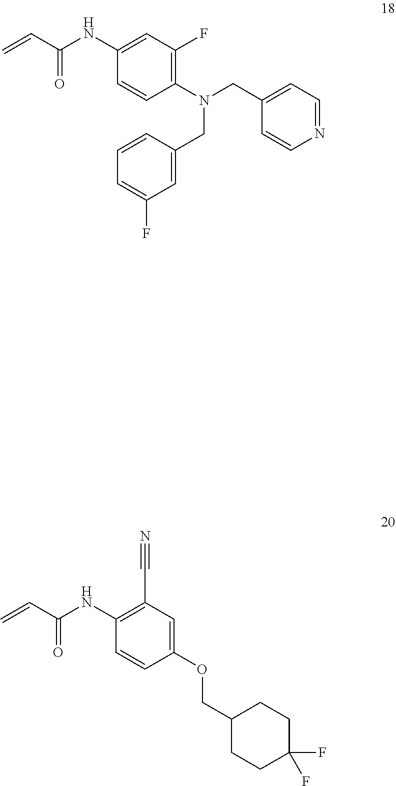 [SMILES: C=CC(=O)Nc1ccc(N(Cc2ccncc2)Cc2cccc(F)c2)c(F)c1 ; C=CC(=O)Nc1ccc(OCC2CCC(F)(F)CC2)cc1C#N]

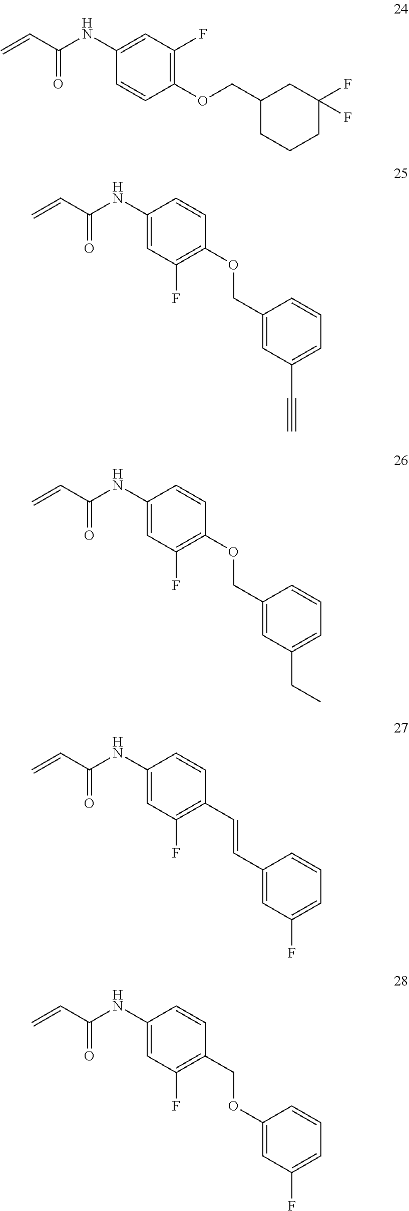

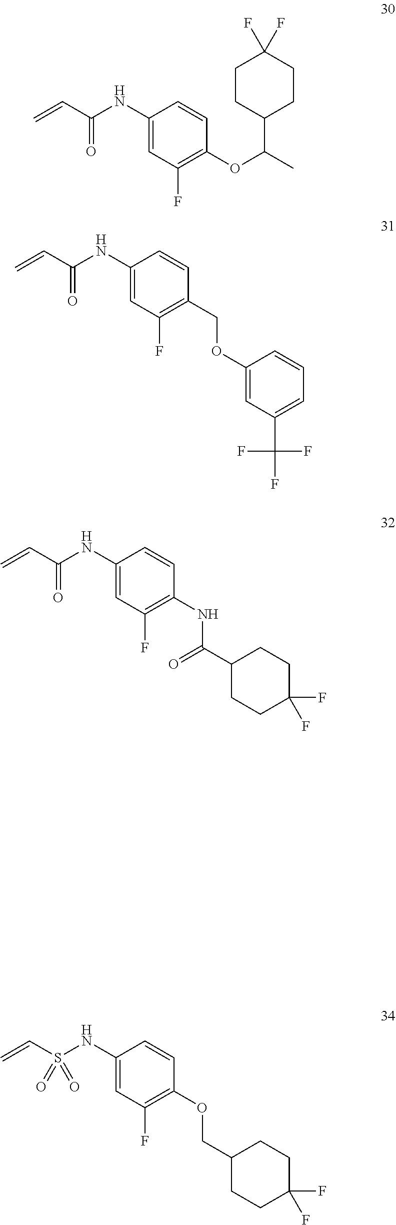 [SMILES: C=CC(=O)Nc1ccc(OC(C)C2CCC(F)(F)CC2)c(F)c1 ; C=CC(=O)Nc1ccc(COc2cccc(C(F)(F)F)c2)c(F)c1 ; C=CC(=O)Nc1ccc(NC(=O)C2CCC(F)(F)CC2)c(F)c1 ; C=CS(=O)(=O)Nc1ccc(OCC2CCC(F)(F)CC2)c(F)c1]

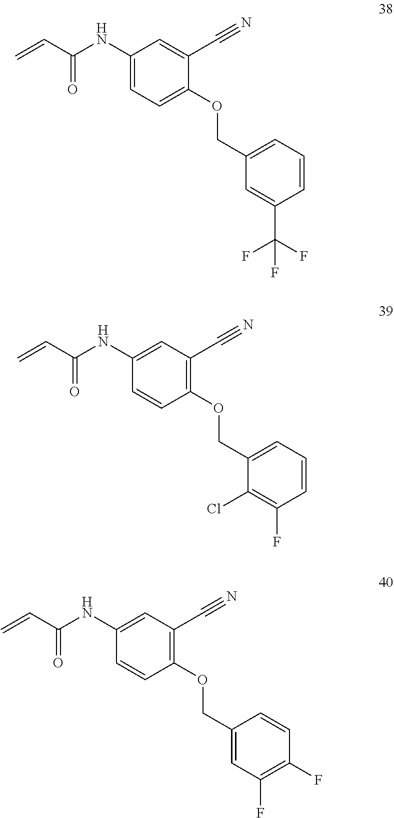 [SMILES: C=CC(=O)Nc1ccc(OCc2cccc(C(F)(F)F)c2)c(C#N)c1 ; C=CC(=O)Nc1ccc(OCc2cccc(F)c2Cl)c(C#N)c1 ; C=CC(=O)Nc1ccc(OCc2ccc(F)c(F)c2)c(C#N)c1]

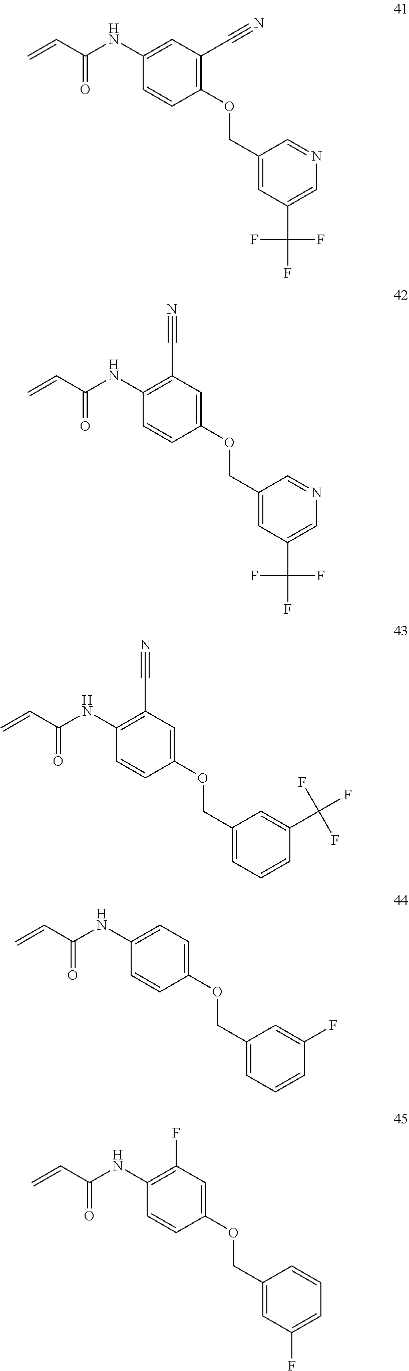 [SMILES: C=CC(=O)Nc1ccc(OCc2cncc(C(F)(F)F)c2)c(C#N)c1 ; C=CC(=O)Nc1ccc(OCc2cncc(C(F)(F)F)c2)cc1C#N ; C=CC(=O)Nc1ccc(OCc2cccc(C(F)(F)F)c2)cc1C#N ; C=CC(=O)Nc1ccc(OCc2cccc(F)c2)cc1 ; C=CC(=O)Nc1ccc(OCc2cccc(F)c2)cc1F]

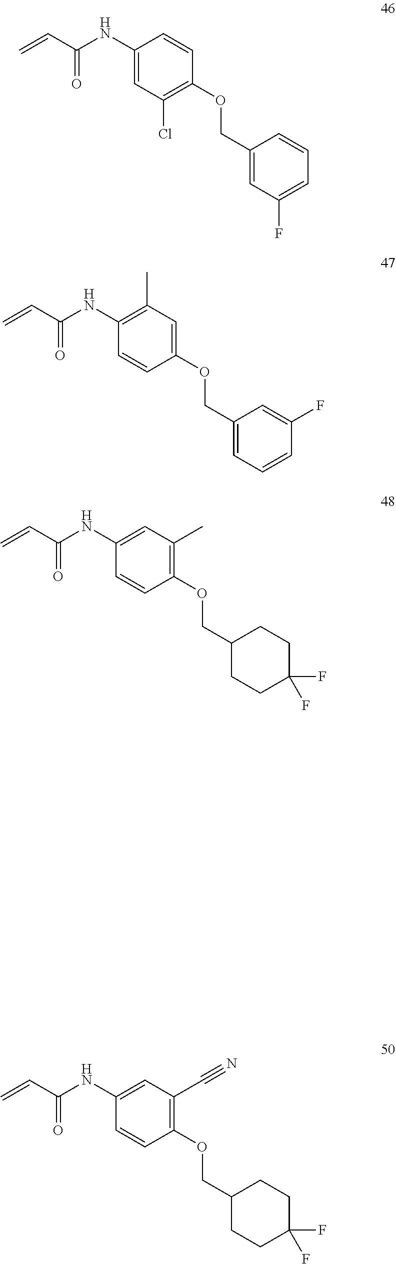 [SMILES: C=CC(=O)Nc1ccc(OCc2cccc(F)c2)c(Cl)c1 ; C=CC(=O)Nc1ccc(OCc2cccc(F)c2)cc1C ; C=CC(=O)Nc1ccc(OCC2CCC(F)(F)CC2)c(C)c1 ; C=CC(=O)Nc1ccc(OCC2CCC(F)(F)CC2)c(C#N)c1]

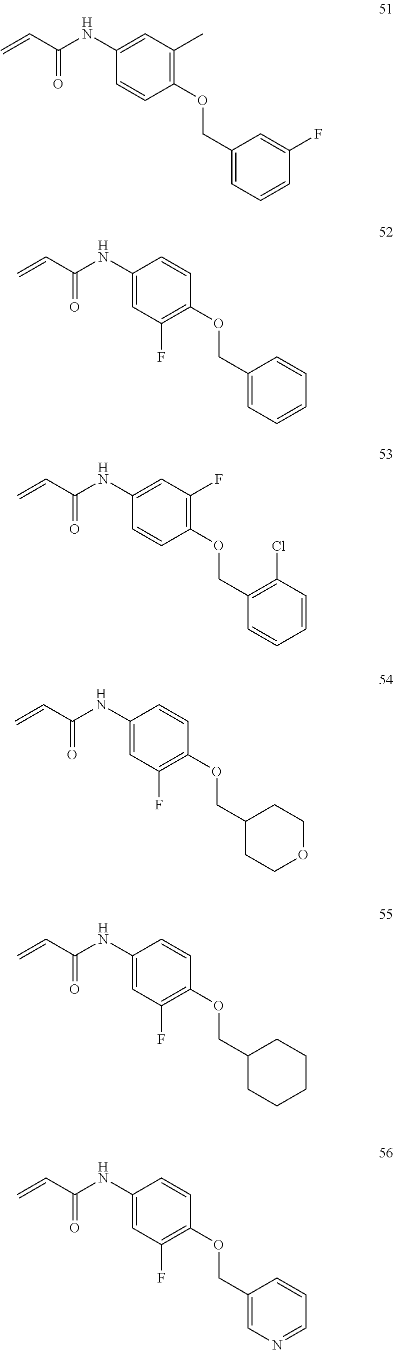 [SMILES: C=CC(=O)Nc1ccc(OCc2cccc(F)c2)c(C)c1 ; C=CC(=O)Nc1ccc(OCc2ccccc2)c(F)c1 ; C=CC(=O)Nc1ccc(OCc2ccccc2Cl)c(F)c1 ; C=CC(=O)Nc1ccc(OCC2CCOCC2)c(F)c1 ; C=CC(=O)Nc1ccc(OCC2CCCCC2)c(F)c1 ; C=CC(=O)Nc1ccc(OCc2cccnc2)c(F)c1]

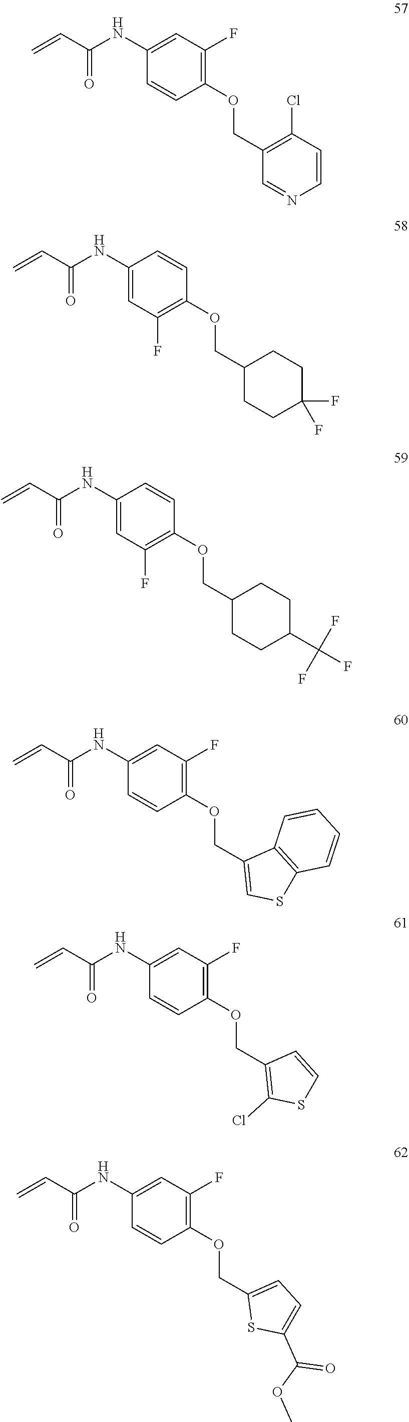 [SMILES: C=CC(=O)Nc1ccc(OCc2cnccc2Cl)c(F)c1 ; C=CC(=O)Nc1ccc(OCC2CCC(F)(F)CC2)c(F)c1 ; C=CC(=O)Nc1ccc(OCC2CCC(C(F)(F)F)CC2)c(F)c1 ; C=CC(=O)Nc1ccc(OCc2csc3ccccc23)c(F)c1 ; C=CC(=O)Nc1ccc(OCc2ccsc2Cl)c(F)c1 ; C=CC(=O)Nc1ccc(OCc2ccc(C(=O)OC)s2)c(F)c1]

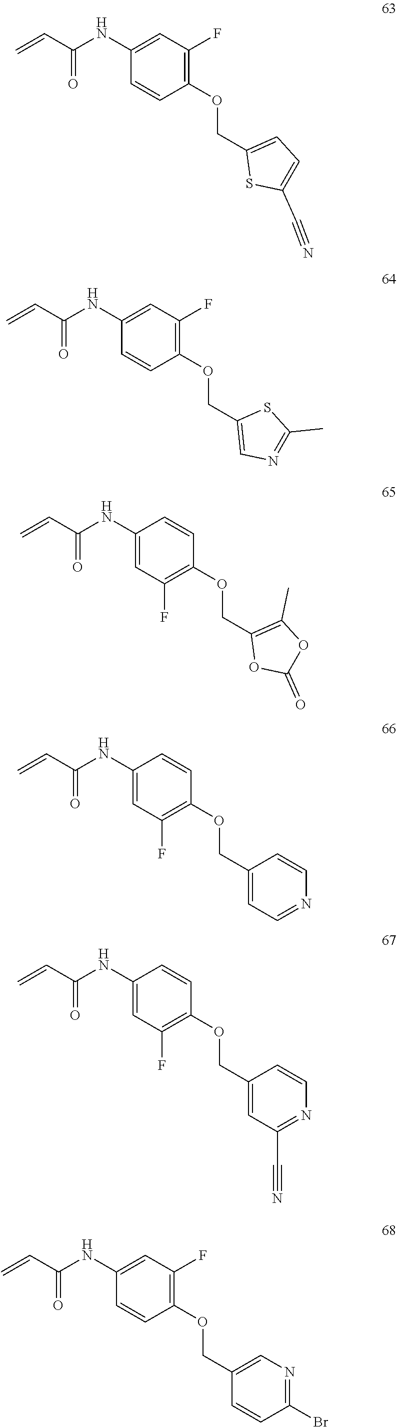

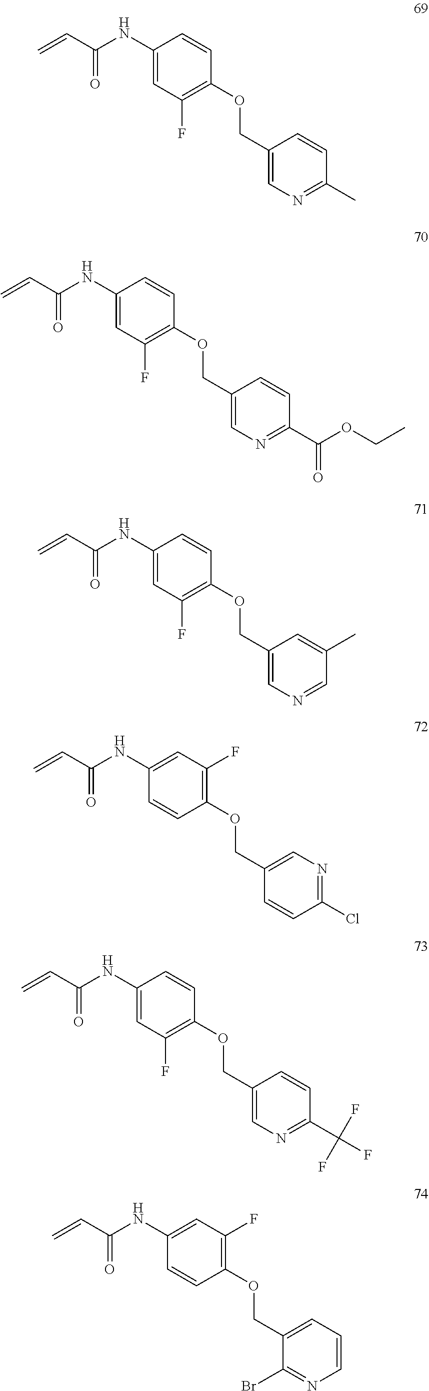

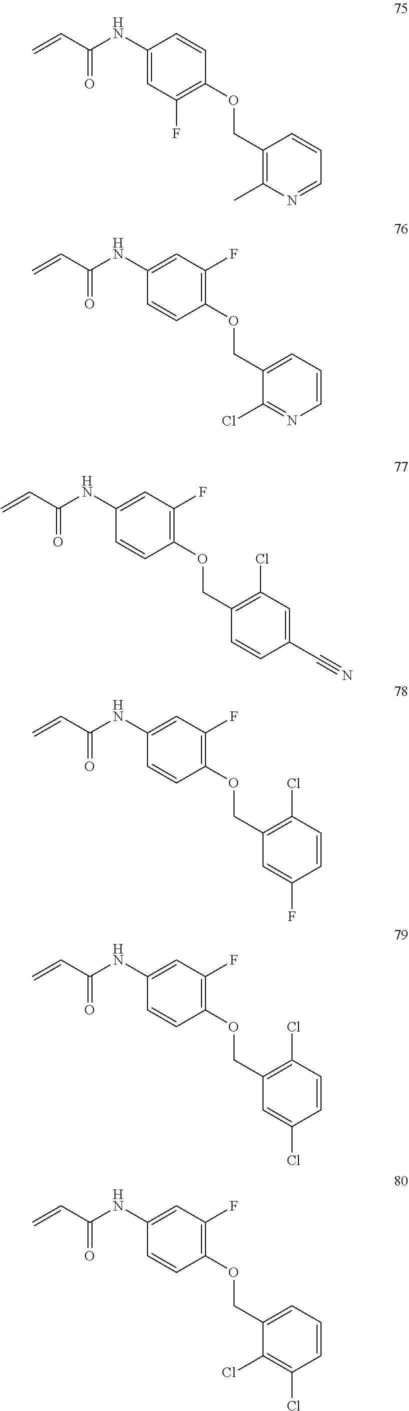 [SMILES: C=CC(=O)Nc1ccc(OCc2cccnc2C)c(F)c1 ; C=CC(=O)Nc1ccc(OCc2cccnc2Cl)c(F)c1 ; C=CC(=O)Nc1ccc(OCc2ccc(C#N)cc2Cl)c(F)c1 ; C=CC(=O)Nc1ccc(OCc2cc(F)ccc2Cl)c(F)c1 ; C=CC(=O)Nc1ccc(OCc2cc(Cl)ccc2Cl)c(F)c1 ; C=CC(=O)Nc1ccc(OCc2cccc(Cl)c2Cl)c(F)c1]

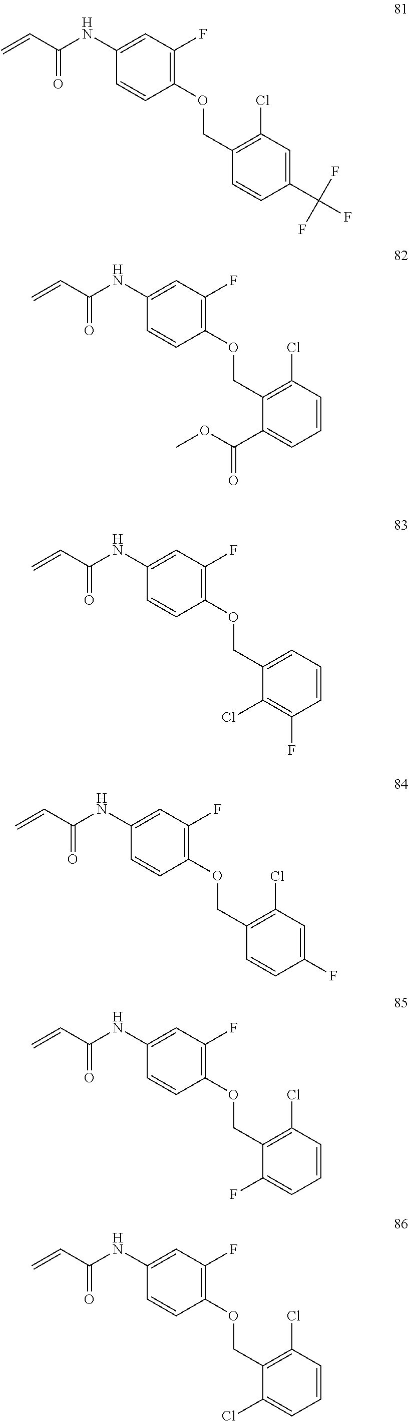 [SMILES: C=CC(=O)Nc1ccc(OCc2ccc(C(F)(F)F)cc2Cl)c(F)c1 ; C=CC(=O)Nc1ccc(OCc2c(Cl)cccc2C(=O)OC)c(F)c1 ; C=CC(=O)Nc1ccc(OCc2cccc(F)c2Cl)c(F)c1 ; C=CC(=O)Nc1ccc(OCc2ccc(F)cc2Cl)c(F)c1 ; C=CC(=O)Nc1ccc(OCc2c(F)cccc2Cl)c(F)c1 ; C=CC(=O)Nc1ccc(OCc2c(Cl)cccc2Cl)c(F)c1]

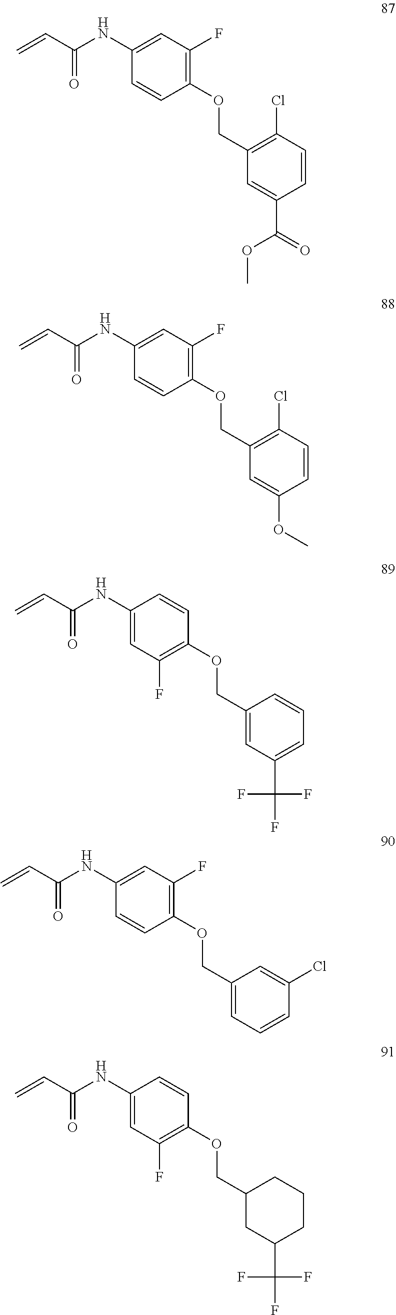

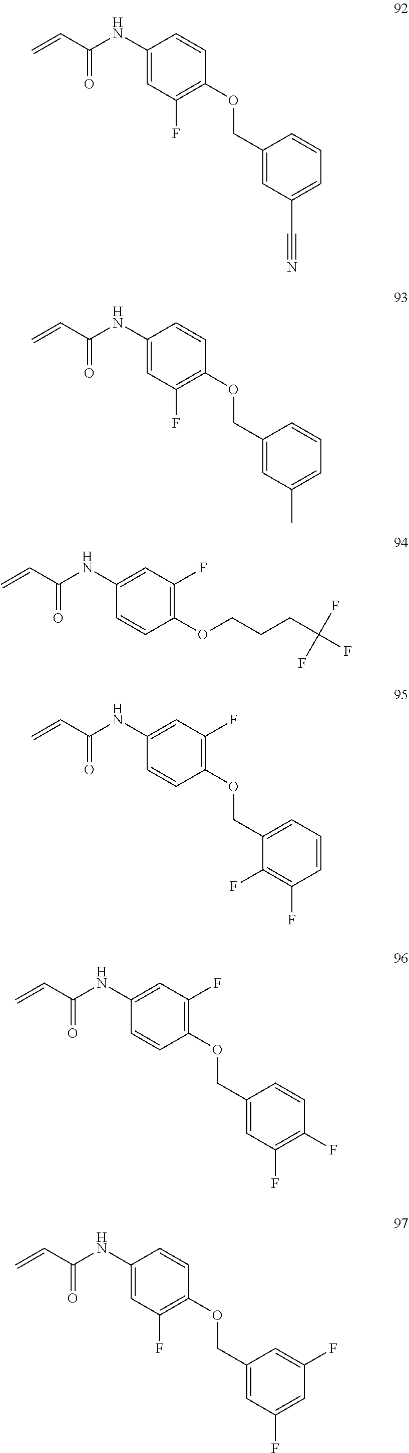 [SMILES: C=CC(=O)Nc1ccc(OCc2cccc(C#N)c2)c(F)c1 ; C=CC(=O)Nc1ccc(OCc2cccc(C)c2)c(F)c1 ; C=CC(=O)Nc1ccc(OCCCC(F)(F)F)c(F)c1 ; C=CC(=O)Nc1ccc(OCc2cccc(F)c2F)c(F)c1 ; C=CC(=O)Nc1ccc(OCc2ccc(F)c(F)c2)c(F)c1 ; C=CC(=O)Nc1ccc(OCc2cc(F)cc(F)c2)c(F)c1]

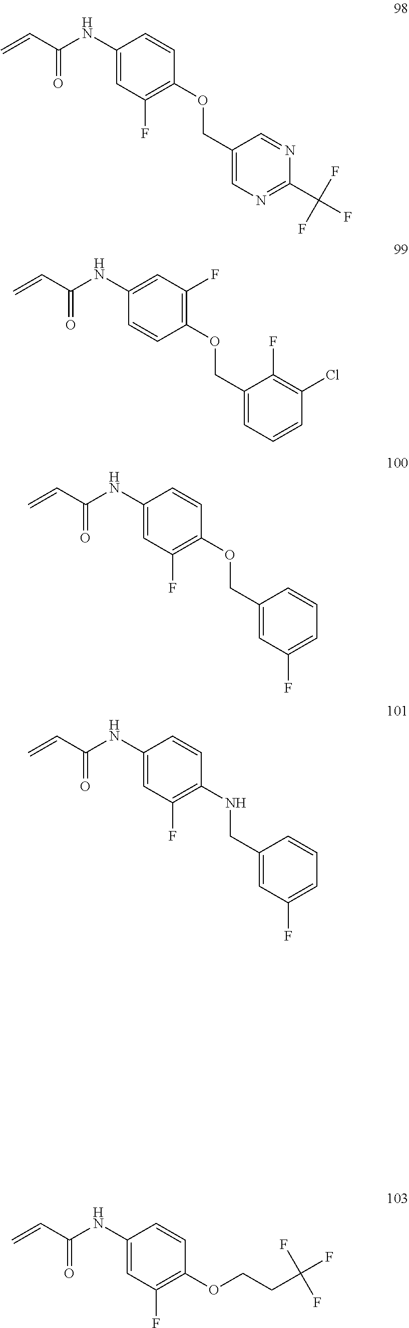 [SMILES: C=CC(=O)Nc1ccc(OCc2cnc(C(F)(F)F)nc2)c(F)c1 ; C=CC(=O)Nc1ccc(OCc2cccc(Cl)c2F)c(F)c1 ; C=CC(=O)Nc1ccc(OCc2cccc(F)c2)c(F)c1 ; C=CC(=O)Nc1ccc(NCc2cccc(F)c2)c(F)c1 ; C=CC(=O)Nc1ccc(OCCC(F)(F)F)c(F)c1]

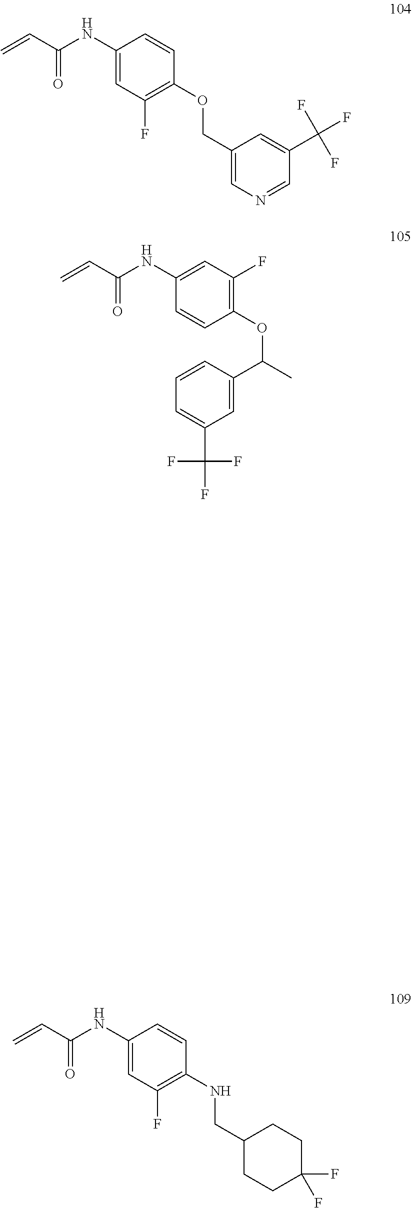

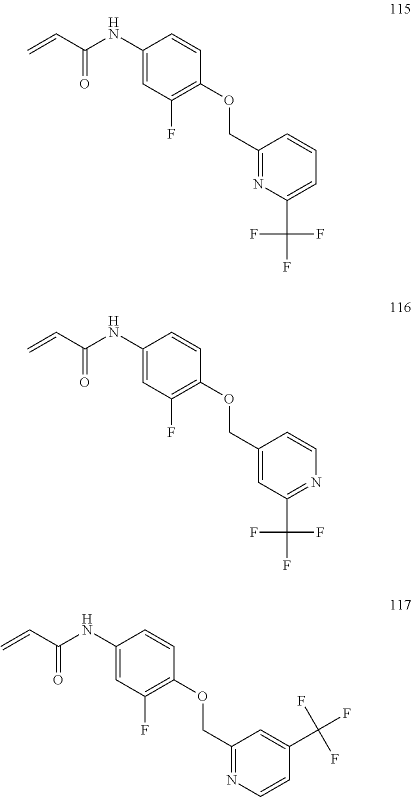 [SMILES: C=CC(=O)Nc1ccc(OCc2cccc(C(F)(F)F)n2)c(F)c1 ; C=CC(=O)Nc1ccc(OCc2ccnc(C(F)(F)F)c2)c(F)c1 ; C=CC(=O)Nc1ccc(OCc2cc(C(F)(F)F)ccn2)c(F)c1]

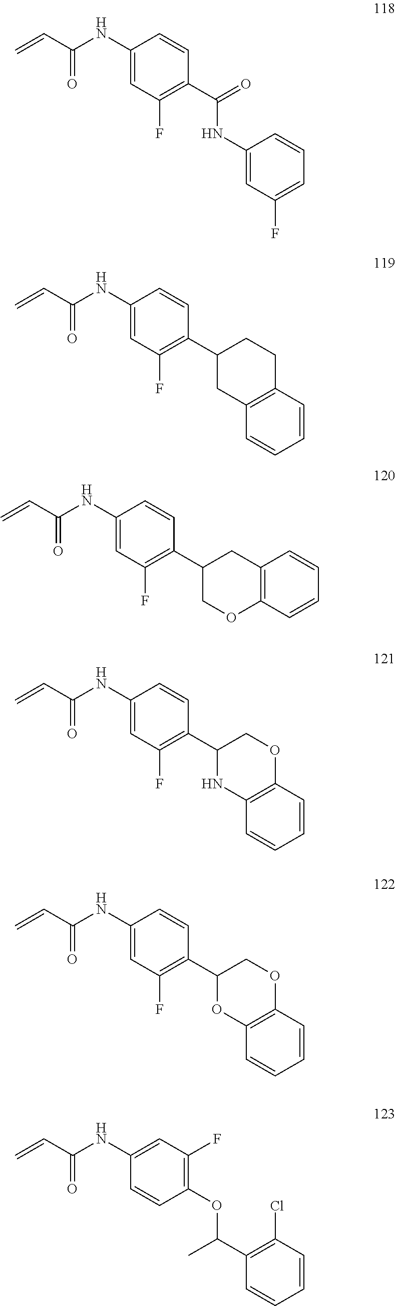 [SMILES: C=CC(=O)Nc1ccc(C(=O)Nc2cccc(F)c2)c(F)c1 ; C=CC(=O)Nc1ccc(C2CCc3ccccc3C2)c(F)c1 ; C=CC(=O)Nc1ccc(C2COc3ccccc3C2)c(F)c1 ; C=CC(=O)Nc1ccc(C2COc3ccccc3N2)c(F)c1 ; C=CC(=O)Nc1ccc(C2COc3ccccc3O2)c(F)c1 ; C=CC(=O)Nc1ccc(OC(C)c2ccccc2Cl)c(F)c1]

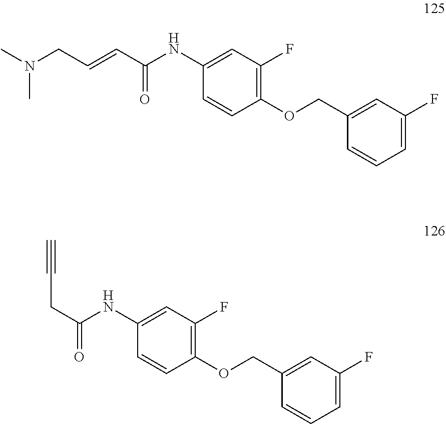 [SMILES: CN(C)C/C=C/C(=O)Nc1ccc(OCc2cccc(F)c2)c(F)c1 ; C#CCC(=O)Nc1ccc(OCc2cccc(F)c2)c(F)c1]

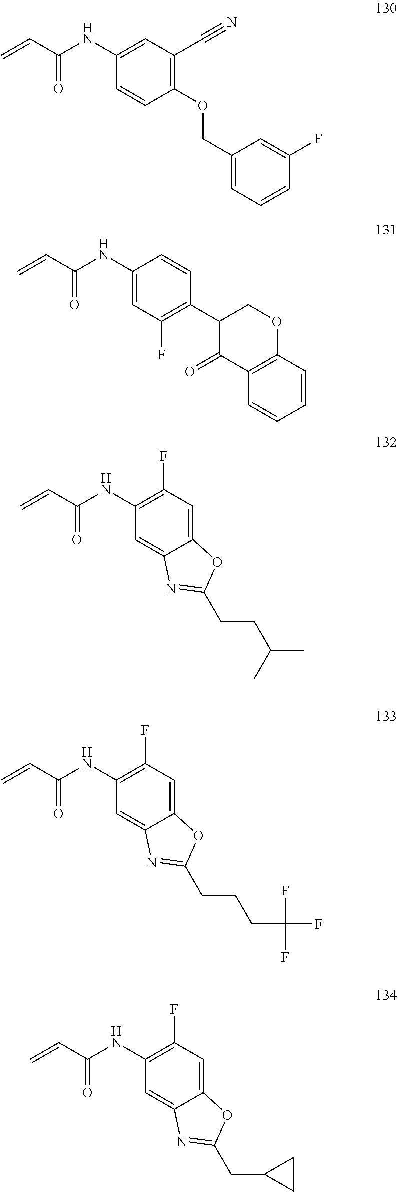

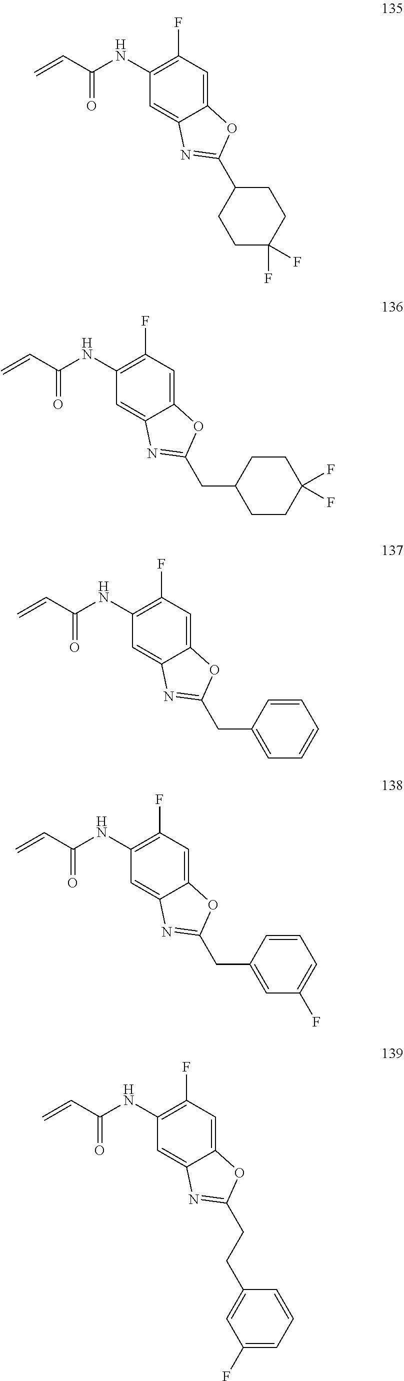 [SMILES: C=CC(=O)Nc1cc2nc(C3CCC(F)(F)CC3)oc2cc1F ; C=CC(=O)Nc1cc2nc(CC3CCC(F)(F)CC3)oc2cc1F ; C=CC(=O)Nc1cc2nc(Cc3ccccc3)oc2cc1F ; C=CC(=O)Nc1cc2nc(Cc3cccc(F)c3)oc2cc1F ; C=CC(=O)Nc1cc2nc(CCc3cccc(F)c3)oc2cc1F]

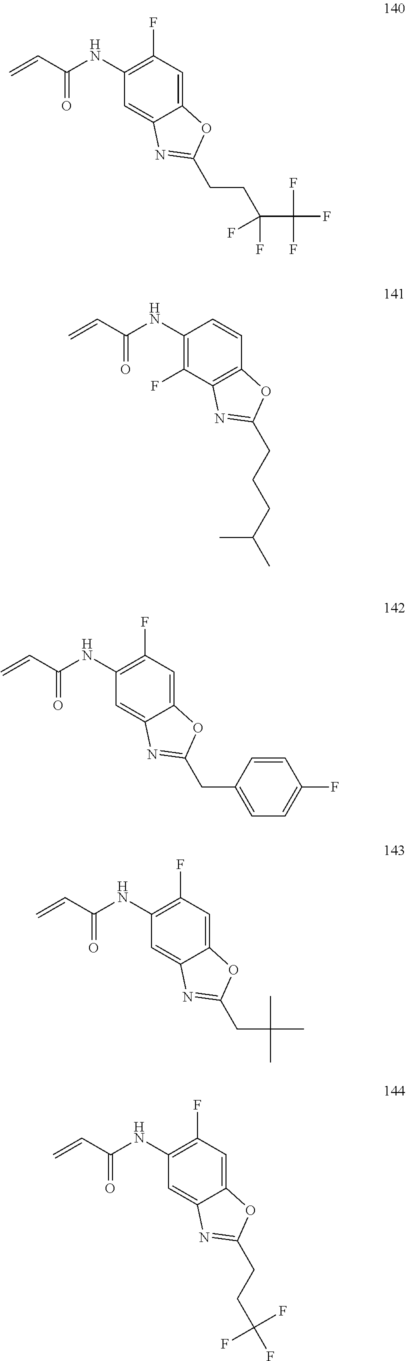

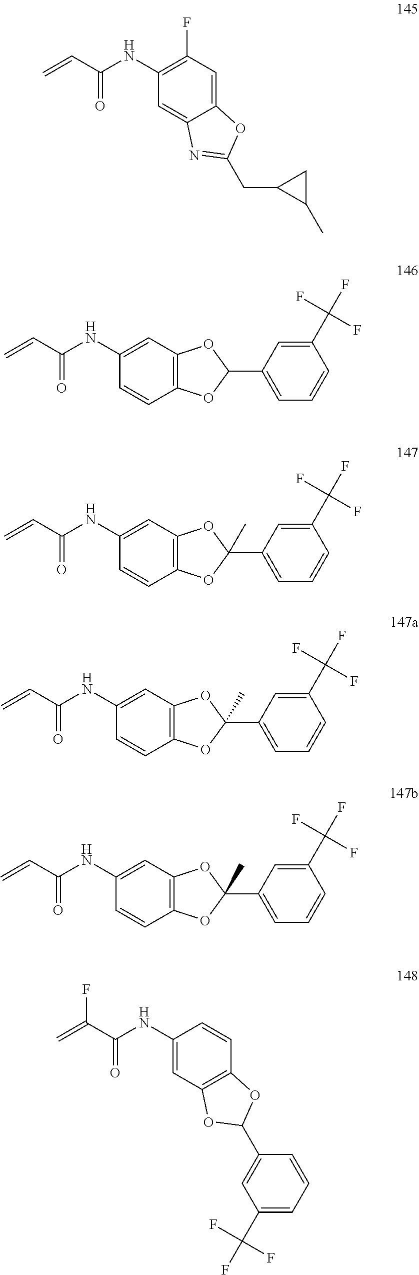

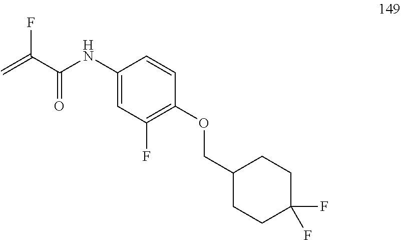 [SMILES: C=C(F)C(=O)Nc1ccc(OCC2CCC(F)(F)CC2)c(F)c1]

or a pharmaceutically acceptable salt thereof.

In some embodiments, a provided compound is selected from the group consisting of:

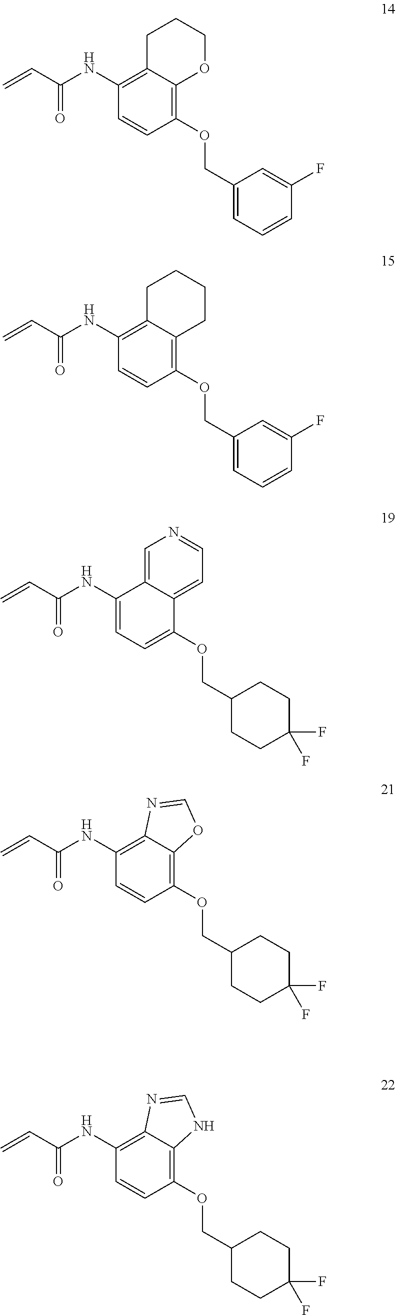 [SMILES: C=CC(=O)Nc1ccc(OCc2cccc(F)c2)c2c1CCCO2 ; C=CC(=O)Nc1ccc(OCc2cccc(F)c2)c2c1CCCC2 ; C=CC(=O)Nc1ccc(OCC2CCC(F)(F)CC2)c2ccncc12 ; C=CC(=O)Nc1ccc(OCC2CCC(F)(F)CC2)c2ocnc12 ; C=CC(=O)Nc1ccc(OCC2CCC(F)(F)CC2)c2[nH]cnc12]

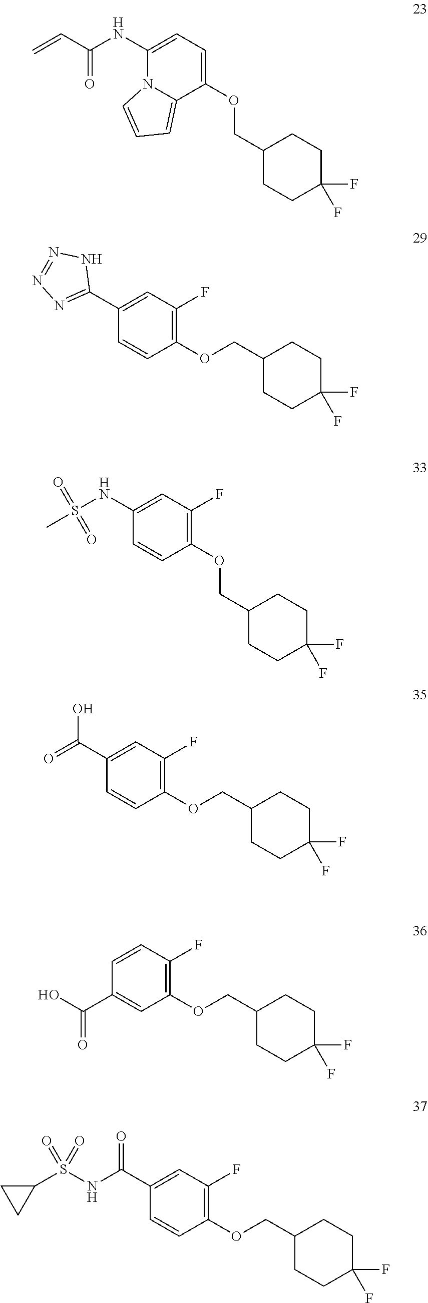 [SMILES: C=CC(=O)Nc1ccc(OCC2CCC(F)(F)CC2)c2cccn12 ; Fc1cc(-c2nnn[nH]2)ccc1OCC1CCC(F)(F)CC1 ; CS(=O)(=O)Nc1ccc(OCC2CCC(F)(F)CC2)c(F)c1 ; O=C(O)c1ccc(OCC2CCC(F)(F)CC2)c(F)c1 ; O=C(O)c1ccc(F)c(OCC2CCC(F)(F)CC2)c1 ; O=C(NS(=O)(=O)C1CC1)c1ccc(OCC2CCC(F)(F)CC2)c(F)c1]

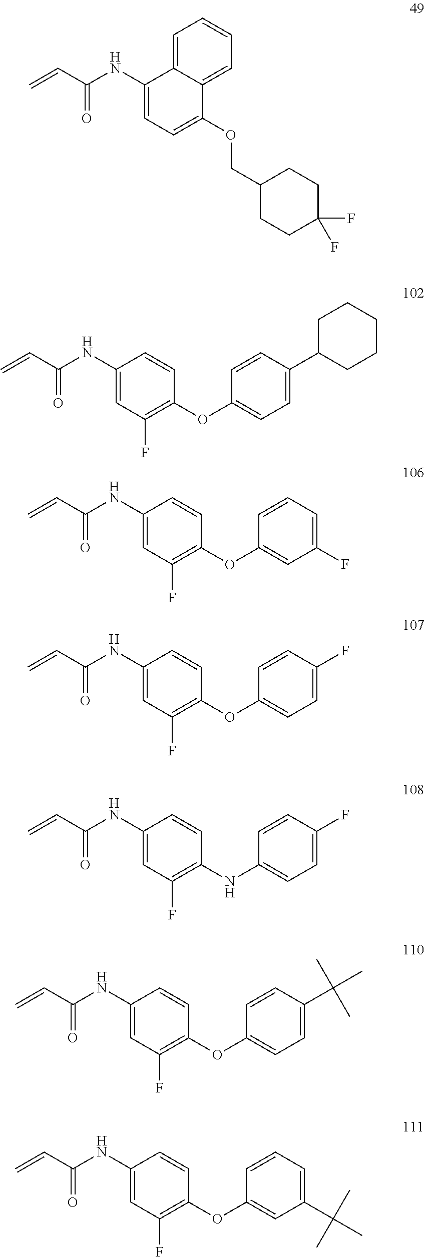

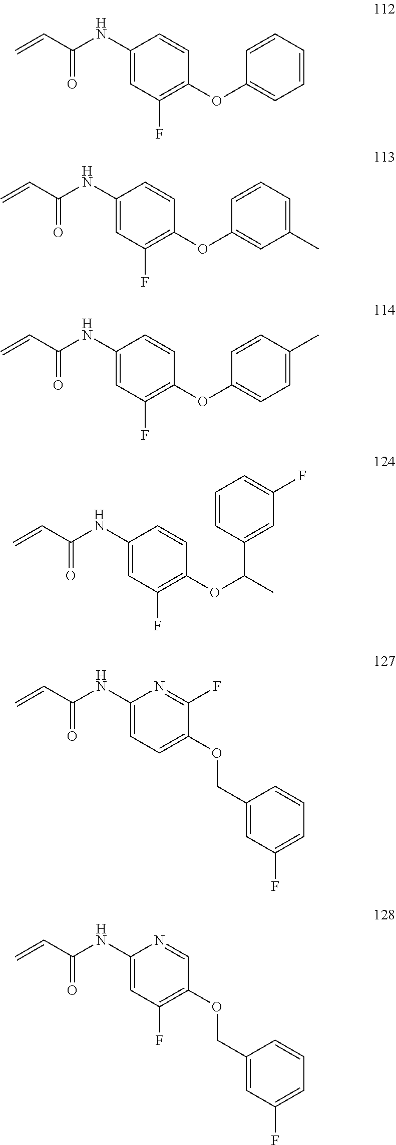

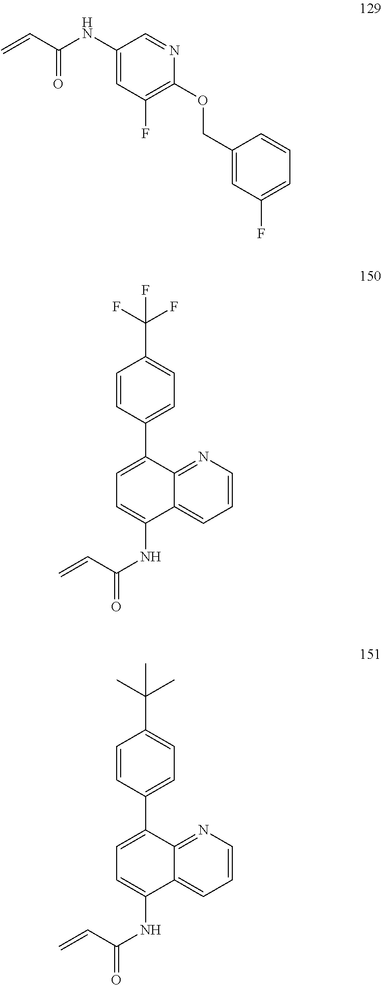 [SMILES: C=CC(=O)Nc1cnc(OCc2cccc(F)c2)c(F)c1 ; C=CC(=O)Nc1ccc(-c2ccc(C(F)(F)F)cc2)c2ncccc12 ; C=CC(=O)Nc1ccc(-c2ccc(C(C)(C)C)cc2)c2ncccc12]

or a pharmaceutically acceptable salt thereof.

### 4. Uses, Formulation, and Administration

Pharmaceutically Acceptable Compositions

According to another embodiment, the present disclosure provides a composition comprising a compound described herein or a pharmaceutically acceptable salt thereof and a pharmaceutically acceptable carrier, adjuvant, or vehicle. In certain embodiments, the amount of compound in compositions described herein is such that it is effective to measurably inhibit activity of a TEAD transcription factor, or a mutant thereof, in a biological sample or in a patient. In certain embodiments, a composition described herein is formulated for administration to a patient in need of such composition. In some embodiments, a composition described herein is formulated for oral administration to a patient.

Compounds and compositions, according to method of the present disclosure, are administered using any amount and any route of administration effective for treating or lessening the severity of a disorder provided herein (i.e., a TEAD-mediated disease or disorder). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Uses of Compounds and Pharmaceutically Acceptable Compositions

### The Hippo Signaling Pathway

The Hippo signaling pathway (also known as the Salvador/Warts/Hippo (SWH) pathway) is a key regulator of cell proliferation, death, and differentiation. In one aspect, a key function of the Hippo signaling pathway is the regulation of transcriptional co-activators Yes-associated protein (YAP; also known as YAP1 or YAP65) and its paralog, PDZ-binding motif (TAZ; also known as WWTR1). For example, the Hippo signaling pathway phosphorylates and inhibits YAP/TAZ activity by promoting their cytoplasmic retention and degradation, thereby inhibiting the growth promoting function regulated by YAP/TAZ. In an un-phosphorylated/dephosphorylated state, YAP, together with TAZ, are transported into the nucleus where they interact with the TEAD family of transcriptions factors to upregulate genes that promote proliferation and migration, and inhibit apoptosis. Without wishing to be bound by a particular theory, in some instances, unregulated upregulation of these genes involved in proliferation, migration, and anti-apoptosis leads to the development of a disease, disorder, or condition (e.g., cancer). In some embodiments, overexpression of YAP/TAZ is associated with a disease, disorder, or condition (e.g., cancer).

Additional key members of the Hippo signaling pathway include the serine/threonine kinases MST1/2 (homologues of Hippo/Hpo of Drosophilia), Lats1/2 (homologues of Warts/Wts) and their adaptor proteins Sav1 (homologue of Salvador/Sav) and Mob (MOBKL1A and MOBKL1B1; homologues of Mats), respectively. In general, MST1/2 kinases complex with scaffold protein Sav1, which in turn phosphorylate and activate Lats1/2 kinase. Lats1/2 is also activated by the scaffold protein Mob. The activated Lats1/2 then phosphorylates and inactivates YAP or its paralog TAZ. The phosphorylation of YAP/TAZ leads to their nuclear export, retention within the cytoplasm, and degradation by the ubiquitin proteasome system.

In some instances, Lats1/2 phosphorylates YAP at the [HXRXXS] (SEQ ID NO: 1) consensus motifs, wherein X denotes any amino acid residue. YAP comprises five [HXRXXS](SEQ ID NO: 1) consensus motifs. In some instances, Lats1/2 phosphorylates YAP at one or more of the consensus motifs. In some instances, Lats1/2 phosphorylates YAP at all five of the consensus motifs. In some instances, Lats1/2 phosphorylates YAP at S127. In one aspect, the phosphorylation of YAP S127 promotes 14-3-3 protein binding and results in cytoplasmic sequestration of YAP. Mutation of YAP at the S127 position thereby disrupts its interaction with 14-3-3 and subsequently promotes nuclear translocation.

Additional phosphorylation occurs at S381 of YAP. Phosphorylation of YAP at S381 and on the corresponding site in TAZ primes both proteins for further phosphorylation events by CK1δ/ε in the degradation motif, which then signals for interaction with the β-TRCP E3 ubiquitin ligase, leading to polyubiquitination and degradation of YAP.

In some instances, Lats1/2 phosphorylates TAZ at the [HXRXXS] (SEQ ID NO: 1) consensus motifs, wherein X denotes any amino acid residue. TAZ comprises four [HXRXXS](SEQ ID NO: 1) consensus motifs. In some instances, Lats1/2 phosphorylates TAZ at one or more of the consensus motifs. In some instances, Lats1/2 phosphorylates TAZ at all four of the consensus motifs. In some instances, Lats1/2 phosphorylates TAZ at S89. In one aspect, the phosphorylation of TAZ S89 promotes 14-3-3 protein binding and results in cytoplasmic sequestration of TAZ. Mutation of TAZ at the S89 position thereby disrupts its interaction with 14-3-3 and subsequently promotes nuclear translocation.

In some embodiments, phosphorylated YAP/TAZ accumulates in the cytoplasm, and undergoes SCF^{β-TRCP}-mediated ubiquitination and subsequent proteasomal degradation. In some instances, the Skp, Cullin, F-box containing complex (SCF complex) is a multi-protein E3 ubiquitin ligase complex that comprises a F-box family member protein (e.g., Cdc4), Skpl, a bridging protein, and RBX1, which contains a small RING Finger domain which interacts with E2-ubiquitin conjugating enzyme. In some cases, the F-box family comprises more than 40 members, in which exemplary members include F-box/WD repeat-containing protein IA (FBXWIA, β-TrCPl, Fbxwl, hsSlimb, plkappaBalpha-E3 receptor subunit) and S-phase kinase-associated proteins 2 (SKP2). In some embodiments, the SCF complex (e.g., SCF^{β-TRCP}) interacts with an E1 ubiquitin-activating enzyme and an E2 ubiquitin-conjugating enzyme to catalyze the transfer of ubiquitin to the YAP/TAZ substrate. Exemplary E1 ubiquitin-activating enzymes include those encoded by the following genes: UBA1, UBA2, UBA3, UBA5, UBA6, UBA7, ATG7, NAE1, and SAE1. Exemplary E2 ubiquitin-conjugating enzymes include those encoded by the following genes: UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE20, UBE2Q1, UBE2Q2, UBE2R1, UBE2R2, UBE2S, UBE2T, UBE2U, UBE2V1, UBE2V2, UBE2Z, ATG2, BIRC5, and UFCI. In some embodiments, ubiquitinated YAP/TAZ further undergoes the degradation process through the 26S proteasome.

In some embodiments, the Hippo signaling pathway is regulated upstream by several different families of regulators. For example, in some instances, the Hippo signaling pathway is regulated by the G-protein and its coupled receptors, the Crumbs complex, regulators upstream of the MST kinases, and the adherens junction.

In some embodiments, the Hippo signaling pathway is regulated by G protein-coupled receptors (GPCR) and G protein (also known as guanine nucleotide-binding proteins) family of proteins. G proteins are molecular switches that transmit extracellular stimuli into the cell through GPCRs. In some instances, there are two classes of G proteins: monomeric small GTPases and heterotrimeric G protein complexes. In one aspect, the heterotrimeric G protein complexes comprise alpha (G_{α}), beta (G_{β}), and gamma (G_{γ}) subunits. In other aspects, there are several classes of G_{α} subunits: e.g., G_{q/11}α, G_{12/3}α, G_{i/o}α (G inhibitory, G other), and Gₛα (stimulatory).

In some instances, G_{q/11}α, G_{12/13}α, Gᵢα, and Gₒα coupled GPCRs activate YAP/TAZ and promote nuclear translocation. In other instances, Gₛα coupled GPCRs suppress YAP/TAZ activity, leading to YAP/TAZ degradation. In some instances, G_{q/11}α, G_{12/13}α, Gᵢα, and Gₒα coupled GPCRs activate YAP/TAZ through inhibition of Lats1/2 activity. In other instances, Gₛα coupled GPCRs promotes or induces Lats1/2 activity, thereby leading to YAP/TAZ degradation. See Yu et al., Cell. (2012) 150, 780-791.

In some embodiments, the Hippo signaling pathway is regulated by the Crumbs (Crb) complex. The Crumbs complex is a key regulator of cell polarity and cell shape. In some instances, the Crumbs complex comprises transmembrane CRB proteins that assemble multi-protein complexes that function in cell polarity. In some instances, CRB complexes recruit members of the Angiomotin (AMOT) family of adaptor proteins that interact with the Hippo signaling pathway. In some instances, AMOT directly binds to YAP, promotes YAP phosphorylation, and inhibits its nuclear localization. Zhao et al., Genes & Dev. (2011) 25, 51-63.

In some instances, the Hippo signaling pathway is regulated by other components (e.g., TAO kinases and cell polarity kinase PAR-1) that modulate the activity of MST kinases. MST kinases monitor actin cytoskeletal integrity.

In some instances, the Hippo signaling pathway is regulated by molecules of the adherens junction. In some instances, E-Cadherin (E-cad) suppresses YAP nuclear localization and activity through regulating MST activity. In some embodiments, E-cad-associated protein a-catenin regulates YAP through sequestering YAP/14-3-3 complexes in the cytoplasm. In other instances, Ajuba protein family members interact with Lats1/2 kinase activity, thereby preventing inactivation of YAP/TAZ.

In some embodiments, additional proteins that interact with YAP/TAZ either directly or indirectly include, but are not limited to, Merlin, protocadherin Fat 1, MASK1/2, HIPK2, PTPN14, RASSF, PP2A, Salt-inducible kinases (SIKs), Scribble (SCRIB), the Scribble associated proteins Discs large (Dlg), KIBRA, PTPN14, NPHP3, LKB1, Ajuba, and ZO1/2.

### TEAD

In some embodiments, un-phosphorylated and/or dephosphorylated YAP/TAZ accumulates in the nucleus. In one aspect, once within the nucleus, YAP/TAZ interacts with the TEAD family of transcriptions factors (e.g., human TEAD1 (UniProt KB ID P28347-1 (SEQ IDNO: 1)); human TEAD2 (UniProtKB ID Q15562 (SEQ IDNO: 2)); human TEAD3 (UniProtKB ID Q99594 (SEQ ID NO: 3)); and human TEAD4 (UniProtKB ID Q15561 (SEQ ID NO: 4)) to activate genes that promote proliferation and migration, and inhibit apoptosis, such as, e.g., CTFG, Cyr61, and FGF1. In one aspect, without wishing to be bound by a particular theory, since TEAD is a downstream transcription factor of the Hippo pathway, inhibiting the function of TEAD is an attractive therapeutic strategy to reduce aberrant Hippo signaling and gene transcription.

TEAD1-4 are composed of a highly conserved TEA DNA binding domain and YAP binding domain, which is separated by a proline rich region. Despite the high homology shared between human TEAD1-4, the individual TEAD proteins are differentially expressed in a tissue- and development-dependent manner. For example, in some instances, TEAD1 is required for heart biogenesis, TEAD2 for embryonic development, TEAD4 for activating skeletal muscle genes, and TERAD3 has been shown to be specifically expressed in the placenta and several embryonic tissues during development. Holden et al. Cancers (2018) 10, 81, 1-15.

Proteomic and biochemical studies have shown that the TEAD family of transcription factors are palmitoylated at evolutionarily conserved cysteine residues. Three cysteine residues were found that are evolutionarily conserved and mutated to serine in human TEAD1 (C53S, C327S and C359S) to test whether the mutation affects TEAD1 palmitoylation. The C359S mutant showed the greatest loss of palmitoylation, and C327S and C53S also showed decreased palmitoylation. These results suggest that C359 plays a key role in TEAD1 palmitoylation. Furthermore, combination mutation of all three cysteine residues, C53/327/359S (3CS), completely ablated TEAD1 palmitoylation, indicating that these residues are involved in TEAD1 palmitoylation. In one aspect, it has been found that TEADs undergo PAT-independent autopalmitoylation, under physiological concentrations of palmitoyl-CoA. Furthermore, autopalmitoylation plays key roles in regulating TEAD-YAP association and their physiological functions in vitro and in vivo. Chan, et al. Nature Chem. Biol. (2016) 12, 282-289; Noland, et al. Structure, (2016) 24, 1-8; Gibault et al. J. Med. Chem. (2018) 61, 5057-5072. Therefore, in one aspect, palmitoylation of TEADs play important roles in regulating Hippo signaling pathway transcriptional complexes.

It will be understood that the term “YAP/TAZ” refers to YAP, TAZ, or both YAP and TAZ.

In some embodiments, compounds disclosed herein modulate the interaction between YAP/TAZ and TEAD. In some embodiments, compounds disclosed herein bind to TEAD and/or prevent interaction between YAP/TAZ and TEAD.

In some embodiments, compounds disclosed herein irreversibly bind to a TEAD transcription factor (e.g., TEAD1, TEAD2, TEAD3, or TEAD4). In some embodiments, compounds disclosed herein covalently bind to a TEAD transcription factor (e.g., TEAD1, TEAD2, TEAD3, or TEAD4). In some embodiments, compounds disclosed covalently inhibit the activity of a TEAD transcription factor (e.g., TEAD1, TEAD2, TEAD3, or TEAD4). In some embodiments, compounds disclosed irreversibly inhibit the activity of a TEAD transcription factor (e.g., TEAD1, TEAD2, TEAD3, or TEAD4).

In some embodiments, compounds disclosed herein bind to TEAD1 at C53. In some embodiments, compounds disclosed herein bind to TEAD1 at C327. In some embodiments, compounds disclosed herein bind to TEAD1 at C359. In some embodiments, compounds disclosed herein bind to TEAD1 at C405. In some embodiments, compounds disclosed herein bind to TEAD1 at C53 and C327. In some embodiments, compounds disclosed herein bind to TEAD1 at C53 and C359. In some embodiments, compounds disclosed herein bind to TEAD1 at C53 and C405. In some embodiments, compounds disclosed herein bind to TEAD1 at C327 and C359. In some embodiments, compounds disclosed herein bind to TEAD1 at C327 and C405. In some embodiments, compounds disclosed herein bind to TEAD1 at C359 and C405. In some embodiments, compounds disclosed herein bind to TEAD1 at C53, C327, and C359. In some embodiments, compounds disclosed herein bind to TEAD1 at C53, C327, and C405. In some embodiments, compounds disclosed herein bind to TEAD1 at C53, C359, and C405. In some embodiments, compounds disclosed herein bind to TEAD1 at C327, C359, and C405. In some embodiments, compounds disclosed herein bind to TEAD1 at C53, C327, C359, and C405.

In some embodiments, compounds disclosed herein bind to TEAD2 at C368. In some embodiments, compounds disclosed herein bind to TEAD2 at C380. In some embodiments, compounds disclosed herein bind to TEAD2 at C368 and C380

In some embodiments, compounds disclosed herein bind to TEAD3 at C368. In some embodiments, compounds disclosed herein bind to TEAD3 at C371. In some embodiments, compounds disclosed herein bind to TEAD3 at C368 and C368.

In some embodiments, compounds disclosed herein bind to TEAD4 at C367.

In some embodiments, compounds disclosed herein bind to a TEAD transcription factor (e.g., TEAD1, TEAD2, TEAD3, or TEAD4) and disrupt or inhibit the interaction between YAP/TAZ and the TEAD transcription factor. In some embodiments, compounds disclosed herein bind to TEAD1 and disrupt or inhibit the interaction between YAP/TAZ and TEAD1. In some embodiments, compounds disclosed herein bind to TEAD2 and disrupt or inhibit the interaction between YAP/TAZ and TEAD2. In some embodiments, compounds disclosed herein bind to TEAD3 and disrupt or inhibit the interaction between YAP/TAZ and TEAD3. In some embodiments, compounds disclosed herein bind to TEAD4 and disrupt or inhibit the interaction between YAP/TAZ and TEAD4.

In some embodiments, compounds disclosed herein bind to TEAD1 at C53, and disrupt or inhibit the interaction between YAP/TAZ and TEAD1. In some embodiments, compounds disclosed herein bind to TEAD1 at C327, and disrupt or inhibit the interaction between YAP/TAZ and TEAD1. In some embodiments, compounds disclosed herein bind to TEAD1 at C359, and disrupt or inhibit the interaction between YAP/TAZ and TEAD1. In some embodiments, compounds disclosed herein bind to TEAD1 at C405, and disrupt or inhibit the interaction between YAP/TAZ and TEAD1. In some embodiments, compounds disclosed herein bind to TEAD1 at C53 and C327, and disrupt or inhibit the interaction between YAP/TAZ and TEAD1. In some embodiments, compounds disclosed herein bind to TEAD1 at C53 and C359, and disrupt or inhibit the interaction between YAP/TAZ and TEAD1. In some embodiments, compounds disclosed herein bind to TEAD1 at C53 and C405, and disrupt or inhibit the interaction between YAP/TAZ and TEAD1. In some embodiments, compounds disclosed herein bind to TEAD1 at C327 and C359, and disrupt or inhibit the interaction between YAP/TAZ and TEAD1. In some embodiments, compounds disclosed herein bind to TEAD1 at C327 and C405, and disrupt or inhibit the interaction between YAP/TAZ and TEAD1. In some embodiments, compounds disclosed herein bind to TEAD1 at C359 and C405, and disrupt or inhibit the interaction between YAP/TAZ and TEAD1. In some embodiments, compounds disclosed herein bind to TEAD1 at C53, C327, and C359, and disrupt or inhibit the interaction between YAP/TAZ and TEAD1. In some embodiments, compounds disclosed herein bind to TEAD1 at C53, C327, and C405, and disrupt or inhibit the interaction between YAP/TAZ and TEAD1. In some embodiments, compounds disclosed herein bind to TEAD1 at C53, C359, and C405, and disrupt or inhibit the interaction between YAP/TAZ and TEAD1. In some embodiments, compounds disclosed herein bind to TEAD1 at C327, C359, and C405, and disrupt or inhibit the interaction between YAP/TAZ and TEAD1. In some embodiments, compounds disclosed herein bind to TEAD1 at C53, C327, C359, and C405, and disrupt or inhibit the interaction between YAP/TAZ and TEAD1.

In some embodiments, compounds disclosed herein bind to TEAD2 at C368, and disrupt or inhibit the interaction between YAP/TAZ and TEAD2. In some embodiments, compounds disclosed herein bind to TEAD2 at C380, and disrupt or inhibit the interaction between YAP/TAZ and TEAD2. In some embodiments, compounds disclosed herein bind to TEAD2 at C368 and C380, and disrupt or inhibit the interaction between YAP/TAZ and TEAD2.

In some embodiments, compounds disclosed herein bind to TEAD3 at C368, and disrupt or inhibit the interaction between YAP/TAZ and TEAD3. In some embodiments, compounds disclosed herein bind to TEAD3 at C371, and disrupt or inhibit the interaction between YAP/TAZ and TEAD3. In some embodiments, compounds disclosed herein bind to TEAD3 at C368 and C368, and disrupt or inhibit the interaction between YAP/TAZ and TEAD3.

In some embodiments, compounds disclosed herein bind to TEAD4 at C367, and disrupt or inhibit the interaction between YAP/TAZ and TEAD4.

In some embodiments, compounds disclosed herein bind to a TEAD transcription factor (e.g., TEAD1, TEAD2, TEAD3, or TEAD4) and prevent TEAD transcription palmitoylation. In some embodiments, compounds disclosed herein bind to TEAD1 and prevent TEAD1 palmitoylation. In some embodiments, compounds disclosed herein bind to TEAD1 and prevent TEAD1 palmitoylation at C53. In some embodiments, compounds disclosed herein bind to TEAD1 and prevent TEAD1 palmitoylation at C327. In some embodiments, compounds disclosed herein bind to TEAD1 and prevent TEAD1 palmitoylation at C359. In some embodiments, compounds disclosed herein bind to TEAD1 and prevent TEAD1 palmitoylation at C405. In some embodiments, compounds disclosed herein bind to TEAD1 and prevent TEAD1 palmitoylation at C53 and C327. In some embodiments, compounds disclosed herein bind to TEAD1 and prevent TEAD1 palmitoylation at C53 and C359. In some embodiments, compounds disclosed herein bind to TEAD1 and prevent TEAD1 palmitoylation at C53 and C459. In some embodiments, compounds disclosed herein bind to TEAD1 and prevent TEAD1 palmitoylation at C327 and C359. In some embodiments, compounds disclosed herein bind to TEAD1 and prevent TEAD1 palmitoylation at C327 and C405. In some embodiments, compounds disclosed herein bind to TEAD1 and prevent TEAD1 palmitoylation at C359 and C405. In some embodiments, compounds disclosed herein bind to TEAD1 and prevent TEAD1 palmitoylation at C53, C327, and C359. In some embodiments, compounds disclosed herein bind to TEAD1 and prevent TEAD1 palmitoylation at C53, C327, and C405. In some embodiments, compounds disclosed herein bind to TEAD1 and prevent TEAD1 palmitoylation at C327, C359, and C405. In some embodiments, compounds disclosed herein bind to TEAD1 and prevent TEAD1 palmitoylation at C53, C327, C359, and C405.

In some embodiments, compounds disclosed herein bind to TEAD2 and prevent TEAD2 palmitoylation at C368. In some embodiments, compounds disclosed herein bind to TEAD2 and prevent TEAD2 palmitoylation at C380. In some embodiments, compounds disclosed herein bind to TEAD2 and prevent TEAD2 palmitoylation at C368 and C380.

In some embodiments, compounds disclosed herein bind to TEAD3 and prevent TEAD3 palmitoylation at C368. In some embodiments, compounds disclosed herein bind to TEAD3 and prevent TEAD3 palmitoylation at C371. In some embodiments, compounds disclosed herein bind to TEAD3 and prevent TEAD3 palmitoylation at C368 and C371.

In some embodiments, compounds disclosed herein bind to TEAD4 and prevent TEAD4 palmitoylation at C367.

In some embodiments, compounds disclosed herein bind to a TEAD transcription factor (e.g., TEAD1, TEAD2, TEAD3, or TEAD4), prevent TEAD transcription factor palmitoylation, and disrupt or inhibit the interaction between YAP/TAZ and the TEAD transcription factor. In some embodiments, compounds disclosed herein bind to TEAD1, prevent TEAD1 palmitoylation, and disrupt or inhibit the interaction between YAP/TAZ and TEAD1. In some embodiments, compounds disclosed herein bind to TEAD1, prevent TEAD1 palmitoylation at C53, and disrupt or inhibit the interaction between YAP/TAZ and TEAD1. In some embodiments, compounds disclosed herein bind to TEAD1, prevent TEAD1 palmitoylation at C327, and disrupt or inhibit the interaction between YAP/TAZ and TEAD1. In some embodiments, compounds disclosed herein bind to TEAD1, prevent TEAD1 palmitoylation at C359, and disrupt or inhibit the interaction between YAP/TAZ and TEAD1. In some embodiments, compounds disclosed herein bind to TEAD1, prevent TEAD1 palmitoylation at C405, and disrupt or inhibit the interaction between YAP/TAZ and TEAD1. In some embodiments, compounds disclosed herein bind to TEAD1, prevent TEAD1 palmitoylation at C53 and C327, and disrupt or inhibit the interaction between YAP/TAZ and TEAD1. In some embodiments, compounds disclosed herein bind to TEAD1, prevent TEAD1 palmitoylation at C53 and C359, and disrupt or inhibit the interaction between YAP/TAZ and TEAD1. In some embodiments, compounds disclosed herein bind to TEAD1, prevent TEAD1 palmitoylation at C53 and C459, and disrupt or inhibit the interaction between YAP/TAZ and TEAD1. In some embodiments, compounds disclosed herein bind to TEAD1, prevent TEAD1 palmitoylation at C327 and C359, and disrupt or inhibit the interaction between YAP/TAZ and TEAD1. In some embodiments, compounds disclosed herein bind to TEAD1, prevent TEAD1 palmitoylation at C327 and C405, and disrupt or inhibit the interaction between YAP/TAZ and TEAD1. In some embodiments, compounds disclosed herein bind to TEAD1, prevent TEAD1 palmitoylation at C359 and C405, and disrupt or inhibit the interaction between YAP/TAZ and TEAD1. In some embodiments, compounds disclosed herein bind to TEAD1, prevent TEAD1 palmitoylation at C53, C327, and C359, and disrupt or inhibit the interaction between YAP/TAZ and TEAD1. In some embodiments, compounds disclosed herein bind to TEAD1, prevent TEAD1 palmitoylation at C53, C327, and C405, and disrupt or inhibit the interaction between YAP/TAZ and TEAD1. In some embodiments, compounds disclosed herein bind to TEAD1, prevent TEAD1 palmitoylation at C327, C359, and C405, and disrupt or inhibit the interaction between YAP/TAZ and TEAD1. In some embodiments, compounds disclosed herein bind to TEAD1, prevent TEAD1 palmitoylation at C53, C327, C359, and C405, and disrupt or inhibit the interaction between YAP/TAZ and TEAD1.

In some embodiments, compounds disclosed herein bind to TEAD2, prevent TEAD2 palmitoylation at C368, and disrupt or inhibit the interaction between YAP/TAZ and TEAD1. In some embodiments, compounds disclosed herein bind to TEAD2, prevent TEAD2 palmitoylation at C380, and disrupt or inhibit the interaction between YAP/TAZ and TEAD1. In some embodiments, compounds disclosed herein bind to TEAD2, prevent TEAD2 palmitoylation at C368 and C380, and disrupt or inhibit the interaction between YAP/TAZ and TEAD1.

In some embodiments, compounds disclosed herein bind to TEAD3, prevent TEAD3 palmitoylation at C368, and disrupt or inhibit the interaction between YAP/TAZ and TEAD1. In some embodiments, compounds disclosed herein bind to TEAD3, prevent TEAD3 palmitoylation at C371, and disrupt or inhibit the interaction between YAP/TAZ and TEAD1. In some embodiments, compounds disclosed herein bind to TEAD3, prevent TEAD3 palmitoylation at C368 and C371, and disrupt or inhibit the interaction between YAP/TAZ and TEAD1.

In some embodiments, compounds disclosed herein bind to TEAD4, prevent TEAD4 palmitoylation at C367, and disrupt or inhibit the interaction between YAP/TAZ and TEAD1.

The activity of a compound described herein as an inhibitor of TEAD (e.g., TEAD1, TEAD2, TEAD3, and/or TEAD4), or a variant or mutant thereof, can be assayed in vitro, in vivo, or in a cell line. In vitro assays include assays that determine inhibition of TEAD (e.g., TEAD1, TEAD2, TEAD3, and/or TEAD4), or a variant or mutant thereof. Alternate in vitro assays quantitate the ability of the inhibitor to bind to TEAD (e.g., TEAD1, TEAD2, TEAD3, and/or TEAD4) or a variant or mutant thereof. Detailed conditions for assaying a compound described herein as an inhibitor of TEAD (e.g., TEAD1, TEAD2, TEAD3, and/or TEAD4), or a variant or mutant thereof, are set forth in the Examples below. See, for example, Example 2.

The provided compounds are inhibitors of TEAD (e.g., TEAD1, TEAD2, TEAD3, and/or TEAD4) and are therefore useful for treating one or more disorders associated with activity of TEAD (e.g., TEAD1, TEAD2, TEAD3, and/or TEAD4). Thus, in some aspects and embodiments, the present disclosure provides a method for treating a TEAD-mediated disease, disorder, or condition comprising the step of administering to a patient in need thereof a compound of the present disclosure, or pharmaceutically acceptable composition thereof.

In some embodiments, the present disclosure provides a method of inhibiting TEAD (e.g., TEAD1, TEAD2, TEAD3, and/or TEAD4) comprising contacting a cell with a compound provided herein.

As used herein, the term “TEAD-mediated” disorders or conditions as used herein means any disease or other deleterious condition in which TEAD (e.g., TEAD1, TEAD2, TEAD3, and/or TEAD4), or a mutant thereof, is known to play a role. Accordingly, another embodiment of the present disclosure relates to treating or lessening the severity of one or more diseases in which TEAD (e.g., TEAD1, TEAD2, TEAD3, and/or TEAD4), or a mutant thereof, is known to play a role.

In some embodiments, the present disclosure provides methods of treating, reducing the severity of, delaying the onset of, or inhibiting the progress of a disease or disorder, or one or more symptoms thereof of a disease or disorder characterized by or associated with increased TEAD (e.g., TEAD1, TEAD2, TEAD3, and/or TEAD4) expression and/or increased TEAD (e.g., TEAD1, TEAD2, TEAD3, and/or TEAD4) activity, comprising the step of administering to a patient in need thereof a therapeutically effective a compound of the present disclosure, or pharmaceutically acceptable composition thereof. In some embodiments, the present disclosure provides methods of treating, reducing the severity of, delaying the onset of, or inhibiting the progress of a disease or disorder, or one or more symptoms thereof of a disease or disorder in which inhibition or antagonizing of TEAD (e.g., TEAD1, TEAD2, TEAD3, and/or TEAD4) activity is beneficial comprising the step of administering to a patient in need thereof a compound described herein, or pharmaceutically acceptable composition thereof. In some aspects and embodiments, provided herein are methods of treating, reducing the severity of, delaying the onset of, or inhibiting the progress of a disease or disorder, or one or more symptoms thereof of a disease or disorder in which inhibition or antagonizing of the Hippo signaling pathway is beneficial comprising the step of administering to a patient in need thereof a therapeutically effective compound of the present disclosure, or pharmaceutically acceptable composition thereof.

In some aspects and embodiments, the present disclosure provides a method for treating one or more disorders, diseases, and/or conditions wherein the disorder, disease, or condition includes, but is not limited to, a cellular proliferative disorder, comprising administering to a patient in need thereof, a TEAD inhibitor compound as described herein, or a pharmaceutical salt or composition thereof. In some embodiments, a cellular proliferative disorder is cancer. In some embodiments, the cancer is characterized by increased TEAD (e.g., TEAD1, TEAD2, TEAD3, and/or TEAD4) expression and/or increased TEAD (e.g., TEAD1, TEAD2, TEAD3, and/or TEAD4) activity.

In some embodiments, provided methods include the co-administration of a provided compound and at least one mitogen-activated protein kinase (MAPK) inhibitor. In some embodiments, provided methods include the co-administration of a provided compound and at least one inhibitor of the RAS/MAPK pathway. In some embodiments, provided methods include the co-administration of a provided compound and at least one epidermal growth factor receptor (EGFR) inhibitor. In some embodiments, an inhibitor of the RAS/MAPK pathway is a KRAS inhibitor, RAF inhibitor (e.g., a BRAF monomer or RAF dimer inhibitor), a MEK inhibitor, an ERK inhibitor, an EGFR inhibitor, or a MAPK inhibitor, or a combination thereof. In some embodiments, an inhibitor of the RAS/MAPK pathway is an EGFR inhibitor or a MAPK inhibitor, or a combination thereof. Examples of EGFR inhibitors, MAPK inhibitors, and/or RAS/MAPK pathway inhibitors are disclosed in Moore A. R. Rosenberg, S. C., McCormock, F. et al. Nat. Rev. Discov. (2020) and include, e.g., Osimertinib (TAGRISSO®, AstraZeneca), sotorasib (AMG 510 from Amgen), MRTX849 (from Mirati Therapeutics), JNJ-74699157/ARS-3248 (from J&J Wellspring Biosciences), LY3499446 (from Eli Lilly), GDCBI 1701963 (from Boehringer Ingelheim), mRNA-5671 (from Moderna Therapeutics), G12D inhibitor (from Mirati Therapeutics), RAS(ON) inhibitors (from Revolution Medicines), BBP-454 (from BridgeBio Pharma), SP600125, PLX4032, GW5074, AZD6244, PD98059, simvastatin, alisertib, teriflunomide, NSC95397, PD325901, PD98059, lovastatin, sorafenib (NEXAVAR®, Bayer Labs), vermurafenib (ZELBORAF®, Hoffman La Roche Inc.), dabrafenib (TAFLINAR®, Novartis Pharmaceuticals Corporation), selumetinib (KOSELUGO™, AstraZeneca Pharmaceuticals LP), trametinib (MEKINIST®, Novartis Pharmaceuticals Corporation), uxliertinib, silimarin, sirolimus (RAPAMUNE®, PV Prism CV), lapatinib (TYKERB®/TYVERB®, GlaxoSmithKline), crizotinib (XALKORI®, PF Prism CV), taselisib (Roche), PF-0491502, pF502, enterolactone, PLX4720, PD0325901, PD184352, SC-514, alisterib (MLN8237), SB415286, PLX4720, obtaoclax (GX15-070), pimasterib, venetoclax (ABT-199/VENCLEXTA®/VENCLYXTO®), eprenetapopt (APR-246), gemcitabine (GEMZAR®), birinapant (TL32711), pexmetinib (ARRY-614), afuresertib, ralimetinib (LY2228820, Eli Lilly), cobimetinib (COTELLIC®, Exelixis/Genentech), prexasertib (LY2606368), erlotinib (TARCEVA®, OSI Pharmaceuticals), bevacizumab (AVASTIN®, Genentech), belvarafenib (Hanmi Pharm./Genentech, Inc.) and binimetinib (MEKTOVI®, Array Biopharma Inc.).

As used herein, the terms “increased expression” and/or “increased activity” of a substance, such as TEAD, in a sample or cancer or patient refers to an increase in the amount of the substance, such as TEAD, of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount of the substance, such as TEAD, in a control sample or control samples, such as an individual or group of individuals who are not suffering from the disease or disorder (e.g., cancer) or an internal control, as determined by techniques known in the art. A subject can also be determined to have an “increased expression” or “increased activity” of TEAD if the expression and/or activity of TEAD is increased by one standard deviation, two standard deviations, three standard deviations, four standard deviations, five standard deviations, or more, relative to the mean (average) or median amount of TEAD in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

In some embodiments, the present disclosure provides a method for treating or lessening the severity of a cancer including, without limitation, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer. In some embodiments, the cancer is or has metastasized. In some embodiments, the cancer is relapsed or refractory cancer. In some embodiments, the cancer is a relapsed or refractory solid tumor. In some embodiments, the cancer is a relapsed or refractory hematological malignancy. In some embodiments, the cancer is or has been characterized by or has been established to have one or more genetic alterations in the Hippo pathway (e.g., NF2, LATS1/2, AMOTL2, SAV1, TAOK1-3, etc.). In some embodiments, the cancer is or has been characterized by or has been established to have one or more genetic alterations that affect or alter the stability of Hippo pathway components (e.g., BAP1, SOCS6, etc.). In some embodiments, the cancer is or has been characterized by or has been established to have a YAP/TAZ gene translocation (e.g., WWTR1(TAZ)-CAMTA1, YAP1-TFE3, etc.). In some embodiments, the cancer is selected from those disclosed in WO 2019/113236, the entire contents of which are hereby incorporated by reference.

In some embodiments, the cancer is mediated by activation YAP/TAZ. In some embodiments of the methods and uses described herein, the cancer is mediated by modulation of the interaction of YAP/TAZ with TEAD (e.g., TEADI, TEAD2, TEAD3, and/or TEAD4). In some embodiments, the cancer is characterized by or associated with increased TEAD (e.g., TEAD1, TEAD2, TEAD3, and/or TEAD4) expression and/or increased TEAD (e.g., TEAD1, TEAD2, TEAD3, and/or TEAD4) activity. In some embodiments, the cancer being treated is a cancer in which YAP/TAZ is localized in the nucleus of the cancer cells. In some embodiments, the cancer being treated is or has been characterized or established by one or more YAP/TAZ genetic amplifications or mutations.

In some embodiments, the cancer is characterized by a mutant Gα-protein. In some embodiments, a mutant Gα-protein is G₁₂, G₁₃, G_{q}, G₁₁, Gᵢ, Gₒ, or Gₛ. In some embodiments, a mutant Gα-protein is G₁₂. In some embodiments, a mutant Gα-protein is G₁₃. In some embodiments, a mutant Gα-protein is G_{q}. In some embodiments, a mutant Gα-protein is G₁₁. In some embodiments, a mutant Gα-protein is Gᵢ. In some embodiments, a mutant Gα-protein is Gₒ. In some embodiments, a mutant Gα-protein is Gₛ.

In some embodiments, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma. In some embodiments, the cancer is leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell-involved cancers (including cervical squamous cell carcinoma, lung squamous cell carcinoma, esphageal squamous cell carcinoma, head and neck squamous cell carcinoma, bladder urothelial carcinoma), basal cell carcinoma, adenocarcinoma, sweat gland carcinoma, sebaceous gland carcmoma, papillary carcmoma, papillary adenocarcinomas, cystadenocarcinoma, medullary carcinoma, bronchogenic carcinoma, renal cell carcinoma, hepatoma, bile duct carcinoma (i.e. cholangiocarcinoma), choriocarcinoma, seminoma, embryonal carcinoma, Wilm's tumor, cervical cancer, endometrial/uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, epithelioid hemangioendothelioma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g., Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

In some embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcmoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcmoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcmoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is being treated adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells; as well as virus-associated tumors in gastric cancer, nasopharyngeal carcinoma, cervical cancer, vaginal cancer, vulvar cancer, squamous cell carcinoma of the head and neck, and Merkel cell carcinoma.

In some embodiments, the cancer is melanoma cancer. In some embodiments, the cancer is breast cancer. In some embodiments, the cancer is lung cancer. In some embodiments, the cancer is small cell lung cancer (SCLC). In some embodiments, the cancer is non-small cell lung cancer (NSCLC).

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1. Synthesis of Exemplary Compounds

### Example 1.1. Synthesis of N-(3-fluoro-4-(3-fluorophenethyl)phenyl)acrylamide (1)

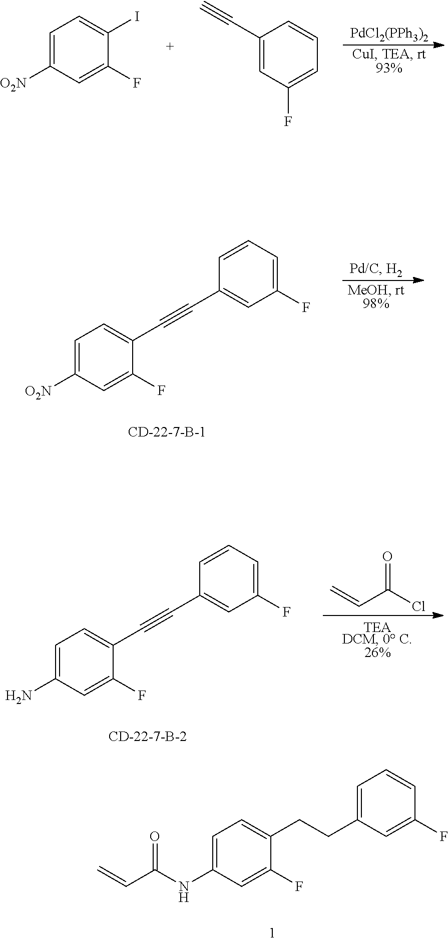

2-fluoro-1-((3-fluorophenyl)ethynyl)-4-nitrobenzene (CD-22-7-B-1). To a stirred solution of 2-fluoro-1-iodo-4-nitrobenzene (0.900 g, 3.37 mmol), 1-ethynyl-3-fluorobenzene (1.21 g, 10.11 mmol), and CuI (0.064 g, 0.337 mmol) in triethylamine (10 mL) was added bis(triphenylphosphine)palladium(II) chloride (236.5 mg, 0.337 mmol) at room temperature under nitrogen atmosphere; the mixture was degassed with nitrogen three times. The resulting mixture was stirred for 5 h. The reaction mixture was poured into water (20 mL) and extracted with ethyl acetate (25 mL×2). Combined organic extracts were washed with brine (25 mL), dried over anhydrous Na₂SO₄, and concentrated in vacuo. The resulting crude was purified by silica gel column chromatography using a 0.5% ethyl acetate/hexane gradient to afford 2-fluoro-1-((3-fluorophenyl)ethynyl)-4-nitrobenzene (CD-22-7-B-1) (0.816 g, 93%) as a colorless oil.

3-fluoro-4-(3-fluorophenethyl)aniline (CD-22-7-B-2). To a solution of 2-fluoro-1-((3-fluorophenyl)ethynyl)-4-nitrobenzene (CD-22-7-B-1) (0.816 g, 3.13 mmol) in methanol (30 mL) was added palladium on carbon (10%, 123 mg) at room temperature, and the resulting mixture was stirred at room temperature under H₂ atmosphere (H₂ balloon) for 4 hours. The catalyst was removed through filtration and the filtrate cake was washed with MeOH (10 mL×2). The combined filtrates were concentrated under reduced pressure to give crude product, which was purified by silica gel flash column chromatography using a 1% methanol/dichloromethane gradient to afford 3-fluoro-4-(3-fluorophenethyl)aniline (CD-22-7-B-2) (0.725 g, 98%) as brown oil. MS [MH]⁺ 234.1.

N-(3-fluoro-4-(3-fluorophenethyl)phenyl)acrylamide (1). To a stirred solution of 3-fluoro-4-(3-fluorophenethyl)aniline (CD-22-7-B-2) (0.050 g, 0.21 mmol) and triethylamine (0.065 g, 0.64 mmol) in dichloromethane (1 mL) was added acryloyl chloride (0.045 g, 0.49 mmol) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford N-(3-fluoro-4-(3-fluorophenethyl)phenyl)acrylamide (1) (0.016 g, 26%) as a white solid. ¹H NMR (400 MHz, CD₃OD) δ 7.45-7.40 (m, 1H), 7.15-7.08 (m, 2H), 7.00 (t, J=8.4 Hz, 1H), 6.86 (d, J=7.6 Hz, 1H), 6.83-6.74 (m, 2H), 6.34-6.22 (m, 2H), 5.67-5.62 (m, 1H), 2.80 (s, 4H). MS [MH]⁺ 288.2.

### Example 1.2. Synthesis of N-(3-cyclopropyl-4-((3-fluorobenzyl)oxy)phenyl)acrylamide (2)

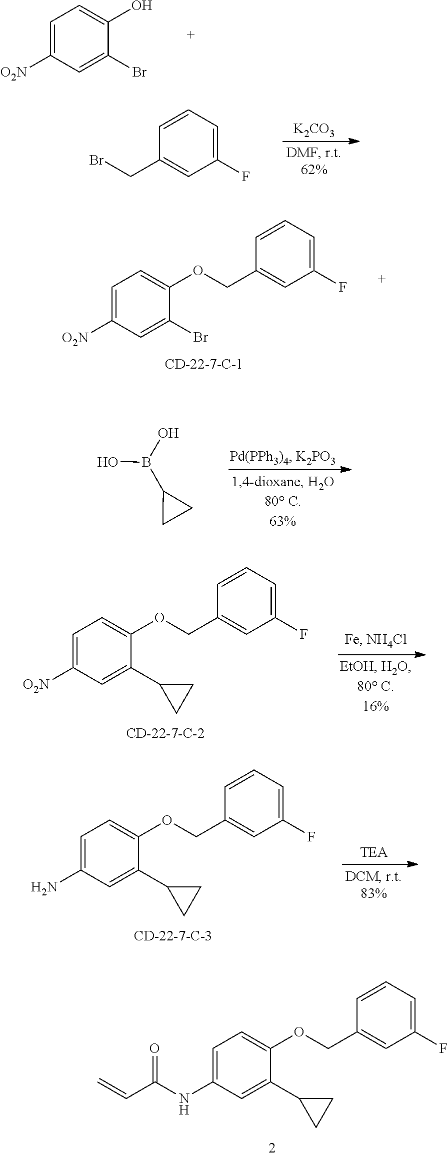

2-bromo-1-((3-fluorobenzyl)oxy)-4-nitrobenzene (CD-22-7-C-1). To a solution of 2-bromo-4-nitrophenol (2.000 g, 9.21 mmol) and 1-(bromomethyl)-3-fluorobenzene (1.900 g, 10.10 mmol) was added potassium carbonate (2.800 g, 18.40 mmol) in DMF (20 mL) at room temperature under nitrogen, and the resulting mixture was stirred at room temperature for 6 h. The reaction mixture was poured into water (50 mL) and extracted with ethyl acetate (35 mL×2). Combined organic extracts were washed with brine (25 mL), dried over anhydrous Na₂SO₄, and concentrated in vacuo. The resulting crude was purified by silica gel column chromatography using a 5% ethyl acetate/hexane gradient to afford 2-bromo-1-((3-fluorobenzyl)oxy)-4-nitrobenzene (CD-22-7-C-1) (1.800 g, 62%) as a yellow solid.

2-cyclopropyl-1-((3-fluorobenzyl)oxy)-4-nitrobenzene (CD-22-7-C-2). To a mixture of 2-bromo-1-((3-fluorobenzyl)oxy)-4-nitrobenzene (CD-22-7-C-1) (0.250 g, 0.76 mmol), cyclopropylboronic acid (0.073 g, 0.83 mmol), and potassium phosphate (0.325 g, 1.52 mmol) in 1,4-dioxane (4 mL)-water (1 mL), was added tetrakis(triphenylphosphine)palladium (0.088 g, 0.07 mmol) at room temperature under nitrogen atmosphere; the mixture was degassed with nitrogen three times. The resulting mixture was refluxed for 2 h. The resulting mixture was filtered and the filtrate was concentrated in vacuo. The resulting crude was purified by silica gel column chromatography using a 2.5% ethyl acetate/hexane gradient to afford 2-cyclopropyl-1-((3-fluorobenzyl)oxy)-4-nitrobenzene (CD-22-7-C-2) (0.140 g, 63%) as a white solid.

3-cyclopropyl-4-((3-fluorobenzyl)oxy)aniline (CD-22-7-C-3). A mixture of 2-cyclopropyl-1-((3-fluorobenzyl)oxy)-4-nitrobenzene (CD-22-7-C-2) (0.090 g, 0.31 mmol) and ammonium chloride (0.083 g, 1.5 mmol) in ethanol (9 mL)-water (3 mL) was added iron powder (0.048 g, 1.5 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 10%-15% ethyl acetate/hexane gradient to afford 3-cyclopropyl-4-((3-fluorobenzyl)oxy)aniline (CD-22-7-C-3) (0.020 g, 16%) as a brown oil. MS [MH]⁺ 258.3.

N-(3-cyclopropyl-4-((3-fluorobenzyl)oxy)phenyl)acrylamide (2). To a solution of 3-cyclopropyl-4-((3-fluorobenzyl)oxy)aniline (CD-22-7-C-3) (20 mg, 0.07 mmol) and triethylamine (0.024 g, 0.21 mmol) in dichloromethane (2 mL) was added acryloyl chloride (0.011 g, 0.11 mmol) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(3-cyclopropyl-4-((3-fluorobenzyl)oxy)phenyl)acrylamide (2) (0.025 g, 83%) as a white solid. ¹H NMR (400 MHz, DMSO-d6): δ 9.92 (s, 1H), 7.48-7.42 (m, 2H), 7.33-7.28 (m, 2H), 7.28-7.12 (m, 2H), 6.97 (d, J=8.8 Hz, 1H), 6.37-6.33 (m, 1H), 6.24-6.20 (m, 1H), 5.73-5.70 (m, 1H), 5.14 (s, 2H), 2.21-2.14 (m, 1H), 0.96-0.91 (m, 2H), 0.60-0.55 (m, 2H). MS [MH]⁺ 312.3.

The following compounds were prepared in a manner analogous to the procedures described above for N-(3-cyclopropyl-4-((3-fluorobenzyl)oxy)phenyl)acrylamide (2):

N-(3-cyclohexyl-4-((3-fluorobenzyl)oxy)phenyl)acrylamide (3) (0.022 g, 37%) as a white solid. ¹H NMR (400 MHz, DMSO-d6): δ 9.96 (s, 1H), 7.75-7.42 (m, 3H), 7.28-7.23 (m, 2H), 7.27-7.12 (m, 1H), 6.96 (d, J=8.8 Hz, 1H), 6.39-6.34 (m, 1H), 6.25-6.22 (m, 1H), 5.74-5.70 (m, 1H), 5.13 (s, 2H), 2.98-2.89 (m, 1H), 1.79-1.70 (m, 5H), 1.38-1.32 (m, 5H). [MH]⁺ 354.2.

N-(2-cyclopropyl-4-((3-fluorobenzyl)oxy)phenyl)acrylamide (4) (0.070 g, 89%) as a gray solid. ¹HNMR (400 MHz, CDCl₃): δ 8.08 (d, J=8.8 Hz, 1H), 7.62 (br, 1H), 7.37-7.31 (m, 1H), 7.21-7.11 (m, 2H), 7.04-6.97 (m, 1H), 6.87-6.80 (m, 1H), 6.80-6.72 (m, 1H), 6.47-6.37 (m, 1H), 6.35-6.24 (m, 1H), 5.76 (d, J=10.0 Hz, 1H), 5.03 (s, 2H), 1.85-1.75 (m, 1H), 1.03-0.95 (m, 2H), 0.71-0.62 (m, 2H). [MH]⁺ 312.4.

### Example 1.3. Synthesis of N-(2-(3-fluorophenyl)chroman-6-yl)acrylamide (5)

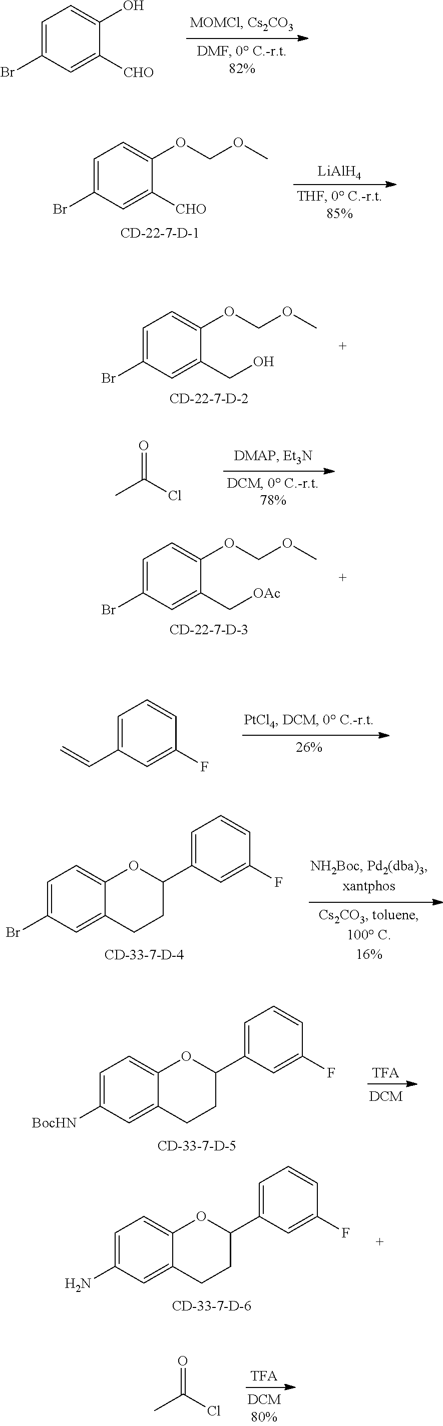

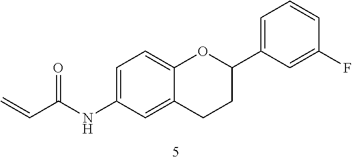 [SMILES: C=CC(=O)Nc1ccc2c(c1)CCC(c1cccc(F)c1)O2]

5-bromo-2-(methoxymethoxy)benzaldehyde (CD-22-7-D-1). To a mixture of 5-bromo-2-hydroxybenzaldehyde (0.500 g, 2.50 mmol) and chloromethyl methyl ether (0.300 g, 3.80 mmol) in DMF (5 mL) was added cesium carbonate (1.200 g, 3.80 mmol) at room temperature under nitrogen, and the resulting mixture was stirred at room temperature for 4 h. The reaction mixture was poured into water (20 mL) and extracted with ethyl acetate (15 mL×2). Combined organic extracts were washed with brine (25 mL), dried over anhydrous Na₂SO₄, and concentrated in vacuo. The resulting crude was purified by silica gel column chromatography using a 5% ethyl acetate/hexane gradient to afford 5-bromo-2-(methoxymethoxy)benzaldehyde (CD-22-7-D-1) (0.500 g, 82%) as a colorless oil.

(5-bromo-2-(methoxymethoxy)phenyl)methanol (CD-22-7-D-2). To a solution of 5-bromo-2-(methoxymethoxy)benzaldehyde (CD-22-7-D-1) (0.500 g, 2.00 mmol) in THF (10 mL) was added lithium aluminum hydride (0.150 g, 4.00 mmol) at 0° C. under nitrogen, and the resulting mixture was stirred at room temperature for 2 h. The reaction mixture was quenched with sodium sulfate decahydrate (1 g), and the resulting mixture was stirred at room temperature for 1 h. The solid was removed through filtration, the filtrate cake was washed with THF (10 mL×2), and the combined filtrates were concentrated under reduced pressure to give crude product which was purified by silica gel flash column chromatography using a 10% ethyl acetate/hexane gradient to afford 5-bromo-2-(methoxymethoxy)phenyl)methanol (CD-22-7-D-2) (0.420 g, 85%) as a colorless oil.

5-bromo-2-(methoxymethoxy)benzyl acetate (CD-22-7-D-3). To a solution of (5-bromo-2-(methoxymethoxy)phenyl)methanol (CD-22-7-D-2) (0.100 g, 0.40 mmol), 4-dimethylaminopyridine (0.075 g, 0.60 mmol), and triethylamine (0.061 g, 1.50 mmol) in dichloromethane (4 mL) was added acetyl chloride (48 mg, 1.5 mmol) dropwise at 0° C. under nitrogen, and the resulting mixture was stirred at 0° C. for 30 min. The organic layer was collected, and the aqueous layer was extracted with DCM (10 mL×2). Combined organic extracts were washed with brine (10 mL), dried over anhydrous Na₂SO₄, and concentrated in vacuo. The resulting crude was purified by silica gel column chromatography using a 10% ethyl acetate/hexane gradient to afford 5-bromo-2-(methoxymethoxy)benzyl acetate (CD-22-7-D-3) (0.100 g, 78%) as a colorless oil.

6-bromo-2-(3-fluorophenyl)chromane (CD-22-7-D-4). To a mixture of 5-bromo-2-(methoxymethoxy)benzyl acetate (CD-22-7-D-3) (0.180 g, 0.60 mmol) and 1-fluoro-3-vinylbenzene (0.316 g, 3.00 mmol) in DCM (5 mL) was added platinum tetrachloride (34 mg) at room temperature and stirred for 2 hours. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 10% ethyl acetate/hexane gradient to afford 6-bromo-2-(3-fluorophenyl)chromane (CD-22-7-D-4) (0.100 g, 26%) as a colorless oil.

tert-butyl (2-(3-fluorophenyl)chroman-6-yl)carbamate (CD-22-7-D-5). To a solution of 6-bromo-2-(3-fluorophenyl)chromane (CD-22-7-D-4) (0.090 g, 0.30 mmol), tert-butyl carbamate (0.038 g, 0.33 mmol), xantphos (0.030 g, 0.06 mmol), and cesium carbonate (0.140 g, 0.45 mmol) in toluene (5 mL) was added tris(dibenzylideneacetone)dipalladium (0.027 g, 0.03 mmol) at room temperature under nitrogen atmosphere; the mixture was degassed with nitrogen three times. The resulting mixture was refluxed for 2 h. The resulting mixture was filtered and the filtrate was concentrated in vacuo. The resulting crude was purified by silica gel column chromatography using a 10% ethyl acetate/hexane gradient to afford tert-butyl (2-(3-fluorophenyl)chroman-6-yl)carbamate (CD-22-7-D-5) (0.018 mg, 16%) as a colorless oil.

2-(3-fluorophenyl)chroman-6-amine (CD-22-7-D-6). A mixture of tert-butyl (2-(3-fluorophenyl)chroman-6-yl)carbamate (CD-22-7-D-5) (0.018 g, 0.05 mmol) and trifluoroacetic acid (0.5 mL) in dichloromethane (0.5 mL) was stirred at room temperature for 1 h. The reaction mixture was concentrated to afford 2-(3-fluorophenyl)chroman-6-amine (CD-22-7-D-6) (0.012 g, 80%) as a yellow solid which was used in next step without further purification.

N-(2-(3-fluorophenyl)chroman-6-yl)acrylamide (5). To a solution of 2-(3-fluorophenyl)chroman-6-amine (CD-22-7-D-6) (0.012 g, 0.07 mmol) and triethylamine (0.007 g, 0.10 mmol) in dichloromethane (2 mL) was added acryloyl chloride (0.006 g, 0.10 mmol) at 0° C., and the resulting mixture was stirred at room temperature for 1.5 h. The reaction mixture was poured into water (5 mL) and extracted with DCM (5 mL). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford N-(2-(3-fluorophenyl)chroman-6-yl)acrylamide (5) (0.012 g, 80% of 2 steps) as a colorless solid. ¹H NMR (400 MHz, CDCl₃): δ 7.51 (s, 1H), 7.37-7.31 (m, 1H), 7.18-7.13 (m, 3H), 7.03-6.99 (m, 1H), 6.87 (d, J=8.8 Hz, 1H), 6.46-6.42 (m, 1H), 6.25-6.22 (m, 1H), 5.74 (d, J=10.4 Hz, 1H), 5.05 (d, J=8.4 Hz, 1H), 2.98-2.93 (m, 1H), 2.82-2.75 (m, 1H), 2.24-2.19 (m, 1H), 2.07-1.91 (m, 2H). MS: [MH]⁺ 298.1.

### Example 1.4. Synthesis of N-(4-((3-fluorobenzyl)oxy)-3-methoxyphenyl)acrylamide (6)

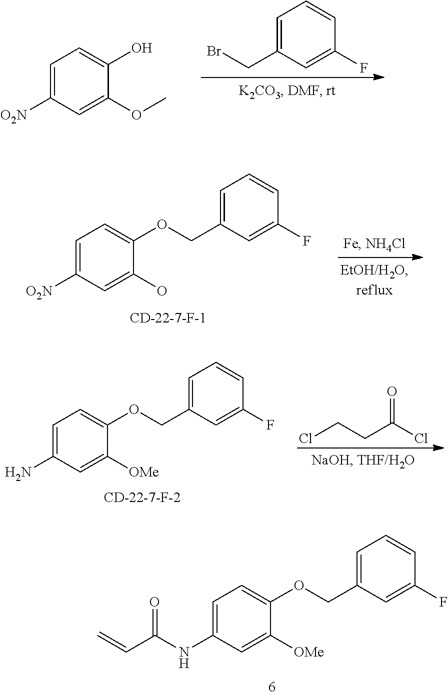

1-((3-fluorobenzyl)oxy)-2-methoxy-4-nitrobenzene (CD-22-7-F-1). To a solution of 2-methoxy-4-nitrophenol (0.500 g, 2.96 mmol) and 1-(bromomethyl)-3-fluorobenzene (0.586 g, 3.10 mmol) in N,N-dimethylformamide (10 mL) was added potassium carbonate (0.612 g, 4.44 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 1-((3-fluorobenzyl)oxy)-2-methoxy-4-nitrobenzene (CD-22-7-F-1) (0.800 g, yield 97%) as a light yellow solid.

4-((3-fluorobenzyl)oxy)-3-methoxyaniline (CD-22-7-F-2). To a mixture of 1-((3-fluorobenzyl)oxy)-2-methoxy-4-nitrobenzene (CD-22-7-F-1) (0.800 g, 2.89 mmol) and ammonium chloride (0.772 g, 14.44 mmol) in ethanol (12 mL)-water (4 mL) was stirred iron power (0.809 g, 14.44 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 20%-30% ethyl acetate/hexane gradient to afford 4-((3-fluorobenzyl)oxy)-3-methoxyaniline (CD-22-7-F-2) (0.640 g, 89%) as a yellow solid. MS: [MH]⁺ 248.4.

N-(4-((3-fluorobenzyl)oxy)-3-methoxyphenyl)acrylamide (6). To a stirred solution of 4-((3-fluorobenzyl)oxy)-3-methoxyaniline (CD-22-7-F-2) (100 mg, 0.40 mmol) and sodium hydroxide (80.8 mg, 2.02 mmol) in tetrahydrofuran-water (6 mL-2 mL) was added 3-chloropropanoyl chloride (77.0 mg, 0.61 mmol) dropwise at 0° C., and the resulting mixture was stirred at room temperature overnight. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford N-(4-((3-fluorobenzyl)oxy)-3-methoxyphenyl)acrylamide (6) (0.100 g, 82%) as an off-white solid. ¹HNMR (400 MHz, CD₃OD): δ 7.44 (d, J=2.4 Hz, 1H), 7.40-7.33 (m, 1H), 7.26-7.17 (m, 2H), 7.08-6.99 (m, 2H), 6.93 (d, J=8.4 Hz, 1H), 6.44-6.30 (m, 2H), 5.77-5.72 (m, 1H), 5.09 (s, 2H), 3.87 (s, 3H). [MH]⁺ 302.1.

The following compound was prepared in a manner analogous to the procedures described above for N-(4-((3-fluorobenzyl)oxy)-3-methoxyphenyl)acrylamide (6):

N-(4-((3-fluorobenzyl)oxy)-2-methoxyphenyl)acrylamide (7) (0.026 g, 35%) as an off-white solid. ¹H NMR (400 MHz, CDCl₃): δ 8.36 (d, J=8.8 Hz, 1H), 7.69 (s, 1H), 7.37-7.32 (m, 1H), 7.20-7.15 (m, 2H), 7.04-6.99 (m, 1H), 6.57-6.53 (m, 2H), 6.42-6.37 (m, 1H), 6.30-6.23 (m, 1H), 5.74-5.72 (m, 1H), 5.04 (s, 2H), 3.87 (s, 3H). [MH]⁺ 302.2.

### Example 1.5. Synthesis of N-(2-(3-fluorophenyl)-2,3-dihydrobenzo[b][1,4]dioxin-6-yl)acrylamide (8)

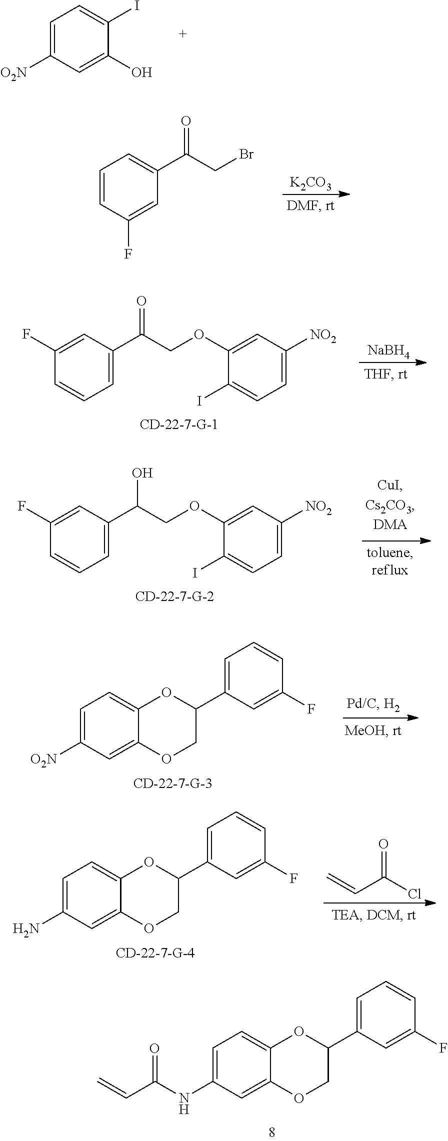

1-(3-fluorophenyl)-2-(2-iodo-5-nitrophenoxy)ethenone (CD-22-7-G-1). To a solution of 2-iodo-5-nitrophenol (0.500 g, 1.89 mmol) and 2-bromo-1-(3-fluorophenyl)ethanone (0.409 g, 1.89 mmol) in N,N-dimethylformamide (5 mL) was added potassium carbonate (522 mg, 3.77 mmol) at room temperature under nitrogen, and the resulting mixture was stirred at room temperature for 1 h. After cooling to room temperature, the reaction mixture was poured into water (15 mL) and extracted ethyl acetate (30 mL×2). Combined organic extracts were washed with brine (20 mL), dried over Na₂SO₄, and concentrated in vacuo. The resulting crude was purified by silica gel column chromatography using a 10%-20% ethyl acetate/hexane gradient to afford 1-(3-fluorophenyl)-2-(2-iodo-5-nitrophenoxy)ethanone (CD-22-7-G-1) (0.652 g, 86%) as a yellow solid.

1-(3-fluorophenyl)-2-(2-iodo-5-nitrophenoxy)ethanol (CD-22-7-G-2). To a solution of 1-(3-fluorophenyl)-2-(2-iodo-5-nitrophenoxy)ethanone (CD-22-7-G-1) (0.705 g, 1.76 mmol) in tetrahydrofuran (8 ml) was added sodium borohydride (100 mg, 2.64 mmol) at 0-5° C., and the resulting mixture was stirred at room temperature for 2 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 10% ethyl acetate/hexane gradient to afford 1-(3-fluorophenyl)-2-(2-iodo-5-nitrophenoxy)ethanol (CD-22-7-G-2) (0.570 mg, 80%) as a yellow solid.

2-(3-fluorophenyl)-6-nitro-2,3-dihydrobenzo[b][1,4]dioxine (CD-22-7-G-3). To a solution of 1-(3-fluorophenyl)-2-(2-iodo-5-nitrophenoxy)ethanol (CD-22-7-G-2) (0.570 g, 1.41 mmol), cesium carbonate (0.921 g, 2.83 mmol), and N,N′-Dimethyl-1,2-ethanediamine (0.062 g, 0.71 mmol) in toluene (7 mL) was added CuI (0.269 g, 1.41 mmol) at room temperature under nitrogen atmosphere; the mixture was degassed with nitrogen three times. The resulting mixture was refluxed for 2 h. The resulting mixture was filtered and the filtrate was concentrated in vacuo. The resulting crude was purified by silica gel column chromatography using a 2.5% ethyl acetate/hexane gradient to afford 2-(3-fluorophenyl)-6-nitro-2,3-dihydrobenzo[b][1,4]dioxine (CD-22-7-G-3) (0.212 g, 52%) as an off-white solid.

2-(3-fluorophenyl)-2,3-dihydrobenzo[b][1,4]dioxin-6-amine (CD-22-7-G-4). To a solution of 2-(3-fluorophenyl)-6-nitro-2,3-dihydrobenzo[b][1,4]dioxine (CD-22-7-G-3) (0.100 g, 0.063 mmol) in methanol (5 ml)-ethyl acetate (3 mL) was added palladium on carbon (10%, 123 mg) at room temperature, and the resulting mixture was stirred at room temperature under H₂ atmosphere (H₂ balloon) for 4 hours. The catalyst was removed through filtration and the filtrate cake was washed with MeOH (10 mL×2), and the combined filtrates were concentrated under reduced pressure to give crude product, which was purified by silica gel flash column chromatography using a 1% methanol/dichloromethane gradient to afford 2-(3-fluorophenyl)-2,3-dihydrobenzo[b] [1,4]dioxin-6-amine (CD-22-7-G-4) (0.089 g, 99%) as a brown solid. MS: [MH]+246.1.

N-(2-(3-fluorophenyl)-2,3-dihydrobenzo[b][1,4]dioxin-6-yl)acrylamide (8). To a solution of 2-(3-fluorophenyl)-2,3-dihydrobenzo[b][1,4]dioxin-6-amine (CD-22-7-G-4) (0.050 g, 0.20 mmol) and triethylamine (0.062 g, 0.61 mmol) in dichloromethane (2 mL) was added a solution of acryloyl chloride (0.024 g, 0.27 mmol) in dichloromethane (0.5 ml) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(2-(3-fluorophenyl)-2,3-dihydrobenzo[b][1,4]dioxin-6-yl)acrylamide (8) (0.041 g, 67%) as a light brown solid. ¹H NMR (400 MHz, DMSO-d₆) δ 10.02 (s, 1H), 7.52-7.43 (m, 1H), 7.41 (d, J=2.4 Hz, 1H), 7.34-7.31 (m, 2H), 7.25-7.20 (m, 1H), 7.09-7.03 (m, 1H), 6.94 (d, J=8.4 Hz, 1H), 6.39-6.35 (m, 1H), 6.27-6.23 (m, 1H), 5.77-5.69 (m, 1H), 5.27-5.23 (m, 1H), 4.45-4.41 (m, 1H), 4.14-4.09 (m, 1H). MS: [MH]⁺ 300.0.

### Example 1.6. Synthesis of N-(4-((3-fluorobenzyl)oxy)-3-(trifluoromethyl)phenyl)acrylamide (9)

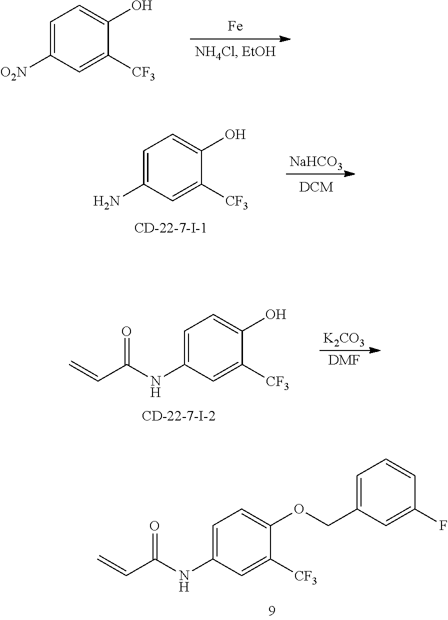

4-amino-2-(trifluoromethyl)phenol (CD-22-7-I-1). A mixture of 4-nitro-2-(trifluoromethyl)phenol (0.300 g, 1.45 mmol) and ammonium chloride (0.931 g, 17.4 mmol) in ethanol (10 ml)-water (5 ml) was added iron powder (0.405 g, 7.25 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 20%-30% ethyl acetate/hexane gradient to afford 4-amino-2-(trifluoromethyl)phenol (CD-22-7-I-1) (0.220 g, 87%) as brown solid. MS: [MH]⁺ 178.2.

N-(4-hydroxy-3-(trifluoromethyl)phenyl)acrylamide (CD-22-7-I-2). To a stirred solution of 4-amino-2-(trifluoromethyl)phenol (CD-22-7-I-1) (0.100 g, 0.94 mmol), in a mixture of sat aq. NaHCO₃ (10 mL)-DCM (2 mL) was added acryloyl chloride (0.039 g, 0.94 mmol) dropwise at 0° C. under nitrogen, and the resulting mixture was stirred at 0° C. for 30 min. The organic layer was collected, and the aqueous layer was extracted with DCM (10 mL×2). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(4-hydroxy-3-(trifluoromethyl)phenyl)acrylamide (CD-22-7-I-2) (0.030 g, 23%) as a white solid. MS: [MH]+232.1.

N-(4-((3-fluorobenzyl)oxy)-3-(trifluoromethyl)phenyl)acrylamide (9). To a solution of N-(4-hydroxy-3-(trifluoromethyl)phenyl)acrylamide (0.030 g, 0.13 mmol) (CD-22-7-I-2) and 1-(bromomethyl)-3-fluorobenzene (0.025 g, 0.13 mmol) in N,N-dimethylformamide (2 mL) was added potassium carbonate (0.054 g, 0.39 mmol). The reaction mixture was poured into water (10 mL) and extracted with ethyl acetate (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(4-((3-fluorobenzyl)oxy)-3-(trifluoromethyl)phenyl)acrylamide (9) (0.040 g, 90%) as a white solid. ¹H NMR (400 MHz, CDCl₃): δ 5.16 (s, 2H), 5.85-5.80 (m, 1H), 6.23-6.20 (m, 1H), 6.45-6.41 (m, 1H), 7.05-6.93 (m, 2H), 7.18-7.14 (m, 2H), 7.35 (s, 1H), 7.71 (s, 1H), 7.83 (s, 1H). MS: [MH]⁺ 340.1.

### Example 1.7. Synthesis of N-(2-(3-fluorophenyl)-3,4-dihydro-2H-benzo[b][1,4]oxazin-6-yl)acrylamide (10)

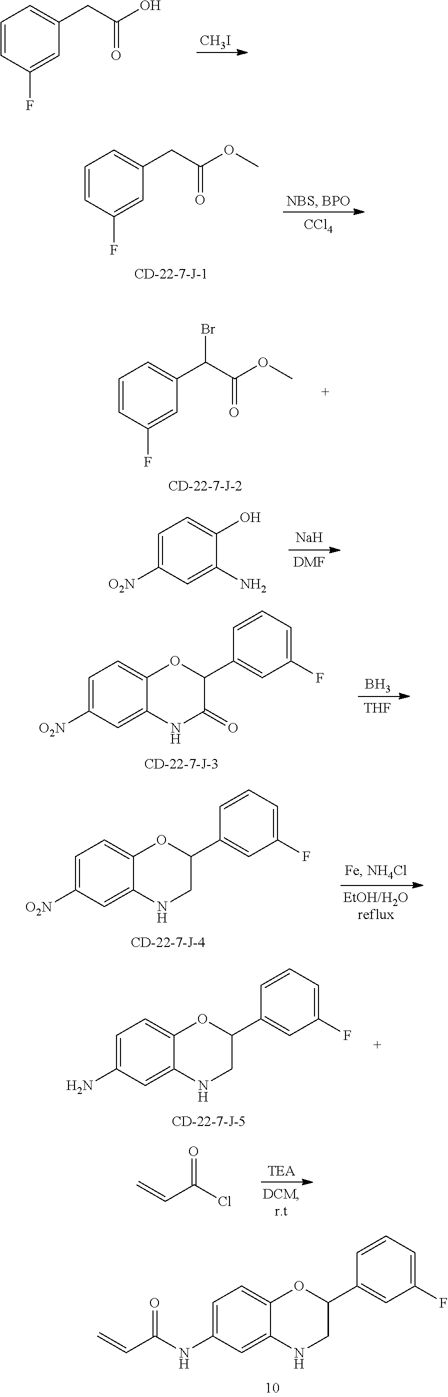

methyl 2-(3-fluorophenyl) acetate (CD-22-7-J-1). To a solution of 2-(3-fluorophenyl) acetic acid (10.000 g, 64.90 mmol) and potassium carbonate (18.400 g, 129.80 mmol) in N, N-dimethylformamide (100 mL) was added iodomethane (17.900 g, 129.80 mmol) at 0° C., and the resulting mixture was stirred at room temperature for 2.5 h. The reaction mixture was poured into water (100 mL) and extracted with ethyl acetate (50 mL×2). Combined organic extracts were washed with brine (75 mL), dried over anhydrous Na₂SO₄ and concentrated in vacuo. The resulting crude was purified by silica gel column chromatography using a 3% ethyl acetate/hexane gradient to afford methyl 2-(3-fluorophenyl) acetate (CD-22-7-J-1) (9.970 g, 91%) as a light yellow oil.

methyl 2-bromo-2-(3-fluorophenyl) acetate (CD-22-7-J-2). To a solution of methyl 2-(3-fluorophenyl) acetate (CD-22-7-J-1) (10.000 g, 59.50 mmol), NBS (12.700 g, 71.40 mmol) in CCl₄ (100 mL) was added benzoyl peroxide (3.840 g, 11.90 mmol) at room temperature, and the resulting mixture was stirred at 90° C. for 12 h. The reaction mixture was concentrated under reduced pressure. The resulting crude was purified by silica gel column chromatography using a 1% ethyl acetate/hexane gradient to afford methyl 2-bromo-2-(3-fluorophenyl) acetate (CD-22-7-J-2) (13.500 g, 92%) as a light yellow oil.

2-(3-fluorophenyl)-6-nitro-2H-benzo[b][1,4]oxazin-3(4H)-one (CD-22-7-J-3). To a suspension of sodium hydride (60% in mineral oil, 1.040 g, 26.00 mmol) in N,N-dimethylformamide (10 mL) was added a solution of 2-amino-4-nitrophenol (2.000 g, 13.00 mmol) in N,N-dimethylformamide (5 mL), and the resulting mixture was stirred at 0° C. for 30 min. Subsequently, methyl 2-bromo-2-(3-fluorophenyl)acetate (CD-22-7-J-2) (3.500 g, 13.00 mmol) in N,N-dimethylformamide (5 mL) were added to the reaction mixture at 0° C., and the resulting mixture was stirred at 0° C. for 20 min. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5% ethyl acetate/hexane gradient to afford 2-(3-fluorophenyl)-6-nitro-2H-benzo[b][1,4]oxazin-3(4H)-one (CD-22-7-J-3) (0.312 g, 8%) as pale yellow solid.

2-(3-fluorophenyl)-6-nitro-3,4-dihydro-2H-benzo[b][1,4]oxazine (CD-22-7-J-4). To a solution of 2-(3-fluorophenyl)-6-nitro-2H-benzo[b] [1,4]oxazin-3(4H)-one (CD-22-7-J-3) (0.500 g, 1.73 mmol) in THF (10 mL) was added borane-tetrahydrofuran complex (17 ml, 1 mol/L) at 0° C. under nitrogen atmosphere, and the resulting mixture was stirred at 0° C. for 3 h. The reaction mixture was quenched with HCl (5 mL, 6 N), and the mixture was basified with 4 N NaOH to pH=10 at 0° C. The reaction mixture was extracted with DCM (10 mL×2). Combined organic extracts were washed with brine (5 mL), dried over anhydrous Na₂SO₄, and concentrated in vacuo. The resulting crude was purified by silica gel column chromatography using a 12-15% ethyl acetate/hexane gradient to afford 2-(3-fluorophenyl)-6-nitro-3,4-dihydro-2H-benzo[b][1,4]oxazine (CD-22-7-J-4) (0.500 g, 100%) as a light yellow solid. MS: [M+42]⁺ 316.1.

2-(3-fluorophenyl)-3,4-dihydro-2H-benzo[b][1,4]oxazin-6-amine (CD-22-7-J-5). To a solution of 2-(3-fluorophenyl)-6-nitro-3,4-dihydro-2H-benzo[b][1,4]oxazine (CD-22-7-J-4) (0.051 g, 0.19 mmol) and ammonium chloride (0.050 g, 0.93 mmol) in EtOH (4 mL)-H₂O (1 mL) was added iron powered (0.052 g, 0.93 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 20%-30% ethyl acetate/hexane gradient to afford 2-(3-fluorophenyl)-3,4-dihydro-2H-benzo[b][1,4]oxazin-6-amine (CD-22-7-J-5) (0.031 g, 69%) as an off-white solid. MS: [MH]+245.4.

N-(2-(3-fluorophenyl)-3,4-dihydro-2H-benzo[b][1,4]oxazin-6-yl)acrylamide (10). To a solution of 2-(3-fluorophenyl)-3,4-dihydro-2H-benzo[b][1,4]oxazin-6-amine (CD-22-7-J-5) (0.130 g, 0.53 mmol) and triethanolamine (0.162 g, 1.59 mmol) in dichloromethane (10 mL) at 0° C. was added acryloyl chloride (0.053 g, 0.58 mmol) in dichloromethane (1 mL) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(2-(3-fluorophenyl)-3,4-dihydro-2H-benzo[b][1,4]oxazin-6-yl)acrylamide (10) (0.027 g, 17%) as an off-white solid. ¹H NMR (400 MHz, CDCl₃): δ 7.39-7.34 (m, 2H), 7.19-7.14 (m, 2H), 7.07-7.02 (m, 1H), 6.86 (d, J=8.4 Hz, 1H), 6.67-6.65 (m, 1H), 6.40 (d, J=16.8 Hz, 1H), 6.26-6.19 (m, 1H), 5.74 (d, J=10.4 Hz, 1H), 5.11-5.08 (m, 1H), 3.57-3.53 (m, 1H), 3.37-3.32 (m, 1H). MS: [MH]⁺ 299.1.

### Example 1.8. Synthesis of N-(2-(2-amino-2-oxoethyl)-4-((3-fluorobenzyl)oxy)phenyl)acrylamide (11)

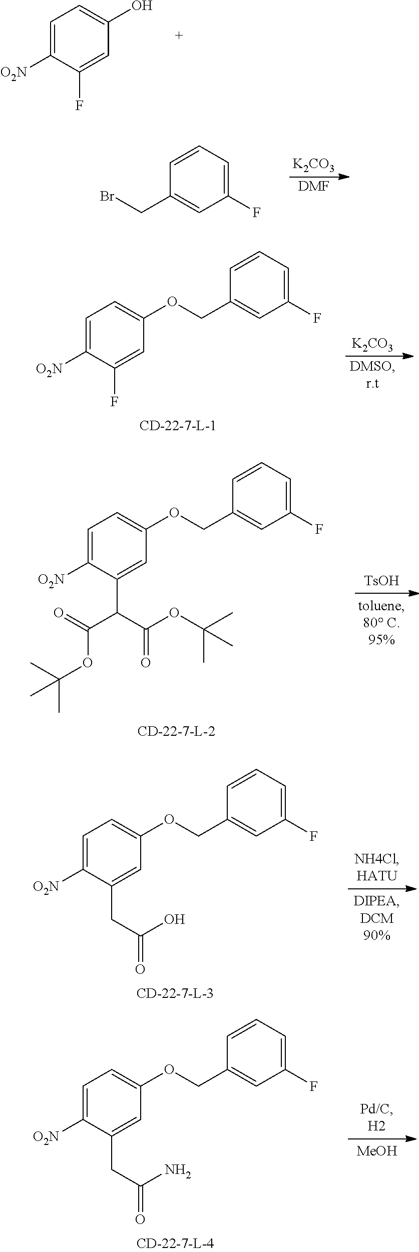

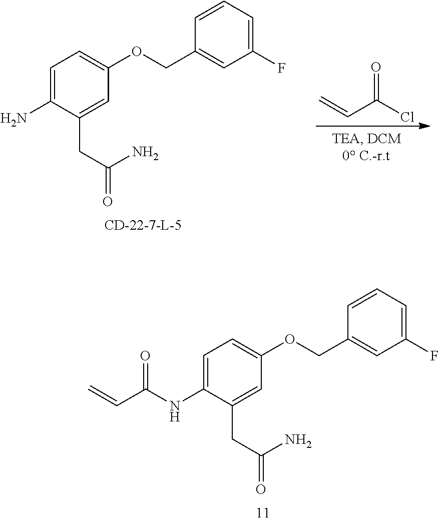

2-fluoro-4-((3-fluorobenzyl)oxy)-1-nitrobenzene (CD-22-7-L-1). To a solution of 3-fluoro-4-nitrophenol (0.500 g, 3.18 mmol) and 1-(bromomethyl)-3-fluorobenzene (0.599 g, 3.18 mmol) in N,N-dimethylformamide (10 mL) was added potassium carbonate (0.612 g, 4.44 mmol) at room temperature under nitrogen, and the resulting mixture was stirred at room temperature for 4 h. The reaction mixture was poured into water (20 mL) and extracted with ethyl acetate (10 mL×2). Combined organic extracts were washed with brine (15 mL), dried over anhydrous Na₂SO₄, and concentrated in vacuo. The resulting crude was purified by silica gel column chromatography using a 30-50% ethyl acetate/hexane gradient to afford 2-fluoro-4-((3-fluorobenzyl)oxy)-1-nitrobenzene (CD-22-7-L-1) (0.675 g, 80%) as a yellow solid.

di-tert-butyl 2-(5-((3-fluorobenzyl)oxy)-2-nitrophenyl)malonate (CD-22-7-L-2). To a solution of 2-fluoro-4-((3-fluorobenzyl)oxy)-1-nitrobenzene (CD-22-7-L-1) (1.000 g, 3.77 mmol) and di-tert-butyl malonate (1.220 g, 5.66 mmol) in dimethyl sulfoxide (10 mL) was added potassium carbonate (0.966 g, 7.00 mmol) at 0° C. under nitrogen, and resulting mixture was stirred at 50° C. for 5 h under nitrogen. The reaction was poured into water (30 mL) and extracted with ethyl acetate (30 mL). Combined organic filtrates were concentrated in vacuo. The resulting crude was purified by silica gel column chromatography using a 1-3% ethyl acetate/hexane gradient to afford di-tert-butyl 2-(5-((3-fluorobenzyl)oxy)-2-nitrophenyl)malonate (CD-22-7-L-2) (0.760 g, 45%) as a brown oil.

2-(5-((3-fluorobenzyl)oxy)-2-nitrophenyl)acetic acid (CD-22-7-L-3). A mixture of di-tert-butyl 2-(5-((3-fluorobenzyl)oxy)-2-nitrophenyl)malonate (CD-22-7-L-2) (0.760 g, 1.64 mmol) and p-toluenesulfonic acid (0.141 g, 0.82 mmol) in toluene (10 mL) was stirred at 80° C. under nitrogen for 12 h. The reaction mixture was concentrated under reduced pressure. The resulting crude was purified by silica gel column chromatography using a 33% ethyl acetate/hexane gradient to afford 2-(5-((3-fluorobenzyl)oxy)-2-nitrophenyl)acetic acid (CD-22-7-L-3) (0.180 g, 36%) as a white solid.

2-(5-((3-fluorobenzyl)oxy)-2-nitrophenyl)acetamide (CD-22-7-L-4). To a stirred solution of 2-(5-((3-fluorobenzyl)oxy)-2-nitrophenyl)acetic acid (CD-22-7-L-3) (0.180 g, 0.59 mmol), ammonium chloride (0.063 g, 1.18 mmol), and N-ethyl-N-isopropylpropan-2-amine (0.304 g, 2.36 mmol) in anhydrous N,N-dimethylformamide (3 mL) was added HATU (0.291 g, 0.767 mmol) at 0° C., the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 2-(5-((3-fluorobenzyl)oxy)-2-nitrophenyl)acetamide (CD-22-7-L-4) (0.156 g, 87%) as a yellow solid. MS: [MH]⁺ 305.0

2-(2-amino-5-((3-fluorobenzyl)oxy)phenyl)acetamide (CD-22-7-L-5). To a solution of 2-(5-((3-fluorobenzyl)oxy)-2-nitrophenyl)acetamide (CD-22-7-L-4) (0.140 g, 0.46 mmol) and ammonium chloride (0.148 g, 2.76 mmol) in ethanol (6 mL)-water (2 mL) was added iron powder (0.155 g, 2.76 mmol) at room temperature under nitrogen and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 50% ethyl acetate/hexane gradient to afford 2-(2-amino-5-((3-fluorobenzyl)oxy)phenyl)acetamide (CD-22-7-L-5) (0.124 g, 98%) as a brown solid. MS: [MH]⁺ 275.1.

N-(2-(2-amino-2-oxoethyl)-4-((3-fluorobenzyl)oxy)phenyl)acrylamide (11). To a stirred solution of 2-(2-amino-5-((3-fluorobenzyl)oxy)phenyl)acetamide (CD-22-7-L-5) (0.060 g, 0.22 mmol) and triethylamine (0.060 g, 0.66 mmol) in dichloromethane (2 mL) was added acryloyl chloride (0.030 g, 0.33 mmol) dropwise under nitrogen atmosphere at 0° C. Then the reaction mixture was stirred at room temperature for 2 hours. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(2-(2-amino-2-oxoethyl)-4-((3-fluorobenzyl)oxy)phenyl)acrylamide (11) (0.020 g, 28%) as a yellow solid. ¹HNMR (400 MHz, DMSO-d₆): δ 7.46 (d, J=8.8 Hz, 1H), 7.41-7.35 (m, 1H), 7.25 (d, J=7.6 Hz, 1H), 7.18 (d, J=8.0 Hz, 1H), 7.07-7.00 (m, 1H), 6.98-6.92 (m, 2H), 6.45-6.31 (m, 2H), 5.78-7.74 (m, 1H), 5.11 (s, 2H), 3.51 (s, 2H). MS: [MH]⁺ 329.1.

### Example 1.9. Synthesis of N-(2-(3-fluorophenyl)-4-methyl-3,4-dihydro-2H-benzo[b][1,4]oxazin-6-yl)acrylamide (12)

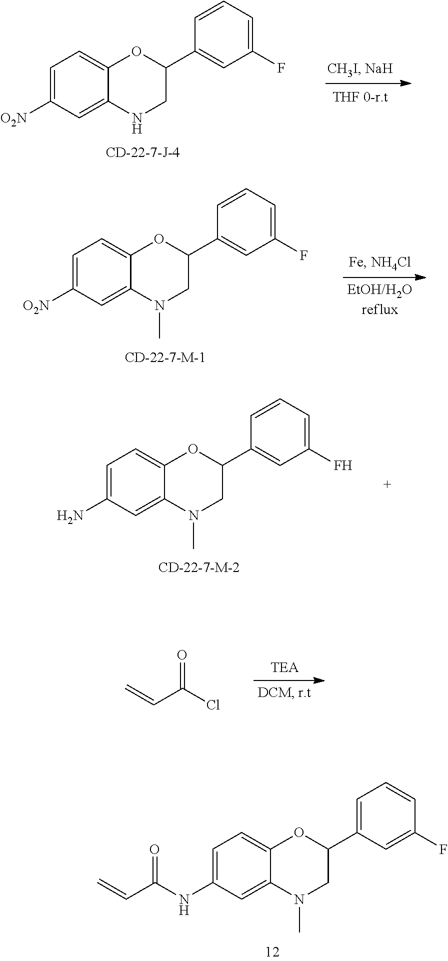

2-(3-fluorophenyl)-4-methyl-6-nitro-3,4-dihydro-2H-benzo[b][1,4]oxazine (CD-22-7-M-1). To a solution of sodium hydride (60% in mineral oil, 0.015 g, 0.36 mmol) in tetrahydrofuran (5 mL) was added 2-(3-fluorophenyl)-6-nitro-3,4-dihydro-2H-benzo[b][1,4]oxazine (CD-22-7-J-4) (0.050 g, 0.18 mmol) and CH₃I (0.052 g, 0.36 mmol), and the resulting mixture was stirred at 0° C. for 1 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 2-(3-fluorophenyl)-4-methyl-6-nitro-3,4-dihydro-2H-benzo[b][1,4]oxazine (CD-22-7-M-1) (0.028 g, 54%) as a pale yellow solid.

2-(3-fluorophenyl)-4-methyl-3,4-dihydro-2H-benzo[b][1,4]oxazin-6-amine (CD-22-7-M-2). To a solution of 2-(3-fluorophenyl)-4-methyl-6-nitro-3,4-dihydro-2H-benzo[b][1,4]oxazine (CD-22-7-M-1) (0.150 g, 0.52 mmol) and ammonium chloride (0.140 g, 2.60 mmol) in EtOH (4 mL)-H₂O (1 mL) was added iron powder (0.146 g, 2.60 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 10%-20% ethyl acetate/hexane gradient to afford 2-(3-fluorophenyl)-4-methyl-3,4-dihydro-2H-benzo[b][1,4]oxazin-6-amine (CD-22-7-M-2) (0.074 g, 55%) as a colorless oil. MS: [MH]⁺ 259.2.

N-(2-(3-fluorophenyl)-4-methyl-3,4-dihydro-2H-benzo[b][1,4]oxazin-6-yl)acrylamide (12). To a solution of 2-(3-fluorophenyl)-4-methyl-3,4-dihydro-2H-benzo[b][1,4]oxazin-6-amine (CD-22-7-M-2) (0.050 g, 0.19 mmol) and TEA (0.020 g, 0.58 mmol) in dichloromethane (5 mL) was added acryloyl chloride (0.019 g, 0.213 mmol) in dichloromethane (1 mL) at 0° C., and the resulting mixture was stirred at room temperature for 1.5 h. The reaction mixture was poured into water (5 mL) and extracted with DCM (5 mL×2). Combined organic extracts were washed with brine (5 mL), dried over anhydrous Na₂SO₄, and concentrated in vacuo. The resulting crude was purified by silica gel column chromatography using a 30% ethyl acetate/hexane gradient to afford N-(2-(3-fluorophenyl)-4-methyl-3,4-dihydro-2H-benzo[b][1,4]oxazin-6-yl)acrylamide (12) (0.065 g, 76%) as a light yellow solid. ¹H NMR (400 MHz, CDCl₃): δ 7.38-7.32 (m, 2H), 7.20-7.12 (m, 3H), 7.06-7.01 (m, 1H), 6.83 (d, J=8.4 Hz, 1H), 6.65-6.61 (m, 1H), 6.42 (d, J=6.4 Hz, 1H), 6.27-6.20 (m, 1H), 5.74 (d, J=10.4 Hz, 1H), 5.17-5.12 (m, 1H), 3.36-3.33 (m, 1H), 3.26-3.21 (m, 1H), 2.93 (s, 3H). MS: [MH]⁺ 313.1.

### Example 1.10. Synthesis of N-(4-((3-fluorobenzyl)oxy)-2-(pyridin-4-ylmethyl)phenyl)acrylamide (13)

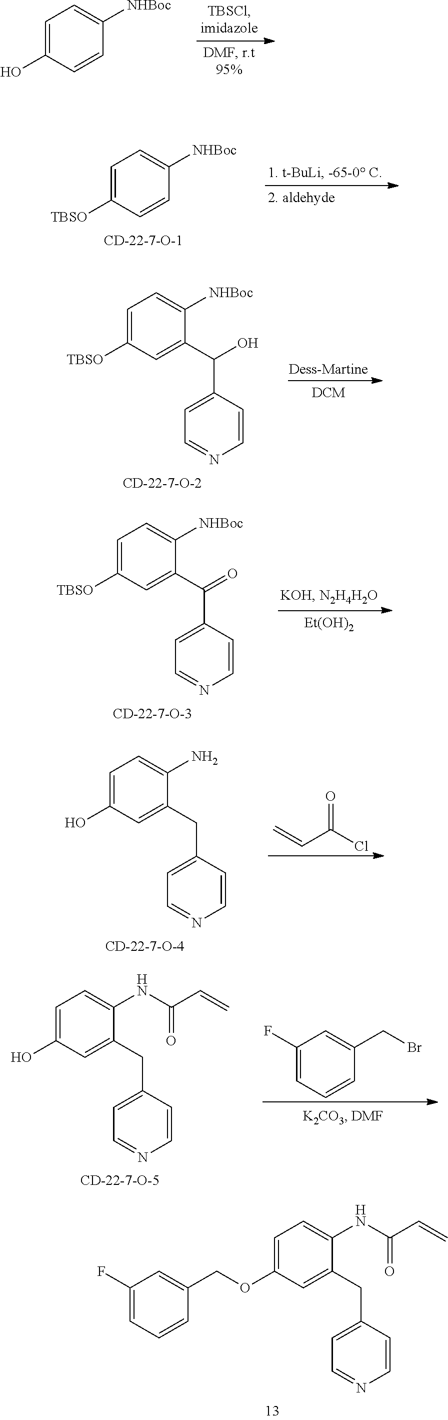

tert-butyl (4-((tert-butyldimethylsilyl)oxy)phenyl)carbamate (CD-22-7-O-1). To a stirred solution of tert-butyl (4-hydroxyphenyl)carbamate (1.000 g, 4.78 mmol) and imidazole (975 mg, 14.34 mmol) in a N,N-dimethylformamide (30 mL) was slowly added tert-butylchlorodimethylsilane (1.430 g, 9.56 mmol) at room temperature. The reaction mixture was stirred at from temperature for 2 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5-10% ethyl acetate/hexane gradient to afford tert-butyl (4-((tert-butyldimethylsilyl)oxy)phenyl)carbamate (CD-22-7-O-1) (1.500 g, 97%) as white solid.

tert-butyl (4-((tert-butyldimethylsilyl)oxy)-2-(hydroxy(pyridin-4-yl)methyl)phenyl)carbamate (CD-22-7-O-2). To a stirred solution of tert-butyl (4-((tert-butyldimethylsilyl)oxy)phenyl)carbamate (CD-22-7-O-1) (0.500 g, 1.50 mmol) in anhydrous tetrahydrofuran (10 mL) was slowly added tert-butyllithium (2.9 mL, 3.75 mmol, 1.3 M in hexane) at −68° C. The resulting mixture was stirred at −10° C. for 2 hours followed by addition of aldehyde (0.199 g, 1.85 mmol) in anhydrous tetrahydrofuran (1 mL) at −68° C., and the resulting mixture was stirred at at −68° C. for 2 h. The reaction mixture was poured into aqueous ammonium chloride (sat. 40 ml) at −68° C. and extracted with ethyl acetate (25 ml×3). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford tert-butyl (4-((tert-butyldimethylsilyl)oxy)-2-(hydroxy(pyridin-4-yl)methyl)phenyl)carbamate (CD-22-7-O-2) (0.130 g, 19%) as a yellow solid. MS: [MH]⁺ 431.4.

tert-butyl (4-((tert-butyldimethylsilyl)oxy)-2-isonicotinoylphenyl)carbamate (CD-22-7-O-3). To a stirred solution of tert-butyl (4-((tert-butyldimethylsilyl)oxy)-2-(hydroxy(pyridin-4-yl)methyl)phenyl)carbamate (CD-22-7-O-2) (0.200 g, 0.46 mmol) in dichloromethane (5 mL) was added Dess-Martin periodinane (0.296 g, 0.7 mmol) at room temperature. The resulting mixture was stirred at room temperature for 2 hours. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 0-5% ethyl acetate/hexane gradient to afford tert-butyl (4-((tert-butyldimethylsilyl)oxy)-2-isonicotinoylphenyl)carbamate (CD-22-7-O-3) (0.180 g, 90%) as a light yellow oil. MS: [MH]⁺ 429.3.

4-amino-3-(pyridin-4-ylmethyl)phenol (CD-22-7-O-4). The mixture of tert-butyl (4-((tert-butyldimethylsilyl)oxy)-2-isonicotinoylphenyl)carbamate (CD-22-7-O-3) (0.180 g, 0.42 mmol) and potassium hydroxide (0.071 g, 1.26 mmol) in hydrazine hydrate (1 ml)-ethane-1,2-diol (5 ml) was stirred at 200° C. overnight under nitrogen atmosphere. The mixture solution was cooled to room temperature, acidified with diluted hydrochloride acid (3N) to pH 7-8, and extracted with ethyl acetate (20 ml×3). Combined organic extracts were washed with brine (10 mL), dried over anhydrous Na₂SO₄, and concentrated in vacuo. The resulting crude was purified by silica gel column chromatography using a 10% MeOH/DCM gradient to afford 4-amino-3-(pyridin-4-ylmethyl)phenol (CD-22-7-O-4) (0.050 g, 59%) as white solid. MS [MH]⁺ 201.1.

N-(4-hydroxy-2-(pyridin-4-ylmethyl)phenyl)acrylamide (CD-22-7-O-5). To a stirred solution of 4-amino-3-(pyridin-4-ylmethyl)phenol (CD-22-7-O-4) (40 mg, 0.2 mmol) in tetrahydrofuran (5 ml) was added acryloyl chloride (18.1 mg, 0.2 mmol) at room temperature. The resulting mixture was stirred at room temperature for 0.5 hour. The mixture solution was concentrated under reduced pressure. The resulting crude was purified by silica gel column chromatography using a 50% ethyl acetate/hexane gradient to afford N-(4-hydroxy-2-(pyridin-4-ylmethyl)phenyl)acrylamide (CD-22-7-O-5) (0.022 g, 43%) as a white solid. MS: [MH]⁺ 255.1.

N-(4-((3-fluorobenzyl)oxy)-2-(pyridin-4-ylmethyl)phenyl)acrylamide (13). To a stirred solution of N-(4-hydroxy-2-(pyridin-4-ylmethyl)phenyl)acrylamide (CD-22-7-O-5) (0.020 g, 0.08 mmol) and potassium carbonate (0.022 g, 0.16 mmol) in N,N-dimethylformamide (1 mL) was added 1-(bromomethyl)-3-fluorobenzene (0.015 g, 0.08 mmol). The resulting mixture was stirred at room temperature for 1 hour. TLC showed the reaction was complete. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 10% MeOH/DCM gradient to afford N-(4-((3-fluorobenzyl)oxy)-2-(pyridin-4-ylmethyl)phenyl)acrylamide (13) (0.007 g, 25%) as a brown solid. ¹H NMR (400 MHz, CDCl₃): δ 8.50 (d, J=5.2 Hz, 2H), 7.48 (d, J=8.8 Hz, 1H), 7.37-7.31 (m, 1H), 7.17-7.11 (m, 2H), 7.06-7.00 (m, 3H), 6.92-6.89 (m, 1H), 6.79 (s, 2H), 6.32-6.27 (m, 1H), 6.14-6.07 (m, 1H), 5.73-5.71 (m, 1H), 5.04 (s, 2H), 3.94 (s, 2H). MS: [MH]⁺ 363.4.

### Example 1.11. Synthesis of N-(8-((3-fluorobenzyl)oxy)chroman-5-yl)acrylamide (14)

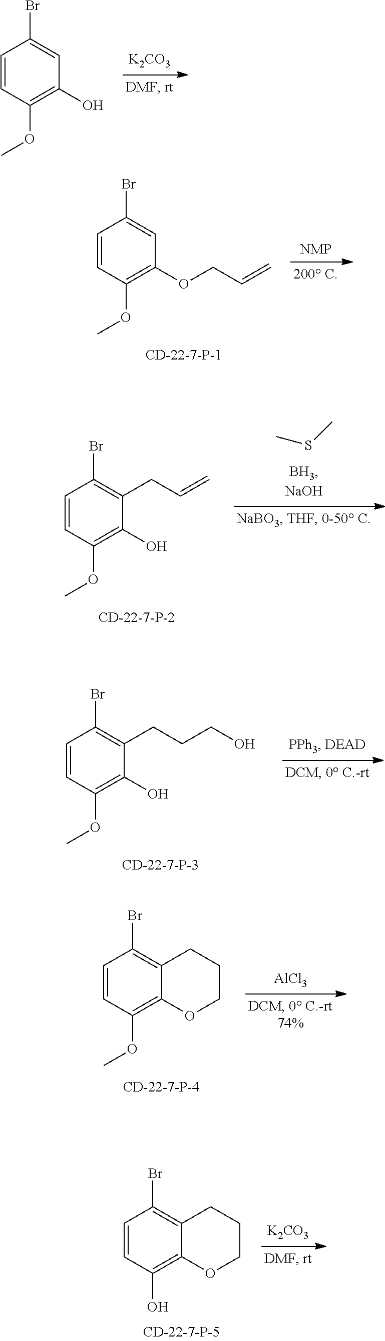

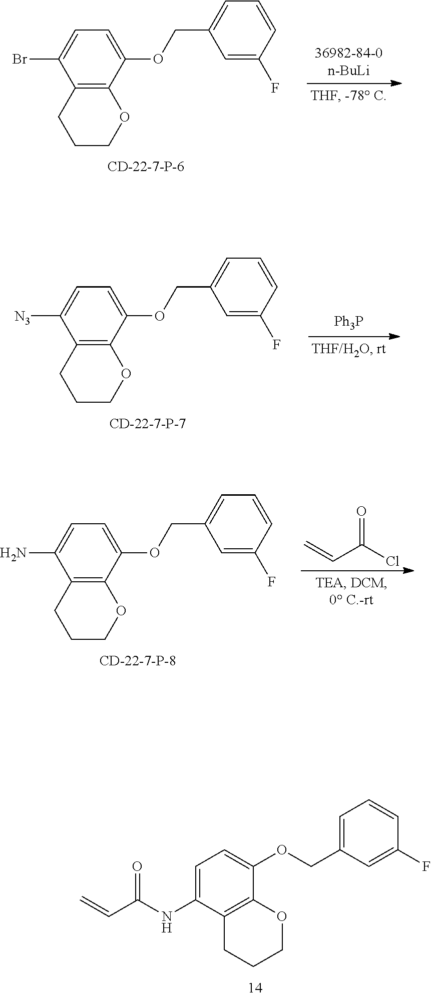

2-(allyloxy)-4-bromo-1-methoxybenzene (CD-22-7-P-1). A mixture of 5-bromo-2-methoxyphenol (1.000 g, 4.93 mmol), allyl bromide (0.596 g, 4.93 mmol), and potassium carbonate (1.36 g, 9.85 mmol) in N,N-dimethylformamide (8 mL) was stirred at room temperature for 2 hours under nitrogen. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 2-(allyloxy)-4-bromo-1-methoxybenzene (CD-22-7-P-1) (1.060 g, 88%) as a colorless oil.

2-allyl-3-bromo-6-methoxyphenol (CD-22-7-P-2). A solution of 2-(allyloxy)-4-bromo-1-methoxybenzene (CD-22-7-P-1) (0.925 g, 3.81 mmol) in 1-methylpyrrolidin-2-one (8 mL) was stirred at 200° C. for 5 hours under nitrogen. After cooling to room temperature, the reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 1% ethyl acetate/hexane gradient to afford 2-allyl-3-bromo-6-methoxyphenol (CD-22-7-P-2) (0.750 g, 81%) as a colorless oil.

3-bromo-2-(3-hydroxypropyl)-6-methoxyphenol (CD-22-7-P-3). To a solution of 2-allyl-3-bromo-6-methoxyphenol (CD-22-7-P-2) (0.725 g, 2.98 mmol) in tetrahydrofuran (10 mL) was added borane-methyl sulfide complex (2.0 M, 3 mL, 5.96 mmol) at 0-5° C. The resulting mixture was stirred at room temperature for 2 h under nitrogen. Followed by the addition of aqueous solution of sodium hydroxide (5 M, 1.8 mL, 8.95 mmol) and sodium perborate tetrahydrate (1.38 g, 8.95 mmol) at 0-5° C., and the resulting mixture was stirred at 50° C. for 1 h under nitrogen. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 25% ethyl acetate/hexane gradient to afford 3-bromo-2-(3-hydroxypropyl)-6-methoxyphenol (CD-22-7-P-3) (0.558 g, 71%) as a white solid.

5-bromo-8-methoxychroman (CD-22-7-P-4). To a solution of 3-bromo-2-(3-hydroxypropyl)-6-methoxyphenol (CD-22-7-P-3) (0.458 g, 1.75 mmol) in dichloromethane (10 mL) at 0-5° C. was added triphenylphosphine (0.552 g, 2.10 mmol) and diethyl azodicarboxylate (0.367 g, 2.10 mmol). The reaction mixture was stirred at room temperature under nitrogen for 12 h. The mixture was concentrated under reduced pressure. The resulting crude was purified by silica gel column chromatography using a 5% ethyl acetate/hexane gradient to afford 5-bromo-8-methoxychroman (CD-22-7-P-4) (0.368 g, 71%) as a white solid.

5-bromochroman-8-ol (CD-22-7-P-5). To a solution of 5-bromo-8-methoxychroman (CD-22-7-P-4) (0.200 g, 0.82 mmol) in dichloromethane (5 mL) at 0-5° C. was added aluminum chloride (0.765 g, 5.74 mmol) in portions. The reaction mixture was stirred at room temperature for 5 hours under nitrogen. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 3% ethyl acetate/hexane gradient to afford 5-bromochroman-8-ol (CD-22-7-P-5) (0.160 g, 85%) as a yellow oil.

5-bromo-8-((3-fluorobenzyl)oxy)chroman (CD-22-7-P-6). A mixture of 5-bromochroman-8-ol (CD-22-7-P-5) (0.148 g, 0.65 mmol), 1-(bromomethyl)-3-fluorobenzene (0.134 g, 0.71 mmol) and potassium carbonate (0.179 g, 1.29 mmol) in N,N-dimethylformamide (2 mL) was stirred at room temperature for 1 h under nitrogen. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 3-5% ethyl acetate/hexane gradient to afford 5-bromo-8-((3-fluorobenzyl)oxy)chroman (CD-22-7-P-6) (0.150 g, 68%) as a colorless oil.

5-azido-8-((3-fluorobenzyl)oxy)chroman (CD-22-7-P-7). To a solution of 5-bromo-8-((3-fluorobenzyl)oxy)chroman (CD-22-7-P-6) (0.562 g, 1.67 mmol) in anhydrous tetrahydrofuran (10 mL) at −78° C. was added n-butyllithium (2.5M in hexane, 2 mL, 5.00 mmol) dropwise. The resulting mixture was stirred at −78° C. for 1 h under nitrogen, followed by the addition of a solution of 2,4,6-triisopropylbenzene-sulfonyl azide (0.774 g, 2.50 mmol) in anhydrous tetrahydrofuran (5 mL) at −78° C. The resulting mixture was stirred at −78° C. for 1 h. The reaction mixture was poured into aqueous NH₄Cl solution (sat. 10 mL) and extracted with ethyl acetate (10 mL×2). Combined organic extracts were washed with brine (15 mL), dried over anhydrous Na₂SO₄, and concentrated in vacuo. The resulting crude was purified by silica gel column chromatography using a 30-50% ethyl acetate/hexane gradient to afford 5-azido-8-((3-fluorobenzyl)oxy)chroman (CD-22-7-P-7) (0.117 g, 22%) as a yellow oil.

8-((3-fluorobenzyl)oxy)chroman-5-amine (CD-22-7-P-8). A mixture of 5-azido-8-((3-fluorobenzyl)oxy)chroman (CD-22-7-P-7) (0.102 g, 0.34 mmol) and triphenylphosphine (0.179 g, 0.68 mmol) in tetrahydrofuran (2 mL)-water (0.4 mL) was stirred at room temperature for 2 h. The mixture was concentrated under reduced pressure. The resulting crude was purified by silica gel column chromatography using a 10% ethyl acetate/hexane gradient to afford 8-((3-fluorobenzyl)oxy)chroman-5-amine (CD-22-7-P-8) (0.080 g, 86%) as a white solid.

N-(8-((3-fluorobenzyl)oxy)chroman-5-yl)acrylamide (14). To a solution of 8-((3-fluorobenzyl)oxy)chroman-5-amine (CD-22-7-P-8) (0.077 g, 0.28 mmol) and triethylamine (0.036 g, 0.85 mmol) in dichloromethane (2 mL) was added a solution of acryloyl chloride (0.033 g, 0.37 mmol) in dichloromethane (0.5 mL) at 0-5° C. The reaction mixture was stirred at room temperature for 30 min under nitrogen. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 25% ethyl acetate/hexane gradient to afford N-(8-((3-fluorobenzyl)oxy)chroman-5-yl)acrylamide (14) (0.015 g, 16%) as a yellow solid. ¹H NMR (400 MHz, CDCl₃) δ 8.28 (s, 1H), 7.24-7.12 (m, 3H), 6.86-6.70 (m, 3H), 6.46-6.41 (m, 1H), 6.34-6.27 (m, 1H), 5.76 (d, J=10.0 Hz, 1H), 5.06 (s, 2H), 4.27 (t, J=5.2 Hz, 2H), 2.81 (t, J=6.4 Hz, 2H), 2.07-2.00 (m, 2H). MS [MH]⁺ 328.1.

### Example 1.12. Synthesis of N-(4-((3-fluorobenzyl)oxy)-5,6,7,8-tetrahydronaphthalen-1-yl)acrylamide (15)

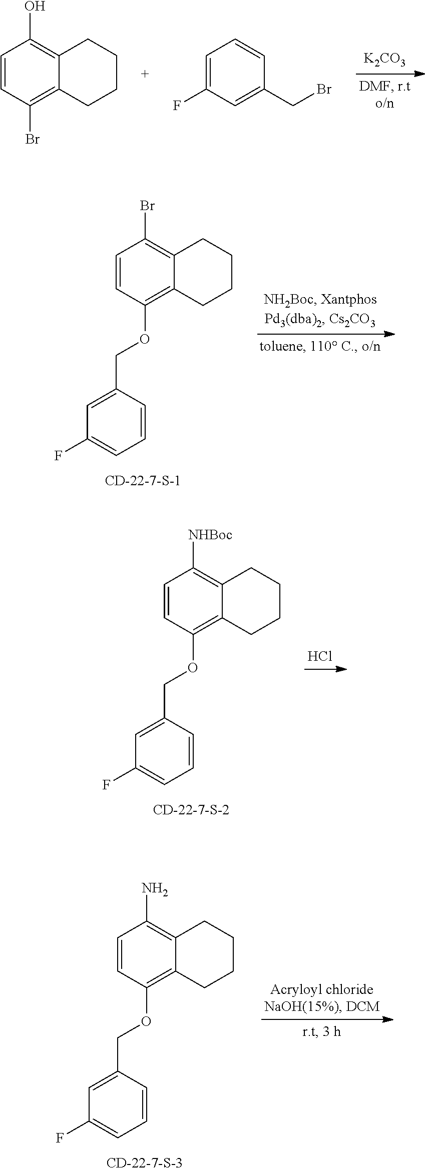

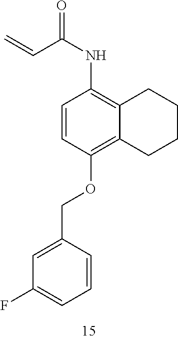 [SMILES: C=CC(=O)Nc1ccc(OCc2cccc(F)c2)c2c1CCCC2]

5-bromo-8-((3-fluorobenzyl)oxy)-1,2,3,4-tetrahydronaphthalene (CD-22-7-S-1). To a stirred solution of 4-bromo-5,6,7,8-tetrahydronaphthalen-1-ol (0.457 g, 2.00 mmol) and 1-(bromomethyl)-3-fluorobenzene (0.380 g, 2.0 mmol) in DMF (5 mL) was added K₂CO₃ (0.553 g, 4.00 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5% ethyl acetate/hexane gradient to afford 5-bromo-8-((3-fluorobenzyl)oxy)-1,2,3,4-tetrahydronaphthalene (CD-22-7-S-1) (0.435 g, 64%) as a yellow oil.

tert-butyl (4-((3-fluorobenzyl)oxy)-5,6,7,8-tetrahydronaphthalen-1-yl)carbamate (CD-22-7-S-2). To a stirred solution of 5-bromo-8-((3-fluorobenzyl)oxy)-1,2,3,4-tetrahydronaphthalene (CD-22-7-S-1) (0.335 g, 1.00 mmol), Cs₂CO₃ (1.170 g, 3.60 mmol), NH₂Boc (0.153 g, 1.3 mmol), and XantPhos (0.138 g, 0.24 mmol) in toluene (10 mL) was added Pd₂(dba)₃ (0.109 g, 0.12 mmol) at room temperature under nitrogen atmosphere; the mixture was degassed with nitrogen three times. The resulting mixture was refluxed for 8 h. The resulting mixture was filtered and the filtrate was concentrated in vacuo. The resulting crude was purified by silica gel column chromatography using a 30-50% ethyl acetate/hexane gradient to afford tert-butyl (4-((3-fluorobenzyl)oxy)-5,6,7,8-tetrahydronaphthalen-1-yl)carbamatel (CD-22-7-S-2) (0.235 g, 63%) as a yellow solid.

4-((3-fluorobenzyl)oxy)-5,6,7,8-tetrahydronaphthalen-1-amine (CD-22-7-S-3). To a stirred solution of tert-butyl (4-((3-fluorobenzyl)oxy)-5,6,7,8-tetrahydronaphthalen-1-yl)carbamate (CD-22-7-S-2) (0.235 g, 0.63 mmol) was added dioxane/HCl (4 M, 8 mL). The resulting mixture was stirred at room temperature for 3 h. The reaction mixture was concentrated and taken up in aqueous Na₂CO₃ solution (10 mL) and the extracted with DCM (10 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 4-((3-fluorobenzyl)oxy)-5,6,7,8-tetrahydronaphthalen-1-amine (CD-22-7-S-3) (0.100 g, 58%) as yellow solid. MS: [MH]⁺ 271.1.

N-(4-((3-fluorobenzyl)oxy)-5,6,7,8-tetrahydronaphthalen-1-yl)acrylamide (15). To a stirred solution of 4-((3-fluorobenzyl)oxy)-5,6,7,8-tetrahydronaphthalen-1-amine (CD-22-7-S-3) (0.050 g, 0.18 mol) in a mixture of sat aq. NaHCO₃ (1 mL)-DCM (2 mL) was added acryloyl chloride (0.018 g, 0.20 mmol) dropwise at 0° C. under nitrogen, and the resulting mixture was stirred at 0° C. for 30 min. The organic layer was collected and the aqueous layer was extracted with DCM (10 mL×2). Combined organic extracts were washed with brine (10 mL), dried over anhydrous Na₂SO₄, and concentrated in vacuo. The resulting crude was purified by silica gel column chromatography using a 30% ethyl acetate/hexane gradient to afford N-(4-((3-fluorobenzyl)oxy)-5,6,7,8-tetrahydronaphthalen-1-yl)acrylamide (15) (0.035 g, 59%) as a white solid. ¹H NMR (400 MHz, DMSO-d₆): δ 9.27 (s, 1H), 7.47-7.42 (m, 1H), 7.31-7.26 (m, 2H), 7.15-7.13 (m, 2H), 6.82-6.80 (m, 1H), 6.51-6.44 (m, 1H), 6.21-6.17 (m, 1H), 5.71-5.68 (m, 1H), 5.12 (s, 2H), 2.67-2.64 (m, 2H), 2.55-2.52 (m, 2H), 1.69-1.66 (m, 4H). MS: [MH]⁺ 325.1.

### Example 1.13. Synthesis of N-(2-(cyclopropylmethyl)-4-((3-fluorobenzyl)oxy)phenyl)acrylamide (16)

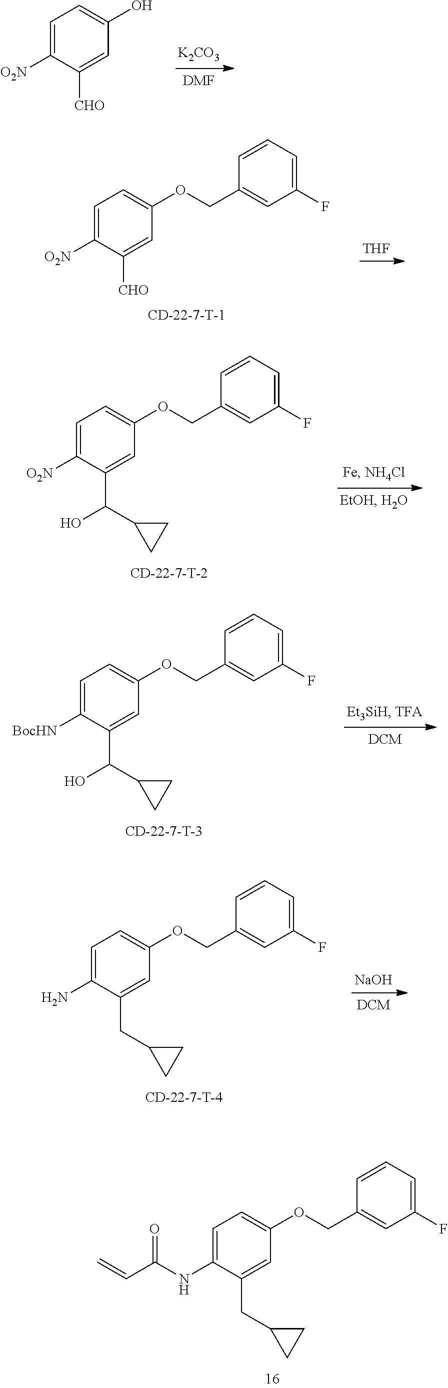

5-((3-fluorobenzyl)oxy)-2-nitrobenzaldehyde (CD-22-7-T-1). To a solution of 5-hydroxy-2-nitrobenzaldehyde (1.070 g, 6.40 mmol), 1-(bromomethyl)-3-fluorobenzene (1.330 g, 7.04 mmol) in N,N-dimethylformamide (10 mL) was added potassium carbonate (1.770 g, 12.80 mmol) at room temperature under nitrogen and the resulting mixture was stirred at room temperature for 4 h. The reaction mixture was poured into water (30 mL) and extracted with ethyl acetate (15 mL×2). Combined organic extracts were washed with brine (25 mL), dried over anhydrous Na₂SO₄ and concentrated in vacuo. The resulting crude was purified by silica gel column chromatography using a 30-50% ethyl acetate/hexane gradient to afford (CD-22-7-T-1) (1.322 g, 75%) as a yellow solid.

cyclopropyl(5-((3-fluorobenzyl)oxy)-2-nitrophenyl)methanol (CD-22-7-T-2). To a stirred solution of 5-((3-fluorobenzyl)oxy)-2-nitrobenzaldehyde (CD-22-7-T-1) (0.500 g, 1.81 mmol) in anhydrous tetrahydrofuran (5 mL) was added cyclopropylmagnesium bromide (3.6 mL, 3.62 mmol) drop wise at −78° C. over 30 mins and stirred at −78° C. for 4 h. The reaction mixture was poured into aqueous NH₄Cl (sat.10 mL) and extracted with ethyl acetate (5 mL×2). Combined organic extracts were washed with brine (5 mL), dried over anhydrous Na₂SO₄ and concentrated in vacuo. The resulting crude was purified by silica gel column chromatography using a 5% ethyl acetate/hexane gradient to afford cyclopropyl(5-((3-fluorobenzyl)oxy)-2-nitrophenyl)methanol (CD-22-7-T-2) (0.340 g, 59%) as a brown oil.

tert-butyl (2-(cyclopropyl(hydroxy)methyl)-4-((3-fluorobenzyl)oxy)phenyl)carbamate (CD-22-7-T-3). To a stirred solution of cyclopropyl(5-((3-fluorobenzyl)oxy)-2-nitrophenyl)methanol (CD-22-7-T-2) (0.100 g, 0.95 mmol) and ammonium chloride (0.202 g, 11.35 mmol) in ethanol-water (5 mL-5 mL) was added Iron power (0.088 g, 4.73 mmol) at room temperature under nitrogen and the resulting mixture was stirred at 80° C. for 2 h. After cooling to room temperature, Iron was removed through filtration and washed with methanol (10 mL×2). Combined organic filtrates were concentrated in vacuo to give a residue which was taken up in DCM (2 mL). Followed by the addition of di-tert-butyl decarbonate (0.3 mL, 1.04 mmol) and sodium carbonate (0.200 g, 1.88 mmol), and the resulting mixture was stirred at room temperature for 2 h. The reaction mixture was poured into water (10 mL) and extracted with DCM (10 mL×2). Combined organic extracts were washed with brine (10 mL), dried over anhydrous Na₂SO₄ and concentrated in vacuo. The resulting crude was purified by silica gel column chromatography using a 10% ethyl acetate/hexane gradient to afford tert-butyl (2-(cyclopropyl(hydroxy)methyl)-4-((3-fluorobenzyl)oxy)phenyl)carbamate (CD-22-7-T-3) (0.080 g, 65%) as a white solid.

2-(cyclopropylmethyl)-4-((3-fluorobenzyl)oxy)aniline (CD-22-7-T-4). To a stirred solution of tert-butyl (2-(cyclopropyl(hydroxy)methyl)-4-((3-fluorobenzyl)oxy)phenyl)carbamate (CD-22-7-T-3) (0.075 g, 0.19 mmol) in dichloromethane (2 mL) was added Trifluoroacetic acid (1 mL)-triethylsilane (1 mL), and the resulting mixture was stirred at room temperature for 2 h. The reaction mixture was quenched with sodium hydroxide (10 mL, 20% in water) and extracted with dichloromethane (10 mL×3). The organic layers were combined, and concentrated under reduced pressure to afford a crude residue which was purified by silica gel flash column chromatography (eluted with 50% ethyl acetate in hexane) to afford 2-(cyclopropylmethyl)-4-((3-fluorobenzyl)oxy)aniline (0.020 g, 38% yield) as a colorless oil. MS [MH]⁺ 272.2.

N-(2-(cyclopropylmethyl)-4-((3-fluorobenzyl)oxy)phenyl)acrylamide (16). To a stirred solution of 2-(cyclopropylmethyl)-4-((3-fluorobenzyl)oxy)aniline (CD-22-7-T-4) (0.020 g, 0.074 mmol) and sodium hydroxide (1 mL, 20% in water) in DCM (1 mL) was added acryloyl chloride (0.045 g, 0.50 mmol) and stirred at room temperature for 30 minutes. The organic layer was collected and the aqueous layer was extracted with DCM (5 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(2-(cyclopropylmethyl)-4-((3-fluorobenzyl)oxy)phenyl)acrylamide (16) (0.006 g, 2%) as a white solid. ¹H NMR (400 MHz, CDCl₃): δ 7.70-7.68 (m, 1H), 7.37-7.31 (m, 1H), 7.20-7.14 (m, 2H), 7.03-6.99 (m, 3H), 6.85-6.82 (m, 1H), 6.39 (d, J=8.0 Hz, 1H), 6.23-6.30 (m, 1H), 5.75 (d, J=12.0 Hz, 1H), 5.06 (s, 2H), 2.51 (d, J=4.0 Hz, 2H), 0.95-0.92 (m, 1H), 0.60-0.58 (m, 2H), 0.19-0.18 (m, 1H). MS [MH]⁺ 326.2.

### Example 1.14. Synthesis of N-(4-((cyclopropylmethyl)(3-fluorobenzyl)amino)-3-fluorophenyl)acrylamide (17)

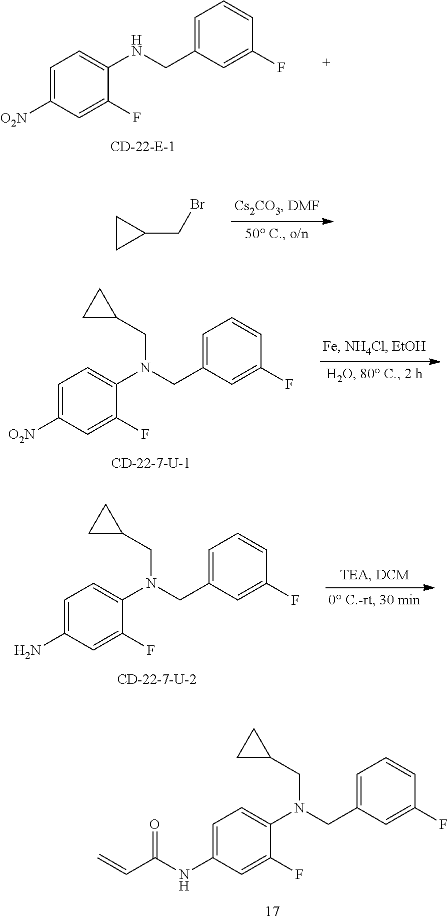

N-(cyclopropylmethyl)-2-fluoro-N-(3-fluorobenzyl)-4-nitroaniline (CD-22-7-U-1). To a solution of 2-fluoro-N-(3-fluorobenzyl)-4-nitroaniline (CD-22-E-1) (0.400 g, 1.50 mmol) and Cs₂CO₃ (0.977 g, 3.0 mmol) in DMF (10 mL) was added (bromomethyl)cyclopropane (0.223 g, 1.65 mmol) under at room temperature under nitrogen, and the resulting mixture was stirred at 50° C. for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford N-(cyclopropylmethyl)-2-fluoro-N-(3-fluorobenzyl)-4-nitroaniline (CD-22-7-U-1) (0.264 g, 55%) as a yellow oil.

N¹-(cyclopropylmethyl)-2-fluoro-N¹-(3-fluorobenzyl)benzene-1,4-diamine (CD-22-7-U-2). To a solution of N-(cyclopropylmethyl)-2-fluoro-N-(3-fluorobenzyl)-4-nitroaniline (CD-22-7-U-2) (0.250 g, 0.78 mmol) and NH₄Cl (0.209 g, 3.9 mmol) in EtOH (6 mL)-H₂O (2 mL) was added iron powder (0.218 g, 3.9 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 30% ethyl acetate/hexane gradient to afford N¹-(cyclopropylmethyl)-2-fluoro-N¹-(3-fluorobenzyl)benzene-1,4-diamine (CD-22-7-U-2) as a yellow oil (0.248 g, 95%). MS: [MH]⁺ 288.9.

N-(4-((cyclopropylmethyl)(3-fluorobenzyl)amino)-3-fluorophenyl)acrylamide (17). To a solution of N¹-(cyclopropylmethyl)-2-fluoro-N¹-(3-fluorobenzyl)benzene-1,4-diamine (CD-22-7-U-2) (0.235 g, 0.80 mmol) and TEA (0.162 g, 1.60 mmol) in DCM (5 mL) was added acryloyl chloride (0.080 g, 0.88 mmol) at 0° C. under nitrogen, and the resulting mixture was stirred at 0° C. for 30 min. The organic layer was collected and the aqueous layer was extracted with DCM (10 mL×2). Combined organic extracts were washed with brine (10 mL), dried over anhydrous Na₂SO₄, and concentrated in vacuo. The resulting crude was purified by silica gel column chromatography using a 50-60% ethyl acetate/hexane gradient to afford N-(4-((cyclopropylmethyl)(3-fluorobenzyl)amino)-3-fluorophenyl)acrylamide (17) as a yellow solid (0.199 g, 73%). ¹HNMR (400 MHz, CDCl₃) δ 7.48 (d, J=14.0 Hz, 1H), 7.29 (br, 1H), 7.24-7.20 (m, 1H), 7.10-7.06 (m, 3H), 6.95-6.87 (m, 2H), 6.45-6.40 (m, 1H), 6.24-6.17 (m, 1H), 5.74-5.70 (m, 1H), 4.38 (s, 2H), 2.98 (d, J=6.4 Hz, 2H), 0.97-0.86 (m, 1H), 0.43-0.39 (m, 2H) MS: [MH]⁺ 343.5.

The following compound was prepared in a manner analogous to the procedures described above for N-(4-((cyclopropylmethyl)(3-fluorobenzyl)amino)-3-fluorophenyl)acrylamide (17):

N-(3-fluoro-4-((3-fluorobenzyl)(pyridin-4-ylmethyl)amino)phenyl)acrylamide (18) as a yellow solid (0.171 g, 40%). ¹HNMR (400 MHz, CDCl₃) δ 8.67 (d, J=6.4 Hz, 2H), 7.73 (d, J=6.0 Hz, 2H), 7.66-7.60 (m, 2H), 7.31-7.20 (m, 1H), 7.07-7.02 (m, 4H), 6.83-6.78 (m, 2H), 6.44-6.40 (m, 1H), 6.25-6.17 (m, 1H), 5.78-5.75 (m, 1H), 4.45 (s, 2H), 4.30 (s, 2H) MS: [MH]⁺ 380.5.

### Example 1.15. Synthesis of N-(5-((4,4-difluorocyclohexyl)methoxy)isoquinolin-8-yl)acrylamide (19)

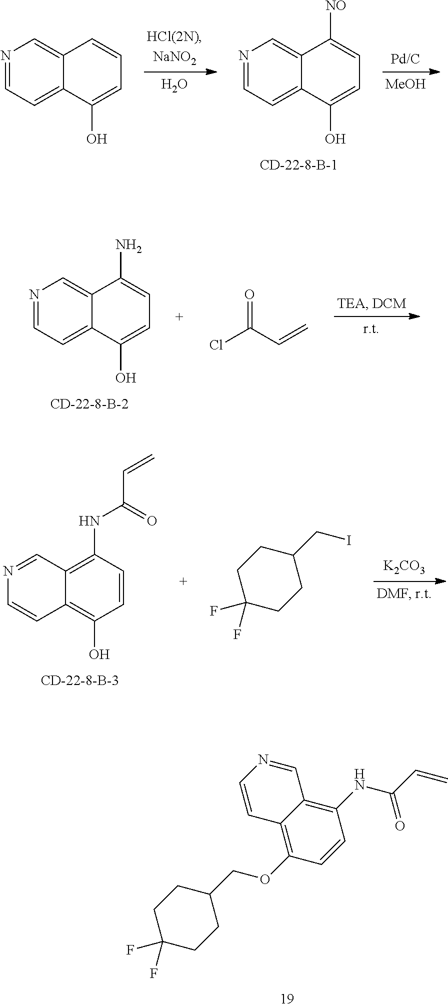

8-nitrosoisoquinolin-5-ol (CD-22-8-B-1). A mixture of isoquinolin-5-ol (3.000 g, 20.60 mmol) in in hydrochloric acid (30 mL, 2 N) was stirred at 0° C., and sodium nitrite (1.500 g, 22.60 mmol) in water was added dropwise into the mixture and stirred at room temperature overnight. TLC showed the reaction was complete. The mixture was filtered, and the filter cake was washed with water (30 mL) and dried to afford 8-nitrosoisoquinolin-5-ol (CD-22-8-B-1) (2.400 g, 66%) as a yellow solid. MS: [MH]⁺ 175.0.

8-aminoisoquinolin-5-ol (CD-22-8-B-2). A mixture of 8-nitrosoisoquinolin-5-ol (CD-22-8-B-1) (0.200 g, 1.10 mmol) and Pd/C (0.060 g) in methanol (5 mL) was stirred at room temperature under hydrogen for 2 h. The mixture was filtered, and the filtrate was concentrated to afford 8-aminoisoquinolin-5-ol (CD-22-8-B-2) (0.150 g, yield 71%) as a brown solid. MS: [MH]⁺ 161.0.

N-(5-hydroxyisoquinolin-8-yl)acrylamide (CD-22-8-B-3). To a solution of 8-aminoisoquinolin-5-ol (CD-22-8-B-2) (0.420 g, 2.60 mmol) and triethylamine (0.410 g, 3.10 mmol) in dichloromethane (5 mL) was added acryloyl chloride (0.310 g, 2.60 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 30% ethyl acetate/hexane gradient to afford N-(5-hydroxyisoquinolin-8-yl)acrylamide (CD-22-8-B-3) (0.430 g, 77%) as a light yellow oil.

N-(5-((4,4-difluorocyclohexyl)methoxy)isoquinolin-8-yl)acrylamide (19). To a solution of N-(5-hydroxyisoquinolin-8-yl)acrylamide (CD-22-8-B-3) (0.730 g, 2.00 mmol) and 1,1-difluoro-4-(iodomethyl)cyclohexane (0.700 g, crude) was added potassium carbonate (0.560 g, 4.10 mmol) in N,N-dimethylformamide (2 mL) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 10% dichloromethane and 10% ethyl acetate in hexane gradient to afford N-(5-((4,4-difluorocyclohexyl)methoxy)isoquinolin-8-yl)acrylamide (19) (0.001 g, 1% of 2 steps) as a white solid. ¹H NMR (400 MHz, CDCl₃): δ 9.22 (s, 1H), 8.73 (d, J=9.2 Hz, 1H), 8.53-8.51 (m, 1H), 7.98 (s, 1H), 7.80 (d, J=9.2 Hz, 1H), 7.71 (d, J=6.0 Hz, 1H), 6.52 (d, J=16.8 Hz, 1H), 6.35-6.30 (m, 1H), 5.89 (d, J=10.0 Hz, 1H), 3.87 (d, J=6.0 Hz, 2H), 2.28-2.19 (m, 2H), 2.12-2.00 (m, 3H), 1.92-1.77 (m, 2H), 1.64-1.61 (m, 2H). MS: [MH]⁺ 347.4.

### Example 1.16. Synthesis of N-(2-cyano-4-((4,4-difluorocyclohexyl)methoxy)phenyl)acrylamide (20)

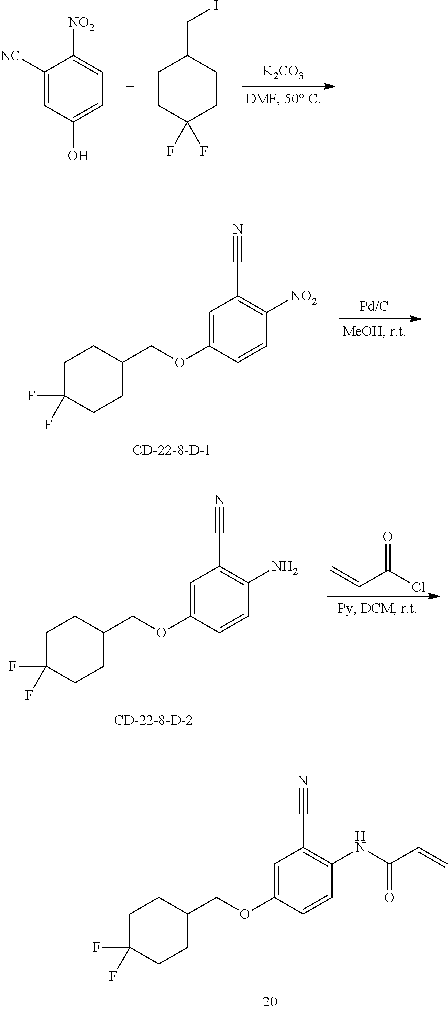

5-((4,4-difluorocyclohexyl)methoxy)-2-nitrobenzonitrile (CD-22-8-D-1). To a mixture of 2-bromo-4-nitrophenol (2.000 g, 9.20 mmol) and 1-(bromomethyl)-3-fluorobenzene (1.900 g, 10.10 mmol) in DMF (20 mL) was added potassium carbonate (2.800 g, 18.40 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 40-50% ethyl acetate/hexane gradient to afford 2-bromo-1-((3-fluorobenzyl)oxy)-4-nitrobenzene (CD-22-8-D-1) (1.800 g, 62%) as a yellow solid.

2-amino-5-((4,4-difluorocyclohexyl)methoxy)benzonitrile (CD-22-8-D-2). A mixture of 5-((4,4-difluorocyclohexyl)methoxy)-2-nitrobenzonitrile (CD-22-8-D-1) (0.185 g, 0.62 mmol) in methanol (30 mL) was added palladium on carbon (10%, 123 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to give crude product, which was purified by silica gel flash column chromatography using a 1% methanol/dichloromethane gradient to afford 2-amino-5-((4,4-difluorocyclohexyl)methoxy)benzonitrile (CD-22-8-D-2) (0.106 g, 63%) as a white solid.

N-(2-cyano-4-((4,4-difluorocyclohexyl)methoxy)phenyl)acrylamide (20). A mixture of 2-amino-5-((4,4-difluorocyclohexyl)methoxy)benzonitrile (CD-22-8-D-2) (0.092 g, 0.35 mmol) and pyridine (0.109 g, 1.38 mmol) in dichloromethane (6 mL) was added acryloyl chloride (0.045 g, 0.50 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford N-(2-cyano-4-((4,4-difluorocyclohexyl)methoxy)phenyl)acrylamide (20) (0.018 g, 16%) as a white solid. ¹H NMR (400 MHz, CD₃OD) δ 7.55 (d, J=8.8 Hz, 1H), 7.32-7.26 (m, 2H), 6.54-6.40 (m, 2H), 5.88-5.85 (m, 1H), 3.94 (d, J=6.0 Hz, 2H), 2.13-2.08 (m, 2H), 1.99-1.79 (m, 5H), 1.47 (d, J=12.4 Hz, 2H). MS: [MH]⁺ 321.3.

### Example 1.17. Synthesis of N-(7-((4,4-difluorocyclohexyl)methoxy)benzo[d]oxazol-4-yl)acrylamide (21)

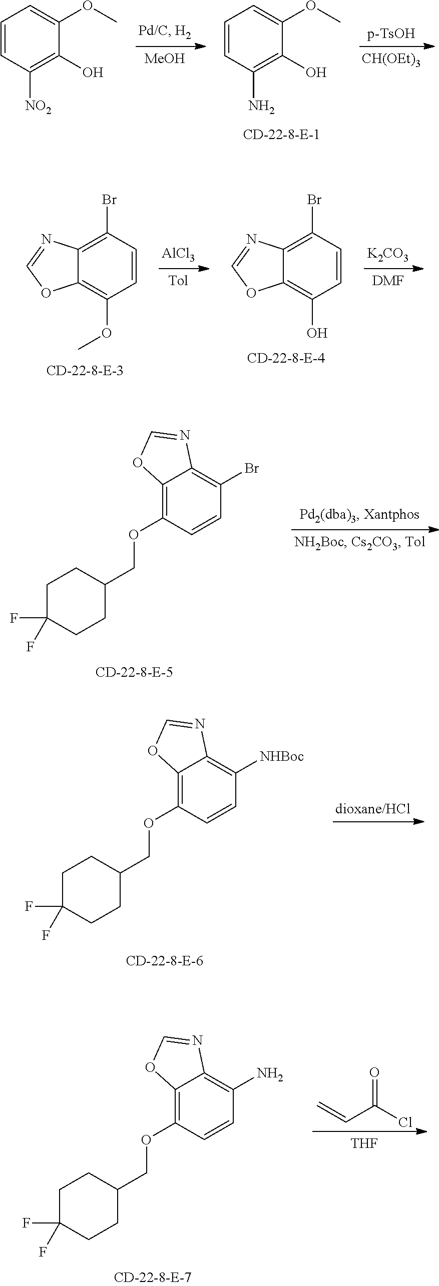

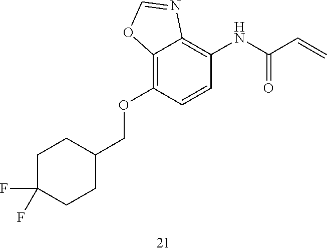 [SMILES: C=CC(=O)Nc1ccc(OCC2CCC(F)(F)CC2)c2ocnc12]

2-amino-6-methoxyphenol (CD-22-8-E-1). To a solution of 2-methoxy-6-nitrophenol (0.450 g, 2.66 mmol) in methanol (30 mL) was added palladium on carbon (10%, 123 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to give crude product, which was purified by silica gel flash column chromatography using a 1% methanol/dichloromethane gradient to afford 2-amino-6-methoxyphenol (CD-22-8-E-1) (0.380 g, crude) as red solid, which was used in next step without further purification.

7-methoxybenzo[d]oxazole (CD-22-8-E-2). To a stirred solution of 2-amino-6-methoxyphenol (CD-22-8-E-1) (0.500 g, 3.60 mmol) in triethoxymethane (10 mL) was added p-toluenesulfonic acid (0.060 g) at room temperature, and the reaction mixture was stirred at 130° C. for 12 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 10% ethyl acetate/hexane gradient to afford 7-methoxybenzo[d]oxazole (CD-22-8-E-2) (0.500 g, 93%) as a colorless oil.

4-bromo-7-methoxybenzo[d]oxazole (CD-22-8-E-3). To a stirred solution of 7-methoxybenzo[d]oxazole (CD-22-8-E-2) (0.100 g, 0.67 mmol) in acetonitrile (10 mL) was added N-bromosuccinimide (0.119 g, 0.67 mmol) at room temperature. The reaction mixture was stirred at 80° C. for 12 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5% ethyl acetate/hexane gradient to afford 4-bromo-7-methoxybenzo[d]oxazole (CD-22-8-E-3) (0.100 g, 65%) as a white solid. MS: [MH]⁺ 228.0, 229.9.

4-bromobenzo[d]oxazol-7-ol (CD-22-8-E-4). To a stirred solution of 4-bromo-7-methoxybenzo[d]oxazole (CD-22-8-E-3) (0.500 g, 2.20 mmol) in toluene (20 mL) was added aluminum chloride (0.880 g, 6.60 mmol) at room temperature. The reaction mixture was stirred at room temperature for 2 h. The reaction mixture was poured into water (10 mL) and extracted with ethyl acetate (15 mL×2). Combined organic extracts were washed with brine (15 mL), dried over anhydrous Na₂SO₄, and concentrated in vacuo. The resulting crude was purified by silica gel column chromatography using a 50% ethyl acetate/hexane gradient to afford 4-bromobenzo[d]oxazol-7-ol (CD-22-8-E-4) (0.400 g, 85%) as a yellow solid. MS: [MH]⁺ 213.8, 215.8.

4-bromo-7-((4,4-difluorocyclohexyl)methoxy)benzo[d]oxazole (CD-22-8-E-5). To a stirred solution of 4-bromobenzo[d]oxazol-7-ol (CD-22-8-E-4) (0.200 g, 1.00 mmol) 1,1-difluoro-4-(iodomethyl)cyclohexane (0.260 g, 1.00 mmol) in N,N-dimethylformamide (10 mL) was added potassium carbonate (0.400 g, 3.00 mmol) at room temperature. The reaction mixture was stirred at 50° C. for 12 h. The reaction mixture was poured into water (20 mL) and extracted with ethyl acetate (15 mL×2). Combined organic extracts were washed with brine (25 mL), dried over anhydrous Na₂SO₄, and concentrated in vacuo. The resulting crude was purified by silica gel column chromatography using a 5% ethyl acetate/hexane gradient to afford 4-bromo-7-((4,4-difluorocyclohexyl)methoxy)benzo[d]oxazole (CD-22-8-E-5) (0.280 g, 81%) as a yellow solid.

tert-butyl (7-((4,4-difluorocyclohexyl)methoxy)benzo[d]oxazol-4-yl)carbamate (CD-22-8-E-6). To a stirred solution of 4-bromo-7-((4,4-difluorocyclohexyl)methoxy)benzo[d]oxazole (CD-22-8-E-5) (0.250 g, 0.72 mmol), tert-butyl carbamate (0.169 g, 1.45 mmol), and cesium carbonate (0.702 g, 2.16 mmol) in toluene (10 mL) was added tris(dibenzylideneacetone)dipalladium(0) (0.064 g, 0.07 mmol) and 9,9-dimethyl-4,5-bisdiphenylphosphinoxanthene (0.081 g, 0.14 mmol) at room temperature under nitrogen atmosphere; the mixture was degassed with nitrogen three times. The resulting mixture was refluxed for 2 hours. The reaction mixture was poured into water (10 mL) and extracted with ethyl acetate (15 mL×2). Combined organic extracts were washed with brine (40 mL), dried over anhydrous Na₂SO₄, and concentrated in vacuo. The resulting crude was purified by silica gel column chromatography using a 30-50% ethyl acetate/hexane gradient to afford tert-butyl (7-((4,4-difluorocyclohexyl)methoxy)benzo[d]oxazol-4-yl)carbamate (CD-22-8-E-6) (0.200 g, 72%) as a yellow solid.

7-((4,4-difluorocyclohexyl)methoxy)benzo[d]oxazol-4-amine (CD-22-8-E-7). A solution of tert-butyl (7-((4,4-difluorocyclohexyl)methoxy)benzo[d]oxazol-4-yl)carbamate (CD-22-8-E-6) (0.090 g, 0.27 mmol) in 4 M hydrogen chloride in dioxane (10 mL) was stirred at room temperature for 1 h. The resulting mixture was concentrated in vacuo. The resulting crude was taken up in aqueous solution of sodium carbonate (10 mL) and extracted with ethyl acetate (10 mL×3). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford 7-((4,4-difluorocyclohexyl)methoxy)benzo[d]oxazol-4-amine (CD-22-8-E-7) (0.050 g, 66%) as a yellow solid. MS: [MH]⁺ 283.2.

N-(7-((4,4-difluorocyclohexyl)methoxy)benzo[d]oxazol-4-yl)acrylamide (21). To a stirred solution of 7-((4,4-difluorocyclohexyl)methoxy)benzo[d]oxazol-4-amine (0.050 g, 0.18 mmol) in tetrahydrofuran (2 mL) was added acryloyl chloride (0.016 g, 0.18 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(7-((4,4-difluorocyclohexyl)methoxy)benzo[d]oxazol-4-yl)acrylamide (21) (0.008 g, 13%) as a white solid. ¹H NMR (400 MHz, CDCl₃): δ 8.31 (d, J=8.8 Hz, 1H), 8.09 (s, 1H), 8.04 (s, 1H), 6.88 (d, J=9.2 Hz, 1H), 6.51-6.46 (m, 1H), 6.39-6.32 (m, 1H), 5.83-5.80 (m, 1H), 4.05 (d, J=6.4 Hz, 2H), 2.20-2.15 (m, 2H), 2.03-2.00 (m, 3H), 1.87-1.70 (m, 2H), 1.53-1.47 (m, 2H). MS: [MH]⁺ 337.3.

### Example 1.18. Synthesis of N-(7-((4,4-difluorocyclohexyl)methoxy)-1H-benzo[d]imidazol-4-yl)acrylamide (22)

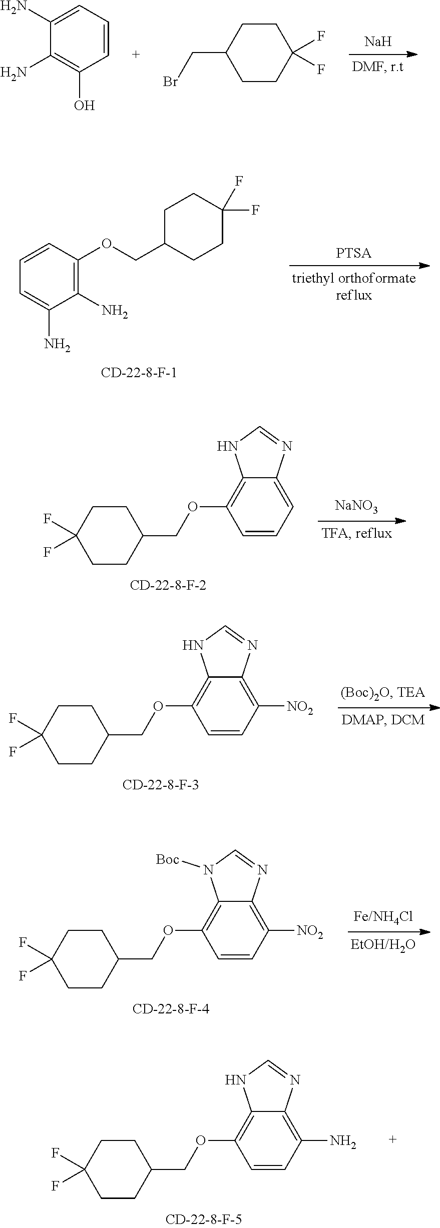

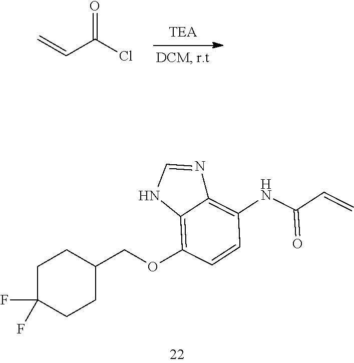

3-((4,4-difluorocyclohexyl) methoxy)benzene-1,2-diamine (CD-22-8-F-1). To a suspension of sodium hydride (0.485 g, 12.00 mmol) in DMF (10 mL) was added a solution of 2,3-diaminophenol (0.752 g, 6.10 mmol) in DMF (5 mL) at 0° C., and the reaction mixture was stirred at 0° C. for 30 min, followed by the addition of a solution of 4-(bromomethyl)-1,1-difluorocyclohexane (1.550 g, 7.30 mmol) in DMF (5 mL) at 0° C. The resulting mixture was stirred at 0° C. for 1 h. TLC showed the reaction was complete. The reaction mixture was poured into water (30 mL) and extracted with ethyl acetate (15 mL×2). Combined organic extracts were washed with brine (35 mL), dried over anhydrous Na₂SO₄, and concentrated in vacuo. The resulting crude was purified by silica gel column chromatography using a 30-50% ethyl acetate/hexane gradient to afford 3-((4,4-difluorocyclohexyl)methoxy)benzene-1,2-diamine (CD-22-8-F-1) (0.308 g, 20%) as a light yellow oil. MS: [MH]⁺ 257.3.

7-((4,4-difluorocyclohexyl)methoxy)-1H-benzo[d]imidazole (CD-22-8-F-2). To a stirred solution of 3-((4,4-difluorocyclohexyl)methoxy)benzene-1,2-diamine (CD-22-8-F-1) (0.308 g, 1.20 mmol) and p-toluene sulfonic acid (0.010 g, 0.06 mmol) in triethyl orthoformate (10 mL) under nitrogen. The resulting mixture was stirred at reflux for 4 hours. The reaction mixture was concentrated under reduced pressure, and the resulting crude was purified by silica gel column chromatography using a 50% ethyl acetate/hexane gradient to afford 7-((4,4-difluorocyclohexyl)methoxy)-1H-benzo[d]imidazole (CD-22-8-F-2) (0.277 g, 87%) as a yellow oil. MS: [MH]⁺ 267.3.

7-((4,4-difluorocyclohexyl)methoxy)-4-nitro-1H-benzo[d]imidazole (CD-22-8-F-3). A solution of 7-((4,4-difluorocyclohexyl)methoxy)-1H-benzo[d]imidazole (CD-22-8-F-2) (0.277 g, 1.04 mmol) and sodium nitrate (0.133 g, 1.56 mmol) in trifluoroacetic acid (8 mL) was stirred under nitrogen at 70° C. for 4 hours. The reaction mixture was quenched with water (10 mL) and adjusted pH to 8 with 10% sodium hydroxide. The resulting mixture was extracted with ethyl acetate (15 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 7-((4,4-difluorocyclohexyl)methoxy)-4-nitro-1H-benzo[d]imidazole (CD-22-8-F-3) (0.136 g, 42%) as white solid. MS: [MH]⁺ 312.1.

tert-butyl 7-((4,4-difluorocyclohexyl)methoxy)-4-nitro-1H-benzo[d]imidazole-1-carboxylate (CD-22-8-F-4). To a stirring mixture of 7-((4,4-difluorocyclohexyl)methoxy)-4-nitro-1H-benzo[d]imidazole (CD-22-8-F-3) (0.136 g, 0.44 mmol), 4-dimethylaminopyridine (0.011 g, 0.09 mmol), and triethylamine (0.088 g, 0.87 mmol) in dichloromethane (10 mL) was added di-tert-butyl dicarbonate (0.143 g, 0.66 mmol), and the resulting mixture was stirred at 0° C. for 2 hours. TLC showed the reaction was complete. The reaction mixture was partitioned between dichloromethane (100 mL) and water (50 mL); the organic layer was collected, and the aqueous layer was extracted with dichloromethane (50 mL×2). The combined organic layers were washed with brine (50 mL), dried over anhydrous sodium sulfate, and concentrated under reduced pressure to give a crude residue, which was purified by silica gel flash chromatography (eluted with 50% ethyl acetate in petroleum ether) to afford tert-butyl 7-((4,4-difluorocyclohexyl)methoxy)-4-nitro-1H-benzo[d]imidazole-1-carboxylate (CD-22-8-F-4) (0.150 g, 83%) as light yellow solid. MS: [MH]⁺ 412.0.

7-((4,4-difluorocyclohexyl)methoxy)-1H-benzo[d]imidazol-4-amine (CD-22-8-F-5). To a solution of 7-((4,4-difluorocyclohexyl)methoxy)-4-nitro-1H-benzo[d]imidazole-1-carboxylate (CD-22-8-F-4) (0.127 g, 0.31 mmol) and ammonium chloride (0.083 g, 1.54 mmol) in EtOH (4 mL)-H₂O (1 mL) was added iron powder (0.086 g, 1.54 mmol). The resulting mixture was stirred at 90° C. overnight. TLC showed the reaction was complete. The reaction mixture was concentrated under reduced pressure to give a crude residue, which was purified by silica gel flash chromatography (eluted with 5% methanol in anhydrous dichloromethane) to afford 7-((4,4-difluorocyclohexyl)methoxy)-1H-benzo[d]imidazol-4-amine (CD-22-8-F-5) (0.053 g, 61%) as light brown solid. MS: [MH]⁺ 282.1.

N-(7-((4,4-difluorocyclohexyl)methoxy)-1H-benzo[d]imidazol-4-yl)acrylamide (22). To a solution of 7-((4,4-difluorocyclohexyl)methoxy)-1H-benzo[d]imidazol-4-amine (0.040 g, 0.14 mmol) and triethylamine (0.029 g, 0.28 mmol) in dichloromethane (5 mL) was added acryloyl chloride (0.014 g, 0.16 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford N-(7-((4,4-difluorocyclohexyl)methoxy)-1H-benzo[d]imidazol-4-yl)acrylamide (22) (0.010 g, 21%) as off white solid. ¹H NMR (400 MHz, DMSO-d₆): δ 12.83 (s, 1H), 9.45 (s, 1H), 8.13 (s, 1H), 7.87 (s, 1H), 6.70 (d, J=8.4 Hz, 2H), 6.25 (d, J=16.8 Hz, 1H), 5.73 (d, J=8.8 Hz, 1H), 4.04 (s, 2H), 2.08-1.78 (m, 7H), 1.42-1.33 (m, 2H). MS: [MH]⁺ 336.3.

### Example 1.19. Synthesis of N-(4-((4,4-difluorocyclohexyl)methoxy)pyrazolo[1,5-a]pyridin-7-yl)acrylamide (23)

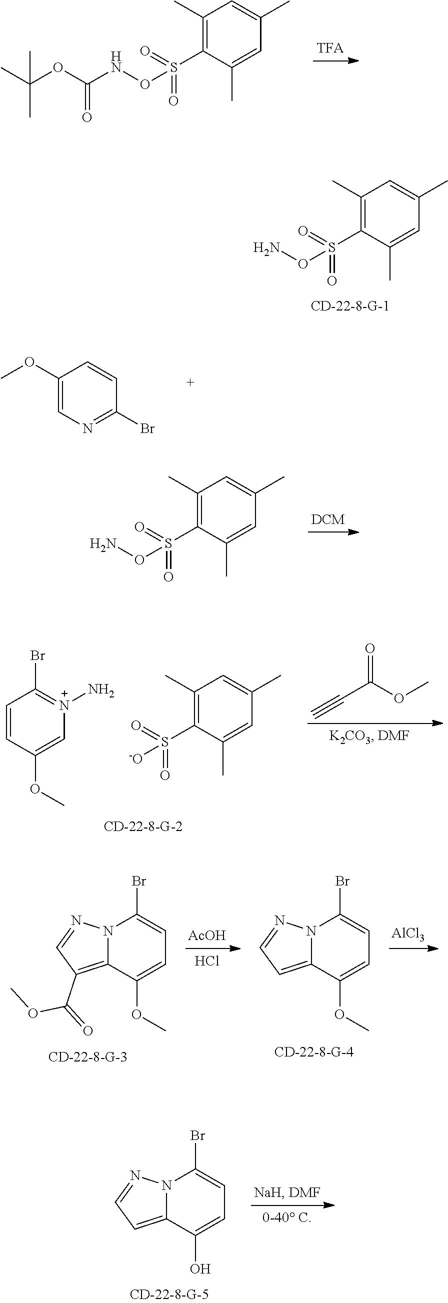

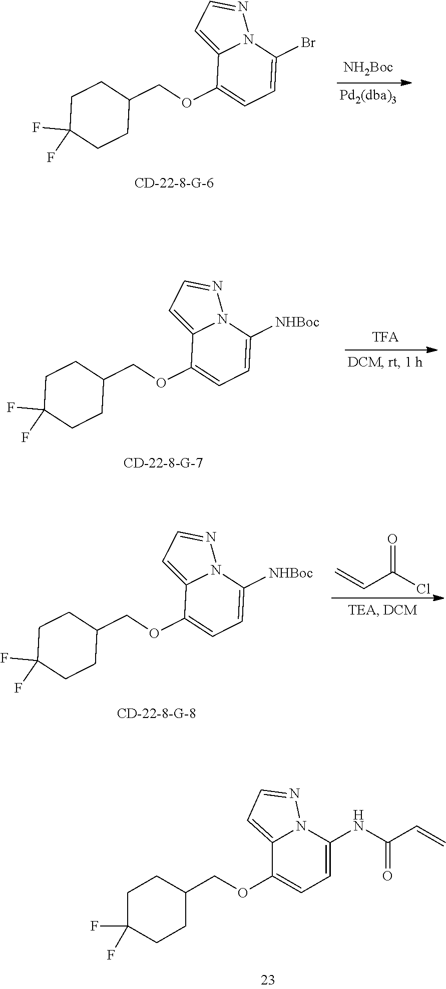

O-(mesitylsulfonyl)hydroxylamine (CD-22-8-G-1). A mixture of tert-butyl ((mesitylsulfonyl)oxy)carbamate (15.000 g, 47.60 mmol) in TFA (20 mL) was stirred at 0° C. under N₂, and the reaction mixture was stirred for 30 min. Water (60 mL) was added slowly, and the resulting mixture was stirred for another 15 min. The precipitate was filtered and washed several times with water until the pH of the filtrate was neutral. The white solid O-(mesitylsulfonyl)hydroxylamine (CD-22-8-G-1) (8.700 g, 86%) was used immediately for next reaction.

1-amino-2-bromo-5-methoxypyridin-1-ium 2,4,6-trimethylbenzenesulfonate (CD-22-8-G-2). A mixture of 2-bromo-5-methoxypyridine (5.100 g, 27.10 mmol) and O-(mesitylsulfonyl)hydroxylamine (CD-22-8-G-1) (8.700 g, 40.60 mmol) in DCM (100 mL) was stirred at 10° C. for 18 h under N₂. The reaction mixture was concentrated to give crude 1-amino-2-bromo-5-methoxypyridin-1-ium 2,4,6-trimethylbenzenesulfonate (CD-22-8-G-2) (13.000 g, crude) as yellow solid which was used immediately for next reaction. MS: [MH]⁺ 202.8, 204.8.

methyl 7-bromo-4-methoxypyrazolo[1,5-a]pyridine-3-carboxylate (CD-22-8-G-3). To a stirring mixture of 1-amino-2-bromo-5-methoxypyridin-1-ium 2,4,6-trimethylbenzenesulfonate (CD-22-8-G-2) (13.000 g, crude) and methyl propiolate (2.300 g, 27.20 mmol) in N,N-dimethylformamide (30 mL) was added potassium carbonate (7.500 g, 54.40 mmol) at 0° C. The resulting mixture was stirred at room temperature for 18 h. The reaction mixture was poured into water (50 mL) and extracted with ethyl acetate (50 mL×2). Combined organic extracts were washed with brine (50 mL), dried over anhydrous Na₂SO₄, and concentrated in vacuo. The resulting crude was purified by silica gel column chromatography using a 30-50% ethyl acetate/hexane gradient to afford methyl 7-bromo-4-methoxypyrazolo[1,5-a]pyridine-3-carboxylate (CD-22-8-G-3) (2.700 g, 34% in two steps) as yellow solid. MS: [MH]⁺ 284.9, 286.8.

7-bromo-4-methoxypyrazolo[1,5-a]pyridine (CD-22-8-G-4). To a stirring mixture of methyl 7-bromo-4-methoxypyrazolo[1,5-a]pyridine-3-carboxylate (CD-22-8-G-3) (13.000 g, crude) in acetic acid (10 mL)-water (10 mL) was added hydrochloric acid (12 N, 7 mL) at 0° C., and the resulting mixture was stirred at 100° C. for 18 h. The reaction mixture was basified with 6 M NaOH to pH 10, then extracted with ethyl acetate (50 mL×2). The organic extracts were washed with brine (30 mL), dried over anhydrous Na₂SO₄, and concentrated in vacuo. The resulting crude was purified by silica gel column chromatography using a 20% ethyl acetate/hexane gradient to afford 7-bromo-4-methoxypyrazolo[1,5-a]pyridine (CD-22-8-G-4) (1.100 g, 65%) as yellow solid. MS: [MH]⁺ 226.8, 228.7.

7-bromopyrazolo[1,5-a]pyridin-4-ol (CD-22-8-G-5). To a stirring mixture of 7-bromo-4-methoxypyrazolo[1,5-a]pyridine (CD-22-8-G-4) (1.100 g, 4.90 mmol) in 1,2-dichloroethane (10 mL) was added aluminum trichloride (1.900 g, 14.60 mmol) at 0° C., and the resulting mixture was stirred at 85° C. for 5 h. The reaction mixture was basified with sat. NaHCO₃ to pH 8-9, then extracted with ethyl acetate (30 mL×2) and water (30 mL); the organic layer was collected, and the aqueous layer was extracted with ethyl acetate (20 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 7-bromopyrazolo[1,5-a]pyridin-4-ol (CD-22-8-G-5) (0.720 g, 70%) as white solid.

7-bromo-4-((4,4-difluorocyclohexyl)methoxy)pyrazolo[1,5-a]pyridine (CD-22-8-G-6). To a solution of sodium hydride (60% in mineral oil, 0.112 g, 4.70 mmol) in DMF (8 mL) was added 7-bromopyrazolo[1,5-a]pyridin-4-ol (CD-22-8-G-5) (0.300 g, 1.40 mmol) at 0° C., and the resulting mixture was stirred at 0° C. for 30 min, followed by the addition of a solution of 4-(bromomethyl)-1,1-difluorocyclohexane (0.448 g, 2.10 mmol) in DMF (3 mL) at room temperature. The resulting mixture was stirred at 40° C. for 4 h. The reaction mixture was poured into water (30 mL) and extracted with ethyl acetate (15 mL×2). Combined organic extracts were washed with brine (25 mL), dried over anhydrous Na₂SO₄, and concentrated in vacuo. The resulting crude was purified by silica gel column chromatography using a 30-50% ethyl acetate/hexane gradient to afford 7-bromo-4-((4,4-difluorocyclohexyl)methoxy)pyrazolo[1,5-a]pyridine (CD-22-8-G-6) (0.160 g, 34%) as white solid. MS: [MH]⁺ 344.9, 346.8.

tert-butyl (4-((4,4-difluorocyclohexyl)methoxy)pyrazolo[1,5-a]pyridin-7-yl)carbamate (CD-22-8-G-7). To a stirred solution of 7-bromo-4-((4,4-difluorocyclohexyl)methoxy)pyrazolo[1,5-a]pyridine (CD-22-8-G-6) (0.160 g, 0.46 mmol), tert-butyl carbamate (0.060 g, 0.51 mmol), and cesium carbonate (0.448 g, 1.38 mmol) in dry toluene (15 mL) was added Pd₂(dba)₃ (0.046 g, 0.05 mmol) and XantPhos (0.052 g, 0.09 mmol) at room temperature under nitrogen; the mixture was degassed with nitrogen three times. The resulting mixture was stirred at 110° C. for 3 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford tert-butyl(4-((4,4-difluorocyclohexyl)methoxy)pyrazolo[1,5-a]pyridin-7-yl)carbamate (CD-22-8-G-7) (0.100 g, 56%) as white solid. MS: [MH]⁺ 382.2.

4-((4,4-difluorocyclohexyl)methoxy)pyrazolo[1,5-a]pyridin-7-amine (CD-22-8-G-8). A mixture of tert-butyl (4-((4,4-difluorocyclohexyl)methoxy)pyrazolo[1,5-a]pyridin-7-yl)carbamate (CD-22-8-G-7) (0.050 g, 0.13 mmol) and 2,2,2-trifluoroacetic acid (2 mL) in anhydrous dichloromethane (4 mL) was stirred at room temperature for 30 min. The volatiles were evaporated under reduced pressure to afford 4-((4,4-difluorocyclohexyl)methoxy)pyrazolo[1,5-a]pyridin-7-amine (CD-22-8-G-8) (0.043 g. crude) as a yellow solid. MS: [MH]⁺ 282.3.

N-(4-((4,4-difluorocyclohexyl)methoxy)pyrazolo[1,5-a]pyridin-7-yl)acrylamide (23). To a stirring mixture of 4-((4,4-difluorocyclohexyl)methoxy)pyrazolo[1,5-a]pyridin-7-amine (0.040 g, crude) and triethylamine (0.029 g, 0.28 mmol) in dichloromethane (10 mL) was added acryloyl chloride (0.019 g, 0.21 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(4-((4,4-difluorocyclohexyl)methoxy)pyrazolo[1,5-a]pyridin-7-yl)acrylamide (23) (0.018 g, two steps yield 41%) as white solid. ¹HNMR (400 MHz, DMSO-d₆): δ 10.54 (s, 1H), 8.03 (d, J=2.0 Hz, 1H), 7.44 (d, J=8.4 Hz, 1H), 6.91-6.84 (m, 1H), 6.74-6.70 (m, 2H), 6.35-6.30 (m, 1H), 5.82 (d, J=10 Hz, 1H), 4.03 (d, J=6.0 Hz, 2H), 2.07-1.79 (m, 7H), 1.42-1.32 (m, 2H). MS: [MH]⁺ 336.3.

### Example 1.20. Synthesis of N-(4-((3,3-difluorocyclohexyl)methoxy)-3-fluorophenyl)acrylamide (24)

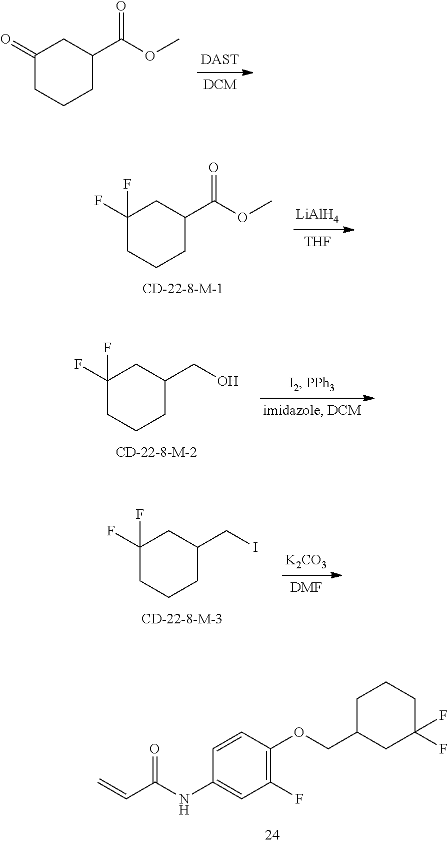

methyl 3,3-difluorocyclohexane-1-carboxylate (CD-22-8-M-1). To a stirred solution of methyl 3-oxocyclohexane-1-carboxylate (1.000 g, 6.40 mmol) in dichloromethane (20 mL) was added diethylaminosulfur trifluoride (2.100 g, 12.80 mmol). The resulting mixture was stirred at room temperature for 2 h. The reaction mixture was poured into water (50 mL) and extracted with ethyl acetate (20 mL×3). Combined organic extracts were washed with brine (5 mL), dried over anhydrous Na₂SO₄, and concentrated in vacuo. The resulting crude was purified by silica gel column chromatography using a 5-10% ethyl acetate/hexane gradient to afford methyl 3,3-difluorocyclohexane-1-carboxylate (CD-22-8-M-1) (0.600 g, 53%) as a colorless oil.

(3,3-difluorocyclohexyl)methanol (CD-22-8-M-2). To a stirred solution of lithium aluminum hydride (0.427 g, 11.20 mmol) in anhydrous tetrahydrofuran (20 mL) was added methyl 3,3-difluorocyclohexane-1-carboxylate (CD-22-8-M-1) (0.500 g, 2.80 mmol) in 5 minutes at room temperature. The resulting mixture was stirred at room temperature for 8 h. The mixture was quenched with water (0.42 mL) at 0° C. and stirred at this temperature for 10 minutes. To the resulting mixture was added sodium hydroxide (0.42 mL, 15% in water) followed by water (1.28 mL) and stirred at room temperature for 0.5 h. To the resulting mixture was added anhydrous sodium sulfate and stirred at room temperature for 0.2 h. Solid powder was removed through filtration, and the filter cake was washed with tetrahydrofuran (30 mL×2). Combined organic filtrates were concentrated in vacuo. The resulting crude was purified by silica gel column chromatography using a 20%-30% ethyl acetate/hexane gradient to afford (3,3-difluorocyclohexyl)methanol (CD-22-8-M-2) (0.250 g, 59%) as a colorless oil.

1,1-difluoro-3-(iodomethyl)cyclohexane (CD-22-8-M-3). To a stirred solution of triphenylphosphine (0.565 g, 2.16 mmol) and imidazole (0.191 g, 2.80 mmol) in dichloromethane (5 mL) was added iodine (0.546 g, 2.16 mmol) at room temperature. The resulting mixture was stirred at room temperature for 10 min, followed by the addition of (3,3-difluorocyclohexyl)methanol (CD-22-8-M-2) (0.250 g, 1.66 mmol) at 0° C. The resulting mixture was stirred at room temperature for 16 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford 1,1-difluoro-3-(iodomethyl)cyclohexane (CD-22-8-M-3) (0.300 g, 70%) as a colorless oil.

N-(4-((3,3-difluorocyclohexyl)methoxy)-3-fluorophenyl)acrylamide (24). To a stirred solution of 1,1-difluoro-3-(iodomethyl)cyclohexane (CD-22-8-M-3) (0.086 g, 0.33 mmol) and N-(3-fluoro-4-hydroxyphenyl)acrylamide (0.050 g, 0.28 mmol) in N,N-dimethylformamide (5 mL) was added potassium carbonate (0.116 g, 0.84 mmol). The resulting mixture was stirred at room temperature overnight. TLC showed the reaction was complete. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5% MeOH/DCM gradient to afford N-(4-((3,3-difluorocyclohexyl)methoxy)-3-fluorophenyl)acrylamide (24) (0.018 g, 21%) as white solid. ¹H NMR (400 MHz, DMSO-d₆): δ 7.50-7.46 (m, 1H), 7.17-7.14 (m, 1H), 6.94 (t, J=8.8 Hz, 1H), 6.33-6.21 (m, 2H), 5.67-5.64 (m, 1H), 3.87-3.78 (m, 2H), 2.20-1.91 (m, 3H), 1.79-1.75 (m, 2H), 1.69-1.43 (m, 3H), 1.19-1.09 (m, 1H). MS: [MH]⁺ 314.1.

### Example 1.21. Synthesis of N-(4-((3-ethynylbenzyl)oxy)-3-fluorophenyl)acrylamide (25)

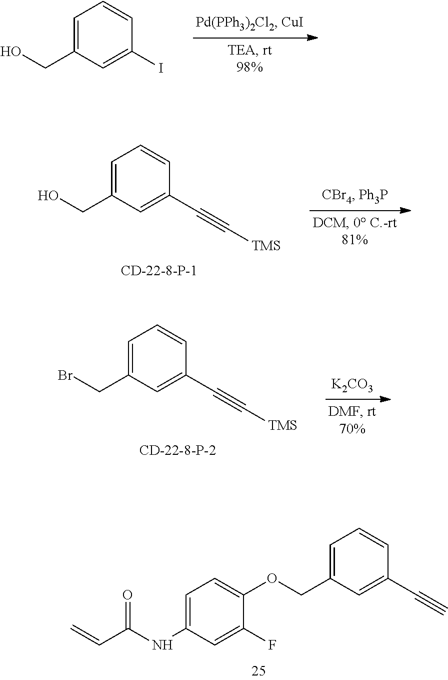 [SMILES: OCc1cccc(I)c1 ; C[Si](C)(C)C#Cc1cccc(CO)c1 ; C[Si](C)(C)C#Cc1cccc(CBr)c1 ; "K2CO3" ; C#Cc1cccc(COc2ccc(NC(=O)C=C)cc2F)c1]

(3-((trimethylsilyl)ethynyl)phenyl)methanol (CD-22-8-P-1). To a mixture of (3-iodophenyl)methanol (0.200 g, 0.85 mmol) and ethynyltrimethylsilane (2 mL) in triethylamine (2 mL) was added bis(triphenylphosphine)palladium(II) chloride (0.060 g, 0.09 mmol) and cuprous iodide (0.016 g, 0.09 mmol) under nitrogen, and the reaction mixture was stirred at room temperature for 3 h under nitrogen. The catalyst was removed through filtration, the filtrate cake was washed with MeOH (10 mL×2), and the combined filtrates were concentrated under reduced pressure to give crude product, which was purified by silica gel flash column chromatography using a 10% ethyl acetate/hexane gradient to afford (3-((trimethylsilyl)ethynyl)phenyl)methanol (CD-22-8-P-1) (0.172 g, 98%) as a brown oil.

((3-(bromomethyl)phenyl)ethynyl)trimethylsilane (CD-22-8-P-2). To a solution of (3-((trimethylsilyl)ethynyl)phenyl)methanol (CD-22-8-P-1) (0.100 g, 0.49 mmol) and carbon tetrabromide (0.195 g, 0.59 mmol) in dichloromethane (2 mL) at 0-5° C. was added triphenylphosphine (0.141 g, 0.54 mmol). The mixture was stirred at room temperature under nitrogen atmosphere overnight. The mixture was concentrated under reduced pressure, and the resulting crude was purified by silica gel flash column chromatography using a hexane gradient to afford ((3-(bromomethyl)phenyl)ethynyl)trimethylsilane (CD-22-8-P-2) (0.106 g, 81%) as a colorless oil.

N-(4-((3-ethynylbenzyl)oxy)-3-fluorophenyl)acrylamide (25). To a solution of N-(3-fluoro-4-hydroxyphenyl)acrylamide (CD-22-8-P-2) (0.064 g, 0.35 mmol) and ((3-(bromomethyl)phenyl)ethynyl)trimethylsilane (0.104 g, 0.39 mmol) in N,N-dimethylformamide (1 mL) was added potassium carbonate (0.193 g, 1.40 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 33% ethyl acetate/hexane gradient to afford N-(4-((3-ethynylbenzyl)oxy)-3-fluorophenyl)acrylamide (25) (0.071 g, 70%) as an off-white solid. ¹H NMR (400 MHz, CDCl₃) δ 7.57-7.53 (m, 2H), 7.46-7.41 (m, 2H), 7.36-7.32 (m, 1H), 7.19 (s, 1H), 7.14-7.12 (m, 1H), 6.92 (t, J=9.0 Hz, 1H), 6.43 (dd, J=16.8, 1.2 Hz, 1H), 6.21 (dd, J=16.8, 10.0 Hz, 1H), 5.77 (dd, J=10.4, 1.2 Hz, 1H), 5.09 (s, 2H), 3.08 (s, 1H). MS: [MH]⁺ 296.1.

### Example 1.22. Synthesis of N-(4-((3-ethylbenzyl)oxy)-3-fluorophenyl)acrylamide (26)

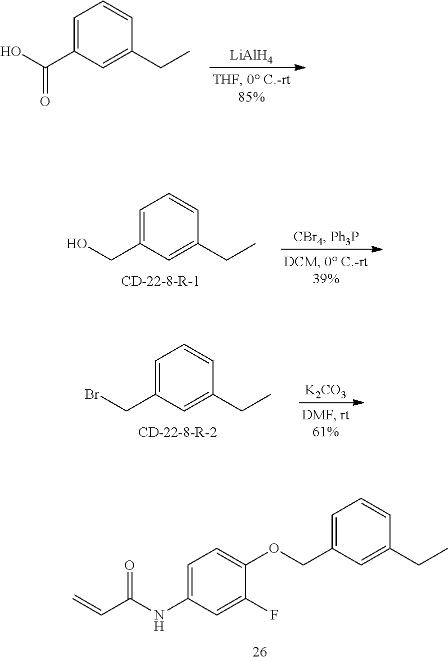

(3-ethylphenyl)methanol (CD-22-8-R-1). To a solution of 3-ethylbenzoic acid (0.100 g, 0.67 mmol) in tetrahydrofuran (3 mL) at 0-5° C. was added lithium aluminum hydride (0.049 g, 1.33 mmol). The reaction mixture was stirred at room temperature for 3 h under nitrogen. The mixture was quenched with water (0.05 mL), followed by the addition of aqueous solution of sodium hydroxide (15%, 0.05 mL) in water (0.15 mL). The catalyst was removed through filtration, the filtrate cake was washed with DCM (5 mL×3), and the combined filtrates were concentrated under reduced pressure to give crude product, which was purified by silica gel flash column chromatography using a 30% ethyl acetate/hexane gradient to afford (3-ethylphenyl)methanol (CD-22-8-R-1) (0.077 g, 85%) as a yellow oil.

1-(bromomethyl)-3-ethylbenzene (CD-22-8-R-2). To a solution of (3-ethylphenyl)methanol (CD-22-8-R-1) (0.077 g, 0.56 mmol) and carbon tetrabromide (0.281 g, 0.85 mmol) in dichloromethane (2 mL) at 0-5° C. was added triphenylphosphine (0.222 g, 0.85 mmol). The mixture was stirred at room temperature under nitrogen for 2 h. The reaction mixture was concentrated under reduced pressure. The resulting crude was purified by silica gel column chromatography using a hexane gradient to afford 1-(bromomethyl)-3-ethylbenzene (CD-22-8-R-2) (0.044 g, 39%) as a colorless oil.

N-(4-((3-ethylbenzyl)oxy)-3-fluorophenyl)acrylamide (26). To a solution of N-(3-fluoro-4-hydroxyphenyl)acrylamide (CD-22-8-R-2) (0.044 g, 0.22 mmol) and 1-(bromomethyl)-3-ethylbenzene (0.044 g, 0.24 mmol) in N,N-dimethylformamide (0.5 mL) was added potassium carbonate (0.061 g, 0.44 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 33% ethyl acetate/hexane gradient to afford N-(4-((3-ethylbenzyl)oxy)-3-fluorophenyl)acrylamide (26) (0.040 g, 61%) as an off-white solid. ¹H NMR (400 MHz, CDCl₃): δ 7.55-7.52 (m, 1H), 7.31-7.27 (m, 1H), 7.24-7.12 (m, 4H), 6.95 (t, J=8.8 Hz, 1H), 6.45-6.40 (m, 1H), 6.24-6.17 (m, 1H), 5.78-5.75 (m, 1H), 5.09 (s, 2H), 2.66 (q, J=7.6 Hz, 2H), 1.24 (t, J=7.6 Hz, 3H). MS: [MH]⁺ 300.1.

### Example 1.23. Synthesis of (E)-N-(3-fluoro-4-(3-fluorostyryl)phenyl)acrylamide (27)

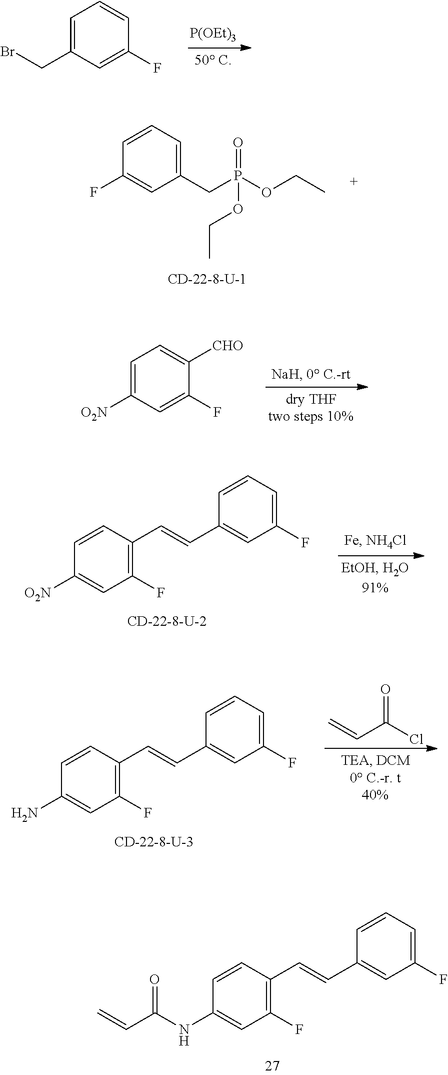

diethyl 3-fluorobenzylphosphonate (CD-22-8-U-1). A solution of 1-(bromomethyl)-3-fluorobenzene (2.000 g, 10.64 mmol) in triethyl phosphite (9.300 mL, 53.20 mmol) was stirred at 50° C. under nitrogen for 12 h. The reaction was concentrated under reduced pressure to afford crude diethyl 3-fluorobenzylphosphonate (CD-22-8-U-1) as a colorless oil, which was used in next step without further operation.

(E)-2-fluoro-1-(3-fluorostyryl)-4-nitrobenzene (CD-22-8-U-2). To a solution of diethyl 3-fluorobenzylphosphonate (11.470 g, 43.80 mmol) in anhydrous tetrahydrofuran (20 mL) was added sodium hydride (60% in mineral oil) (11.120 g, 43.80 mmol) at 0° C. under nitrogen, and the resulting mixture was stirred for 30 minutes, followed by the addition of a solution of 2-fluoro-4-nitrobenzaldehyde (1.380 g, 8.18 mmol) in anhydrous tetrahydrofuran (10 mL) dropwise. Then the resulting mixture was stirred at room temperature for 3 h. TLC showed the reaction was complete. The reaction mixture was poured into water (10 mL) and extracted with ethyl acetate (15 mL×2). Combined organic extracts were washed with brine (15 mL), dried over anhydrous Na₂SO₄, and concentrated in vacuo. The resulting crude was purified by silica gel column chromatography using a 2% ethyl acetate/hexane gradient to afford (E)-2-fluoro-1-(3-fluorostyryl)-4-nitrobenzene (CD-22-8-U-2) (0.290 g, 10% over two steps) as a yellow solid.

(E)-3-fluoro-4-(3-fluorostyryl)aniline (CD-22-8-U-3). To a solution of (E)-2-fluoro-1-(3-fluorostyryl)-4-nitrobenzene (CD-22-8-U-2) (0.100 g, 0.38 mmol) and ammonium chloride (0.142 g, 2.68 mmol) in ethanol-water (2 mL-0.5 mL) was added iron powder (0.150 g, 2.68 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 20%-30% ethyl acetate/hexane gradient to afford (E)-3-fluoro-4-(3-fluorostyryl)aniline (CD-22-8-U-3) (0.080 g, 91%) as a yellow solid. MS: [MH]⁺ 232.0.

(E)-N-(3-fluoro-4-(3-fluorostyryl)phenyl)acrylamide (27). To a stirred solution of (E)-3-fluoro-4-(3-fluorostyryl)aniline (CD-22-8-U-3) (0.080 g, 0.35 mmol) and triethylamine (0.140 g, 1.38 mmol) in dichloromethane (3 mL) was added acryloyl chloride (0.034 g, 0.38 mmol) dropwise under nitrogen at 0° C., and the resulting mixture was stirred at room temperature for 2 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford (E)-N-(3-fluoro-4-(3-fluorostyryl)phenyl)acrylamide (27) (0.075 g, 40%) as a white solid. ¹HNMR (400 MHz, CD₃OD) δ 7.72-7.69 (m, 2H), 7.41-7.19 (m, 6H), 7.03-6.69 (m, 1H), 5.85-5.81 (m, 1H), 6.50-6.34 (m, 2H). MS: [MH]⁺ 286.0.

### Example 1.24. Synthesis of N-(3-fluoro-4-((3-fluorophenoxy)methyl)phenyl)acrylamide (28)

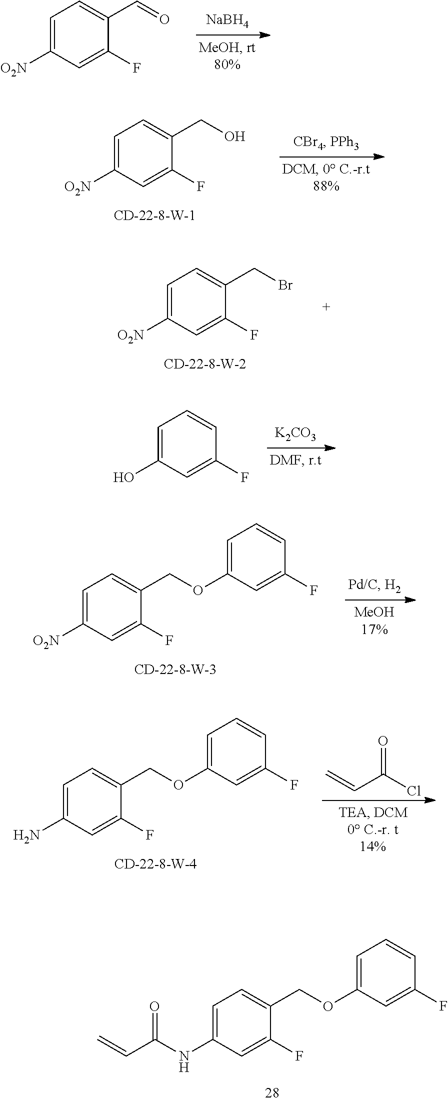

(2-fluoro-4-nitrophenyl)methanol (CD-22-8-W-1). To a solution of 2-fluoro-4-nitrobenzaldehyde (1.000 g, 5.91 mmol) in methanol (20 mL) was added sodium borohydride (1.120 g, 29.60 mmol) slowly at 0° C. under nitrogen. Then the mixture was stirred at room temperature for 1.5 hours. The reaction mixture was poured into water (20 mL) and extracted with DCM (20 mL×2). Combined organic extracts were washed with brine (30 mL), dried over anhydrous Na₂SO₄, and concentrated in vacuo. The resulting crude was purified by silica gel column chromatography using a 20% ethyl acetate/hexane gradient to afford 2-fluoro-4-nitrophenyl)methanol (CD-22-8-W-1) (0.810 g, 80%) as a yellow solid.

1-(bromomethyl)-2-fluoro-4-nitrobenzene (CD-22-8-W-2). To a mixture of 2-fluoro-4-nitrophenyl)methanol (0.800 g, 4.67 mmol) and carbon tetrabromide (2.010 g, 6.07 mmol) in dichloromethane (10 mL) was added triphenylphosphine (1.590 g, 6.07 mmol) at 0° C. under nitrogen. The reaction mixture was stirred at room temperature for 3 hours. TLC showed the reaction was complete. The mixture was concentrated under reduced pressure. The resulting crude was purified by silica gel column chromatography using a 2% ethyl acetate/hexane gradient to afford 1-(bromomethyl)-2-fluoro-4-nitrobenzene (CD-22-8-W-2) (0.840 g, 88%) as a white solid.

2-fluoro-1-((3-fluorophenoxy)methyl)-4-nitrobenzene (CD-22-8-W-3). To a mixture of 3-fluorophenol (0.404 g, 3.60 mmol) and potassium carbonate (0.004 g, 7.20 mmol) in N,N-dimethylformamide (5 mL) was added 1-(bromomethyl)-2-fluoro-4-nitrobenzene (CD-22-8-W-2) (0.840 g, 3.60 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford 2-fluoro-1-((3-fluorophenoxy)methyl)-4-nitrobenzene (CD-22-8-W-3) (0.464 g, 55%) as a yellow oil.

3-fluoro-4-((3-fluorophenoxy)methyl)aniline (CD-22-8-W-4). To a solution of 2-fluoro-1-((3-fluorophenoxy)methyl)-4-nitrobenzene (CD-22-8-W-3) (0.464 g, 1.75 mmol) in methanol (30 mL) was added palladium on carbon (10%, 123 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to give crude product, which was purified by silica gel flash column chromatography using a 12% ethyl acetate/hexane gradient to afford 3-fluoro-4-((3-fluorophenoxy)methyl)aniline (CD-22-8-W-4) (0.070 g, 17%) as a yellow oil. MS: [MH]⁺ 236.0.

N-(3-fluoro-4-((3-fluorophenoxy)methyl)phenyl)acrylamide (28). To a stirred solution of 3-fluoro-4-((3-fluorophenoxy)methyl)aniline (CD-22-8-W-4) (0.035 g, 0.15 mmol) and triethylamine (0.089 g, 0.60 mmol) in dichloromethane (3 mL) was added acryloyl chloride (0.014 g, 0.15 mmol) dropwise at 0° C. under nitrogen. Then reaction mixture was stirred at room temperature for 2 hours. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(3-fluoro-4-((3-fluorophenoxy)methyl)phenyl)acrylamide (28) (0.006 g, 14%) as a yellow oil. ¹HNMR (400 MHz, DMSO-d₆) δ 6.81-6.77 (m, 1H), 6.57-6.53 (t, J=6.4 Hz, 1H), 6.45-6.34 (m, 2H), 5.93-5.76 (m, 3H), 5.56-5.45 (m, 2H), 4.91-4.88 (m, 1H), 4.18 (s, 2H). MS: [MH]⁺ 290.0.

### Example 1.25. Synthesis of 5-(4-((4,4-difluorocyclohexyl)methoxy)-3-fluorophenyl)-1H-tetrazole (29)

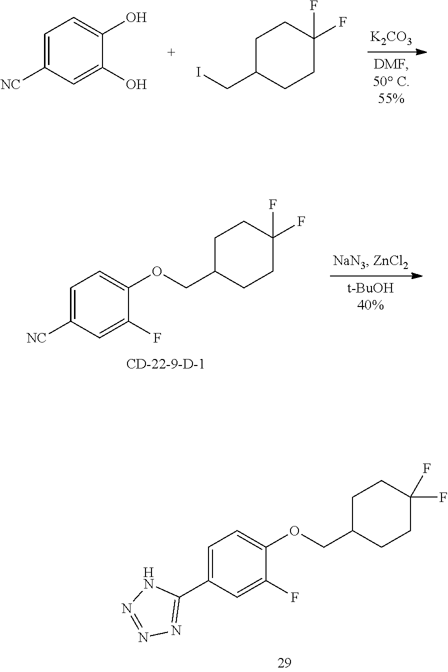

4-((4,4-difluorocyclohexyl)methoxy)-3-fluorobenzonitrile (CD-22-9-D-1). To a solution of 3-fluoro-4-hydroxybenzonitrile (0.100 g, 0.73 mmol) and potassium carbonate (0.302 g, 2.19 mmol) in N,N-dimethylformamide (3 mL) was added 1,1-difluoro-4-(iodomethyl)cyclohexane (0.474 g, 1.82 mmol) at room temperature under nitrogen, and the resulting mixture was stirred at 50° C. for 12 h. The reaction mixture was poured into water (15 mL) and extracted with ethyl acetate (10 mL×2). Combined organic extracts were washed with brine (15 mL), dried over anhydrous Na₂SO₄, and concentrated in vacuo. The resulting crude was purified by silica gel column chromatography using a 20% ethyl acetate/hexane gradient to afford 4-((4,4-difluorocyclohexyl)methoxy)-3-fluorobenzonitrile (CD-22-9-D-1) (0.107 g, 55%) as a yellow solid.

5-(4-((4,4-difluorocyclohexyl)methoxy)-3-fluorophenyl)-1H-tetrazole (29). To a solution of 4-((4,4-difluorocyclohexyl)methoxy)-3-fluorobenzonitrile (CD-22-9-D-1) (0.107 g, 0.40 mmol) and zinc chloride (0.027 g, 0.20 mmol) in tert-butanol (5 mL) was added NaN₃ (0.078 g, 1.20 mmol) at room temperature under nitrogen, and the resulting mixture was stirred at 80° C. for 72 h. The reaction mixture was poured into aqueous Na₂SO₃ solution (20 mL) and extracted with ethyl acetate (10 mL×2). Combined organic extracts were washed with brine (10 mL), dried over anhydrous Na₂SO₄, and concentrated in vacuo. The resulting crude was purified by silica gel column chromatography using a 50-75% ethyl acetate/hexane gradient to afford 5-(4-((4,4-difluorocyclohexyl)methoxy)-3-fluorophenyl)-1H-tetrazole (29) (0.050 g, 40%) like as yellow solid. ¹HNMR (400 MHz, CD₃OD): 7.79-7.74 (m, 2H), 7.25 (t, J=7.2 Hz, 1H), 4.01-3.99 (d, J=4.0 Hz, 2H), 2.11-2.09 (m, 2H), 2.00-1.97 (m, 3H), 1.87-1.79 (m, 2H), 1.48-1.45 (m, 2H). MS: [MH]⁺ 313.0.

### Example 1.26. Synthesis of N-(4-(1-(4,4-difluorocyclohexyl)ethoxy)-3-fluorophenyl)acrylamide (30)

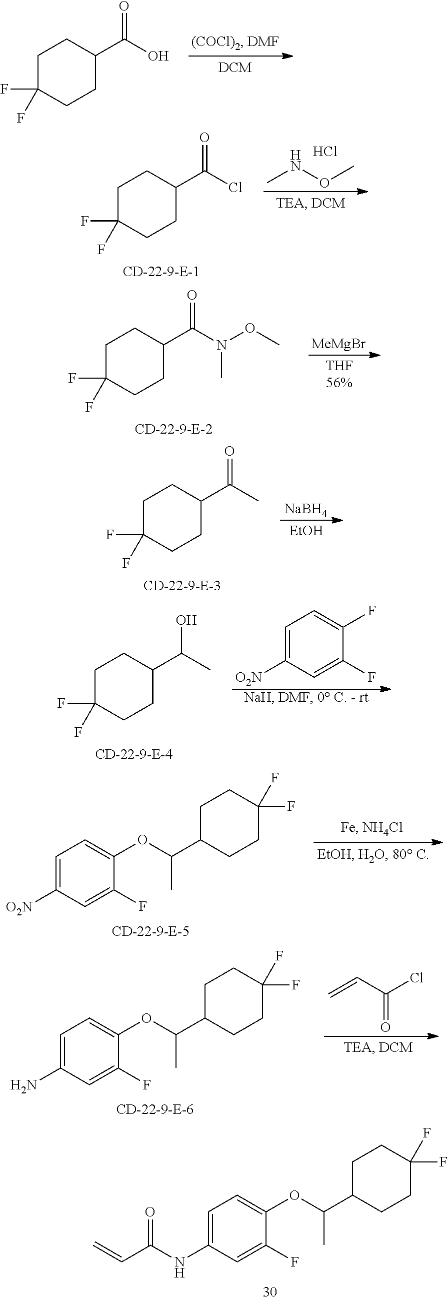

4,4-difluorocyclohexanecarbonyl chloride (CD-22-9-E-1). To a stirred solution of 4,4-difluorocyclohexanecarboxylic acid (1.000 g, 6.10 mmol) and oxalyl chloride (1.160 g, 9.10 mmol) in dichloromethane (20 mL) was added N,N-dimethylformamide (1 drop). The resulting mixture was stirred at room temperature overnight. The reaction mixture was concentrated under reduced pressure to give a crude 4,4-difluorocyclohexanecarbonyl chloride (CD-22-9-E-1), which was used into next step without further purification.

4,4-difluoro-N-methoxy-N-methylcyclohexanecarboxamide (CD-22-9-E-2). To a stirred solution of N,O-dimethylhydroxylamine hydrochloride (0.595 g, 6.10 mmol) and triethylamine (2.5 mL, 6.10 mmol) in dichloromethane (10 mL) was added 4,4-difluorocyclohexanecarbonyl chloride (CD-22-9-E-1) (crude) in dichloromethane (10 mL) dropwise at 5-10° C., and the resulting mixture was stirred at room temperature overnight. This mixture was concentrated under reduced pressure, and the residue was taken up in dichloromethane (10 mL), washed with brine (10 mL), and concentrated under reduced pressure. The resulting crude was purified by silica gel column chromatography using a 20% ethyl acetate/hexane gradient to afford 4,4-difluoro-N-methoxy-N-methylcyclohexanecarboxamide (CD-22-9-E-2) (0.900 g, 71% in two steps) as a colorless oil.

1-(4,4-difluorocyclohexyl)ethenone (CD-22-9-E-3). To a stirred solution of 4,4-difluoro-N-methoxy-N-methylcyclohexanecarboxamide (CD-22-9-E-2) (0.746 g, 4.34 mmol) in anhydrous tetrahydrofuran (10 mL) was added methyl magnesium bromide (3 M, 4 mL) at 0° C., and the resulting mixture was stirred at room temperature for 36 hours. The reaction mixture was poured into aqueous ammonium chloride solution (15 mL) and extracted with ethyl acetate (20 mL×3). Combined organic filtrates were concentrated in vacuo. The resulting crude was purified by silica gel column chromatography using a 20%-30% ethyl acetate/hexane gradient to afford 1-(4,4-difluorocyclohexyl)ethenone (CD-22-9-E-3) (0.502 g, 72%) as a light yellow oil.

1-(4,4-difluorocyclohexyl)ethanol (CD-22-9-E-4). To a stirred solution of 1-(4,4-difluorocyclohexyl)ethenone (CD-22-9-E-3) (0.100 g, 0.62 mmol) in ethanol (2 mL) was added sodium borohydride (0.047 g, 1.23 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 2% ethyl acetate/hexane gradient to afford 1-(4,4-difluorocyclohexyl)ethanol (CD-22-9-E-4) (0.120 g, 98%) as a colorless oil.

1-(1-(4,4-difluorocyclohexyl)ethoxy)-2-fluoro-4-nitrobenzene (CD-22-9-E-5). To a stirred solution of 1-(4,4-difluorocyclohexyl)ethanol (CD-22-9-E-4) (0.100 g, 0.63 mmol) in N,N-dimethylformamide (2 mL) was added NaH (0.050 g, 1.26 mmol) and stirred at 0° C. for 30 minutes. 1,2-difluoro-4-nitrobenzene (0.100 g, 0.63 mmol) was added at 0° C., and this mixture was stirred at room temperature overnight. The reaction mixture was poured into aqueous ammonium chloride solution (15 mL) and extracted with ethyl acetate (20 mL×3). Combined organic filtrates were concentrated in vacuo. The resulting crude was purified by silica gel column chromatography using a 25% ethyl acetate/hexane gradient to afford 1-(1-(4,4-difluorocyclohexyl)ethoxy)-2-fluoro-4-nitrobenzene (CD-22-9-E-5) (0.160 g, 83%).

4-(1-(4,4-difluorocyclohexyl)ethoxy)-3-fluoroaniline (CD-22-9-E-6). To a stirred solution of 1-(1-(4,4-difluorocyclohexyl)ethoxy)-2-fluoro-4-nitrobenzene (CD-22-9-E-5) (0.160 g, 0.53 mmol) and ammonium chloride (0.336 g, 6.33 mmol) in ethanol-water (9 mL-3 mL) was added iron powder (0.145 g, 2.64 mmol). This mixture was stirred at 80° C. for 15 minutes. After cooling to room temperature, iron was removed through filtration and washed with methanol (10 mL×2). Combined organic filtrates were concentrated in vacuo. The resulting crude was purified by silica gel column chromatography using a 40% ethyl acetate/hexane gradient to afford 4-(1-(4,4-difluorocyclohexyl)ethoxy)-3-fluoroaniline (CD-22-9-E-6) (0.050 g, 34%). MS: [MH]+274.0.

N-(4-(1-(4,4-difluorocyclohexyl)ethoxy)-3-fluorophenyl)acrylamide (30). To a stirred solution of 4-(1-(4,4-difluorocyclohexyl)ethoxy)-3-fluoroaniline (CD-22-9-E-6) (0.050 g, 0.18 mmol) and triethylamine (0.051 g, 0.54 mmol) in dichloromethane (2 mL) was added acryloyl chloride (0.018 g, 0.20 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 25% ethyl acetate/hexane gradient to afford N-(4-(1-(4,4-difluorocyclohexyl)ethoxy)-3-fluorophenyl)acrylamide (30) (0.023 g, 39%) as white solid. ¹H NMR (400 MHz, CDCl₃): δ 7.52 (d, J=12.0 Hz, 1H), 7.24-7.08 (m, 2H), 6.91 (t, J=8.0 Hz, 1H), 6.43-6.40 (m, 1H), 6.25-6.21 (m, 1H), 5.78-5.72 (m, 1H), 4.15-4.09 (m, 1H), 2.16-2.13 (m, 2H), 2.01-2.05 (m, 1H), 1.90-1.64 (m, 4H), 1.52-1.40 (m, 2H), 1.26 (d, J=4.0 Hz, 3H). MS: [MH]⁺ 328.1.

### Example 1.27. Synthesis of N-(3-fluoro-4-((3-(trifluoromethyl)phenoxy)methyl)phenyl)acrylamide (31)

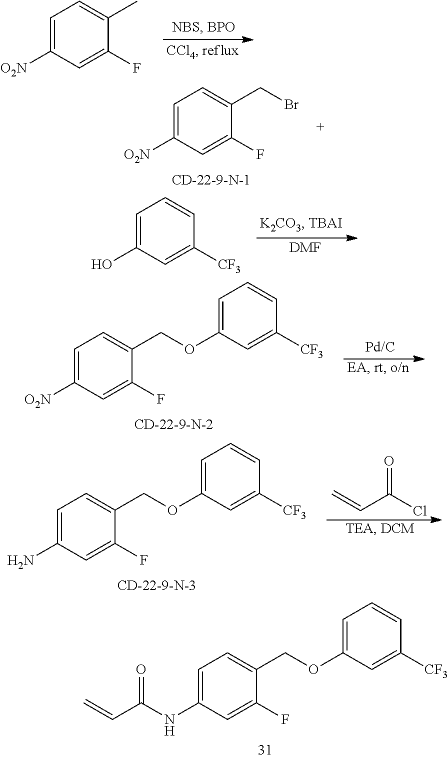

1-(bromomethyl)-2-fluoro-4-nitrobenzene (CD-22-9-N-1). To a stirring mixture of 2-fluoro-1-methyl-4-nitrobenzene (1.000 g, 6.45 mmol) in carbon tetrachloride (20 mL) was added N-bromosuccinimide (1.49 g, 8.39 mmol) and benzoyl peroxide (0.416 g, 1.29 mmol) under N₂ at room temperature, and the resulting mixture was stirred at 90° C. for 16 h. The reaction mixture was concentrated and purified by silica gel flash chromatography (eluted with 2% ethyl acetate in hexane) to afford 1-(bromomethyl)-2-fluoro-4-nitrobenzene (CD-22-9-N-1) (0.978 g, 65%) as a light yellow solid.

2-fluoro-4-nitro-1-((3-(trifluoromethyl)phenoxy)methyl)benzene (CD-22-9-N-2). To a stirring mixture of 1-(bromomethyl)-2-fluoro-4-nitrobenzene (CD-22-9-N-1) (0.300 g, 1.28 mmol), 3-(trifluoromethyl)phenol (0.207 g, 1.28 mmol), and TBAI (0.284 g, 0.77 mmol) in N,N-dimethylformamide (10 mL) was added K₂CO₃ (0.354 g, 2.56 mmol) at room temperature. The resulting mixture was stirred at room temperature for 2 h. The reaction mixture was poured into water (30 mL) and extracted with ethyl acetate (25 mL×2). Combined organic extracts were washed with brine (25 mL), dried over anhydrous Na₂SO₄, and concentrated in vacuo. The resulting crude was purified by silica gel column chromatography using a 2% ethyl acetate/hexane gradient to afford 2-fluoro-4-nitro-1-((3-(trifluoromethyl)phenoxy)methyl)benzene (CD-22-9-N-2) (0.360 g, 89%) as a yellow solid.

3-fluoro-4-((3-(trifluoromethyl)phenoxy)methyl)aniline (CD-22-9-N-3). To a stirring mixture of 2-fluoro-4-nitro-1-((3-(trifluoromethyl)phenoxy)methyl)benzene (CD-22-9-N-2) (0.100 g, 0.32 mmol) in ethyl acetate (15 mL) was added Pd/C (10 mg) at room temperature, the resulting mixture was stirred at room temperature under H₂ atmosphere (H₂ balloon) for 4 hours. The catalyst was removed through filtration, the filtrate cake was washed with ethyl acetate (10 mL×2), and the combined filtrates were concentrated under reduced pressure to give crude product, which was purified by silica gel flash column chromatography using a 1% methanol/dichloromethane gradient to afford 3-fluoro-4-((3-(trifluoromethyl)phenoxy)methyl) aniline (CD-22-9-N-3) (0.103 g, 99%) as a yellow oil. MS: [MH]⁺ 286.0.

N-(3-fluoro-4-((3-(trifluoromethyl)phenoxy)methyl)phenyl)acrylamide (31). To a stirring mixture of 3-fluoro-4-((3-(trifluoromethyl)phenoxy)methyl)aniline (CD-22-9-N-3) (103.4 mg, 0.16 mmol) and triethylamine (49 mg, 0.48 mmol) in dichloromethane (5 mL) was added acryloyl chloride (22 mg, 0.24 mmol) at 0° C. The resulting mixture was stirred at room temperature for 2 h. TLC showed the reaction was complete. The reaction mixture was partitioned between dichloromethane (30 mL) and water (20 mL); the organic layer was collected, and the aqueous layer was extracted with dichloromethane (10 mL×2). The combined organic layers were washed with brine (30 mL), dried over anhydrous sodium sulfate, and concentrated under reduced pressure to give a crude residue, which was purified by pre-TLC (eluted with 17% ethyl acetate in hexane) to afford N-(3-fluoro-4-((3-(trifluoromethyl)phenoxy)methyl)phenyl)acrylamide (31) (0.023 g, 42%) as a white solid. ¹H NMR (400 MHz, DMSO-d₆): δ 10.42 (s, 1H), 7.75-7.70 (m, 1H), 7.56-7.50 (m, 2H), 7.39-7.30 (m, 4H), 6.46-6.40 (m, 1H), 6.29-6.25 (m, 1H), 5.83-5.80 (m, 1H), 5.17 (s, 2H). MS: [MH]⁺ 340.0.

### Example 1.28. Synthesis of N-(4-acrylamido-2-fluorophenyl)-4,4-difluorocyclohexanecarboxamide (32)

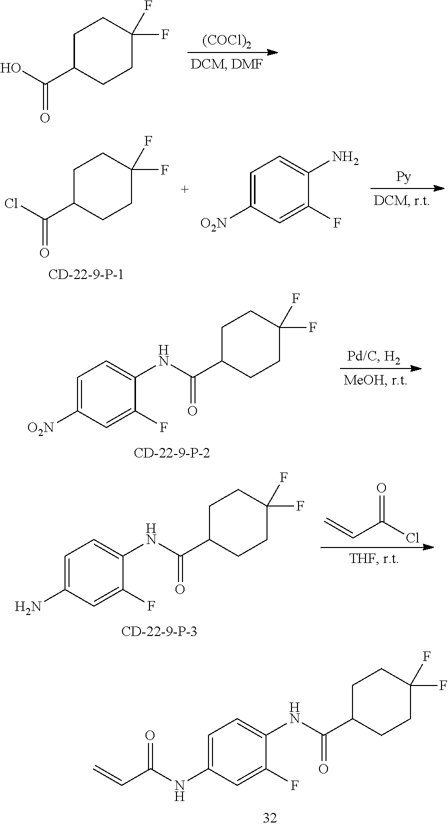

4,4-difluoro-N-(2-fluoro-4-nitrophenyl)cyclohexanecarboxamide (CD-22-8-P-2). To a solution of 4,4-difluorocyclohexanecarboxylic acid (1.000 g, 6.09 mmol) in dichloromethane (10 mL) was added oxalyl chloride (2.300 g, 18.27 mmol) and DMF (1 drop) at room temperature, and the mixture was stirred at room temperature under nitrogen for 2 hours. The mixture was concentrated in vacuo, and the residue was taken up in dichloromethane (10 mL), followed by the addition of 2-fluoro-4-nitroaniline (0.300 g, 1.92 mmol) and pyridine (1.5 mL) at 0° C. The resulting mixture was stirred at room temperature under nitrogen for 2 h. The reaction mixture was poured into water (20 mL) and extracted with ethyl acetate (10 mL×2). Combined organic extracts were washed with HCl (2M) (10 mL), dried over anhydrous Na₂SO₄, and concentrated in vacuo. The resulting crude was purified by silica gel column chromatography using a 10% ethyl acetate/hexane gradient to afford 4,4-difluoro-N-(2-fluoro-4-nitrophenyl)cyclohexanecarboxamide (CD-22-8-P-2) (0.388 g, 67%) as a white solid.

N-(4-amino-2-fluorophenyl)-4,4-difluorocyclohexanecarboxamide (CD-22-9-P-3). To a solution of 4,4-difluoro-N-(2-fluoro-4-nitrophenyl)cyclohexanecarboxamide (CD-22-8-P-2) (0.285 g, 0.94 mmol) in methanol (30 mL) was added palladium on carbon (10%, 123 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to give crude product, which was purified by silica gel flash column chromatography using a 1% methanol/dichloromethane gradient to afford N-(4-amino-2-fluorophenyl)-4,4-difluorocyclohexanecarboxamide (CD-22-9-P-3) (0.213 g, 81%) as a yellow solid.

N-(4-acrylamido-2-fluorophenyl)-4,4-difluorocyclohexanecarboxamide (32). To a solution of N-(4-amino-2-fluorophenyl)-4,4-difluorocyclohexanecarboxamide (CD-22-9-P-3) (0.100 g, 0.37 mmol) in tetrahydrofuran (2.5 mL) was added acryloyl chloride (0.040 g, 0.44 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(4-acrylamido-2-fluorophenyl)-4,4-difluorocyclohexanecarboxamide (32) (0.067 g, 57%) as a white solid. ¹H NMR (400 MHz, CD₃OD): δ 7.78-7.73 (m, 2H), 7.28-7.25 (m, 1H), 6.46-6.36 (m, 2H), 5.82-5.79 (m, 1H), 2.59 (t, J=5.2 Hz, 1H), 2.16 (d, J=7.2 Hz, 2H), 2.01-1.82 (m, 6H). MS: [MH]⁺ 327.0.

### Example 1.29. Synthesis of N-(4-((4,4-difluorocyclohexyl)methoxy)-3-fluorophenyl)methane sulfonamide (33)

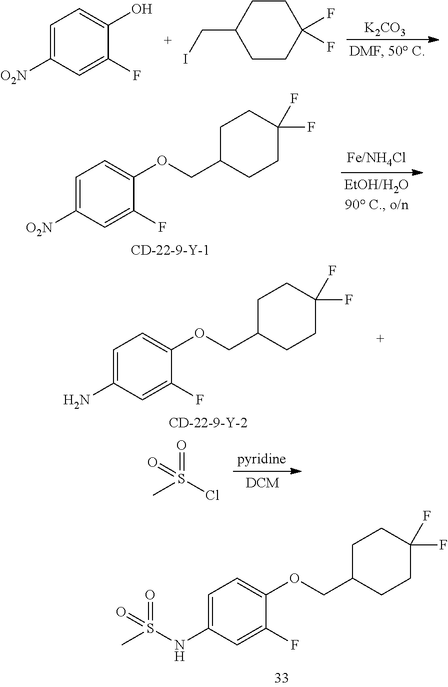

[1-((4,4-difluorocyclohexyl)methoxy)-2-fluoro-4-nitrobenzene (CD-22-9-Y-1). To a solution of 2-fluoro-4-nitrophenol (0.050 g, 0.32 mmol) and 1,1-difluoro-4-(iodomethyl) cyclohexane (0.091 g, 0.35 mmol) in N, N-dimethylformamide (3 mL) was added K₂CO₃ (0.088 g, 0.64 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 5% ethyl acetate/hexane gradient to afford 1-((4,4-difluorocyclohexyl)methoxy)-2-fluoro-4-nitrobenzene (CD-22-9-Y-1) (0.025 g, 27%) as a light yellow oil.

4-((4,4-difluorocyclohexyl)methoxy)-3-fluoroaniline (CD-22-9-Y-2). To a solution of 1-((4,4-difluorocyclohexyl)methoxy)-2-fluoro-4-nitrobenzene (CD-22-9-Y-1) (0.126 g, 0.44 mmol) and ammonium chloride (0.118 g, 2.18 mmol) in EtOH (8 mL)-H₂O (2 mL) was added iron powder (0.122 g, 2.18 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 10% ethyl acetate/hexane gradient to afford 4-((4,4-difluorocyclohexyl)methoxy)-3-fluoroaniline (CD-22-9-Y-2) (0.095 g, 84%) as a brown solid. MS: [MH]⁺ 301.3.

N-(4-((4,4-difluorocyclohexyl)methoxy)-3-fluorophenyl)methane sulfonamide (33). To a solution of 4-((4,4-difluorocyclohexyl)methoxy)-3-fluoroaniline (CD-22-9-Y-2) (0.040 g, 0.15 mmol) and pyridine (0.022 g, 0.28 mmol) in dichloromethane (3 mL) was added methane sulfonyl chloride (0.019 g, 0.17 mmol) in dichloromethane (1 mL) at 0° C., and the resulting mixture was stirred at room temperature for 1.5 h. The reaction mixture was poured into water (10 mL) and extracted with DCM (10 mL×2). Combined organic extracts were washed with 10% HCl solution (10 mL) then brine (10 mL), dried over anhydrous Na₂SO₄, and concentrated in vacuo. The resulting crude was purified by silica gel column chromatography using a 30-50% ethyl acetate/hexane gradient to afford N-(4-((4,4-difluorocyclohexyl)methoxy)-3-fluorophenyl)methane sulfonamide (33) (0.031 g, 60%) as an off-white solid. ¹H NMR (400 MHz, CDCl₃): δ 7.08-7.05 (m, 1H), 6.95-6.89 (m, 2H), 6.27 (s, 1H), 3.86 (d, J=8 Hz, 2H), 2.98 (s, 3H), 2.11-2.19 (m, 2H), 1.90-2.01 (m, 3H), 1.72-1.86 (m, 2H), 1.38-1.48 (m, 2H). MS: [MH]⁺ 338.1.

The following compound was prepared in a manner analogous to the procedures described above for N-(4-((4,4-difluorocyclohexyl)methoxy)-3-fluorophenyl)methane sulfonamide (33):

N-(4-((4,4-difluorocyclohexyl)methoxy)-3-fluorophenyl) ethene sulfonamide (34) (0.021 g, 54%) as an off-white solid. ¹H NMR (400 MHz, CDCl₃): δ 7.03-6.99 (m, 1H), 6.91-6.87 (m, 2H), 6.50-6.54 (m, 1H), 6.23-6.20 (m, 2H), 5.97 (d, J=10.0 Hz, 1H), 3.85 (d, J=6.4 Hz, 2H), 2.19-2.11 (m, 2H), 1.99-1.89 (m, 3H), 1.68-1.84 (m, 2H), 1.47-1.40 (m, 2H). MS: [M-H]⁺ 348.1

### Example 1.30. Synthesis of 4-((4,4-difluorocyclohexyl)methoxy)-3-fluorobenzoic acid (35)

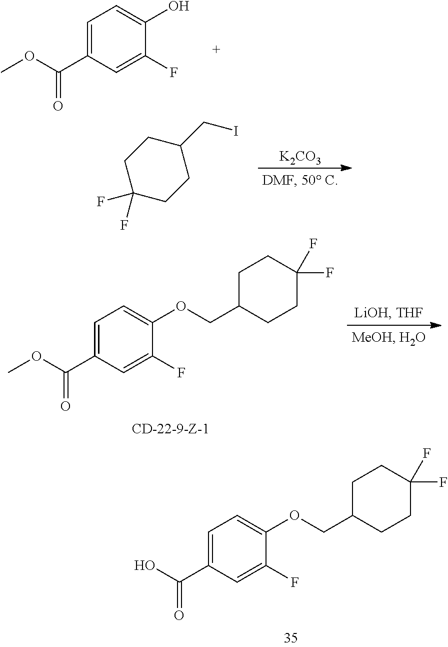

methyl 4-((4,4-difluorocyclohexyl)methoxy)-3-fluorobenzoate (CD-22-9-Z-1). To a solution of methyl 3-fluoro-4-hydroxybenzoate (0.100 g, 0.58 mmol) and 1,1-difluoro-4-(iodomethyl)cyclohexane (0.300 g, 1.16 mmol) in N,N-dimethylformamide (1 mL) was added potassium carbonate (122 mg, 0.87 mmol) at room temperature under nitrogen, and the resulting mixture was stirred at 50° C. for 5 h. The reaction mixture was poured into water (10 mL) and extracted with ethyl acetate (5 mL×2). Combined organic extracts were washed with brine (5 mL), dried over anhydrous Na₂SO₄ and concentrated in vacuo. The resulting crude was purified by silica gel column chromatography using a 5% ethyl acetate/hexane gradient to afford methyl 4-((4,4-difluorocyclohexyl)methoxy)-3-fluorobenzoate (CD-22-9-Z-1) (0.110 g, 64%) as a white solid.

4-((4,4-difluorocyclohexyl)methoxy)-3-fluorobenzoic acid (35). A mixture of methyl 2-((4-cyclopentylbenzamido)methyl)benzoate (CD-22-9-Z-1) (0.110 g, 0.36 mmol) and lithium hydroxide (0.025 g, 1.09 mmol) in tetrahydrofuran (2 mL)-water (1 mL)-methanol (1 mL) was stirred at room temperature for 12 h. The reaction mixture was acidified with diluted hydrochloride acid (2 N) to pH 5-6 and extracted with ethyl acetate (10 mL×2). Combined organic extracts were washed with brine (10 mL), dried over anhydrous Na₂SO₄, and concentrated in vacuo. The resulting crude was purified by silica gel column chromatography using a 5% methanol/dichloromethane gradient to afford 4-((4,4-difluorocyclohexyl)methoxy)-3-fluorobenzoic acid (35) (0.060 g, 57%) as a white solid. ¹H NMR (400 MHz, DMSO-d₆): δ 12.90 (s, 1H), 7.76-7.74 (m, 1H), 7.66-7.62 (m, 1H), 7.28 (t, J=8.8 Hz, 1H), 4.03 (d, J=6.4 Hz, 2H), 2.10-1.98 (m, 2H), 1.98-1.82 (m, 4H), 1.80-1.77 (m, 1H), 1.40-1.30 (m, 2H). MS: [MH]⁺ 286.9.

The following compound was prepared in a manner analogous to the procedures described above for 4-((4,4-difluorocyclohexyl)methoxy)-3-fluorobenzoic acid (35):

3-((4,4-difluorocyclohexyl)methoxy)-4-fluorobenzoic acid (36) (0.048 g, 80%) as a white solid. ¹HNMR (400 MHz, DMSO-d₆): δ 13.00 (s, 1H), 7.67-7.61- (m, 1H), 7.59-7.53- (m, 1H), 7.37-7.29 (m, 1H), 4.00 (d, J=6.4 Hz, 2H), 2.10-1.99 (m, 2H), 1.98-1.77 (m, 5H), 1.42-1.30 (m, 2H). MS: [MH]⁺ 289.1.

### Example 1.31. Synthesis of N-(cyclopropylsulfonyl)-4-((4,4-difluorocyclohexyl)methoxy)-3-fluorobenzamide (37)

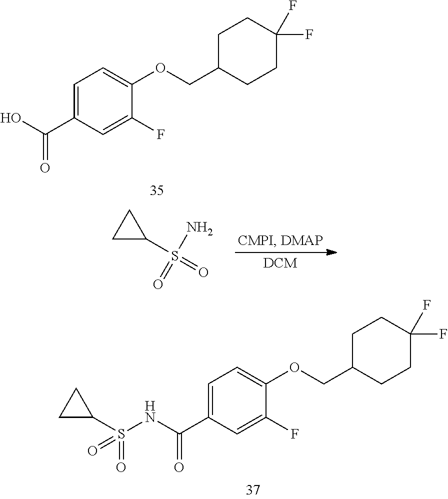 [SMILES: O=C(O)c1ccc(OCC2CCC(F)(F)CC2)c(F)c1 ; NS(=O)(=O)C1CC1 ; O=C(NS(=O)(=O)C1CC1)c1ccc(OCC2CCC(F)(F)CC2)c(F)c1]

To a solution of 4-((4,4-difluorocyclohexyl)methoxy)-3-fluorobenzoic acid (35) (0.030 g, 0.10 mmol), cyclopropanesulfonamide (0.026 g, 0.20 mmol), 4-dimethylaminopyridine (0.006 g, 0.05 mol), and triethylamine (0.032 g, 0.30 mmol) in dichloromethane (1 mL) was added 2-chloro-1-methylpyridinium iodide (0.032 g, 0.12 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 1% methanol/dichloromethane gradient to afford N-(cyclopropylsulfonyl)-4-((4,4-difluorocyclohexyl)methoxy)-3-fluorobenzamide (37) (0.032 g, 72%) as a white solid. ¹H NMR (400 MHz, DMSO-d₆): δ 11.99 (s, 1H), 7.82-7.78 (m, 1H), 7.31 (t, J=8.8 Hz, 1H), 4.05 (d, J=6.0 Hz, 2H), 3.14-3.08 (m, 1H), 2.10-1.96 (m, 2H), 1.97-1.78 (m, 4H), 1.81-1.77 (m, 1H), 1.39-1.29 (m, 2H), 1.14-1.07 (m, 4H). MS: [MH]⁺ 392.1.

### Example 1.32. Synthesis of N-(3-cyano-4-((3-(trifluoromethyl)benzyl)oxy)phenyl)acrylamide (38)

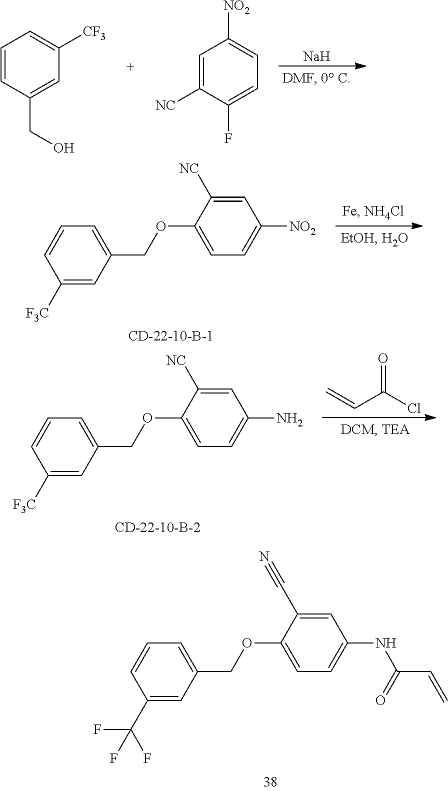

5-nitro-2-((3-(trifluoromethyl)benzyl)oxy)benzonitrile (CD-22-10-B-1) To a suspension of NaH (0.080 g, 2.00 mmol, 60%) in DMF (5 mL) was added (3-(trifluoromethyl)phenyl)methanol (0.176 g, 1.00 mmol) in DMF (2 mL) at 0° C.; and the resulting mixture was stirred for 30 min. To the reaction mixture was added 2-fluoro-5-nitrobenzonitrile (0.200 g, 1.20 mmol) at 0° C., and the resulting mixture was stirred at 0° C. for 1 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 5-nitro-2-((3-(trifluoromethyl)benzyl)oxy)benzonitrile (CD-22-10-B-1) (0.258 g, 80%) as a white solid. ¹H NMR (400 MHz, CDCl₃): δ 8.53 (d, J=2.8 Hz, 1H), 8.44-8.40 (m, 1H), 7.70-7.66 (m, 3H), 7.61-7.57 (m, 1H), 7.13 (d, J=9.6 Hz, 1H), 5.78 (s, 2H).

5-amino-2-((3-(trifluoromethyl)benzyl)oxy)benzonitrile (CD-22-10-B-2). To a solution of 5-nitro-2-((3-(trifluoromethyl)benzyl)oxy)benzonitrile (CD-22-10-B-1) (0.100 g, 0.31 mmol) and ammonium chloride (166 mg, 3.10 mmol) in ethanol (8 mL)-water (2 mL) was added iron powder (0.173 g, 3.10 mmol); the resulting mixture was stirred at 85° C. for 2 h. After cooling to room temperature, iron was removed through filtration and washed with methanol (10 mL×2). Combined organic filtrates were concentrated in vacuo. The resulting crude was purified by silica gel column chromatography using a 30% ethyl acetate/hexane gradient to afford 5-amino-2-((3-(trifluoromethyl)benzyl)oxy)benzonitrile (CD-22-10-B-2) (0.085 g, 94%) as a pink solid. MS: [MH]⁺ 292.9.

N-(3-cyano-4-((3-(trifluoromethyl)benzyl)oxy)phenyl)acrylamide (38). To a solution of 5-amino-2-((3-(trifluoromethyl)benzyl)oxy)benzonitrile (CD-22-10-B-2) (0.080 g, 0.27 mmol) and triethylamine (0.083 g, 0.82 mmol) in dichloromethane (5 mL) was added acryloyl chloride (0.037 g, 0.41 mmol) in dichloromethane (2 mL) at 0° C. The reaction mixture was stirred at 0° C. for 1 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(3-cyano-4-((3-(trifluoromethyl)benzyl)oxy)phenyl)acrylamide (38) (0.055 g, 58%) as a white solid. ¹H NMR (400 MHz, DMSO-d₆): δ 10.32 (s, 1H), 8.07 (d, J=2.4 Hz, 1H), 7.86-7.77 (m, 3H), 7.75-7.73 (m, 1H), 7.70-7.66 (m, 1H), 7.36 (d, J=9.2 Hz, 1H), 6.43-6.36 (m, 1H), 6.30-6.25 (m, 1H), 5.79-5.73 (m, 1H), 5.37 (s, 2H). MS: [MH]⁺ 320.9.

The following compounds were prepared in a manner analogous to the procedures described above for N-(3-cyano-4-((3-(trifluoromethyl)benzyl)oxy)phenyl)acrylamide (38):

N-(4-((2-chloro-3-fluorobenzyl)oxy)-3-cyanophenyl)acrylamide (39) (0.055 g, 58%) as an off-white solid. ¹H NMR (400 MHz, DMSO-d₆): δ 10.33 (s, 1H), 8.08 (d, J=2.8 Hz, 1H), 7.83-7.80 (m, 1H), 7.50-7.45 (m, 3H), 7.40 (d, J=9.2 Hz, 1H), 6.43-6.36 (m, 1H), 6.30-6.25 (m, 1H), 5.79-5.73 (m, 1H), 5.35 (s, 1H). MS: [MH]⁺ 330.9.

N-(3-cyano-4-((3,4-difluorobenzyl)oxy)phenyl)acrylamide (40) (0.040 g, 40%) as a white solid. ¹H NMR (400 MHz, DMSO-d₆): δ 10.31 (s, 1H), 8.06 (d, J=2.4 Hz, 1H), 7.81 (d, J=11.2 Hz, 1H), 7.58-7.47 (m, 2H), 7.35-7.32 (m, 2H), 6.42-6.25 (m, 2H), 5.79 (d, J=8.4 Hz, 1H), 5.25 (s, 2H). MS: [MH]⁺ 314.9.

N-(3-cyano-4-((5-(trifluoromethyl)pyridin-3-yl)methoxy)phenyl)acrylamide (41) (0.062 g, 63%) as an off-white solid. ¹H NMR (400 MHz, DMSO-d₆): δ 10.34 (s, 1H), 9.00 (s, 2H), 8.33 (s, 1H), 8.07 (d, J=2.4 Hz, 1H), 7.87-7.84 (m, 1H), 7.40 (d, J=9.2 Hz, 1H), 6.43-6.36 (m, 1H), 6.30-6.25 (m, 1H), 5.81-5.78 (m, 1H), 5.42 (s, 2H). MS: [MH]⁺ 348.1.

N-(2-cyano-4-((5-(trifluoromethyl)pyridin-3-yl)methoxy)phenyl)acrylamide (42) (0.022 g, 63%) as white solid. ¹H NMR (400 MHz, DMSO-d₆): δ 10.23 (s, 1H), 9.00-8.99 (m, 2H), 8.33 (s, 1H), 7.59 (d, J=2.8 Hz, 1H), 7.55-7.52 (m, 1H), 7.42-7.40 (m, 1H), 6.51-6.45 (m, 1H), 6.31-6.26 (m, 1H), 5.85-5.82 (m, 1H), 5.33 (s, 2H). MS: [MH]⁺ 348.2.

N-(3-fluoro-4-((6-(trifluoromethyl)pyridin-2-yl)methoxy)phenyl)acrylamide (43) (0.033 g, yield 32%) as white solid. ¹H NMR (400 MHz, DMSO-d₆): δ 10.22 (s, 1H), 7.83 (s, 1H), 7.78 (d, J=7.6 Hz, 1H), 7.77 (d, J=8.0 Hz, 1H), 7.66 (t, J=7.8 Hz, 1H), 7.55 (d, J=3.2 Hz, 1H), 5.51 (d, J=8.8 Hz, 1H), 7.39-7.35 (m, 1H), 6.51-6.44 (m, 1H), 6.28-6.24 (m, 1H), 5.85-5.82 (m, 1H), 5.28 (s, 2H). MS: [MH]⁺ 347.1.

### Example 1.33. Synthesis of N-(4-((3-fluorobenzyl)oxy)phenyl)acrylamide (44)

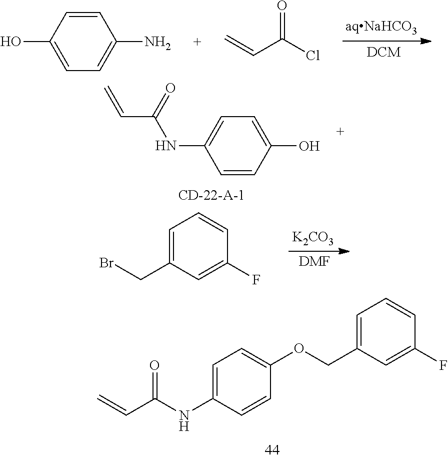

N-(4-hydroxyphenyl)acrylamide (CD-22-A-1). To a stirred solution of 4-aminophenol (0.300 g, 2.75 mmol) in a mixture of sat aq. NaHCO₃ (4 mL)-DCM (4 mL) was added acryloyl chloride (0.500 g, 5.50 mmol) dropwise at room temperature under nitrogen, and the resulting mixture was stirred at room temperature for 10 min. The organic layer was collected and the aqueous layer was extracted with DCM (5 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(4-hydroxyphenyl)acrylamide (CD-22-A-1) (0.18 g, 40%) as a white solid. MS [MH]+164.1.

N-(4-((3-fluorobenzyl)oxy)phenyl)acrylamide (44). To a solution of N-(4-hydroxyphenyl)acrylamide (CD-22-A-1) (0.047 g, 0.292 mmol) and 1-(bromomethyl)-3-fluorobenzene (0.050 g, 0.27 mmol) in DMF (1 mL) was added K₂CO₃ (0.072 g, 0.53 mmol) at room temperature under nitrogen and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄ and concentrated in vacuo. The resulting crude was purified by silica gel column chromatography using a 30-50% ethyl acetate/hexane gradient to afford N-(4-((3-fluorobenzyl)oxy)phenyl)acrylamide (44) (0.040 g, 55%) as a white solid. ¹HNMR (400 MHz, CDCl₃) δ 7.50 (d, J=8.8 Hz, 2H), 7.35-7.33 (m, 1H), 7.22-7.14 (m, 3H), 7.04-6.99 (m, 1H), 6.93 (d, J=8.8 Hz, 2H), 5.75 (d, J=10.0 Hz, 1H), 5.05 (s, 1H). MS: [MH]⁺ 272.3.

The following compounds were prepared in a manner analogous to the procedures described above for N-(4-((3-fluorobenzyl)oxy)phenyl)acrylamide (44):

N-(2-fluoro-4-((3-fluorobenzyl)oxy)phenyl)acrylamide (45) (0.065 g, 81%) as a white solid. ¹HNMR (400 MHz, CDCl₃): δ 8.26 (t, J=9.2 Hz, 1H), 7.38-7.33 (m, 1H), 7.27 (s, 1H), 7.19-7.12 (m, 2H), 7.05-7.00 (m, 1H), 6.78-6.73 (m, 2H), 6.44 (d, J=16.8 Hz, 1H), 6.26 (dd, J=16.8, 10.2 Hz, 1H), 5.79 (d, J=10.2 Hz, 1H), 5.04 (s, 2H). MS: [MH]⁺ 290.3.

N-(3-chloro-4-((3-fluorobenzyl)oxy)phenyl)acrylamide (46) (0.056 g, 73%) as a white solid. ¹HNMR (400 MHz, CDCl₃): δ 7.67 (s, 1H), 7.43 (d, J=8.6 Hz, 1H), 7.32-7.38 (m, 1H), 7.20 (t, J=9.6 Hz, 2H), 7.12 (s, 1H), 6.99-7.04 (m, 1H), 6.91 (d, J=8.8 Hz, 1H), 6.44 (d, J=16.8 Hz, 1H), 6.21 (dd, J=16.8, 10.2 Hz, 1H), 5.79 (dd, J=10.2, 1.0 Hz, 1H), 5.13 (s, 2H). MS: [MH]⁺ 306.0.

N-(4-((3-fluorobenzyl)oxy)-2-methylphenyl)acrylamide (47) (0.040 g, 64%) as a yellow solid. ¹H NMR (400 MHz, DMSO-d₆): δ 9.38 (s, 1H), 7.46-7.41 (m, 1H), 7.31-7.25 (m, 3H), 7.17-7.13 (m, 1H), 6.90 (d, J=2.4 Hz, 1H), 6.84-6.81 (m, 1H), 6.51-6.45 (m, 1H), 6.23-6.18 (m, 1H), 5.72-5.69 (m, 1H), 5.11 (s, 2H), 2.16 (s, 3H). MS: [MH]⁺ 286.1.

N-(4-((4,4-difluorocyclohexyl)methoxy)-3-methylphenyl)acrylamide (48) (0.030 g, 19%) as a white solid. ¹H NMR (400 MHz, CDCl₃): δ 1.42-1.33 (m, 2H), 1.90-1.77 (m, 5H), 2.04-2.03 (m, 2H), 2.14 (s, 3H), 3.83-3.81 (d, J=8.0 Hz, 2H), 5.75-5.70 (m, 1H), 6.25-6.17 (m, 1H), 6.37-6.33 (m, 1H), 6.87-6.85 (m, 1H), 7.45-7.41 (m, 2H), 9.90 (s, 1H). MS: [MH]⁺ 310.2.

N-(4-((4,4-difluorocyclohexyl)methoxy)naphthalen-1-yl)acrylamide (49) (0.015 g, 14%) as a white solid. ¹HNMR (400 MHz, CD₃OD): δ 8.23-8.17 (m, 1H), 7.77 (d, J=8.4 Hz, 1H), 7.47-7.36 (m, 3H), 6.86-6.81 (m, 1H), 6.55-6.48 (m, 1H), 6.35-6.30 (m, 1H), 5.77-5.73 (m, 1H), 3.98 (d, J=6.0 Hz, 2H), 2.09-1.92 (m, 5H), 1.85-1.68 (m, 2H), 1.52-1.40 (m, 2H) MS: [MH]⁺ 346.1.

N-(3-cyano-4-((4,4-difluorocyclohexyl)methoxy)phenyl)acrylamide (50) (0.025 g, 14.7%) as a white solid. ¹HNMR (400 MHz, CDCl₃): δ 7.83-7.72 (m, 2H), 7.22 (br, 1H), 6.92 (d, J=8.8 Hz, 1H), 6.49-6.41 (m, 1H), 6.26-6.17 (m, 1H), 5.84-5.78 (m, 1H), 3.90 (d, J=6.4 Hz, 2H), 2.21-2.10 (m, 2H), 2.06-1.93 (m, 3H), 1.86-1.69 (m, 2H), 1.49-1.38 (m, 2H). MS: [MH]⁺ 321.1.

### Example 1.34. Synthesis of N-(4-((3-fluorobenzyl)oxy)-3-methylphenyl)acrylamide (51)

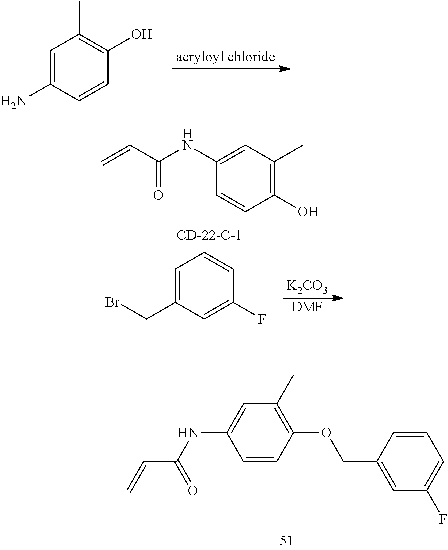

N-(4-hydroxy-3-methylphenyl)acrylamide (CD-22-C-1). To a stirred solution of 4-amino-2-methylphenol (5.000 g, 40.40 mmol) in a mixture of sat aq. NaHCO₃ (20 mL)-DCM (20 mL) was added acryloyl chloride (4.94 g, 55.10 mmol) dropwise at 0° C. under nitrogen, and the resulting mixture was stirred at 0° C. for 30 min. The organic layer was collected and the aqueous layer was extracted with DCM (10 mL×2). Combined organic extracts were washed with brine (10 mL), dried over anhydrous Na2SO4, and concentrated in vacuo. The resulting crude was purified by silica gel column chromatography using a 30-50% ethyl acetate/hexane gradient to afford N-(4-hydroxy-3-methylphenyl)acrylamide (CD-22-C-1) (5.705 g, 80%) as a white solid. MS: [MH]⁺ 182.1.

N-(4-((3-fluorobenzyl)oxy)-3-methylphenyl)acrylamide (51). To a stirred solution of N-(4-hydroxy-3-methylphenyl)acrylamide (CD-22-C-1) (100 mg, 0.56 mmol) and potassium carbonate (154.56 mg, 1.12 mmol) in N,N-dimethylformamide (5 ml) was added 1-(bromomethyl)-3-fluorobenzene (107 mg, 0.56 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford N-(4-((3-fluorobenzyl)oxy)-3-methylphenyl)acrylamide (51) (0.145 g, 88%) as a yellow solid. ¹H NMR (400 MHz, CDCl₃): δ 7.39-7.32 (m, 3H), 7.20-7.13 (m, 3H), 7.03-6.99 (m, 1H), 6.81 (d, J=8.4 Hz, 1H), 6.44-6.40 (m, 1H), 6.25-6.19 (m, 1H), 5.76-5.74 (m, 1H), 5.06 (s, 2H), 2.29 (s, 3H). MS: [MH]⁺ 286.3.

The following compounds were prepared in a manner analogous to the procedures described above for N-(3-fluoro-4-((3-fluorobenzyl)oxy)phenyl)acrylamide (51):

N-(4-(benzyloxy)-3-fluorophenyl)acrylamide (52) (0.040 g, 33%) as a white solid. ¹HNMR (400 MHz, CDCl₃): δ 7.54 (d, J=12.8 Hz, 1H), 7.44-7.42 (m, 2H), 7.38 (t, J=7.2 Hz, 2H), 7.34-7.30 (m, 1H), 7.22 (m, 1H), 7.31 (d, J=8.8 Hz, 1H), 6.94 (t, J=8.8 Hz, 1H), 6.45-6.40 (m, 1H), 6.24-6.17 (m, 1H), 5.77 (d, J=10.0 Hz, 1H), 5.12 (s, 2H). MS: [MH]⁺ 272.4.

N-(4-((2-chlorobenzyl)oxy)-3-fluorophenyl)acrylamide (53) (0.060 g, 44%) as a white solid. ¹HNMR (400 MHz, CDCl₃): δ 7.62-7.57 (m, 2H), 7.41-7.38 (m, 1H), 7.30-7.28 (m, 2H), 7.20 (s, 1H), 7.14-7.12 (m, 1H), 6.96 (t, J=8.8 Hz, 1H), 6.46-6.41 (m, 1H), 6.25-6.18 (m, 1H), 5.78 (d, J=10.2 Hz, 1H), 5.22 (s, 2H). MS: [MH]⁺ 306.2.

N-(3-fluoro-4-((tetrahydro-2H-pyran-4-yl)methoxy)phenyl)acrylamide (54) (0.025 g, 20%) as a white solid. ¹HNMR (400 MHz, CDCl₃): δ 7.51 (d, J=12.8 Hz, 1H), 7.22-7.17 (m, 2H), 6.91 (t, J=8.8 Hz, 1H), 6.45-6.41 (m, 1H), 6.25-6.18 (m, 1H), 5.78 (d, J=10.4 Hz, 1H), 4.02 (dd, J=3.6 Hz, 11.2 Hz, 2H), 3.85 (d, J=6.4 Hz, 2H), 3.44 (t, J=11.6 Hz, 2H), 2.18-2.03 (m, 1H), 1.78 (d, J=12.8 Hz, 2H), 1.50-1.41 (m, 2H). MS: [MH]⁺ 280.3.

N-(4-(cyclohexylmethoxy)-3-fluorophenyl)acrylamide (55) (0.040 g, 32%) as a white solid. ¹HNMR (400 MHz, CDCl₃): δ 7.49 (d, J=12.8 Hz, 1H), 7.20-7.16 (m, 2H), 6.90 (t, J=8.8 Hz, 1H), 6.45-6.41 (m, 1H), 6.25-6.18 (m, 1H), 5.77 (d, J=10.4 Hz, 1H), 4.35-4.11 (m, 1H), 3.80 (d, J=6.4 Hz, 2H), 1.89-1.86 (m, 2H), 1.78-1.68 (m, 3H), 1.32-1.24 (m, 3H), 1.09-1.02 (m, 2H). MS: [MH]⁺ 278.1.

N-(3-fluoro-4-(pyridin-3-ylmethoxy)phenyl)acrylamide (56) (0.059 g, 46%) as a white solid. ¹H NMR (400 MHz, DMSO-d₆): δ 10.22 (s, 1H), 8.67 (d, J=2.0 Hz, 1H), 8.56 (dd, J=4.8, 1.2 Hz, 1H), 7.93-7.83 (m, 1H), 7.70 (dd, J=13.6, 2.0 Hz, 1H), 7.44 (dd, J=7.6, 4.8 Hz, 1H), 7.37-7.21 (m, 2H), 6.39 (m, 1H), 6.27-6.23 (m, 1H), 5.76 (dd, J=10.0, 2.0 Hz, 1H), 5.20 (s, 2H). MS: [MH]⁺ 273.4.

N-(4-((4-chloropyridin-3-yl)methoxy)-3-fluorophenyl)acrylamide (57) (0.123 g, 92%) as a white solid. ¹H NMR (400 MHz, DMSO-d₆): δ 10.23 (s, 1H), 8.75 (s, 1H), 8.57 (d, J=5.2 Hz, 1H), 7.74-7.68 (m, 1H), 7.65 (d, J=5.2 Hz, 1H), 7.36-7.27 (m, 2H), 6.42-6.37 (m, 1H), 6.28-6.24 (m, 1H), 5.77 (dd, J=10.0, 2.0 Hz, 1H), 5.25 (s, 2H). MS: [MH]⁺ 308.1.

N-(4-((4,4-difluorocyclohexyl)methoxy)-3-fluorophenyl)acrylamide (58) (0.022 g, 23%) as a white solid. ¹HNMR (400 MHz, CDCl₃): δ 7.52 (d, J=12.6 Hz, 1H), 7.18-7.14 (m, 2H), 6.90 (t, J=8.8 Hz, 1H), 6.44 (d, J=16.7 Hz, 1H), 6.21-6.17 (m, 1H), 5.78 (d, J=10.2 Hz, 1H), 3.86 (d, J=6.4 Hz, 2H), 2.19-2.12 (m, 2H), 2.00-1.89 (m, 3H), 1.85-1.68 (m, 2H), 1.47-1.38 (m, 2H). MS: [MH]⁺ 314.3.

N-(3-fluoro-4-((4-(trifluoromethyl)cyclohexyl)methoxy)phenyl)acrylamide (59) (0.022 g, 12%) as a pink solid. ¹H NMR (400 MHz, DMSO-d₆): δ 10.15 (s, 1H), 7.66 (d, J=14.0 Hz, 1H), 7.27 (d, J=9.2 Hz, 1H), 7.13 (t, J=9.0 Hz, 1H), 6.42-6.35 (m, 1H), 6.24 (d, J=16.4 Hz, 1H), 5.75 (d, J=10.4 Hz, 1H), 3.84 (d, J=6.4 Hz, 2H), 2.28-2.20 (m, 1H), 1.91 (d, J=10.4 Hz, 4H), 1.74-1.70 (m, 1H), 1.34-1.25 (m, 2H), 1.17-1.08 (m, 2H). MS: [MH]⁺ 346.4.

N-(4-(benzo[b]thiophen-3-ylmethoxy)-3-fluorophenyl)acrylamide (60) (0.045 g, 83%) as a white solid. ¹H NMR (400 MHz, DMSO-d₆): δ 10.20 (s, 1H), 8.04-8.02 (m, 1H), 7.95-7.93 (m, 1H), 7.89 (s, 1H), 7.70-7.67- (m, 1H), 7.47-7.40 (m, 2H), 7.37-7.30 (m, 2H), 6.43-6.36 (m, 1H), 6.28-6.23 (m, 1H), 5.78-5.75 (m, 1H), 5.41 (s, 2H). MS: [MH]⁺ 328.3.

N-(4-((2-chlorothiophen-3-yl)methoxy)-3-fluorophenyl)acrylamide (61) (0.095 g, 75%) as a white solid. ¹H NMR (400 MHz, DMSO-d₆): δ 10.20 (s, 1H), 7.69-7.64 (m, 1H), 7.50 (d, J=5.6 Hz, 1H), 7.33-7.27 (m, 1H), 7.23 (t, J=9.2 Hz, 1H), 7.15 (d, J=5.6 Hz, 1H), 6.41-6.36 (m, 1H), 6.27-6.23 (m, 1H), 5.76-5.72 (m, 1H), 5.06 (s, 2H). MS: [MH]⁺ 312.2.

methyl 5-((4-acrylamido-2-fluorophenoxy)methyl)thiophene-2-carboxylate (62) (0.040 g, 81%) as an off-white solid. ¹H NMR (400 MHz, DMSO-d₆): δ 10.21 (s, 1H), 7.73-7.67 (m, 2H), 7.31-7.23 (m, 3H), 6.42-6.35 (m, 1H), 6.27-6.23 (m, 1H), 5.78-5.75 (m, 1H), 5.40 (s, 2H), 3.81 (s, 3H). MS: [MH]⁺ 336.0.

N-(4-((5-cyanothiophen-2-yl)methoxy)-3-fluorophenyl)acrylamide (63) (0.045 g, 90%) as a yellow solid. ¹H NMR (400 MHz, DMSO-d₆): δ 10.23 (s, 1H), 7.91 (d, J=3.6 Hz, 1H), 7.73-7.69 (m, 1H), 7.36-7.35 (m, 1H), 7.33-7.24 (m, 2H), 6.42-6.36 (m, 1H), 6.28-6.23 (m, 1H), 5.78-5.75 (m, 1H), 5.44 (s, 2H). MS: [MH]⁺ 303.0.

N-(3-fluoro-4-((2-methylthiazol-5-yl)methoxy)phenyl)acrylamide (64) (0.091 g, 57%) as an off-white solid. ¹H NMR (400 MHz, DMSO-d₆): δ 10.20 (s, 1H), 7.69-7.66 (m, 2H), 7.31-7.24 (m, 2H), 6.41-6.35 (m, 1H), 6.25-6.20 (m, 1H), 5.76-5.71 (m, 1H), 5.34 (s, 2H), 2.63 (s, 3H). MS: [MH]⁺ 293.4.

N-(3-fluoro-4-((5-methyl-2-oxo-1,3-dioxol-4-yl)methoxy)phenyl)acrylamide (65) (0.009 g, 5%) as a white solid. ¹H NMR (400 MHz, CDCl₃): δ 7.66 (d, J=12.8 Hz, 1H), 7.17-7.11 (m, 2H), 6.99 (t, J=8.8 Hz, 1H), 6.48-6.42 (m, 1H), 6.27-6.22 (m, 1H), 5.86-5.81 (m, 1H), 4.83 (s, 2H), 2.11 (s, 3H). MS: [MH]⁺ 294.3.

N-(3-fluoro-4-(pyridin-3-ylmethoxy)phenyl)acrylamide (66) (0.068 g, 65%) as a white solid. ¹H NMR (400 MHz, DMSO-d₆): δ 10.20 (s, 1H), 8.60 (d, J=6.0 Hz, 2H), 7.74-7.70 (m, 1H), 7.44 (d, J=6.0 Hz, 2H), 7.30-7.28 (m, 1H), 7.18 (t, J=9.2 Hz, 1H), 6.42-6.35 (m, 1H), 6.28-6.23 (m, 1H), 5.78-5.75 (m, 1H), 5.23 (s, 2H). MS: [MH]⁺ 273.5.

N-(4-((2-cyanopyridin-4-yl)methoxy)-3-fluorophenyl)acrylamide (67) (0.110 g, 91%) as a white solid. ¹H NMR (400 MHz, DMSO-d₆): δ 10.23 (s, 1H), 8.78 (d, J=5.0 Hz, 1H), 8.07 (s, 1H), 7.83-7.69 (m, 2H), 7.31 (s, 1H), 7.19 (t, J=9.2 Hz, 1H), 6.39-6.34 (m, 1H), 6.26-6.22 (m, 1H), 5.77-5.71 (m, 1H), 5.30 (s, 2H). MS: [MH]⁺ 298.2.

N-(4-((6-bromopyridin-3-yl)methoxy)-3-fluorophenyl)acrylamide (68) (0.099 g, 85%) as a brown solid. ¹H NMR (400 MHz, DMSO-d₆): δ 10.21 (s, 1H), 8.48 (d, J=2.4 Hz, 1H), 7.85-7.82 (m, 1H), 7.72-7.68 (m, 2H), 7.32-7.21 (m, 2H), 6.35-6.42 (m, 1H), 6.23-6.27 (m, 1H), 5.74-5.77 (m, 1H), 5.18 (s, 2H). MS: [MH]⁺ 352.9.

N-(3-fluoro-4-((6-methylpyridin-3-yl)methoxy)phenyl)acrylamide (69) (0.064 g, 63%) as a brown solid. ¹H NMR (400 MHz, DMSO-d₆): δ 10.18 (s, 1H), 8.52 (s, 1H), 7.76-7.66 (m, 2H), 7.31-7.21 (m, 3H), 6.42-6.35 (m, 1H), 6.27-6.22 (m, 1H), 5.77-5.74 (m, 1H), 5.14 (s, 2H), 2.47 (s, 3H). MS: [MH]⁺ 287.5.

methyl 5-((4-acrylamido-2-fluorophenoxy)methyl)picolinate (70) (0.161 g, 89%) as an off-white solid. ¹H NMR (400 MHz, DMSO-d₆): δ 10.20 (s, 1H), 8.79 (s, 1H), 8.11-8.04 (m, 2H), 7.77-7.71 (m, 1H), 7.32-7.22 (m, 2H), 6.42-6.35 (m, 1H), 6.25-6.21 (m, 1H), 5.76-5.70 (m, 1H), 5.31 (s, 2H), 3.89 (s, 3H). MS: [MH]⁺ 331.4.

N-(3-fluoro-4-((5-methylpyridin-3-yl)methoxy)phenyl)acrylamide (71) (0.073 mg, 44%) as a brown solid. ¹H NMR (400 MHz, DMSO-d₆): δ 10.19 (s, 1H), 8.46 (d, J=2.00 Hz, 1H), 8.40 (d, J=2.00 Hz, 1H), 7.73-7.64 (m, 2H), 7.32-7.20 (m, 2H), 6.42-6.38 (m, 1H), 6.34-6.31 (m, 1H), 5.74-5.70 (m, 1H), 5.15 (s, 2H). MS: [MH]⁺ 287.2.

N-(4-((6-chloropyridin-3-yl)methoxy)-3-fluorophenyl)acrylamide (72) (0.068 g, 67%) as a brown solid. ¹H NMR (400 MHz, DMSO-d₆): δ 10.21 (s, 1H), 8.51 (d, J=2.4 Hz, 1H), 7.96-7.93 (m, 1H), 7.72-7.68 (m, 1H), 7.57 (d, J=8.4 Hz, 1H), 7.32-7.22 (m, 2H), 6.42-6.35 (m, 1H), 6.27-6.23 (m, 1H), 5.78-5.75- (m, 1H), 5.20 (s, 2H). MS: [MH]⁺ 307.1.

N-(3-fluoro-4-((6-(trifluoromethyl)pyridin-3-yl)methoxy)phenyl)acrylamide (73) (0.077 g, 68%) as a brown solid. ¹H NMR (400 MHz, DMSO-d₆): δ 10.22 (s, 1H), 8.86 (s, 1H), 8.16 (d, J=8.0 Hz, 1H), 7.97 (d, J=8.0 Hz, 1H), 7.74-7.70 (m, 1H), 7.33-7.24 (m, 2H), 6.42-6.35 (m, 1H), 6.28-6.23 (m, 1H), 5.78-5.75 (m, 1H), 5.33 (s, 2H). MS: [MH]⁺ 341.3.

N-(4-((2-bromopyridin-3-yl)methoxy)-3-fluorophenyl)acrylamide (74) (0.070 g, 72%) as white solid. ¹H NMR (400 MHz, CDCl₃): δ 8.35-8.33 (m, 1H), 7.95-7.92 (m, 1H), 7.66-7.63 (m, 1H), 7.35-7.32 (m, 1H), 7.27 (br, 1H), 7.16-7.14 (m, 1H), 7.00-6.95 (m, 1H), 6.47-6.42 (m, 1H), 6.26-6.19 (m, 1H), 5.81-5.78 (m, 1H), 5.15 (s, 2H) MS: [MH]⁺ 350.9.

N-(3-fluoro-4-((2-methylpyridin-3-yl)methoxy)phenyl)acrylamide (75) (0.020 g, 70%) as a brown solid. ¹HNMR (400 MHz, CDCl₃): δ 8.46-8.41 (m, 1H), 7.72 (d, J=6.4 Hz, 1H), 7.56 (d, J=12.0 Hz, 1H), 7.31 (s, 1H), 7.17-7.14 (m, 2H), 6.96 (t, J=9.2 Hz, 1H), 6.49-6.43 (m, 1H), 6.25-6.18 (m, 1H), 5.78-5.73 (m, 1H), 5.09 (s, 2H), 2.60 (s, 3H). MS: [MH]⁺ 287.4.

N-(4-((2-chloropyridin-3-yl)methoxy)-3-fluorophenyl)acrylamide (76) (0.025 g, 15%) as a white solid. ¹HNMR (400 MHz, CDCl₃): δ 8.36 (d, J=4.4 Hz, 1H), 7.98 (d, J=7.6 Hz, 1H), 7.63 (d, J=12.0 Hz, 1H), 7.33-7.29 (m, 1H), 7.25 (s, 1H), 7.15-7.13 (m, 1H), 6.98 (t, J=9.2 Hz, 1H), 6.46-6.42 (m, 1H), 6.25-6.19 (m, 1H), 5.79 (d, J=10.0 Hz, 1H), 5.19 (s, 2H). MS: [MH]⁺ 308.2.

N-(4-((2-chloro-4-cyanobenzyl)oxy)-3-fluorophenyl)acrylamide (77) (0.040 g, 55%) as a gray solid. ¹H NMR (400 MHz, DMSO-d₆) δ 10.23 (s, 1H), 8.14 (d, J=1.2 Hz, 1H), 7.90 (d, J=7.6 Hz, 1H), 7.77 (d, J=8.0 Hz, 1H), 7.73 (d, J=13.6 Hz, 1H), 7.32-7.21 (m, 2H), 6.38 (d, J=8.8 Hz, 1H), 6.26 (d, J=16.8 Hz, 1H), 5.76 (d, J=9.6 Hz, 1H), 5.29 (s, 2H). MS: [MH]⁺ 331.0.

N-(4-((2-chloro-5-fluorobenzyl)oxy)-3-fluorophenyl)acrylamide (78) (0.073 g, 53%) as a white solid. ¹H NMR (400 MHz, DMSO-d₆) δ 10.23 (s, 1H), 7.72-7.70 (m, 1H), 7.59-7.55 (m, 1H), 7.46-7.40 (m, 1H), 7.32-7.23 (m, 3H), 6.39-6.36 (m, 1H), 6.26-6.20 (m, 1H), 5.76-5.73 (m, 1H), 5.19 (s, 2H). MS: [MH]⁺ 323.9.

N-(4-((2,5-dichlorobenzyl)oxy)-3-fluorophenyl)acrylamide (79) (0.023 g, 30%) as a white solid. ¹H NMR (400 MHz, DMSO-d₆): δ 10.22 (s, 1H), 7.74-7.71 (m, 1H), 7.70 (d, J=2.0 Hz, 1H), 7.61 (d, J=8.4 Hz, 1H), 7.55-7.50 (m, 1H), 7.32-7.21 (m, 2H), 6.39-6.34 (m, 1H), 6.28-6.25 (m, 1H), 5.80-5.76 (m, 1H), 5.19 (s, 2H). MS: [MH]⁺ 339.9.

N-(4-((2,3-dichlorobenzyl)oxy)-3-fluorophenyl)acrylamide (80) (0.100 g, 89%) as a gray solid. ¹HNMR (400 MHz, CDCl₃): δ 7.65-7.56 (m, 1H), 7.54-7.49 (m, 1H), 7.46-7.41 (m, 1H), 7.27-7.22 (m, 1H), 7.20 (br, 1H), 7.13 (d, J=8.4 Hz, 1H), 6.94 (t, J=8.8 Hz, 1H), 6.47-6.40 (m, 1H), 6.26-6.17 (m, 1H), 5.81-5.76 (m, 1H), 5.22 (s, 2H). MS: [MH]⁺ 339.9.

N-(4-((2-chloro-4-(trifluoromethyl)benzyl)oxy)-3-fluorophenyl)acrylamide (81) (0.102 g, 81%) as a gray solid. ¹HNMR (400 MHz, DMSO-d₆): δ 10.23 (s, 1H), 7.98 (s, 1H), 7.82-7.76 (m, 2H), 7.75-7.69 (m, 1H), 7.35-7.26 (m, 2H), 6.43-6.35 (m, 1H), 6.28-6.22 (m, 1H), 5.78-5.74 (m, 1H), 5.28 (s, 2H). MS: [MH]⁺ 374.0.

2-((4-acrylamido-2-fluorophenoxy)methyl)-3-chlorobenzoate (82) (0.093 g, 77%) as a gray solid. ¹HNMR (400 MHz, DMSO-d₆): δ 10.21 (s, 1H), 7.79-7.74 (m, 2H), 7.70-7.64 (m, 1H), 7.56 (t, J=8.0 Hz, 1H), 7.32-7.29 (m, 1H), 7.23 (t, J=9.2 Hz, 1H), 6.43-6.35 (m, 1H), 6.28-6.22 (m, 1H), 5.78-5.73 (m, 1H), 5.42 (s, 2H), 3.77 (s, 3H). MS: [MH]⁺ 364.0.

N-(4-((2-chloro-3-fluorobenzyl)oxy)-3-fluorophenyl)acrylamide (83) (0.155 g, 87%) as a light pink solid. ¹HNMR (400 MHz, CDCl₃): δ 7.61 (d, J=12.4 Hz, 1H), 7.39 (d, J=7.6 Hz, 1H), 7.30-7.27 (m, 1H), 7.25-7.11 (m, 3H), 6.95 (t, J=8.8 Hz, 1H), 6.48-6.44 (m, 1H), 6.26-6.21 (m, 1H), 5.79-5.74 (m, 1H), 5.22 (s, 2H). MS: [MH]⁺ 324.0.

N-(4-((2-chloro-4-fluorobenzyl)oxy)-3-fluorophenyl)acrylamide (84) (0.156 g, 87%) as a grey solid. ¹HNMR (400 MHz, DMSO-d₆): δ 10.21 (s, 1H), 7.73-7.64 (m, 2H), 7.58-7.54 (m, 1H), 7.33-7.22 (m, 3H), 6.46-6.42 (m, 1H), 6.28-6.23 (m, 1H), 5.79-5.74 (m, 1H), 5.17 (s, 2H). MS: [MH]⁺ 324.0.

N-(4-((2-chloro-6-fluorobenzyl)oxy)-3-fluorophenyl)acrylamide (85) (0.066 g, 74%) as yellow solid. ¹HNMR (400 MHz, DMSO-d₆): δ 10.23 (s, 1H), 7.71-7.68 (m, 1H), 7.55-7.49 (m, 1H), 7.43 (d, J=8.0 Hz, 1H), 7.35-7.28 (m, 3H), 6.43-6.37 (m, 1H), 6.29-6.24 (m, 1H), 5.78-5.75 (m, 1H), 5.19 (d, J=1.2 Hz, 2H). MS: [MH]⁺ 323.9.

N-(4-((2,6-dichlorobenzyl)oxy)-3-fluorophenyl)acrylamide (86) (0.051 g, 55%) as a pink solid. ¹HNMR (400 MHz, DMSO-d₆): δ 10.23 (s, 1H), 7.71-7.68 (m, 1H), 7.58-7.56 (m, 2H), 7.50-7.46 (m, 1H), 7.36-7.30 (m, 2H), 6.43-6.36 (m, 1H), 6.28-6.24 (m, 1H), 5.78-5.75 (m, 1H), 5.28 (s, 2H). MS: [MH]⁺ 339.9.

methyl 3-((4-acrylamido-2-fluorophenoxy)methyl)-4-chlorobenzoate (87) (0.037 g, 37%) as yellow solid. ¹HNMR (400 MHz, DMSO-d₆): δ 10.23 (s, 1H), 8.18 (d, J=2.0 Hz, 1H), 7.97-7.94 (m, 1H), 7.75-7.68 (m, 2H), 7.33-7.24 (m, 2H), 6.43-6.36 (m, 1H), 6.28-6.24 (m, 1H), 5.78-5.75 (m, 1H), 5.27 (s, 2H), 3.87 (s, 3H). MS: [MH]⁺ 364.0.

N-(4-((2-chloro-5-methoxybenzyl)oxy)-3-fluorophenyl)acrylamide (88) (0.086 g, 93%) as a white solid. ¹HNMR (400 MHz, CDCl₃): δ 7.59 (d, J=12.0 Hz, 1H), 7.28 (s, 1H), 7.22 (s, 1H), 7.15-7.12 (m, 2H), 6.96 (t, J=8.8 Hz, 1H), 6.85-6.80 (m, 1H), 6.45-6.41 (m, 1H), 6.25-6.18 (m, 1H), 5.77 (d, J=10.4 Hz, 1H), 5.18 (s, 2H), 3.79 (s, 3H). MS: [MH]⁺ 336.1.

2-((3-fluorobenzyl)oxy)-5-nitrobenzonitrile (89) (0.077 g, 83%) as a white solid. ¹H NMR (400 MHz, DMSO-d₆): δ 10.19 (s, 1H), 7.81-7.63 (m, 5H), 7.32-7.21 (m, 2H), 6.42-6.35 (m, 1H), 6.27-6.23 (m, 1H), 5.76-5.71 (m, 1H), 5.25 (s, 2H). MS: [MH]⁺ 340.0.

N-(4-((3-chlorobenzyl)oxy)-3-fluorophenyl)acrylamide (90) (0.120 g, 71%) as a gray solid. ¹HNMR (400 MHz, DMSO-d₆): δ 10.18 (s, 1H), 7.72-7.66 (m, 1H), 7.54-7.49 (m, 1H), 7.47-7.38 (m, 3H), 7.32-7.26 (m, 1H), 7.20 (d, J=9.2 Hz, 1H), 6.42-6.34 (m, 1H), 6.29-6.21 (m, 1H), 5.78-5.72 (m, 1H), 5.26 (s, 2H). MS: [MH]⁺ 306.0.

N-(3-fluoro-4-((3-(trifluoromethyl)cyclohexyl)methoxy)phenyl)acrylamide (91) (0.010 g, 22%) as a white solid. ¹H NMR (400 MHz, CDCl₃): δ 7.51-7.49 (m, 1H), 7.18-7.11 (m, 2H), 6.94-6.87 (m, 1H), 6.45-6.40 (m, 1H), 6.24-6.17 (m, 1H), 5.76-5.79 (d, J=12 Hz, 1H), 3.96-3.93 (m, 1H), 3.86-3.83 (m, 1H), 2.32-2.12 (s, 2H), 1.95-1.64 (m, 5H), 1.37-1.25 (m, 2H), 1.07-1.13 (m, 1H). MS: [MH]⁺ 346.20.

N-(4-((3-cyanobenzyl)oxy)-3-fluorophenyl)acrylamide (92) (0.117 g, 72%) as a white solid. ¹H NMR (400 MHz, DMSO-d₆): δ 10.19 (s, 1H), 7.91 (s, 1H), 7.84-7.79 (m, 2H), 7.72-7.68 (m, 1H), 7.63 (t, J=7.6 Hz, 1H), 7.30 (d, J=8.8 Hz, 1H), 7.22 (t, J=9.2 Hz, 1H), 6.42-6.35 (m, 1H), 6.27-6.23 (m, 1H), 5.77-5.74 (m, 1H), 5.21 (s, 2H). MS: [MH]⁺ 297.1.

N-(3-fluoro-4-((3-methylbenzyl)oxy)phenyl)acrylamide (93) as a white solid (0.078 g, 51%). ¹HNMR (400 MHz, DMSO-d₆): δ 10.16 (s, 1H), 7.68-7.62 (m, 1H), 7.29-7.14 (m, 6H), 6.41-6.35 (m, 1H), 6.24-6.21 (m, 1H), 5.75-5.72 (m, 1H), 5.09 (s, 2H), 2.31 (s, 3H). MS: [MH]⁺ 286.1.

N-(3-fluoro-4-(4,4,4-trifluorobutoxy)phenyl)acrylamide (94) (0.040 g, 24%) as an off-white solid. ¹H NMR (400 MHz, DMSO-d₆): δ 10.17 (s, 1H), 7.70-7.66 (m, 1H), 7.31-7.29 (m, 1H), 7.14 (t, J=9.2 Hz, 1H), 6.42-6.35- (m, 1H), 6.27-6.22 (m, 1H), 5.77-5.74 (m, 1H), 4.08 (t, J=6.2 Hz, 2H), 2.44-2.39 (m, 2H), 1.96-1.92 (m, 2H). MS: [MH]⁺ 292.1.

N-(4-((2,3-difluorobenzyl)oxy)-3-fluorophenyl)acrylamide (95) (0.063 g, 37%) as a white solid. ¹HNMR (400 MHz, DMSO-d₆) δ 10.19 (s, 1H), 7.71-7.68 (m, 1H), 7.49-7.42 (m, 1H), 7.38-7.35 (m, 1H), 7.32-7.24 (m, 3H), 6.42-6.35 (m, 1H), 6.27-6.23 (m, 1H), 5.77-5.74 (m, 1H), 5.23 (s, 2H). MS: [MH]⁺ 308.1.

N-(4-((3,4-difluorobenzyl)oxy)-3-fluorophenyl)acrylamide (96) (0.103 g, 61%) as a white solid. ¹H NMR (400 MHz, DMSO-d₆) δ 10.18 (s, 1H), 7.71-7.67 (m, 1H), 7.55-7.43 (m, 2H), 7.33-7.28 (m, 2H), 7.20 (t, J=9.0 Hz, 1H), 6.42-6.35 (m, 1H), 6.27-6.22 (m, 1H), 5.77-5.74 (m, 1H), 5.13 (s, 2H). MS: [MH]⁺ 308.1.

N-(4-((3,5-difluorobenzyl)oxy)-3-fluorophenyl)acrylamide (97) (0.078 g, 57%) as a gray solid. ¹H NMR (400 MHz, DMSO-d₆): δ 10.20 (s, 1H), 7.71 (d, J=13.6 Hz, 1H), 7.31-7.28 (s, 1H), 7.24-7.16 (m, 4H), 6.36-6.31 (m, 1H), 6.25 (d, J=17.2 Hz, 1H), 5.75-5.70 (m, 1H), 5.18 (s, 2H). MS: [MH]⁺ 308.1.

N-(3-fluoro-4-((2-(trifluoromethyl)pyrimidin-5-yl)methoxy)phenyl)acrylamide (98) (0.019 g, 75%) as an off-white solid. ¹H NMR (400 MHz, DMSO-d6): δ 8.99 (s, 1H), 7.61 (d, J=12.0 Hz, 1H), 7.33-7.20 (m, 2H), 7.00 (t, J=8.4 Hz, 1H), 6.45 (d, J=16.4 Hz, 1H), 6.26-6.19 (m, 1H), 5.80 (d, J=10.4 Hz, 1H), 5.35 (s. 1H), 5.32 (s, 2H). MS: [MH]⁺ 342.0.

N-(4-((3-chloro-2-fluorobenzyl)oxy)-3-fluorophenyl)acrylamide (99) (0.094 g, 53%) as a yellow solid. ¹H NMR (400 MHz, CDCl₃): δ 10.2 (s, 1H), 7.70 (d, J=8.0 Hz, 1H), 7.61 (t, J=7.6 Hz, 1H), 7.53 (t, J=7.2 Hz, 1H), 7.32-7.26 (m, 3H), 6.42-6.35 (m, 1H), 6.25 (d, J=6.4 Hz, 1H), 5.76 (d, J=6.0 Hz, 1H), 5.23 (s, 2H). MS: [MH]⁺ 324.1.

N-(3-fluoro-4-((3-fluorobenzyl)oxy)phenyl)acrylamide (100) (0.180 g, 29%) as an off-white solid. ¹H NMR (400 MHz, DMSO-d₆): δ 10.20 (s, 1H), 7.72-7.68 (dd, J=13.6 Hz, 2.4 Hz, 1H), 7.48-7.42 (m, 1H), 7.30-7.27 (m, 3H), 7.23-7.15 (m, 2H), 6.39-6.35 (m, 1H), 6.27-6.22 (dd, J=17.2 Hz, 2.0 Hz, 1H), 5.77-5.74 (dd, J=9.6 Hz, 2.0 Hz, 1H), 5.17 (s, 2H). MS [MH]⁺ 290.4.

### Example 1.35. Synthesis of N-(3-fluoro-4-((3-fluorobenzyl)amino)phenyl)acrylamide (101)

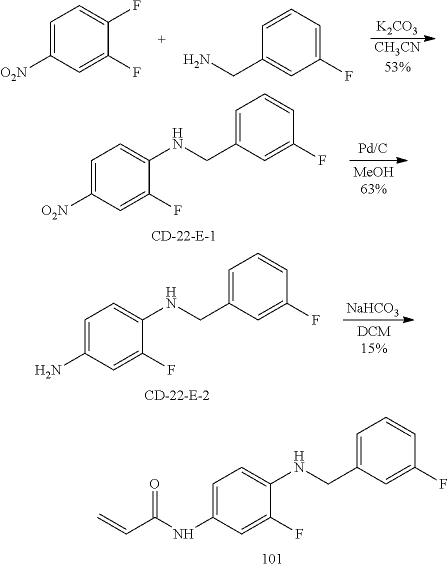

2-fluoro-N-(3-fluorobenzyl)-4-nitroaniline (CD-22-E-1). To a stirred solution of 1,2-difluoro-4-nitrobenzene (1.000 g, 6.29 mmol) and (3-fluorophenyl)methanamine (0.786 g, 6.29 mmol) in acetonitrile (20 mL) was added potassium carbonate (2.604 g, 18.87 mmol) at room temperature, and the resulting mixture was stirred at room temperature for 3 hours. The reaction mixture was poured into water (50 mL) and extracted with ethyl acetate (35 mL×2). Combined organic extracts were washed with brine (25 mL), dried over anhydrous Na₂SO₄, and concentrated in vacuo. The resulting crude was purified by silica gel column chromatography using a 20% ethyl acetate/hexane gradient to afford 2-fluoro-N-(3-fluorobenzyl)-4-nitroaniline (CD-22-E-1) (0.878 g, 53%) as a yellow solid.

2-fluoro-N¹-(3-fluorobenzyl)benzene-1,4-diamine (CD-22-E-2). To a stirred solution of 2-fluoro-N-(3-fluorobenzyl)-4-nitroaniline (CD-22-E-1) (878 mg, 3.3 mmol) in MeOH (20 mL) was added Pd/C (10%) (50 mg) at room temperature, and the resulting mixture was stirred at room temperature under H₂ atmosphere (H₂ balloon) for 4 hours. The catalyst was removed through filtration and the filtrate cake was washed with MeOH (10 mL×2), and the combined filtrates were concentrated under reduced pressure to give crude product, which was purified by silica gel flash column chromatography using a 2.5% methanol/dichloromethane gradient to afford 2-fluoro-N¹-(3-fluorobenzyl)benzene-1,4-diamine (CD-22-E-2) (0.490 g, 63%) as a yellow solid. MS [MH]⁺ 235.1.

N-(3-fluoro-4-((3-fluorobenzyl)amino)phenyl)acrylamide (101). To a stirred solution of 2-fluoro-N¹-(3-fluorobenzyl)benzene-1,4-diamine (CD-22-E-2) (0.100 g, 0.43 mmol) in a mixture of sat aq. NaHCO₃ (4 mL)-DCM (4 mL) was added acryloyl chloride (0.038 g, 0.43 mmol) dropwise at room temperature under nitrogen, and the resulting mixture was stirred at room temperature for 10 min. The reaction mixture was concentrated under reduced pressure. Crude residue was purified by silica gel column chromatography using a 50% ethyl acetate/hexane gradient to afford N-(3-fluoro-4-((3-fluorobenzyl)amino)phenyl)acrylamide (101) (0.018 mg, 15%) as an off-white solid. ¹HNMR (400 MHz, DMSO-d₆) δ 10.01 (s, 1H), 7.65-7.60 (m, 1H), 7.45-7.38 (m, 1H), 7.24-7.20 (m, 2H), 7.08-7.00 (m, 2H), 6.59 (t, J=9.2 Hz, 1H), 6.41-6.33 (m, 1H), 6.33-6.10 (m, 2H), 5.76-5.71 (m, 1H), 4.40 (d, J=6.2 Hz, 2H). MS [MH]⁺ 289.2.

The following compounds were prepared in a manner analogous to the procedures described above for N-(3-fluoro-4-((3-fluorobenzyl)amino)phenyl)acrylamide (101):

N-(4-(4-cyclohexylphenoxy)-3-fluorophenyl)acrylamide (102) (0.030 g, 30%) as white solid. ¹H NMR (400 MHz, DMSO-d₆): δ 10.36 (s, 1H), 7.87-7.83 (m, 1H), 7.37-7.34 (m, 1H), 7.20-7.13 (m, 3H), 6.86-6.84 (m, 2H), 6.45-6.38 (m, 1H), 6.31-6.26 (m, 1H), 5.81-5.78 (m, 1H), 2.47-2.41 (m, 1H), 1.78-1.68 (m, 5H), 1.43-1.25 (m, 5H). MS [MH]⁺ 340.2.

N-(3-fluoro-4-(3,3,3-trifluoropropoxy)phenyl)acrylamide (103) (0.050 g, 54%) as a yellow solid. ¹HNMR (400 MHz, DMSO-d₆): 10.19 (s, 1H), 7.71-7.67 (m, 1H), 7.32-7.30 (m, 1H), 7.21-7.17 (m, 1H), 6.42-6.35 (m, 1H), 6.27-6.23 (m, 1H), 5.77-5.74 (m, 1H), 4.26-4.23 (m, 2H), 2.81-2.78 (m, 2H) MS: [MH]⁺ 278.0.

N-(3-fluoro-4-((5-(trifluoromethyl)pyridin-3-yl)methoxy)phenyl)acrylamide (104) (0.020 g, 33%) as white solid. ¹H NMR (400 MHz, CDCl₃): δ 8.87 (s, 2H), 8.05 (s, 1H), 7.60-7.57 (m, 1H), 7.26-7.17 (m, 2H), 7.00-6.96 (m, 1H), 6.46-6.42 (m, 1H), 6.25-6.18 (m, 1H), 5.99-5.96 (m, 1H), 5.19 (s, 2H). MS: [MH]⁺ 341.2.

N-(3-fluoro-4-(1-(3-(trifluoromethyl)phenyl)ethoxy)phenyl)acrylamide (105) (0.070 g, 57%) as a light yellow solid. ¹H NMR (400 MHz, CDCl₃) δ 7.64 (s, 1H), 7.61-7.42 (m, 4H), 7.18 (s, 1H), 7.01 (d, J=8.4 Hz, 1H), 6.77 (t, J=8.8 Hz, 1H), 6.44-6.40 (m, 1H), 6.18-6.14 (m, 1H), 5.77-5.74 (m, 1H), 5.36-5.33 (m, 1H), 1.67 (d, J=6.4 Hz, 3H). MS: [MH]⁺. 354.2.

N-(3-fluoro-4-(3-fluorophenoxy)phenyl)acrylamide (106) (81 mg, 65%) as a yellow solid. ¹HNMR (400 MHz, CD₃OD): 7.84-7.80 (m, 1H), 7.39-7.30 (m, 2H), 7.19-7.15 (m, 1H), 6.85-6.80 (m, 1H), 6.76-6.73 (m, 1H), 6.70-6.66 (m, 1H), 6.48-6.38 (m, 2H), 5.83-5.80 (m, 1H). MS: [MH]⁺ 276.0.

N-(3-fluoro-4-(4-fluorophenoxy)phenyl)acrylamide (107) (0.072 g, 77%) as a white solid. ¹H NMR (400 MHz, CD₃OD) δ 7.80-7.77 (m, 1H), 7.35-7.32 (m, 1H), 7.10-7.05 (m, 3H), 6.98-6.95 (m, 2H), 6.47-6.37 (m, 2H), 5.82-5.79 (m, 1H). MS: [MH]⁺. 276

N-(3-fluoro-4-((4-fluorophenyl)amino)phenyl)acrylamide (108) (0.014 g, 39%) as a gray solid. ¹H NMR (400 MHz, CD₃OD) δ 7.66-7.62 (m, 1H), 7.20-7.11 (m, 2H), 6.92-7.04 (m, 4H), 6.44-6.31 (m, 2H), 5.75-5.70 (m, 1H). MS: [MH]⁺ 275.1.

N-(4-(((4,4-difluorocyclohexyl)methyl)amino)-3-fluorophenyl)acrylamide (109) (0.020 mg, 33%) as white solid. ¹H NMR (400 MHz, CD₃OD): δ 7.48-7.44 (m, 1H), 7.16-7.13 (m, 1H), 6.72 (t, J=7.2 Hz, 1H), 6.43-6.30 (m, 2H), 5.76-5.73 (m, 1H), 3.08 (d, J=6.8 Hz, 2H), 2.11-2.02 (m, 2H), 1.94-1.91 (m, 2H), 1.86-1.68 (m, 3H), 1.37-1.27 (m, 2H). MS: [MH]⁺. 313.0.

N-(4-(3-(tert-butyl)phenoxy)-3-fluorophenyl)acrylamide (110) (0.050 g, 60%) as an off-white solid. ¹H NMR (400 MHz, CDCl₃): δ 7.69 (d, J=12.4 Hz, 1H), 7.23 (t, J=8.0 Hz, 1H), 7.19-7.14 (m, 1H), 7.13-7.10 (m, 1H), 7.07 (t, J=2.0 Hz, 1H), 7.02 (t, J=8.8 Hz, 1H), 6.73-6.69 (m, 1H), 6.49-6.43 (m, 1H), 6.28-6.19 (m, 1H), 5.83-5.79 (m, 1H), 1.30 (s, 9H). MS: [MH]⁺ 314.1.

N-(4-(4-(tert-butyl)phenoxy)-3-fluorophenyl)acrylamide (111) (0.042 g, 35%) as a white solid ¹H NMR (400 MHz, CDCl₃): δ 7.67 (d, J=12.0 Hz, 1H), 7.46 (s, 1H), 7.34-7.30 (m, 2H), 7.16 (d, J=8.4 Hz, 1H), 7.01 (t, J=8.8 Hz, 1H), 6.90-6.87 (m, 2H), 6.47-6.43 (m, 1H), 6.28-6.21 (m, 1H), 5.81-5.78 (m, 1H), 1.30 (s, 9H). MS: [MH]⁺ 314.1.

N-(3-fluoro-4-phenoxy phenyl)acrylamide (112) (0.143 g, 95%) as a white solid. ¹H NMR (400 MHz, DMSO-d₆): δ 10.37 (s, 1H), 7.86-7.82 (m, 1H), 7.40-7.34 (m, 3H), 7.20 (t, J=9.2 Hz, 1H), 7.10 (t, J=7.4 Hz, 1H), 6.94 (d, J=7.6 Hz, 2H), 6.46-6.39 (m, 1H), 6.29-6.24 (m, 1H), 5.82-5.80 (m, 1H). MS: [MH]⁺ 258.1.

N-(3-fluoro-4-(m-tolyloxy)phenyl)acrylamide (113) (0.104 g, 83%) as a white solid. ¹H NMR (400 MHz, DMSO-d₆): δ 10.37 (s, 1H), 7.86-7.82 (m, 1H), 7.39-7.36 (m, 1H), 7.25-7.15 (m, 2H), 6.91 (d, J=7.6 Hz, 1H), 6.76-6.71 (m, 2H), 6.45-6.39 (m, 1H), 6.29-6.24 (m, 1H), 5.82-5.80 (m, 1H), 2.27 (s, 3H). MS: [MH]⁺ 272.0.

N-(3-fluoro-4-(p-tolyloxy)phenyl)acrylamide (114) (0.108 g, 87%) as a white solid. ¹H NMR (400 MHz, DMSO-d₆): δ 10.35 (s, 1H), 7.84-7.80 (m, 1H), 7.37-7.34 (m, 1H), 7.17-7.11 (m, 3H), 6.86-6.83 (m, 2H), 6.45-6.38 (m, 1H), 6.28-6.26 (m, 1H), 5.79-5.75 (m, 1H), 2.27 (s, 3H). MS: [MH]⁺ 272.1.

N-(3-fluoro-4-((6-(trifluoromethyl)pyridin-2-yl)methoxy)phenyl)acrylamide (115) (0.080 g, 69%) as an off-white solid. ¹H NMR (400 MHz, DMSO-d₆): δ 10.20 (s, 1H), 8.18 (t, J=7.8 Hz, 1H), 7.87-7.82 (m, 2H), 7.73-7.70 (m, 1H), 7.31-7.22 (m, 2H), 6.39-6.34 (m, 1H), 6.25-6.20 (m, 1H), 5.76-5.73 (m, 1H), 5.32 (s, 2H). MS: [MH]⁺ 341.2.

N-(3-fluoro-4-((2-(trifluoromethyl)pyridin-4-yl)methoxy)phenyl)acrylamide (116) (0.020 g, 38%) as an off-white solid. ¹H NMR (400 MHz, CDCl₃): δ 8.75 (d, J=4.8 Hz, 1H), 7.77 (s, 1H), 7.60 (t, J=8.6 Hz, 2H), 7.20 (t, J=12.8 Hz, 2H), 6.94 (t, J=8.8 Hz, 1H), 6.44-6.40 (m, 1H), 6.26-6.22 (m, 1H), 5.85-5.80 (m, 1H), 5.20 (s, 2H). MS: [MH]⁺ 341.2.

N-(3-fluoro-4-((4-(trifluoromethyl)pyridin-2-yl)methoxy)phenyl)acrylamide (117) (0.032 g, 36%) as a white solid. ¹H NMR (400 MHz, DMSO-d₆): δ 10.21 (s, 1H), 8.89 (d, J=5.2 Hz, 1H), 7.85 (s, 1H), 7.78-7.71 (m, 2H), 7.32-7.24 (m, 2H), 6.42-6.36 (m, 1H), 6.25-6.20 (m, 1H), 5.76-5.71 (m, 1H), 5.34 (s, 2H). MS: [MH]⁺ 341.2.

### Example 1.36. Synthesis of 4-acrylamido-2-fluoro-N-(3-fluorophenyl)benzamide (118)

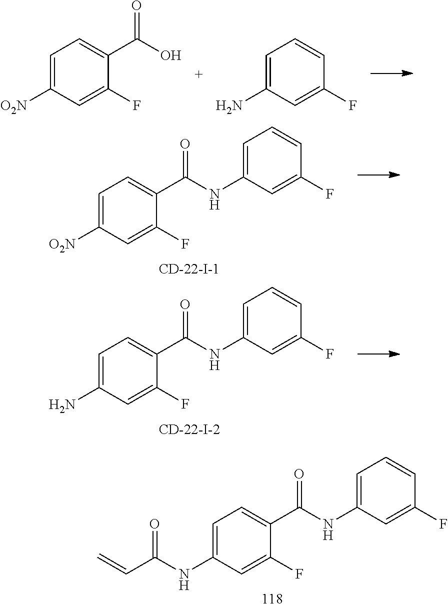

2-fluoro-N-(3-fluorophenyl)-4-nitrobenzamide (CD-22-I-1). To a solution of 2-fluoro-4-nitrobenzoic acid (0.183 g, 0.99 mmol), 3-fluoroaniline (0.100 g, 0.90 mmol), and DIPEA (0.349 g, 2.70 mmol) in DMF (5 mL) was added HATU (0.513 g, 1.35 mmol), and the resulting mixture was stirred at room temperature overnight under nitrogen. The reaction mixture was poured into water (6 mL) and extracted with ethyl acetate (8 mL×2). Combined organic extracts were washed with brine (20 mL), dried over Na₂SO₄, and concentrated in vacuo. The resulting crude was purified by silica gel column chromatography using a 20-50% hexane/ethyl acetate gradient to afford 2-fluoro-N-(3-fluorophenyl)-4-nitrobenzamide (CD-22-I-1) (0.130 g, 52%) as a yellow solid.

4-amino-2-fluoro-N-(3-fluorophenyl)benzamide (CD-22-I-2). To a solution of 2-fluoro-N-(3-fluorophenyl)-4-nitrobenzamide (CD-22-I-1) (0.130 g, 0.467 mmol) in MeOH (10 mL) was added Pd/C (10%) (5 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to afford 4-amino-2-fluoro-N-(3-fluorophenyl)benzamide (CD-22-I-2) (0.120 g, 100%) as a white solid. MS [MH]⁺ 249.3.

4-acrylamido-2-fluoro-N-(3-fluorophenyl)benzamide (118). To a solution of 4-amino-2-fluoro-N-(3-fluorophenyl)benzamide (CD-22-I-2) (0.070 g, 0.28 mmol) in a mixture of sat aq. NaHCO₃ (2 mL)-THF (2 mL) was added acryloyl chloride (0.025 g, 0.28 mmol) dropwise at room temperature under nitrogen, and the resulting mixture was stirred at room temperature for 10 min. The reaction mixture was poured into water (5 mL) and extracted with DCM (5 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford 4-acrylamido-2-fluoro-N-(3-fluorophenyl)benzamide (118) (0.050 g, 58%) as a white solid. ¹H NMR (400 MHz, CDCl₃): δ 8.49 (d, J=16.8 Hz, 1H), 8.13 (t, J=8.4 Hz, 1H), 8.00 (dd, J=2.0 Hz, 15.2 Hz, 1H), 7.68-7.65 (m, 1H), 7.62 (s, 1H), 7.32-7.30 (m, 1H), 7.28-7.24 (m, 1H), 7.16 (dd, J=2.0 Hz, 8.8 Hz, 1H), 6.88-6.83 (m, 1H), 6.52-6.23 (m, 1H). MS [MH]⁺ 303.4.

### Example 1.37. Synthesis of N-(3-fluoro-4-(1,2,3,4-tetrahydronaphthalen-2-yl)phenyl)acrylamide (119)

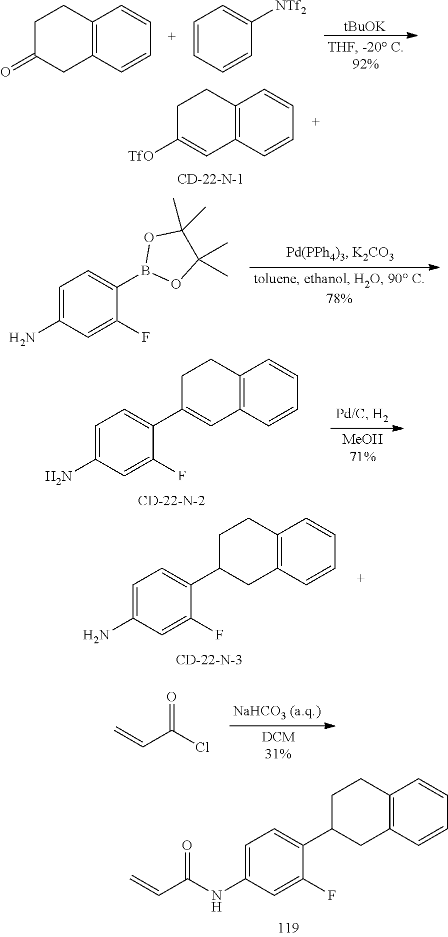

3,4-dihydronaphthalen-2-yl trifluoromethanesulfonate (CD-22-N-1). To a solution of 3,4-dihydronaphthalen-2(1H)-one (2.000 g, 13.6 mmol) in tetrahydrofuran (20 mL) was added a solution of KOtBu (1.993 g, 17.8 mmol) in tetrahydrofuran (20 mL) dropwise at 0° C., and the resulting mixture was stirred at 0° C. for 1 h. Then, 1,1,1-trifluoro-N-phenyl-N-((trifluoromethyl)sulfonyl)methanesulfonamide (6.4 g, 17.8 mmol) was added at −20° C., and the resulting mixture was stirred at 0° C. for 4 h. The reaction mixture was poured into water (50 mL) and extracted with ethyl acetate (50 mL). Combined organic extracts were washed with brine (50 mL), dried over anhydrous Na₂SO₄, and concentrated in vacuo. The resulting crude was purified by silica gel column chromatography using a hexane gradient to afford 3,4-dihydronaphthalen-2-yl trifluoromethanesulfonate (CD-22-N-1) (4.050 g, 92%) as a colorless oil. MS [MH]⁺ 279.0.

4-(3,4-dihydronaphthalen-2-yl)-3-fluoroaniline (CD-22-N-2). To a mixture of 3,4-dihydronaphthalen-2-yl trifluoromethanesulfonate (CD-22-N-1) (0.093 g, 0.39 mmol), 3-fluoro-4-(4,4,5,5-tetramethyl-1,3,2-dioxaborolan-2-yl)aniline (0.100 g, 0.36 mmol), and potassium carbonate (100 mg, 0.72 mmol) in toluene (4 mL)-ethanol (1 mL)-water (1 mL) was added tetrakis(triphenylphosphine)palladium (0.041 mg, 0.03 mmol) at room temperature under nitrogen atmosphere; the mixture was degassed with nitrogen three times. The resulting mixture was refluxed for 2 h. The resulting mixture was filtered, and the filtrate was concentrated in vacuo. The resulting crude was purified by silica gel column chromatography using a 10% ethyl acetate/hexane gradient to afford 4-(3,4-dihydronaphthalen-2-yl)-3-fluoroaniline (CD-22-N-2) (0.066 g, 78%) as a brown oil. MS [MH]⁺ 240.0.

3-fluoro-4-(1,2,3,4-tetrahydronaphthalen-2-yl)aniline (CD-22-N-3). To a solution of 4-(3,4-dihydronaphthalen-2-yl)-3-fluoroaniline (CD-22-N-2) (66 mg, 0.28 mmol) in MeOH (20 mL) was added Pd/C (10%) (5 mg) at room temperature, and the resulting mixture was stirred at room temperature under H₂ atmosphere (H₂ balloon) for 4 hours. The catalyst was removed through filtration, the filtrate cake was washed with MeOH (10 mL×2), and the combined filtrates were concentrated under reduced pressure to give crude product, which was purified by silica gel flash column chromatography using a 2.5% methanol/dichloromethane gradient to afford 3-fluoro-4-(1,2,3,4-tetrahydronaphthalen-2-yl)aniline (CD-22-N-3) (0.047 g, 71%) as a yellow oil. MS [MH]⁺ 242.0.

N-(3-fluoro-4-(1,2,3,4-tetrahydronaphthalen-2-yl)phenyl)acrylamide (119). To a mixture of 3-fluoro-4-(1,2,3,4-tetrahydronaphthalen-2-yl)aniline (CD-22-N-3) (0.047 mg, 0.20 mmol) in a mixture of sat aq. NaHCO₃ (4 mL)-DCM (4 mL) was added acryloyl chloride (0.026 g, 0.30 mmol) dropwise at room temperature under nitrogen, and the resulting mixture was stirred at room temperature for 10 min. The organic layer was collected, and the aqueous layer was extracted with DCM (5 mL×2). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(3-fluoro-4-(1,2,3,4-tetrahydronaphthalen-2-yl)phenyl)acrylamide (119) (0.020 g, 31%) as a white solid. ¹H NMR (400 MHz, DMSO-d6) δ 10.30 (s, 1H), 7.67 (d, J=9.2 Hz, 1H), 7.36-7.32 (m, 2H), 7.12-7.10 (m, 4H), 6.41 (d, J=16.8 Hz, 1H), 6.27 (d, J=16.4 Hz, 1H), 5.78 (d, J=9.6 Hz, 1H), 3.21-3.14 (m, 1H), 2.96-2.82 (m, 4H), 2.02-1.87 (m, 2H). MS [MH]⁺ 296.0.

### Example 1.38. Synthesis of N-(4-(chroman-3-yl)-3-fluorophenyl)acrylamide (120)

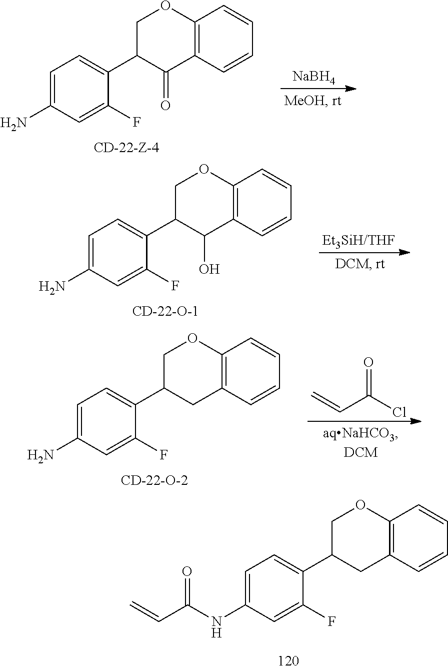

3-(4-amino-2-fluorophenyl)chroman-4-ol (CD-22-O-1). To a solution of 3-(4-amino-2-fluorophenyl)chroman-4-one (CD-22-Z-4) (0.060 g, 0.23 mmol) in methanol (4 mL) was added sodium borohydride (0.035 g, 0.93 mmol) at 0° C. under nitrogen, and the resulting mixture was stirred at room temperature for 1.5 h. The reaction mixture was poured into water (10 mL) and extracted with ethyl acetate (5 mL×2). Combined organic extracts were washed with brine (5 mL), dried over anhydrous Na₂SO₄, and concentrated in vacuo to afford 3-(4-amino-2-fluorophenyl)chroman-4-ol (CD-22-O-1) (0.060 g, crude), which was used next step without further purification. MS [MH]⁺ 260.3.

4-(chroman-3-yl)-3-fluoroaniline (CD-22-O-2). A mixture of 3-(4-amino-2-fluorophenyl)chroman-4-ol was dissolved (CD-22-O-1) (0.060 g, crude) in TFA (1 mL)-triethylsilane (1 mL) was stirred at room temperature under nitrogen for 2 h. The reaction mixture was concentrated under in vacuo. The resulting crude was purified by silica gel column chromatography using a 2-10% ethyl acetate/hexane gradient to afford 4-(chroman-3-yl)-3-fluoroaniline (CD-22-O-2) (0.049 g, two steps 87%) as a light yellow solid. MS [MH]⁺ 244.0.

N-(4-(chroman-3-yl)-3-fluorophenyl)acrylamide (120). To a stirred solution of 4-(chroman-3-yl)-3-fluoroaniline (CD-22-O-2) (0.049 g, 0.201 mmol) in a mixture of sat aq. NaHCO₃ (2 mL)-DCM (2 mL) was added acryloyl chloride (0.027 g, 0.30 mmol) dropwise at room temperature under nitrogen, and the resulting mixture was stirred at room temperature for 10 min. The organic layer was collected, and the aqueous layer was extracted with DCM (5 mL×2). Combined organic extracts were washed with brine (10 mL), dried over anhydrous Na₂SO₄, and concentrated in vacuo. The resulting crude was purified by silica gel column chromatography using a 25-50% ethyl acetate/hexane gradient to afford N-(4-(chroman-3-yl)-3-fluorophenyl)acrylamide (120) (0.024 g, 57%) as a white solid. ¹H NMR (400 MHz, CD₃OD): δ 7.57-7.51 (m, 1H), 7.23-7.14 (m, 2H), 7.00 (t, J=8.0 Hz, 2H), 6.78-6.70 (m, 2H), 6.37-6.25 (m, 2H), 5.75-5.70 (m, 1H), 4.28-4.22 (m, 1H), 4.02 (t, J=10.0 Hz, 1H), 3.04-2.89 (m, 2H), 3.04-2.89 (m, 1H). MS [MH]⁺ 298.1.

### Example 1.39. Synthesis of N-(4-(3,4-dihydro-2H-benzo[b][1,4]oxazin-3-yl)-3-fluorophenyl)acrylamide (121)

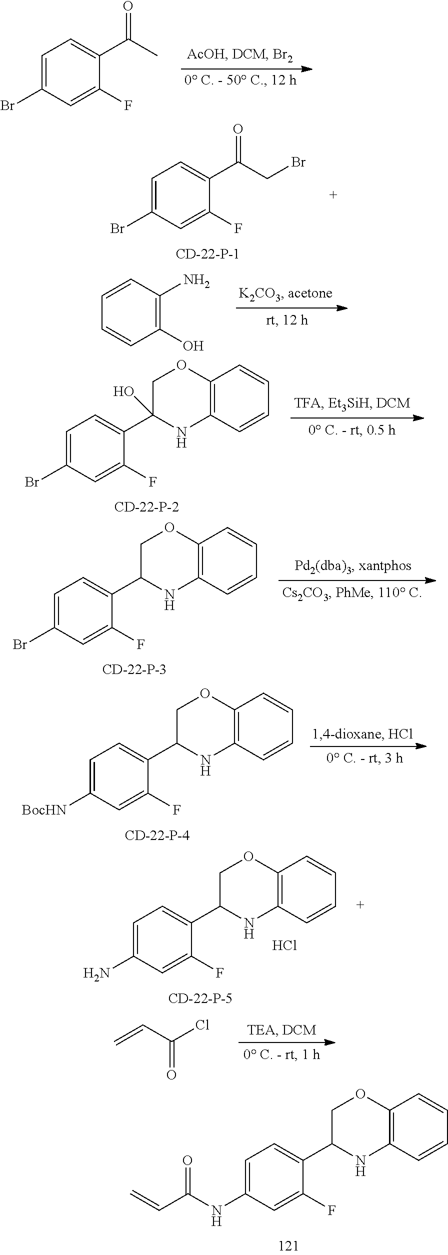

2-bromo-1-(4-bromo-2-fluorophenyl)ethanone (CD-22-P-1). To a solution of 1-(4-bromo-2-fluorophenyl)ethanone (3 g, 13.8 mmol) in DCM (70 mL) was added Br₂ (2.2 g, 13.8 mmol) in AcOH (6 mL) dropwise at 0° C. under N₂, and the resulting mixture was stirred at room temperature for 12 h. The reaction mixture was poured into ice-water (100 mL) and extracted with ethyl acetate (80 ml×3). Combined organic extracts were washed with brine (10 mL), dried over anhydrous Na₂SO₄, and concentrated in vacuo. The resulting crude was purified by silica gel column chromatography using a 10-15% DCM/hexane gradient to afford 2-bromo-1-(4-bromo-2-fluorophenyl)ethanone (CD-22-P-1) as a white solid (3.58 g, 87%).

3-(4-bromo-2-fluorophenyl)-3,4-dihydro-2H-benzo[b][1,4]oxazin-3-ol (CD-22-P-2). To a solution of 2-bromo-1-(4-bromo-2-fluorophenyl)ethanone (CD-22-P-1) (1.5 g, 5.1 mmol) and 2-aminophenol (0.553 g, 5.1 mmol) in acetone (25 mL) was added K₂CO₃ (1.1 g, 7.6 mmol) at room temperature under nitrogen, and the resulting mixture was stirred at room temperature for 12 h. The resulting mixture was filtered, and the filtrate was concentrated in vacuo. The resulting crude was purified by basic Al₂O₃ column chromatography using a 2-3% ethyl acetate/hexane gradient to afford 3-(4-bromo-2-fluorophenyl)-3,4-dihydro-2H-benzo[b][1,4]oxazin-3-ol (CD-22-P-2) (0.250 g, 15%) as a yellow solid. MS [MH-16]⁺ 308.0.

3-(4-bromo-2-fluorophenyl)-3,4-dihydro-2H-benzo[b][1,4]oxazine (CD-22-P-3). To a solution of 3-(4-bromo-2-fluorophenyl)-3,4-dihydro-2H-benzo[b][1,4]oxazin-3-ol (CD-22-P-2) (0.270 g, 0.80 mmol) in DCM (4 mL) was added TFA (0.946 g, 8.30 mmol)-Et₃SiH (0.963 g, 8.30 mmol) at 0° C. dropwise under N₂, and the resulting mixture was stirred at room temperature for 0.5 h. The reaction mixture was poured into aqueous NaHCO₃ solution (saturated, 100 mL) and extracted with DCM (25 ml×2). Combined organic extracts were washed with brine (25 mL), dried over anhydrous Na₂SO₄, and concentrated in vacuo to afford 3-(4-bromo-2-fluorophenyl)-3,4-dihydro-2H-benzo[b][1,4]oxazine (CD-22-P-3) (0.250 g, 95%) as a white solid. MS [MH]⁺ 310.0.

tert-butyl (4-(3,4-dihydro-2H-benzo[b][1,4]oxazin-3-yl)-3-fluorophenyl)carbamate (CD-22-P-4). To a solution of 3-(4-bromo-2-fluorophenyl)-3,4-dihydro-2H-benzo[b][1,4]oxazine (CD-22-P-3) (0.200 g, 0.65 mmol), tert-butyl carbamate (0.113 g, 0.97 mmol), XantPhos (0.075 g, 0.13 mmol), and Cs₂CO₃ (0.619 g, 1.9 mmol) in toluene (10 mL) was added Pd₂(dba)₃ (0.059 g, 0.065 mmol) at room temperature under nitrogen atmosphere; the mixture was degassed with nitrogen three times. The resulting mixture was refluxed for 12 h. The resulting mixture was filtered, and the filtrate was concentrated in vacuo. The resulting crude was purified by silica gel column chromatography using a 20-30% ethyl acetate/hexane gradient to afford tert-butyl (4-(3,4-dihydro-2H-benzo[b][1,4]oxazin-3-yl)-3-fluorophenyl)carbamate (CD-22-P-4) (0.100 g, 44%) as a yellow solid. MS [MH]⁺ 345.1.

4-(3,4-dihydro-2H-benzo[b][1,4]oxazin-3-yl)-3-fluoroaniline hydrochloride (CD-22-P-5). A mixture of tert-butyl (4-(3,4-dihydro-2H-benzo[b][1,4]oxazin-3-yl)-3-fluorophenyl)carbamate (CD-22-P-4) (0.095 g, 0.27 mmol) in 4 N HCl/1,4-dioxane (1 mL) was stirred at room temperature for 2 h. The reaction mixture was concentrated under reduced pressure to afford 4-(3,4-dihydro-2H-benzo[b][1,4]oxazin-3-yl)-3-fluoroaniline hydrochloride (CD-22-P-5) as a yellow solid (0.060 g, 80%).

N-(4-(3,4-dihydro-2H-benzo[b][1,4]oxazin-3-yl)-3-fluorophenyl)acrylamide (121). To a solution of 4-(3,4-dihydro-2H-benzo[b][1,4]oxazin-3-yl)-3-fluoroaniline hydrochloride (CD-22-P-5) (0.065 g, 0.27 mmol) and TEA (0.067 g, 0.67 mmol) in DCM (5 mL) was added acryloyl chloride (0.024 mg, 0.27 mmol) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(4-(3,4-dihydro-2H-benzo[b][1,4]oxazin-3-yl)-3-fluorophenyl)acrylamide (121) (0.035 g, 43%) as a yellow solid. ¹H NMR (400 MHz, CDCl₃) δ 7.66 (d, J=11.6 Hz, 1H), 7.40 (t, J=8.0 Hz, 1H), 7.35 (s, 1H), 7.14 (d, J=7.2 Hz, 1H), 6.88-6.85 (m, 2H), 6.70 (t, J=8.0 Hz, 2H), 6.45 (d, J=16.8 Hz, 1H), 6.26-6.19 (m, 1H), 5.80 (d, J=10.4 Hz, 1H), 4.86-4.82 (m, 1H), 4.38-4.33 (m, 1H), 4.06-4.01 (m, 1H), 3.94 (br, 1H). MS [MH]⁺ 299.3.

### Example 1.40. Synthesis of N-(4-(2,3-dihydrobenzo[b][1,4]dioxin-2-yl)-3-fluorophenyl)acrylamide (122)

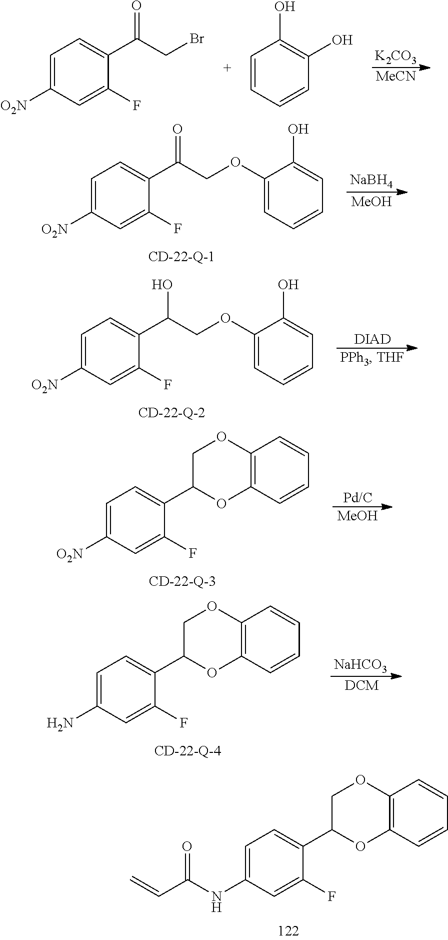

1-(2-fluoro-4-nitrophenyl)-2-(2-hydroxyphenoxy)ethanone (CD-22-Q-1). To a stirred solution of pyrocatechol (0.210 g, 1.90 mmol) and potassium carbonate (0.524 g, 3.81 mmol) in acetonitrile (5 mL) was added 2-bromo-1-(2-fluoro-4-nitrophenyl)ethanone (0.500 g, 1.90 mmol) at room temperature, and the resulting mixture was stirred at room temperature for 2 min. The reaction mixture was poured into water (20 mL) and extracted with ethyl acetate (15 mL×2). Combined organic extracts were washed with brine (25 mL), dried over anhydrous Na₂SO₄, and concentrated in vacuo. The resulting crude was purified by silica gel column chromatography using a 10% ethyl acetate/hexane gradient to afford 1-(2-fluoro-4-nitrophenyl)-2-(2-hydroxyphenoxy)ethenone (CD-22-Q-1) (0.120 g, 21%) as a yellow oil.

2-(2-(2-fluoro-4-nitrophenyl)-2-hydroxyethoxy)phenol (CD-22-Q-2). To a stirred solution of 1-(2-fluoro-4-nitrophenyl)-2-(2-hydroxyphenoxy)ethanone (CD-22-Q-1) (0.120 g, 0.44 mmol) in methanol (5 ml) was added sodium tetrahydroborate (0.017 g, 0.44 mmol) at room temperature, and the resulting mixture was stirred at room temperature for 0.5 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 2-(2-(2-fluoro-4-nitrophenyl)-2-hydroxyethoxy)phenol (CD-22-Q-2) (0.910 g, 75%) as a yellow solid.

2-(2-fluoro-4-nitrophenyl)-2,3-dihydrobenzo[b][1,4]dioxine] (CD-22-Q-3). To a stirred solution of 2-(2-(2-fluoro-4-nitrophenyl)-2-hydroxyethoxy)phenol (CD-22-Q-2) (0.090 g, 0.30 mmol) and triphenylphosphine (157 mg, 0.6 mmol) in anhydrous tetrahydrofuran (5 mL) was added DIAD (0.124 g, 0.60 mmol) at 0° C., and the resulting mixture was stirred at room temperature for 1 h. The reaction mixture was poured into water (10 mL) and extracted with ethyl acetate (10 mL×2). Combined organic extracts were washed with brine (15 mL), dried over anhydrous Na₂SO₄, and concentrated in vacuo. The resulting crude was purified by silica gel column chromatography using a 10% ethyl acetate/hexane gradient to afford 2-(2-fluoro-4-nitrophenyl)-2,3-dihydrobenzo[b][1,4]dioxine (CD-22-Q-3) (0.030 g, 35%) as a yellow solid.

4-(2,3-dihydrobenzo[b][1,4]dioxin-2-yl)-3-fluoroaniline (CD-22-Q-4). To a solution of 2-(2-fluoro-4-nitrophenyl)-2,3-dihydrobenzo[b][1,4]dioxine (CD-22-Q-3) (0.030 g, 0.11 mmol) in methanol (10 ml) was added Pd/C (10%) (5 mg) at room temperature, and the resulting mixture was stirred at room temperature under H₂ atmosphere (H₂ balloon) for 4 h. The catalyst was removed through filtration, the filtrate cake was washed with MeOH (5 mL×2), and the combined filtrates were concentrated under reduced pressure to give crude product, which was purified by silica gel flash column chromatography using a 2.5% methanol/dichloromethane gradient to afford 4-(2,3-dihydrobenzo[b][1,4]dioxin-2-yl)-3-fluoroaniline (CD-22-Q-4) (0.025 g, crude) as a colorless oil, which was used in next step without further purification. MS [MH]⁺ 246.3.

N-(4-(2,3-dihydrobenzo[b][1,4]dioxin-2-yl)-3-fluorophenyl)acrylamide (122). To a stirred solution of 4-(2,3-dihydrobenzo[b][1,4]dioxin-2-yl)-3-fluoroaniline (CD-22-Q-4) (0.020 g, 0.080 mmol) in a mixture of sat aq. NaHCO₃ (1 mL)-DCM (1 mL) was added acryloyl chloride (0.0073 g, 0.080 mmol) dropwise at room temperature under nitrogen, and the resulting mixture was stirred at room temperature for 10 min. The organic layer was collected, and the aqueous layer was extracted with DCM (3 mL×2). Combined organic extracts were washed with brine (5 mL), dried over anhydrous Na₂SO₄, and concentrated in vacuo. The resulting crude was purified by silica gel column chromatography using a 5% methanol/dichloromethane gradient to afford N-(4-(2,3-dihydrobenzo[b][1,4]dioxin-2-yl)-3-fluorophenyl)acrylamide (122) (0.018 g, 59%) as a white solid. ¹H NMR (400 MHz, DMSO-d₆): δ 10.45 (s, 1H), 7.80-7.76 (m, 1H), 7.50-7.46 (m, 1H), 7.41-7.39 (m, 1H), 6.98-6.92 (m, 2H), 6.90-6.86 (m, 2H), 6.46-6.39 (m, 1H), 6.32-6.27 (m, 1H), 5.83-5.80 (m, 1H), 5.40-5.37 (m, 1H), 4.45-4.42 (m, 1H), 4.18-4.13 (m, 1H). MS [MH]⁺ 300.3.

### Example 1.41. Synthesis of N-(4-(1-(2-chlorophenyl)ethoxy)-3-fluorophenyl)acrylamide (123)

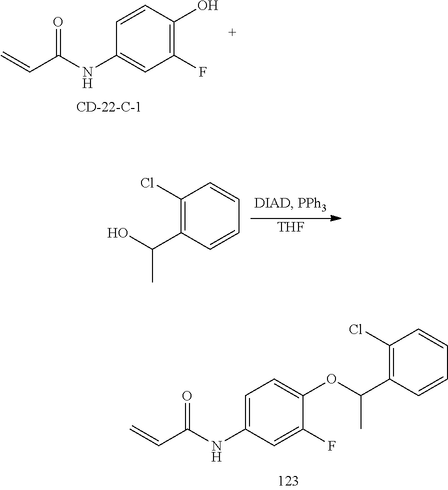

To a stirring mixture of PPh₃ (0.433 g, 1.65 mmol) in THF (6 mL) was added DIAD (0.333 mg, 1.65 mmol) at 0° C., and the resulting mixture was stirred at 5° C. for 20 mins until white solid appeared. Then, 1-(2-Chlorophenyl)ethanol (0.172 g, 1.10 mmol) was added at room temperature and stirred for 5 min. The reaction mixture was added into a solution of N-(3-fluoro-4-hydroxyphenyl)acrylamide (CD-22-C-1) (0.100 g, 0.55 mmol) in THF (4 mL), and the resulting mixture was stirred at room temperature overnight. The reaction mixture was concentrated under reduced pressure, and the resulting crude was purified by silica gel column chromatography using a 25% ethyl acetate/hexane gradient to afford N-(4-(1-(2-chlorophenyl)ethoxy)-3-fluorophenyl)acrylamide (123) (0.078 mg, 45%) as a white solid. ¹H NMR (400 MHz, DMSO-d₆): δ 10.15 (s, 1H), 7.69-7.64 (m, 1H), 7.58-7.53 (m, 1H), 7.46-7.42 (m, 1H), 7.39-7.29 (m, 2H), 7.16-7.14 (m, 1H), 6.87 (t, J=9.2 Hz, 1H), 6.39-6.32 (m, 1H), 6.28-6.23 (m, 1H), 5.75-5.70 (m, 2H), 1.58 (d, J=6.4 Hz, 3H). MS [MH]⁺ 320.4.

The following compound was prepared in a manner analogous to the procedures described above for N-(4-(1-(2-chlorophenyl)ethoxy)-3-fluorophenyl)acrylamide (123):

N-(3-fluoro-4-(1-(3-fluorophenyl)ethoxy)phenyl)acrylamide (124) (0.084 g, 50%) as white solid. ¹H NMR (400 MHz, DMSO-d₆): δ 10.15 (s, 1H), 7.66-7.61 (m, 1H), 7.43-7.37 (m, 1H), 7.25-7.22 (m, 2H), 7.18-7.01 (m, 3H), 6.39-6.32 (m, 1H), 6.25-6.20 (m, 1H), 5.71-5.74 (m, 1H), 5.54-5.49 (m, 1H), 1.56 (d, J=6.4 Hz, 3H). MS [MH]⁺ 304.0.

### Example 1.42. Synthesis of (E)-4-(dimethylamino)-N-(3-fluoro-4-((3-fluorobenzyl)oxy)phenyl)but-2-enamide (125)

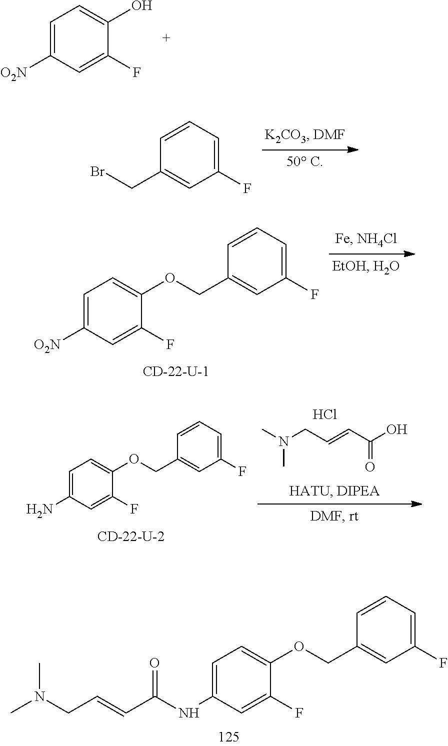

2-fluoro-1-((3-fluorobenzyl)oxy)-4-nitrobenzene (CD-22-U-1). To a solution of 2-fluoro-4-nitrophenol (1.000 g, 6.37 mmol) and 1-(bromomethyl)-3-fluorobenzene (1.260 g, 6.69 mmol) in N,N-dimethylformamide (15 mL) was added potassium carbonate (1.320 g, 9.56 mmol) at room temperature under nitrogen, and the resulting mixture was stirred at 50° C. for 2 h. After cooling to room temperature, the reaction mixture was poured into water (15 mL) and extracted ethyl acetate (30 mL×2). Combined organic extracts were washed with brine (20 mL), dried over Na₂SO₄, and concentrated in vacuo. The resulting crude was purified by silica gel column chromatography using a 10%-20% ethyl acetate/hexane gradient to afford 2-fluoro-1-((3-fluorobenzyl)oxy)-4-nitrobenzene (CD-22-U-1) (1.580 g, 94%) as an off-white solid.

3-fluoro-4-((3-fluorobenzyl)oxy)aniline (CD-22-U-2). To a mixture of 2-fluoro-1-((3-fluorobenzyl)oxy)-4-nitrobenzene (CD-22-U-1) (0.500 g, 1.89 mmol) and ammonium chloride (0.504 g, 9.43 mmol) in ethanol-water (9 mL-3 mL) was added iron powder (0.528 g, 9.43 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 20%-30% ethyl acetate/hexane gradient to afford 3-fluoro-4-((3-fluorobenzyl)oxy)aniline (CD-22-U-2) (0.414 g, 93%) as a yellow solid. MS: [MH]⁺ 236.2.

(E)-4-(dimethylamino)-N-(3-fluoro-4-((3-fluorobenzyl)oxy)phenyl)but-2-enamide (125). To a stirred solution of (E)-4-(dimethylamino)but-2-enoic acid hydrochloride (0.060 g, 0.36 mmol), 3-fluoro-4-((3-fluorobenzyl)oxy)aniline (CD-22-U-2) (0.085 g, 0.36 mmol) and N-ethyl-N-isopropylpropan-2-amine (0.140 g, 1.09 mmol) in N,N-dimethylformamide (3 mL) was added HATU (2-(7-aza-1H-benzotriazole-1-yl)-1,1,3,3-tetramethyluronium hexafluorophosphate) (0.165 mg, 0.43 mmol), and the resulting mixture was stirred at room temperature overnight under nitrogen. The reaction mixture was poured into water (6 mL) and extracted with ethyl acetate (8 mL×2). Combined organic extracts were washed with brine (20 mL), dried over Na₂SO₄, and concentrated in vacuo. The resulting crude was purified by Pre-TLC using a 50% ethyl acetate/dichloromethane containing 1% ammonium hydroxide gradient to afford (E)-4-(dimethylamino)-N-(3-fluoro-4-((3-fluorobenzyl)oxy)phenyl)but-2-enamide (125) (0.035 g, 28%) as an off-white solid. ¹H NMR (400 MHz, DMSO-d₆) δ 10.15 (s, 1H), 7.72-7.66 (m, 1H), 7.48-7.41 (m, 1H), 7.31-7.24 (m, 3H), 7.22-7.13 (m, 2H), 6.76-6.68 (m, 1H), 6.27-6.20 (m, 1H), 5.16 (s, 2H), 3.14 (d, J=5.6 Hz, 2H), 2.23 (s, 6H). MS: [MH]⁺ 347.5.

The following compound was prepared in a manner analogous to the procedures described above for (E)-4-(dimethylamino)-N-(3-fluoro-4-((3-fluorobenzyl)oxy)phenyl)but-2-enamide (125):

N-(3-fluoro-4-((3-fluorobenzyl)oxy)phenyl)but-3-ynamide (126) (45 mg, 8.8%): ¹H NMR (400 MHz, CDCl₃): δ 7.52-7.45 (m, 1H), 7.45-7.38 (m, 1H), 7.37-7.30 (m, 1H), 7.21-7.12 (m, 2H), 7.11-7.05 (m, 1H), 7.04-6.98 (m, 1H), 6.91 (t, J=8.8 Hz, 1H), 5.74 (t, J=5.2 Hz, 1H), 5.36 (d, J=6.8 Hz, 2H), 5.10 (s, 2H). MS: [MH]⁺. 302.0.

### Example 1.43. Synthesis of N-(6-fluoro-5-((3-fluorobenzyl)oxy)pyridin-2-yl)acrylamide (127)

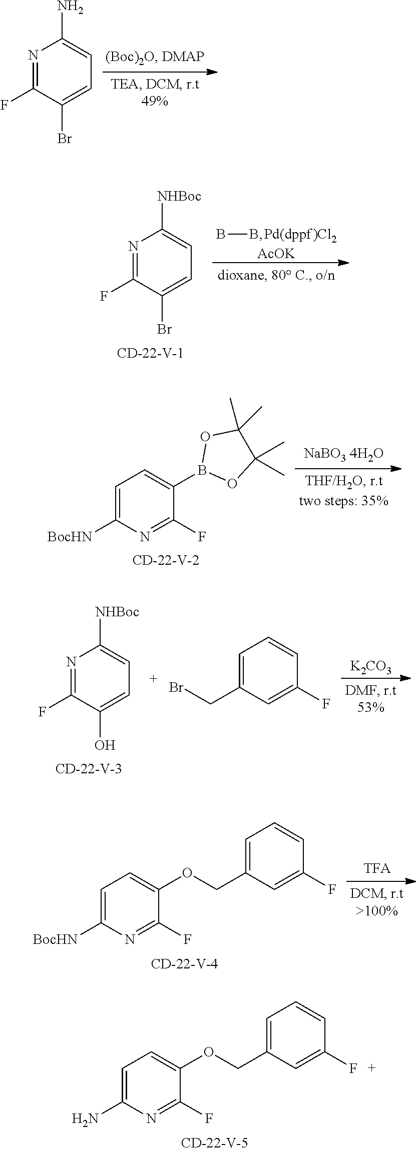

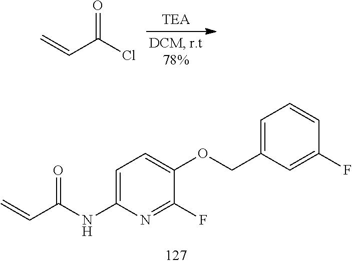

tert-butyl (5-bromo-6-fluoropyridin-2-yl) carbamate (CD-22-V-1). To a solution of 5-bromo-6-fluoropyridin-2-amine (0.500 g, 2.62 mmol), 4-dimethylaminopyridine (0.032 g, 0.26 mmol), and triethylamine (0.530 g, 5.24 mmol) in DCM (10 mL) was added di-tert-butyl oxalate (628 mg, 2.88 mmol) at 0° C. under nitrogen, and the resulting mixture was stirred at 0° C. for 2 h. The reaction mixture was poured into water (30 mL) and extracted with DCM (15 mL×2). Combined organic extracts were washed with brine (25 mL), dried over anhydrous Na₂SO₄, and concentrated in vacuo. The resulting crude was purified by silica gel column chromatography using a 2.5% ethyl acetate/hexane gradient to afford tert-butyl (5-bromo-6-fluoropyridin-2-yl) carbamate (CD-22-V-1) (0.374 g, 49%) as a colorless oil. MS [MH-56]⁺ 234.8.

tert-butyl (6-fluoro-5-(4,4,5,5-tetramethyl-1,3,2-dioxaborolan-2-yl)pyridin-2-yl) carbamate (CD-22-V-2). To a solution of tert-butyl (5-bromo-6-fluoropyridin-2-yl) carbamate (CD-22-V-1) (0.374 g, 1.29 mmol), 4,4,4′,4′,5,5,5′,5′-octamethyl-2,2′-bi(1,3,2-dioxaborolane) (0.653 g, 2.57 mmol), and [1,1′-bis(diphenylphosphino) ferrocene] dichloropalladium(II) (0.094 g, 0.13 mmol) was added potassium acetate (0.211 g, 2.57 mmol) in 1,4-dioxane (10 mL) at room temperature under nitrogen atmosphere; the mixture was degassed with nitrogen three times. The resulting mixture was refluxed for 2 h. The resulting mixture was filtered, and the filtrate was concentrated in vacuo afford tert-butyl (6-fluoro-5-(4,4,5,5-tetramethyl-1,3,2-dioxaborolan-2-yl)pyridin-2-yl) carbamate (CD-22-V-2) (0.405 g, 91%) as a yellow oil.

tert-butyl (6-fluoro-5-hydroxypyridin-2-yl) carbamate (CD-22-V-3). To a solution of tert-butyl (6-fluoro-5-(4,4,5,5-tetramethyl-1,3,2-dioxaborolan-2-yl)pyridin-2-yl) carbamate (CD-22-V-2) (0.400 g, 1.18 mmol) in THF (10 ml)-H₂O (5 mL) was added sodium perborate (0.364 g, 2.36 mmol) at room temperature, and the resulting mixture was stirred at room temperature for 1 h. The reaction concentrated in vacuo. The resulting crude was purified by silica gel column chromatography using a 10% ethyl acetate/hexane gradient to afford tert-butyl (6-fluoro-5-hydroxypyridin-2-yl) carbamate (CD-22-V-3) (0.103 g, 40%) as an colorless oil. MS [MH-56]⁺ 173.0.

tert-butyl (6-fluoro-5-((3-fluorobenzyl)oxy)pyridin-2-yl) carbamate (CD-22-V-4). To a solution of tert-butyl (6-fluoro-5-hydroxypyridin-2-yl) carbamate (CD-22-V-3) (0.100 g, 0.44 mmol) and 1-(bromomethyl)-3-fluorobenzene (0.091 g, 0.48 mmol) in N,N-dimethylformamide (5 mL) was added potassium carbonate (0.121 g, 0.88 mmol) at room temperature, and the resulting mixture was stirred at room temperature for 2 h. The reaction mixture was poured into water (20 mL) and extracted with ethyl acetate (10 mL×2). Combined organic extracts were washed with brine (5 mL), dried over anhydrous Na₂SO₄, and concentrated in vacuo. The resulting crude was purified by silica gel column chromatography using a 2% ethyl acetate/hexane gradient to afford tert-butyl (6-fluoro-5-((3-fluorobenzyl)oxy)pyridin-2-yl) carbamate (CD-22-V-4) (0.078 g, 53%) as a colorless oil. [MH]⁺ 337.1.

6-fluoro-5-((3-fluorobenzyl)oxy)pyridin-2-amine (CD-22-V-5). To a stirring mixture of tert-butyl (6-fluoro-5-((3-fluorobenzyl)oxy)pyridin-2-yl) carbamate (CD-22-V-4) (0.075 g, 0.22 mmol) in DCM (2 mL) was added TFA (1 mL) at room temperature, and the resulting mixture was stirred at room temperature for 1 h. The reaction mixture was concentrated in vacuo to afford 6-fluoro-5-((3-fluorobenzyl)oxy)pyridin-2-amine (CD-22-V-5) (0.071 g, 100%) as an off-white solid. [MH]⁺ 237.2.

N-(6-fluoro-5-((3-fluorobenzyl)oxy)pyridin-2-yl)acrylamide (127). To a solution of 6-fluoro-5-((3-fluorobenzyl)oxy)pyridin-2-amine (CD-22-V-5) (0.071 g, 0.30 mmol) and TEA (0.091 g, 0.90 mmol) in dichloromethane (5 mL) was added acryloyl chloride (0.045 g, 0.50 mmol) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(6-fluoro-5-((3-fluorobenzyl)oxy)pyridin-2-yl)acrylamide (127) (0.068 g, 78%) as yellow solid. ¹H NMR (400 MHz, DMSO-d₆): δ 10.78 (s, 1H), 8.09 (d, J=8.8 Hz, 1H), 7.86 (t, J=9.6 Hz, 1H), 7.55-7.49 (m, 1H), 7.36 (d, J=8.0 Hz, 2H), 7.25 (t, J=8.0 Hz, 1H), 6.62-6.55 (m, 1H), 6.37-6.33 (m, 1H), 5.85-5.82 (m, 1H), 5.28 (s, 2H). [MH]⁺ 291.3.

The following compound was prepared in a manner analogous to the procedures described above for N-(6-fluoro-5-((3-fluorobenzyl)oxy)pyridin-2-yl)acrylamide (127):

N-(4-fluoro-5-((3-fluorobenzyl)oxy)pyridin-2-yl)acrylamide (128) (0.025 g, 50%) as a white solid. ¹H NMR (400 MHz, DMSO-d₆): δ 10.87 (s, 1H), 8.32 (d, J=10.8 Hz, 1H), 8.08 (d, J=13.2 Hz, 1H), 7.49-7.43 (m, 1H), 7.32-7.29 (m, 2H), 7.23-7.17 (m, 1H), 6.56-6.51 (m, 1H), 6.35-6.30 (m, 1H), 5.79-5.72 (m 1H), 5.28 (s, 2H). MS [MH]⁺ 291.6.

### Example 1.44. Synthesis of N-(5-fluoro-6-((3-fluorobenzyl)oxy)pyridin-3-yl)acrylamide (129)

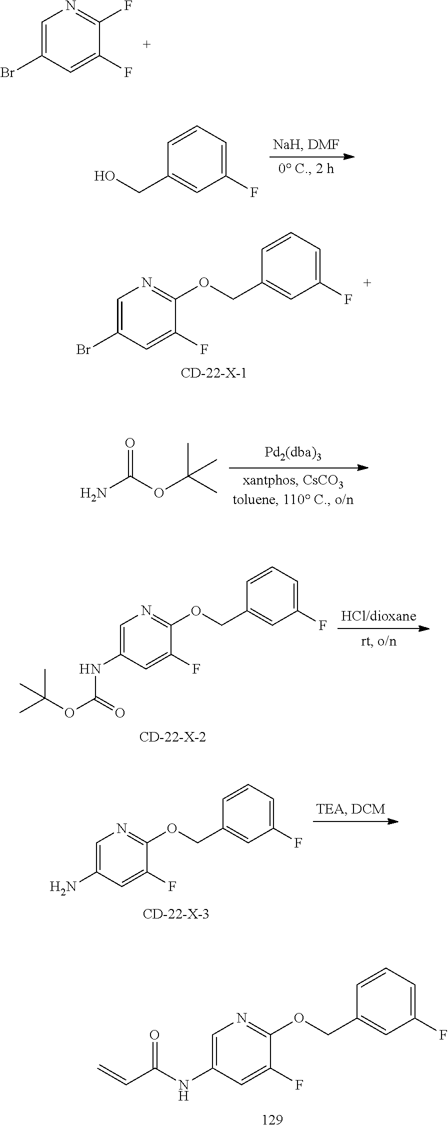

5-bromo-3-fluoro-2-((3-fluorobenzyl)oxy)pyridine (CD-22-X-1). To a solution of 5-bromo-2,3-difluoropyridine (0.200 g, 1.04 mmol) and (3-fluorophenyl)methanol (0.131 g, 1.04 mmol) in N,N-dimethylformamide (2 mL) was added sodium hydride (60% in mineral oil) (0.050 g, 1.25 mmol) at 0° C., and the resulting mixture was stirred under nitrogen for 1 h. The reaction mixture was poured into ice-water (5 mL) and extracted with ethyl acetate (10 mL×2). Combined organic extracts were washed with brine (15 mL), dried over anhydrous Na₂SO₄, and concentrated in vacuo. The resulting crude was purified by silica gel column chromatography using a 80% ethyl acetate/hexane gradient to afford 5-bromo-3-fluoro-2-((3-fluorobenzyl)oxy)pyridine (CD-22-X-1) (0.240 g, 77%) as a white solid. MS [MH]⁺ 301.8.

tert-butyl (5-fluoro-6-((3-fluorobenzyl)oxy)pyridin-3-yl)carbamate (CD-22-X-2). To a mixture of 5-bromo-3-fluoro-2-((3-fluorobenzyl)oxy)pyridine (CD-22-X-1) (0.240 g, 0.8 mmol), tert-butyl carbamate (0.103 g, 0.88 mmol), XantPhos (0.093 g, 0.16 mmol), and Cs₂CO₃ (0.780 g, 2.4 mmol) in toluene (2 mL) was added Pd₂(dba)₃ (0.073 g, 0.08 mmol) and was stirred at 110° C. at room temperature under nitrogen atmosphere; the mixture was degassed with nitrogen three times. The resulting mixture was refluxed for 2 h. The resulting mixture was filtered and the filtrate was concentrated in vacuo. The resulting crude was purified by silica gel column chromatography using a 5% ethyl acetate/hexane gradient to afford tert-butyl (5-fluoro-6-((3-fluorobenzyl)oxy)pyridin-3-yl)carbamate (CD-22-X-2) (0.105 g, 39%) as a yellow solid. MS [MH]⁺ 337.4.

5-fluoro-6-((3-fluorobenzyl)oxy)pyridin-3-amine (CD-22-X-3). A mixture of tert-butyl (5-fluoro-6-((3-fluorobenzyl)oxy)pyridin-3-yl)carbamate (CD-22-X-2) (0.105 g, 0.31 mmol) in HCl/dioxane (4 M, 2 mL) was stirred at room temperature for 2 h. The reaction mixture was concentrated under reduced pressure to give 5-fluoro-6-((3-fluorobenzyl)oxy)pyridin-3-amine (CD-22-X-3) (0.075 g, crude) as a brown solid which get to next step without purification.

N-(5-fluoro-6-((3-fluorobenzyl)oxy)pyridin-3-yl)acrylamide (129). To a solution of 5-fluoro-6-((3-fluorobenzyl)oxy)pyridin-3-amine (CD-22-X-3) (0.075 g, crude) and TEA (0.094 g, 0.93 mmol) in DCM (2 mL) was added acryloyl chloride (0.031 g, 0.34 mmol) at 0° C., and the resulting mixture was stirred at room temperature for 1.5 h. The reaction mixture was poured into water (10 mL) and extracted with DCM (10 mL). Combined organic extracts were washed with brine (10 mL), dried over anhydrous Na₂SO₄, and concentrated in vacuo. The resulting crude was purified by silica gel column chromatography using a 30-50% ethyl acetate/hexane gradient to afford N-(5-fluoro-6-((3-fluorobenzyl)oxy)pyridin-3-yl)acrylamide (129) (0.030 g, two steps: 33%) as a white solid. ¹H NMR (400 MHz, DMSO-d₆): δ 10.41 (s, 1H), 8.20 (d, J=2.2 Hz, 1H), 8.08-8.03 (m, 1H), 7.47-7.41 (m, 1H), 7.31-7.28 (m, 2H), 7.20-7.15 (m, 1H), 6.45-6.40 (m, 1H), 6.28-6.23 (m, 1H), 5.86-5.81 (m, 1H), 5.43 (s, 2H). MS [MH]⁺ 291.3.

### Example 1.45. Synthesis of N-(3-cyano-4-((3-fluorobenzyl)oxy)phenyl)acrylamide (130)

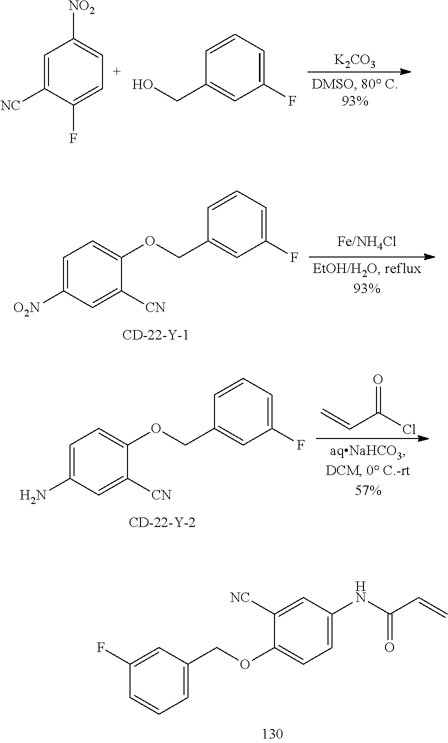

2-((3-fluorobenzyl)oxy)-5-nitrobenzonitrile (CD-22-Y-1). To a mixture of 2-fluoro-5-nitrobenzonitrile (0.500 g, 3.01 mmol) and (3-fluorophenyl)methanol (0.455 g, 3.61 mmol) DMSO (4 mL) was added potassium carbonate (0.830 g, 6.02 mmol) at room temperature, and the resulting mixture was stirred at 80° C. under nitrogen for 10 h. After cooling to room temperature, the reaction mixture was poured into water (15 mL) and stirred for 10 min at room temperature. The solid was collected through filtration, the filter cake was washed with hexane (10 mL×2), and dried up in vacuo to afford 2-((3-fluorobenzyl)oxy)-5-nitrobenzonitrile (CD-22-Y-1) (0.759 g, 93%) as a yellow solid.

5-amino-2-((3-fluorobenzyl)oxy)benzonitrile (CD-22-Y-2). To a solution of 2-((3-fluorobenzyl)oxy)-5-nitrobenzonitrile (CD-22-Y-1) (0.200 g, 0.73 mmol) and ammonium chloride (0.275 g, 5.14 mmol) in EtOH (5 mL)-H₂O (2 mL) was added iron powder (288 mg, 5.14 mmol) at room temperature, and the resulting mixture was refluxed for 2 h. After cooling to room temperature, Iron was removed through filtration and washed with methanol (10 mL×2). Combined organic filtrates were concentrated in vacuo. The resulting crude was purified by silica gel column chromatography using a 20%-30% ethyl acetate/hexane gradient to afford 5-amino-2-((3-fluorobenzyl)oxy)benzonitrile (CD-22-Y-2) (0.165 g, 93%) as yellow solid. MS [MH]⁺ 243.0.

N-(3-cyano-4-((3-fluorobenzyl)oxy)phenyl)acrylamide (130). To a stirred solution of 5-amino-2-((3-fluorobenzyl)oxy)benzonitrile (CD-22-Y-2) (0.080 g, 0.33 mmol) in a mixture of sat aq. NaHCO₃ (2 mL)-DCM (2 mL) was added acryloyl chloride (0.045 g, 0.49 mmol) dropwise at room temperature under nitrogen, and the resulting mixture was stirred at room temperature for 10 min. The organic layer was collected, and the aqueous layer was extracted with DCM (5 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(3-cyano-4-((3-fluorobenzyl)oxy)phenyl)acrylamide (130) (0.056 g, 57%) as a yellow solid. ¹H NMR (400 MHz, DMSO-d₆) δ 10.43 (s, 1H), 8.08 (d, J=2.0 Hz, 1H), 7.83 (dd, J=9.0, 2.0 Hz, 1H), 7.51-7.45 (m, 1H), 7.32 (t, J=8.4 Hz, 3H), 7.20 (t, J=8.0 Hz, 1H), 6.41 (dd, J=16.8, 10.0 Hz, 1H), 6.27 (d, J=16.4 Hz, 1H), 5.82-5.75 (m, 1H), 5.29 (s, 2H). MS [MH]⁺ 297.0.

### Example 1.46. Synthesis of N-(3-fluoro-4-(4-oxochroman-3-yl)phenyl)acrylamide (131)

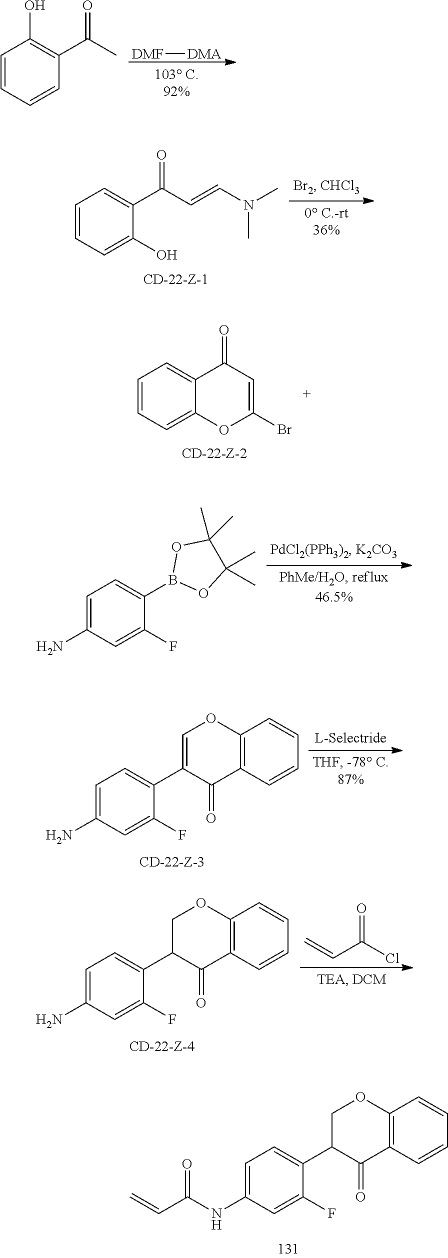

(E)-3-(dimethylamino)-1-(2-hydroxyphenyl)prop-2-en-1-one (CD-22-Z-1). A mixture of 1-(2-hydroxyphenyl)ethanone (1.000 g, 7.34 mmol) in N,N-Dimethylformamide dimethyl acetal (0.963 g, 8.08 mmol) was stirred at 103° C. for 10 h under nitrogen. After cooling to room temperature, the solid was collected through filtration, and the filter cake was washed with hexane (10 mL×2) dried up in vacuo to afford (E)-3-(dimethylamino)-1-(2-hydroxyphenyl)prop-2-en-1-one (CD-22-Z-1) (1.270 g, 91%) as a brown solid. MS [MH]⁺ 192.3.

2-bromo-4H-chromen-4-one (CD-22-Z-2). To a solution of (E)-3-(dimethylamino)-1-(2-hydroxyphenyl)prop-2-en-1-one (CD-22-Z-1) (1.270 g, 6.64 mmol) in chloroform (10 mL) was added bromine (1.060 g, 9.96 mmol) in chloroform (2 mL) dropwise at 0° C., and the resulting mixture was stirred at room temperature for 2 hours under nitrogen. The reaction mixture was poured into water (20 mL) and extracted with ethyl acetate (20 mL). The organic layer was collected, and the aqueous layer was extracted with ethyl acetate (20 mL×2). Combined organic extracts were washed with brine (25 mL), dried over anhydrous Na₂SO₄, and concentrated in vacuo. The resulting crude was purified by silica gel column chromatography using a 10-20% ethyl acetate/hexane gradient to afford 2-bromo-4H-chromen-4-one (CD-22-Z-2) (0.538 g, 36%) as a yellow solid. MS [MH]⁺ 224.9, 226.8.

3-(4-amino-2-fluorophenyl)-4H-chromen-4-one (CD-22-Z-3). To a stirred mixture of 2-bromo-4H-chromen-4-one (CD-22-Z-2) (0.455 g, 2.02 mmol), 3-fluoro-4-(4,4,5,5-tetramethyl-1,3,2-dioxaborolan-2-yl)aniline (0.479 g, 2.02 mmol), and potassium carbonate (0.100 g, 0.72 mmol) in toluene (4 mL)-water (1 mL) was added bis(triphenylphosphine)palladium(II) chloride (0.043 mg, 0.06 mmol) at room temperature under nitrogen atmosphere; the mixture was degassed with nitrogen three times. The resulting mixture was refluxed for 2 h. The resulting mixture was filtered, and the filtrate was concentrated in vacuo. The resulting crude was purified by silica gel column chromatography using a 10-33% ethyl acetate/hexane gradient to afford 3-(4-amino-2-fluorophenyl)-4H-chromen-4-one (CD-22-Z-3) (0.240 g, 46%) as a yellow solid. MS [MH]⁺ 226.2.

3-(4-amino-2-fluorophenyl)chroman-4-one (CD-22-Z-4). To a stirred solution of 3-(4-amino-2-fluorophenyl)-4H-chromen-4-one (CD-22-Z-3) (0.240 g, 0.941 mmol) in anhydrous tetrahydrofuran (4 mL) was added L-selectride (1.0M in THF) (2 mL, 2.06 mmol) dropwise at −78° C. under nitrogen, and the resulting mixture was stirred at −78° C. for 10 min. The reaction mixture was poured into aqueous ammonium chloride solution (10 mL) and extracted with ethyl acetate (10 mL×2). Combined organic extracts were washed with brine (10 mL), dried over anhydrous Na₂SO₄, and concentrated in vacuo. The resulting crude was purified by silica gel column chromatography using a 10-20% ethyl acetate/hexane gradient to afford 3-(4-amino-2-fluorophenyl)chroman-4-one (CD-22-Z-4) (0.210 g, 87%) as a yellow solid. MS [MH]⁺ 228.1.

N-(3-fluoro-4-(4-oxochroman-3-yl)phenyl)acrylamide (131). To a stirred solution of 3-(4-amino-2-fluorophenyl)chroman-4-one (CD-22-Z-4) (0.050 g, 0.19 mmol) and triethylamine (0.039 mg, 0.39 mmol) in dichloromethane (2 mL) was added acryloyl chloride (0.023 mg, 0.25 mmol) at 0° C., and the resulting mixture was stirred at room temperature for 2 hours. The reaction was poured into water (5 mL) and extracted with DCM (5 mL×2). Combined organic extracts were washed with brine (10 mL), dried over anhydrous Na₂SO₄, and concentrated in vacuo. The resulting crude was purified by silica gel column chromatography using a 50% ethyl acetate/hexane gradient to afford N-(3-fluoro-4-(4-oxochroman-3-yl)phenyl)acrylamide (131) (0.021 g, 35%) as a white solid. ¹H NMR (400 MHz, CD₃OD): δ 7.81-7.76 (m, 1H), 7.59-7.55 (m, 1H), 7.50-7.46 (m 1H), 7.22-7.19 (m, 1H), 7.13 (t, J=8.0 Hz, 1H), 7.02-6.94 (m, 2H), 6.36-6.24 (m, 2H), 5.73-5.69 (m, 1H), 4.49-4.58 (m, 2H), 4.27-4.24 (m, 1H). MS [MH]⁺ 312.1.

### Example 1.47. Synthesis of N-(6-fluoro-2-isopentylbenzo[d]oxazol-5-yl)acrylamide (132)

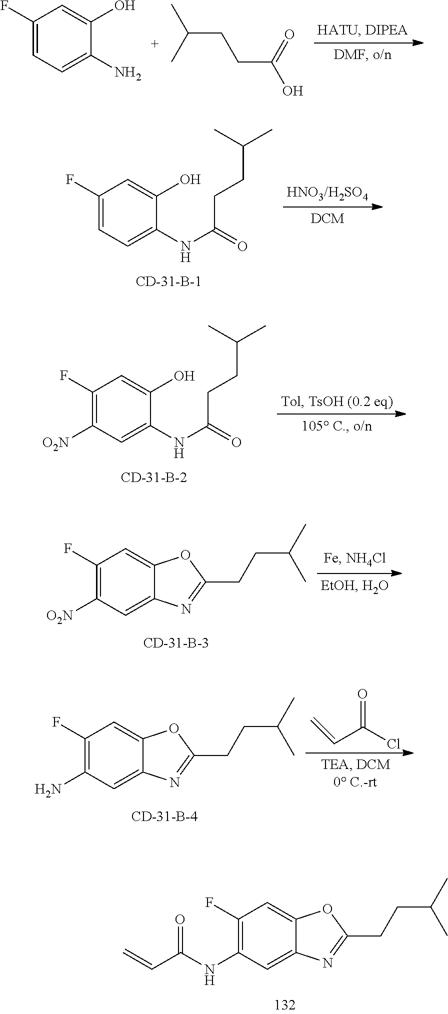

N-(4-fluoro-2-hydroxyphenyl)-4-methylpentanamide (CD-31-B-1). To a stirred solution of N-(4-fluoro-2-hydroxyphenyl)-4-methylpentanamide (1.500 g, 11.80 mmol), 4-methylpentanoic acid (1.400 g, 11.80 mmol), and DIPEA (4.600 g, 35.4 mmol) in DMF (15 mL) was added HATU (5.4 g, 14.2 mmol) at 0° C., and the resulting mixture was stirred at room temperature for 0.5 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 30-50% ethyl acetate/hexane gradient to afford N-(4-fluoro-2-hydroxyphenyl)-4-methylpentanamide (CD-31-B-1) (1.94 g, 3%) as a yellow solid. MS: [MH]⁺ 271.3.

N-(4-fluoro-2-hydroxy-5-nitrophenyl)-4-methylpentanamide (CD-31-B-2). A mixture of N-(4-fluoro-2-hydroxyphenyl)-4-methylpentanamide (CD-31-B-1) (1.94 g, 8.6 mmol) and nitric acid (250 mg, 2.58 mmol) in sulfuric acid (6.6 ml) was stirred at room temperature for 2 h. The mixture was quenched with ice water (20 mL) and adjusted to pH 6-7 with sodium hydroxide. Combined organic extracts were washed with brine (5 mL), dried over anhydrous Na₂SO₄, and concentrated in vacuo. The resulting crude was purified by silica gel column chromatography using a 20% ethyl acetate/hexane gradient to afford N-(4-fluoro-2-hydroxy-5-nitrophenyl)-4-methylpentanamide (CD-31-B-2) (0.410 g, 18%) as a black solid. MS: [MH]⁺ 271.1.

6-fluoro-2-isopentyl-5-nitrobenzo[d]oxazole (CD-31-B-3) To a mixture of N-(4-fluoro-2-hydroxy-5-nitrophenyl)-4-methylpentanamide (CD-31-B-2) (0.410 mg, 1.52 mmol) in toluene (3 ml) was added p-toluene sulfonic acid (0.056 g, 0.30 mmol) at room temperature, and the resulting mixture was stirred at 105° C. overnight. 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 6-fluoro-2-isopentyl-5-nitrobenzo[d]oxazole (CD-31-B-3) (0.220 g, 54%) as a yellow solid. MS: [MH]⁺ 252.9.

6-fluoro-2-isopentylbenzo[d]oxazol-5-amine (CD-31-B-4). To a solution of 6-fluoro-2-isopentyl-5-nitrobenzo[d]oxazole (CD-31-B-3) (0.100 g, 0.40 mmol) and ammonium chloride (0.212 g, 3.96 mmol) in ethanol (2 mL)-water (0.5 mL) was added iron powder (0.077 g, 1.39 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo to afford 6-fluoro-2-isopentylbenzo[d]oxazol-5-amine (CD-31-B-4) (0.060 g) as a yellow solid which was used in next step without further purification. MS: [MH]⁺ 223.1.

N-(6-fluoro-2-isopentylbenzo[d]oxazol-5-yl)acrylamide (132). To a stirred solution of 6-fluoro-2-isopentylbenzo[d]oxazol-5-amine (CD-31-B-4) (0.060 g, 0.27 mmol) and triethylamine (0.161 g, 1.08 mmol) in dichloromethane (2 mL) was added acryloyl chloride (0.024 g, 0.27 mmol) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 20% ethyl acetate/hexane gradient to afford N-(6-fluoro-2-isopentylbenzo[d]oxazol-5-yl)acrylamide (132) (0.075 g, 40%) as a white solid. ¹H NMR (400 MHz, CD₃OD): δ 8.24 (d, J=8.2 Hz, 1H), 7.54-7.51 (m, 1H), 6.61-6.54 (m, 1H), 6.45-6.40 (dd, J=6.4 Hz, 1H), 5.85-5.82 (m, 1H), 3.00-2.96 (m, 2H), 1.81-1.75 (m, 2H), 1.69-1.66 (m, 1H), 1.01 (d, J=1.0 Hz, 6H). MS: [MH]⁺ 277.2.

The following compounds were prepared in a manner analogous to the procedures described above for N-(6-fluoro-2-isopentylbenzo[d]oxazol-5-yl)acrylamide (132):

N-(6-fluoro-2-(4,4,4-trifluorobutyl)benzo[d]oxazol-5-yl)acrylamide (133) (0.030 g, 54% yield) as a brown solid. ¹H NMR (400 MHz, DMSO-d₆): 10.01 (s, 1H), 8.24-8.21 (m, 1H), 7.83-7.80 (m, 1H), 6.66-6.59 (m, 1H), 6.30 (d, J=6.4 Hz, 1H), 5.81-5.78 (m, 1H), 3.06 (t, J=3.2 Hz, 2H), 2.46-2.41 (m, 2H), 2.06-1.99 (m, 2H). MS: [MH]⁺ 317.1.

N-(2-(cyclopropylmethyl)-6-fluorobenzo[d]oxazol-5-yl)acrylamide (134) (0.410 g, 47%) as a white solid. ¹H NMR (400 MHz, DMSO-d₆): δ 10.00 (s, 1H), 8.21 (d, J=7.2 Hz, 1H), 7.80 (d, J=10.0 Hz, 1H), 6.66-6.59 (m, 1H), 6.32-6.27 (m, 1H), 5.61-5.78 (m, 1H), 2.86 (d, J=7.2 Hz, 2H), 1.22-1.14 (m, 1H), 0.59-0.54 (m, 2H), 0.32-0.28 (m, 2H). MS: [MH]⁺ 261.2.

N-(2-(4,4-difluorocyclohexyl)-6-fluorobenzo[d]oxazol-5-yl)acrylamide (135) (0.028 g, 53%) as a yellow solid. ¹H NMR (400 MHz, CDCl₃): δ 8.74-8.72 (m, 1H), 7.44 (s, 1H), 7.29 (d, J=10.0 Hz, 1H), 6.48-6.42 (m, 1H), 6.29-6.23 (m, 1H), 5.83 (d, J=10.4 Hz, 1H), 3.09-3.04 (m, 1H), 2.25-2.10 (m, 6H), 2.01-1.87 (m, 2H). MS: [MH]⁺ 271.1.

N-(2-((4,4-difluorocyclohexyl)methyl)-6-fluorobenzo[d]oxazol-5-yl)acrylamide (136) (0.032 g, 78%) as a yellow solid. ¹H NMR (400 MHz, CDCl₃) δ 8.71 (d, J=6.8 Hz, 1H), 7.44 (s, 1H), 7.28 (d, J=10.0 Hz, 1H), 6.48-6.44 (m, 1H), 6.29-6.24 (m, 1H), 5.83-5.80 (m, 1H), 2.86 (d, J=6.8 Hz, 1H), 2.12-2.01 (s, 3H), 1.87-1.84 (m, 2H), 1.79-1.67 (m, 2H), 1.50-1.40 (m, 2H). MS: [MH]⁺ 339.3.

N-(2-benzyl-6-fluorobenzo[d]oxazol-5-yl)acrylamide (137) (0.015 g, 82%) as a yellow solid. ¹H NMR (400 MHz, CDCl₃): 8.74-8.73 (m, 1H), 7.44 (d, J=7.4 Hz, 1H), 7.39-7.32 (m, 4H), 7.32-7.28 (m, 1H), 7.25-7.23 (m, 1H), 6.50-6.45 (m, 1H), 6.32-6.25 (m, 1H), 5.84-5.81 (m, 1H), 4.26 (s, 2H). MS: [MH]⁺ 297.2.

N-(2-(4,4-difluorocyclohexyl)-6-fluorobenzo[d]oxazol-5-yl)acrylamide (138) (0.028 g, 53%) as a yellow solid. ¹H NMR (400 MHz, CDCl₃): δ 8.74-8.72 (m, 1H), 7.44 (s, 1H), 7.32-7.28 (m, 1H), 6.48-6.44 (m, 1H), 6.29-6.25 (m, 1H), 5.84-5.80 (m, 1H), 3.09-3.04 (m, 1H), 2.25-2.10 (m, 6H), 2.01-1.87 (m, 2H). MS: [MH]⁺ 271.1.

N-(6-fluoro-2-(3-fluorophenethyl)benzo[d]oxazol-5-yl)acrylamide (139) (0.017, 35%) as a yellow solid. ¹H NMR (400 MHz, CDCl₃): δ 8.73-8.72 (m, 1H), 7.44 (s, 1H), 7.24-7.22 (m, 1H), 7.29-7.27 (m, 1H), 7.00-6.97 (m, 1H), 6.93-6.90 (m, 1H), 6.50-6.46 (m, 1H), 6.29-6.26 (m, 1H), 5.84-5.82 (m, 1H), 3.35-3.17 (m, 4H). MS: [MH]⁺ 329.1.

N-(6-fluoro-2-(3,3,4,4,4-pentafluorobutyl)benzo[d]oxazol-5-yl)acrylamide (140) (0.056 g, 84%) as a white solid. ¹H NMR (400 MHz, DMSO-d₆): δ 10.03 (s, 1H), 8.25 (t, J=6.6 Hz, 1H), 7.85 (d, J=10.0 Hz, 1H), 6.66-6.59 (m, 1H), 6.32-6.27 (m, 1H), 5.81-5.78 (m, 1H), 3.28 (t, J=7.8 Hz, 2H), 2.91-2.78 (m, 2H). MS: [MH]⁺ 353.1.

N-(4-fluoro-2-(4-methylpentyl)benzo[d]oxazol-5-yl)acrylamide (141) (0.023 g, 18%) as a white solid. ¹H NMR (400 MHz, CDCl₃): δ 8.31 (t, J=7.6 Hz, 1H), 7.44 (s, 1H), 7.29 (d, J=8.8 Hz, 1H), 6.48 (d, J=16.8 Hz, 1H), 6.32-6.29 (m, 1H), 5.83 (d, J=10.4 Hz, 1H), 2.92 (t, J=7.6 Hz, 2H), 1.93-1.85 (m, 2H), 1.66-1.56 (m, 1H), 1.33-1.28 (m, 2H), 0.91 (d, J=6.8 Hz, 6H). [MH]⁺ 291.1.

N-(6-fluoro-2-(4-fluorobenzyl)benzo[d]oxazol-5-yl)acrylamide (142) (0.008 g, 66%). ¹H NMR (400 MHz, DMSO-d₆): δ 10.02 (s, 1H), 8.22 (d, J=6.8 Hz, 1H), 6.29 (t, J=17.2 Hz, 1H), 6.67-6.61 (m, 1H), 7.21-7.17 (m, 2H), 7.44-7.41 (m, 2H), 7.82-7.79 (m, 1H), 5.79 (d, J=10.2 Hz, 1H), 4.35 (s, 2H). [MH]⁺ 315.2.

N-(6-fluoro-2-neopentylbenzo[d]oxazol-5-yl)acrylamide (143) (0.039 g, 20%) as a white solid. ¹HNMR (400 MHz, DMSO-d₆): δ 9.99 (s, 1H), 8.20 (d, J=7.2 Hz, 1H), 7.80 (d, J=10.2 Hz, 1H), 6.61-6.58 (m, 1H), 6.29-6.27 (m, 1H), 5.77-5.74 (m, 1H), 2.81 (s, 2H), 1.02 (s, 9H). [MH]⁺ 277.1.

N-(6-fluoro-2-(3,3,3-trifluoropropyl)benzo[d]oxazol-5-yl)acrylamide (144) (0.048 g, 98%) as a white solid. ¹H NMR (400 MHz, DMSO-d₆) δ 8.24 (t, J=6.6 Hz, 1H), 7.83 (d, J=10.0 Hz, 1H), 6.66-6.59 (m, 1H), 6.32-6.27 (m, 1H), 5.81-5.78 (m, 1H), 3.23 (t, J=7.6 Hz, 2H), 2.92-2.84 (m, 2H). [MH]⁺ 303.1.

N-(6-fluoro-2-((2-methylcyclopropyl)methyl)benzo[d]oxazol-5-yl)acrylamide (145) (0.020 g, 43%) as an off-white solid. ¹H NMR (400 MHz, CDCl₃): δ 8.71 (d, J=6.4 Hz, 1H), 7.45 (s, 1H), 7.29 (d, J=10.0 Hz, 1H), 6.48-6.46 (m, 1H), 6.30-6.27 (m, 1H), 5.83-5.81 (m, 1H), 2.89-2.86 (m, 1H), 2.76-2.73 (m, 1H), 1.07 (d, J=6.2 Hz, 3H), 0.97-0.86 (m, 1H), 0.76-0.68 (m, 1H), 0.50-0.46 (m, 1H), 0.39-0.35 (m, 1H). [MH]⁺ 275.2.

### Example 1.48. Synthesis of N-(2-(3-(trifluoromethyl)phenyl)benzo[d][1,3]dioxol-5-yl)acrylamide (146)

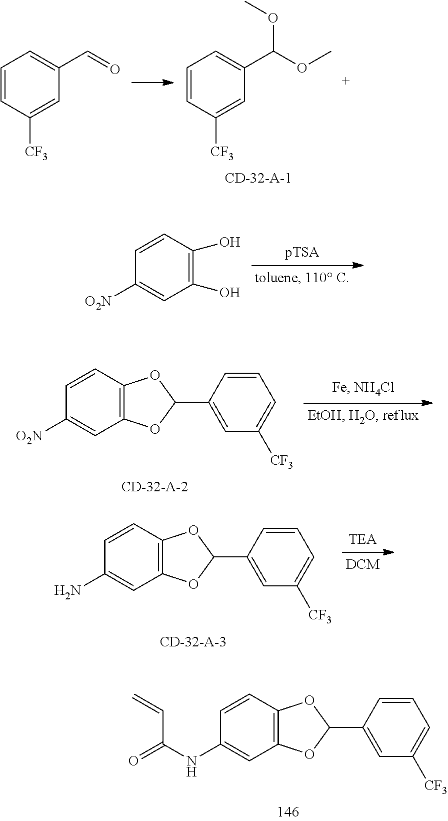 [SMILES: O=Cc1cccc(C(F)(F)F)c1 ; COC(OC)c1cccc(C(F)(F)F)c1 ; O=[N+]([O-])c1ccc(O)c(O)c1 ; O=[N+]([O-])c1ccc2c(c1)OC(c1cccc(C(F)(F)F)c1)O2 ; Nc1ccc2c(c1)OC(c1cccc(C(F)(F)F)c1)O2 ; C=CC(=O)Nc1ccc2c(c1)OC(c1cccc(C(F)(F)F)c1)O2]

1-(dimethoxymethyl)-3-(trifluoromethyl)benzene (CD-32-A-1). To a solution of 3-(trifluoromethyl)benzaldehyde (10.000 g, 57.40 mmol) and 1,1-diethoxy-2-methoxyethane (9.360 g, 63.20 mmol) in methanol (150 mL) was added p-toluenesulfonic acid (2.060 g, 12.00 mmol), and the reaction mixture was stirred at room temperature under nitrogen for 12 h. The resulting mixture concentrated in vacuo. The resulting crude was purified by silica gel column chromatography using a 2% ethyl acetate/hexane gradient to afford 1-(dimethoxymethyl)-3-(trifluoromethyl)benzene (CD-32-A-1) (8.800 g, 69%) as a colorless oil. ¹H NMR (400 MHz, CDCl₃): δ 7.74 (s, 1H), 7.64 (d, J=8.0 Hz, 1H), 7.59 (d, J=8.0 Hz, 1H), 7.49 (t, J=7.6 Hz, 1H), 5.44 (s, 1H), 3.33 (s, 1H).

5-nitro-2-(3-(trifluoromethyl)phenyl)benzo[d][1,3]dioxole (CD-32-A-2). A mixture of 1-(dimethoxymethyl)-3-(trifluoromethyl)benzene (CD-32-A-1) (0.160 g, 0.72 mmol), 4-nitrobenzene-1,2-diol (0.086 g, 0.55 mmol), and p-toluenesulfonic acid (0.011 g, 0.05 mmol) in toluene (2 mL) was stirred at 110° C. under nitrogen for 2 hours. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 2% ethyl acetate/hexane gradient to afford 5-nitro-2-(3-(trifluoromethyl)phenyl)benzo[d][1,3]dioxole (CD-32-A-2) (0.066 g, yield 38%) as a yellow oil.

2-(3-(trifluoromethyl)phenyl)benzo[d][1,3]dioxol-5-amine (CD-32-A-3). To a solution of 5-nitro-2-(3-(trifluoromethyl)phenyl)benzo[d] [1,3]dioxole (CD-32-A-2) (0.060 g, 0.19 mmol) and ammonium chloride (0.051 g, 0.96 mmol) in ethanol (4 mL)-water (1 mL) was added iron powder (0.054 g, 0.96 mmol) at room temperature under nitrogen, and the resulting mixture was stirred at 80° C. for 2 h. After cooling to room temperature, iron was removed through filtration and washed with methanol (10 mL×2). Combined organic filtrates were concentrated in vacuo. The resulting crude was purified by silica gel column chromatography using a 30% DCM/hexane gradient to afford 2-(3-(trifluoromethyl)phenyl)benzo[d] [1,3]dioxol-5-amine (CD-32-A-3) (0.034 g, 62%) as a yellow oil. MS: [MH]⁺ 282.0.

N-(2-(3-(trifluoromethyl)phenyl)benzo[d][1,3]dioxol-5-yl)acrylamide (146). To a solution of 2-(3-(trifluoromethyl)phenyl)benzo[d][1,3]dioxol-5-amine (CD-32-A-3) (0.034 g, 0.12 mmol) and triethylamine (0.019 g, 0.18 mmol) in dichloromethane (2 mL) was added acryloyl chloride (0.012 g, 0.13 mmol) in dichloromethane (1 mL) at 0° C., and the resulting mixture was stirred at room temperature for 1.5 h. The reaction mixture was poured into water (10 mL) and extracted with DCM (10 mL×2). Combined organic extracts were washed with brine (10 mL), dried over anhydrous Na₂SO₄, and concentrated in vacuo. The resulting crude was purified by silica gel column chromatography using a 30% DCM/hexane gradient to afford N-(2-(3-(trifluoromethyl)phenyl)benzo[d][1,3]dioxol-5-yl)acrylamide (146) (0.030 g, 75%) as a white solid. ¹H NMR (400 MHz, CDCl₃): δ 7.84 (s, 1H), 7.77 (d, J=8.0 Hz, 1H), 7.71 (d, J=7.6 Hz, 1H), 7.58 (t, J=7.8 Hz, 1H), 7.39 (s, 1H), 7.17 (s, 1H), 7.02 (s, 1H), 6.90-6.88 (m, 1H), 6.81 (d, J=8.0 Hz, 1H), 6.42-6.38 (m, 1H), 6.25-6.21 (m, 1H), 5.77-5.72 (m, 1H). MS: [MH]⁺ 336.0.

### Example 1.49. Synthesis of N-(2-methyl-2-(3-(trifluoromethyl)phenyl)benzo[d][1,3]dioxol-5-yl)acrylamide (147)

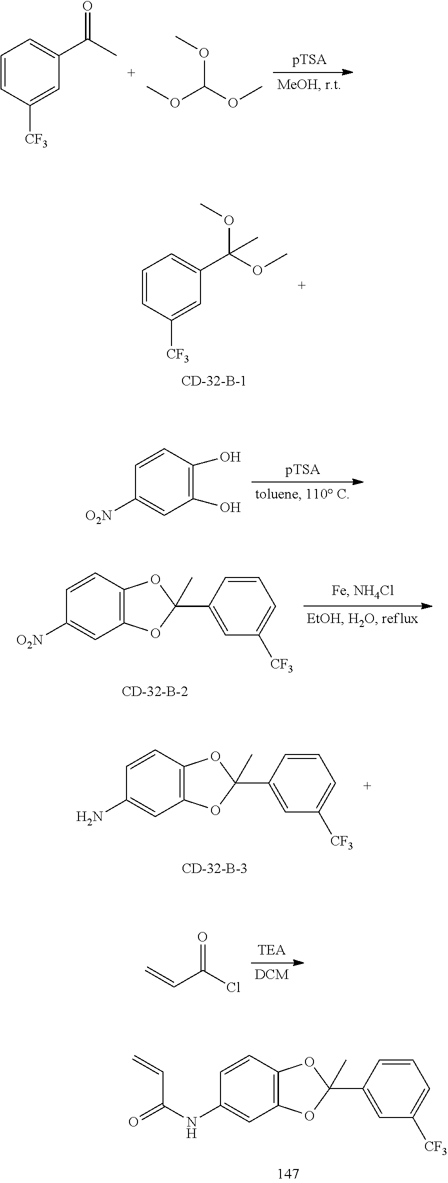

1-(1,1-dimethoxyethyl)-3-(trifluoromethyl)benzene (CD-32-B-1). A mixture of 1-(3-(trifluoromethyl)phenyl)ethan-1-one (10.000 g, 53.00 mmol), trimethoxymethane (8.600 g, 58.00 mmol), and p-toluenesulfonic acid (1.000 g, 5.00 mmol) in methanol (100 mL) was stirred at room temperature under nitrogen for 12 h. The reaction mixture concentrated in vacuo. The resulting crude was purified by silica gel column chromatography using a 2% ethyl acetate/hexane gradient to afford 1-(1,1-dimethoxyethyl)-3-(trifluoromethyl)benzene (CD-32-B-1) (10.10 g, 80%) as a colorless oil. ¹H NMR (400 MHz, CDCl₃) δ 7.78 (s, 1H), 7.67 (d, J=7.6 Hz, 1H), 7.55 (d, J=7.6 Hz, 1H), 7.47 (t, J=7.6 Hz, 1H), 3.19 (s, 6H), 1.54 (s, 3H).

2-methyl-5-nitro-2-(3-(trifluoromethyl)phenyl)benzo[d][1,3]dioxole (CD-32-B-2). A mixture of 1-(1,1-dimethoxyethyl)-3-(trifluoromethyl)benzene (CD-32-B-1) (10.000 g, 17.00 mmol), 4-nitrobenzene-1,2-diol (2.020 g, 13.00 mmol), and p-toluenesulfonic acid (0.378 g, 2.20 mmol) in toluene (100 mL) was stirred at 110° C. under nitrogen for 2 hours. The reaction mixture was poured into water (100 mL) and extracted with ethyl acetate (100 mL×2). Combined organic extracts were washed with brine (100 mL), dried over anhydrous Na₂SO₄, and concentrated in vacuo. The resulting crude was purified by silica gel column chromatography using a 2% ethyl acetate/hexane gradient to afford 2-methyl-5-nitro-2-(3-(trifluoromethyl)phenyl)benzo[d][1,3]dioxole (CD-32-B-2) (2.210 g, 48%) as a colorless oil.

2-methyl-2-(3-(trifluoromethyl)phenyl)benzo[d][1,3]dioxol-5-amine (CD-32-B-3). To a solution of 2-methyl-5-nitro-2-(3-(trifluoromethyl)phenyl)benzo[d][1,3]dioxole (CD-32-B-2) (2.210 g, 6.80 mmol) and ammonium chloride (1.320 g, 23.80 mmol) in ethanol (40 mL)-water (10 mL) was added iron powder (1.400 g, 25.00 mmol) at room temperature under nitrogen, and the resulting mixture was stirred at 80° C. for 2 h. After cooling to room temperature, iron was removed through filtration and washed with methanol (50 mL×2). Combined organic filtrates were concentrated in vacuo. The resulting crude was purified by silica gel column chromatography using a DCM gradient to afford 2-methyl-2-(3-(trifluoromethyl)phenyl)benzo[d][1,3]dioxol-5-amine (CD-32-B-3) (1.610 g, 80%) as a colorless oil. MS: [MH]⁺ 295.9.

N-(2-methyl-2-(3-(trifluoromethyl)phenyl)benzo[d][1,3]dioxol-5-yl)acrylamide (147). To a solution of 2-methyl-2-(3-(trifluoromethyl)phenyl)benzo[d][1,3]dioxol-5-amine (CD-32-B-3) (1.610 g, 5.44 mmol) and triethylamine (0.555 g, 5.50 mmol) in dichloromethane (20 mL) was added acryloyl chloride (0.495 g, 5.50 mmol) at 0° C., and the resulting mixture was stirred at room temperature for 1.5 h. The reaction mixture was poured into water (30 mL) and extracted with DCM (30 mL×2). Combined organic extracts were washed with brine (30 mL), dried over anhydrous Na₂SO₄, and concentrated in vacuo. The resulting crude was purified by silica gel column chromatography using a DCM gradient to afford N-(2-methyl-2-(3-(trifluoromethyl)phenyl)benzo[d][1,3]dioxol-5-yl)acrylamide (147) (1.447 g, 76%) as a white solid. ¹H NMR (400 MHz, CDCl₃): δ 7.84 (s, 1H), 7.78 (d, J=8.0 Hz, 1H), 7.61 (d, J=7.6 Hz, 1H), 7.51 (t, J=7.8 Hz, 1H), 7.34 (s, 1H), 7.26 (s, 1H), 6.83-6.81 (m, 1H), 6.75 (d, J=8.4 Hz, 1H), 6.40-6.35 (m, 1H), 6.19-6.13 (m, 1H), 5.74-5.70 (m, 1H), 1.99 (s, 3H). MS: [MH]⁺ 350.0.

The achiral 147 (2.500 g, 7.14 mmol) was separated by SFC (Column CHIRAL Cellulose-SB; Column Size 3 cm×25 cm, 5 umMobile Phase A CO2, Mobile Phase B IPA; Flow Rate 100 g/min; Wave Length UV 220 nm; Temperature 25° C.) to afford the first eluting isomer (1.043 g, 42%) (Peak-1:ee=99.72%, chemical purity=97.91%) as a white solid and second eluting isomer (1.054 g, 42%) (Peak-2:ee=98.54%, chemical purity=98.79%) as a white solid.

147 first eluting isomer—¹H NMR (400 MHz, CDCl₃): δ 7.84 (s, 1H), 7.78 (d, J=8.0 Hz, 1H), 7.61 (d, J=7.6 Hz, 1H), 7.51 (t, J=7.8 Hz, 1H), 7.34 (s, 1H), 7.26 (s, 1H), 6.82-6.81 (m, 1H), 6.75 (d, J=8.4 Hz, 1H), 6.40-6.35 (m, 1H), 6.19-6.13 (m, 1H), 5.74-5.70 (m, 1H), 1.99 (s, 3H). MS: [MH]⁺ 350.0.

147 second eluting isomer—¹H NMR (400 MHz, CDCl₃): δ 7.84 (s, 1H), 7.78 (d, J=8.0 Hz, 1H), 7.61 (d, J=7.6 Hz, 1H), 7.51 (t, J=7.8 Hz, 1H), 7.34 (s, 1H), 7.26 (s, 1H), 6.83-6.81 (m, 1H), 6.75 (d, J=8.4 Hz, 1H), 6.40-6.34 (m, 1H), 6.19-6.13 (m, 1H), 5.74-5.70 (m, 1H), 1.99 (s, 3H). MS: [MH]⁺ 350.0.

### Example 1.50. Synthesis of 2-fluoro-N-(2-(3-(trifluoromethyl)phenyl)benzo[d][1,3]dioxol-5-yl)acrylamide (148)

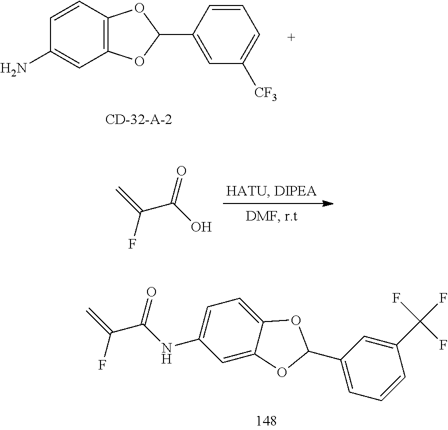

To a solution of 2-(3-(trifluoromethyl)phenyl)benzo[d][1,3]dioxol-5-amine (CD-32-A-2) (0.100 g, 0.35 mmol), 2-fluoroacrylic acid (0.032 g, 0.35 mmol), and N-ethyl-N-isopropylpropan-2-amine (0.090 g, 0.70 mmol) in N,N-dimethylformamide (3 mL) was added HATU (0.159 g, 0.42 mmol) at room temperature under nitrogen, and the resulting mixture was stirred at room temperature for 4 h. The reaction mixture was poured into water (10 mL) and extracted with ethyl acetate (5 mL×2). Combined organic extracts were washed with brine (5 mL), dried over anhydrous Na₂SO₄, and concentrated in vacuo. The resulting crude was purified by silica gel column chromatography using a 20% ethyl acetate/hexane gradient to afford 2-fluoro-N-(2-(3-(trifluoromethyl)phenyl)benzo[d][1,3]dioxol-5-yl)acrylamide (148) (0.124 g, 99%) as a yellow solid. ¹H NMR (400 MHz, DMSO-d₆): δ 10.26 (s, 1H), 7.90 (d, J=7.6 Hz, 3H), 7.75 (t, J=7.2 Hz, 1H), 7.42 (d, J=1.6 Hz, 1H), 7.31 (s, 1H), 7.24-7.22 (m, 1H), 6.98 (d, J=8.4 Hz, 1H), 5.75-5.62 (m, 1H), 5.43-5.38 (m, 1H). MS: [MH]⁺ 354.0.

The following compound was prepared in a manner analogous to the procedures described above for 2-fluoro-N-(2-(3-(trifluoromethyl)phenyl)benzo[d][1,3]dioxol-5-yl)acrylamide (148):

N-(4-((4,4-difluorocyclohexyl)methoxy)-3-fluorophenyl)-2-fluoroacrylamide (149) (0.030 g, 40% yield) as a white solid. ¹H NMR (400 MHz, DMSO-d₆) δ 10.34 (s, 1H), 7.66-7.64 (m, 1H), 7.45 (d, J=8.8 Hz, 1H), 7.18-7.14 (m, 1H), 5.76-5.63 (m, 1H), 5.45-5.40 (m, 1H), 3.92-3.91 (m, 2H), 2.05-2.02 (m, 2H), 1.89-1.86 (m, 5H), 1.23-1.33 (m, 2H). MS: [MH]⁺ 331.9.

### Example 1.51. Synthesis of N-(8-(4-(trifluoromethyl)phenyl)quinolin-5-yl)acrylamide (150)

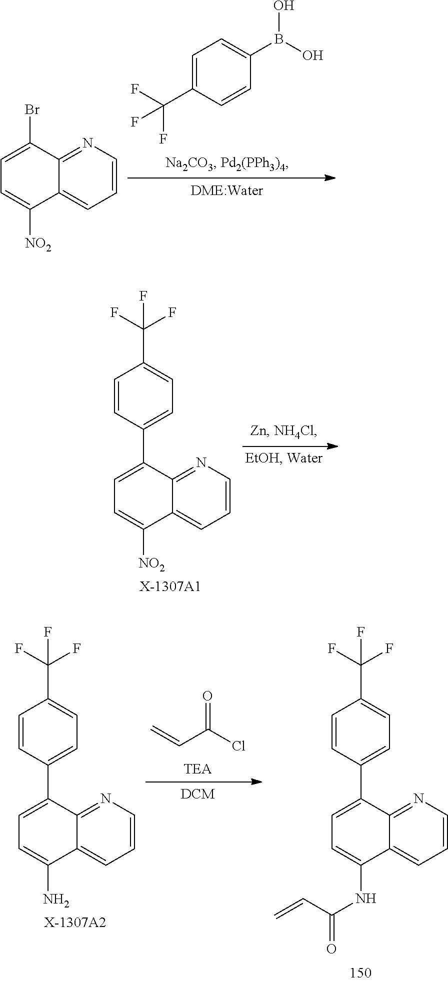

5-Nitro-8-(4-(trifluoromethyl)phenyl)quinoline (X-1307A1). To a stirred solution of 8-bromo-5-nitroquinoline (0.350 g, 1.38 mmol) in a mixture of DME-water (0.25:1; 2.5 mL) were added (4-(trifluoromethyl)phenyl)boronic acid (0.394 g, 2.07 mmol) and Na₂CO₃ (0.439 g, 4.14 mmol) at room temperature under nitrogen. The reaction mixture was degassed (purging with nitrogen) for 20 min followed by the addition of Pd₂(PPh₃)₄ (0.079 g, 0.06 mmol), and the resulting mixture was heated at 100° C. for 1 h. The reaction mixture was cooled to room temperature, was diluted with water (150 mL), and was extracted with ethyl acetate (150 mL×3). Combined organic extracts were washed with brine (100 mL), dried over anhydrous Na₂SO₄, and concentrated in vacuo. The crude product was triturated with diethylether and pentane, to afford 5-nitro-8-(4-(trifluoromethyl)phenyl)quinoline (X-1307A1) (0.410 g, quant.; crude) as an off-white solid. MS: [MH]⁺ 319.0.

8-(4-(Trifluoromethyl)phenyl)quinolin-5-amine (X-1307A2). To a stirred solution of 5-nitro-8-(4-(trifluoromethyl)phenyl)quinoline (X-1307A1) (0.410 g, 1.28 mmol) in a mixture of EtOH—H₂O (8:2; 1 mL) were added Zn dust (0.420 g, 6.44 mmol) and ammonium chloride (0.344 g, 6.44 mmol) at room temperature, and the reaction mixture was stirred at room temperature for 1 h. The reaction mixture was filtered through a celite bed and residue was washed with ethyl acetate (200 mL). Combined filtrates were washed with water (150 mL), dried over anhydrous Na₂SO₄, and concentrated under reduce pressure to afford 8-(4-(trifluoromethyl)phenyl)quinolin-5-amine (X-1307A2) (0.290 g, 83%) as an orange solid, which was used in next step without further purification. MS: [MH]⁺ 289.0.

N-(8-(4-(trifluoromethyl)phenyl)quinolin-5-yl)acrylamide (X-1307). To a stirred solution of 8-(4-(trifluoromethyl)phenyl)quinolin-5-amine (X-1307A2) (0.250 g, 0.86 mmol) in DCM (3.0 mL) were added triethylamine (0.300 g, 2.17 mmol) and acryloyl chloride (0.078 g, 0.86 mmol) at 0° C. under nitrogen, and the resulting mixture was stirred at room temperature for 15 min. The reaction mixture was diluted with water (100 mL) and was extracted with DCM (150 mL×3). Combined organic extracts were dried over anhydrous Na₂SO₄ and concentrated under reduce pressure to provide a crude mass, which was purified by reverse phase (C-18) silica gel column chromatography using acetonitrile-water=0:1→7:3 as gradient, to afford N-(8-(4-(trifluoromethyl)phenyl)quinolin-5-yl)acrylamide (150) (0.055 g, 18%) as a white solid. ¹H NMR (400 MHz, DMSO-d6) δ 10.39 (s, 1H), 8.94-8.93 (d, J=2.8 Hz, 1H), 8.60-8.58 (d, J=7.6 Hz, 1H), 8.04-8.02 (d, J=8.0 Hz, 1H), 7.88-7.82 (m, 5H), 7.66-7.63 (m, 1H), 6.75-6.69 (m, 1H), 6.37-6.35 (d, J=8.8 Hz, 1H), 5.88-5.85 (d, J=11.2 Hz, 1H). MS: [MH]⁺ 343.0.

The following compound was prepared in a manner analogous to the procedures described above for N-(8-(4-(trifluoromethyl)phenyl)quinolin-5-yl)acrylamide (150):

N-(8-(4-(tert-butyl)phenyl)quinolin-5-yl)acrylamide (151) (0.055 g, 14%) as an off-white solid. ¹H NMR (400 MHz, DMSO-d6) δ 10.32 (s, 1H), 8.92-8.90 (dd, J=4.0 Hz, 1.6 Hz, 1H), 8.54-8.51 (dd, J=8.8 Hz, 1.6 Hz, 1H), 7.94-7.92 (d, J=8.0 Hz, 1H), 7.76-7.74 (d, J=8.0 Hz, 1H), 7.62-7.57 (m, 3H), 7.49-7.47 (d, J=8.0 Hz, 2H), 6.70-6.67 (m, 1H), 6.36-6.31 (dd, J=16.8 Hz, 1.6 Hz, 1H), 5.86-5.83 (dd, J=10.4 Hz, J=1.6 Hz, 1H), 1.35 (s, 9H). MS: [MH]⁺ 331.1.

### Example 2. TEAD Compound Displacement and Proliferation Assays

Compound Displacement Assay.

A TEAD1 lipid pocket displacement assay was carried out according to the following protocol. Purified His-tagged TEAD1 protein (YAP Binding Domain) was pre-mixed with a Cy5-probe (Cy5-conjugated to a small molecule that binds in the TEAD1 lipid pocket) and Terbium-labeled anti-His antibody (Cisbio Cat 61HI2TLB). The binding of the Cy5-probe to anti-His-Tb/His-tag TEAD1 complex yielded a TR-FRET signal. Addition of compounds that are TEAD1 lipid pocket binders resulted in the displacement of the Cy5-probe from TEAD1 and a decrease in the TR-FRET signal. After 60 minutes incubation at room temperature of compounds with the His-TEAD1/anti-His-Tb/Cy5-probe complex, the plate was read on a plate reader (BMG ClarioStar Cat 430-1300) using TR-FRET mode with wavelengths of 665 nm/620 nm. The potency of compounds as TEAD1 lipid pocket binders was determined by IC50 value generated using a non-linear 4 parameter curve fit.

72H TEAD Proliferation Assay.

The effect of TEAD inhibition on cell proliferation was assayed using Cell Titer Glo (CTG) 2.0 to measure response in mesothelioma cell lines NCI-H226 (ATCC, #CRL-5826) and NCI-H28 (ATCC, #CRL-5820).

Description

The 72H TEAD Proliferation assay utilizes Cell Titer-Glo 2.0 (Promega, #G9243) to measure the proliferation of cells in the presence or absence of compound. Cell Titer-Glo 2.0 determines the amount of viable cells by quantifying ATP (an indication of metabolically active cells). It utilizes the conversion of Luciferin to Oxyluciferin and a luminescent signal with the use of ATP to report the quantity of viable cells in culture. Within cells that are continually growing, ATP is being synthesized to meet their metabolic demands, meanwhile the opposite is true for cells that are dying or slowing down their proliferation and either no longer using ATP or are using less, respectively. The NF2-deficient NCI-H226 has been genetically validated as a cell line that is sensitive to TEAD inhibition. The NF2-wild type NCI-H28 has been genetically validated as a cell line that is not sensitive to TEAD inhibition and grows independently of TEAD activity.

Application

Monitor for any effects on proliferation with compound treatment.

Compounds were screened against the responsive NCI-H226 cell line to assess the compounds' ability to inhibit TEAD and cell growth. Compounds were also screened against the non-responsive NCI-H28 cell line to ascertain whether the inhibition of cell growth was due to inhibition of the target TEAD or whether the inhibition was due to off-target cytotoxicity.

General Culture Conditions

Thaw Medium 1/Growth Medium 1: RPMI 1640 with GlutaMAX supplement medium (Gibco, #61870036) with 10% FBS (Gibco, #A3160402))

Assay Medium 1: RPMI 1640 medium with L-Glutamine, no phenol red (Gibco, #11835030) with 10% FBS (Gibco, #A3160402)

Both NCI-H226 and NCI-H28 cells were grown at 37° C. with 5% CO₂ using Growth Medium 1.

To recover the cells, frozen stock was thawed quickly in a 37° C. water-bath after removal from liquid nitrogen, transferred to a tube containing 1 ml of pre-warmed Thaw Medium 1, spun down, resuspended with 1 ml of pre-warmed Growth Medium 1 and added into a T75 with 9 ml of Growth Medium 1. The cell culture was grown in an incubator at 37° C. with 5% CO₂. At first passage, cells were transferred into a T150 with 15 mL Growth Medium 1 to allow the cells to continue growing. Cells were split before they reached complete confluency and were not used past passage number 20.

The cells were passaged by first rinsing them with phosphate buffered saline (PBS), and then detaching them from the flask with TrypLE Express (1X) (Gibco, #12604013). Growth Medium 1 was added and the cell suspension was transferred to a tube. The cells were counted and the volume was reduced to get 1M cells was added to another tube. The cells were spun down and resuspended in 2 mL of fresh Growth Medium 1. 1 ml of the cell suspension was added into a new T150 with 14 mL of Growth Medium 1. Subcultivation ratio: 500,000 cells in a T150 weekly.

The cells were frozen by rinsing them with phosphate buffered saline (PBS), and detaching them from the flask with TrypLE Express (1X) (Gibco, #12604013). Growth Medium 1 was added and the cell suspension was transferred to a tube. The cells were spun down and resuspended in freezing medium (95% FBS+5% DMSO). The cells were then added to cryovials and stored at −80° C. overnight then transferred to liquid nitrogen the next day.

Functional Validation and Assay Performance

The following assays were designed for 384-well format. Performing the assay in different tissue culture formats will need the cell number and reagent volume to be scaled up appropriately.

Materials

### Thaw Medium 1/Growth Medium 1 (Gibco, #61870036)+10% FBS (Gibco #A3160402)

Assay Medium 1 (Gibco, #11835030) with 10% FBS (Gibco #A3160402)
Phosphate Buffered Saline (Gibco, #10010023)
TrypLE Express (Gibco, #12604013)
Trypan Blue 0.4% (Invitrogen, #T10282)
Countess II FL Automated Cell Counter (ThermoFisher Scientific, #AMQAF1000)
Multidrop Combi Reagent Dispenser (ThermoFisher Scientific, #5840300)
384-well Low Flange Black Flat Bottom Polystyrene TC-treated Microplates (Corning 3571)
Echo (Beckman)
CTG 2.0 (Promega, #G9243)
Bravo Liquid Handler (Agilent)
EnVision Multilabel Plate Reader (PerkinElmer)

Mycoplasma Testing of NCI-H226 and NCI-H28 cell lines

The 2 cell lines were tested for Mycoplasma by IDEXX BioAnalytics using PCR-based Mycoplasma detection and confirmed to be negative.

Anti-proliferative effect of compounds that inhibits TEAD activity measured by CTG

1) Assay ready plates (ARPs) were prepared by Echo acoustic liquid handler. For each compound, duplicate of 10-point half-log dilution series were dispensed in a 384-well microplate (Corning 3571).

2) Each well had 50 nl of compound and had a final DMSO of 0.1% after cell plating.

3) Before use, ARPs were allowed to warm up to room temperature for 30 min.

4) ARPs were spun for 5 minutes at 1500 RPM before removing the plate seals.

5) NCI-H226 or NCI-H28 cells were harvested from culture in Assay Medium 1 and the multidrop Combi reagent dispenser was used to seed cells at 500 cells per 50 ul in each well of the ARPs and one Corning 3571 plate without compounds for Time 0 (TO) readout.

6) The TO plate was incubated for 2 hours at 37° C. with 5% CO₂ to allow cells to settle, then the CTG Assay was performed.

7) All ARPs were incubated for 72 hours at 37° C. with 5% CO₂, then the CTG Assay was performed for Time 72H (T72) readout.

8) CTG Assay: Bravo liquid handler (Agilent) was used to add 25 ul of CTG 2.0 to all columns of the plate except for Column 24, which was used to subtract out the background. After CTG addition, plates were placed on shaker at 800 RPM for 15 minutes at room temperature and kept in the dark. Luminescence was measured using EnVision multilabel plate reader with ultra-sensitive detection module.

9) Data Analysis: First background luminescence (no CTG wells) was subtracted from luminescence reading of all wells, then TO luminescence was subtracted from T72 luminescence. To compare anti-proliferative effect of compounds, GI₅₀ was obtained by fitting dose response curves with nonlinear regression curve fit.

Results are presented in Table 1. Compounds having an IC₅O less than or equal to 250 nM are represented as “A”; compounds having an IC₅₀ greater than 250 nM but less than or equal to 500 nM are represented as “B”; compounds having an IC₅₀ greater than 500 nM but less than or equal to 2 μM are represented as “C”; and compounds having an IC₅₀ greater than 2 μM are represented as “D”.

Compounds having a GI₅₀ less than or equal to 250 nM are represented as “A”; compounds having a GI₅₀ greater than 250 nM but less than or equal to 1 μM are represented as “B”; compounds having a GI₅₀ greater than 1 μM but less than or equal to 3 μM are represented as “C”; and compounds having a GI₅₀ greater than 3 μM are represented as “D”.

TABLE 1

| Compound | CDA IC₅₀ | GI₅₀ |
|---|---|---|
| 1 | A | C |
| 2 | B | |
| 3 | C | D |
| 4 | B | |
| 5 | D | |
| 6 | B | |
| 7 | A | |
| 8 | B | |
| 9 | A | D |
| 10 | D | |
| 11 | D | |
| 12 | D | |
| 13 | D | |
| 14 | D | |
| 15 | B | |
| 16 | D | |
| 17 | A | |
| 18 | D | |
| 19 | D | D |
| 20 | A | B |
| 21 | A | A |
| 22 | C | B |
| 23 | A | C |
| 24 | A | A |
| 25 | B | |
| 26 | A | B |
| 27 | C | C |
| 28 | B | B |
| 29 | C | |
| 30 | B | |
| 31 | A | A |
| 32 | A | |
| 33 | C | |
| 34 | A | |
| 35 | B | |
| 36 | B | |
| 37 | C | |
| 38 | C | C |
| 39 | B | A |
| 40 | C | C |
| 41 | D | D |
| 42 | D | B |
| 43 | A | C |
| 44 | D | |
| 45 | | D |
| 46 | A | |
| 47 | C | |
| 48 | A | |
| 49 | A | A |
| 50 | A | A |
| 51 | B | D |
| 52 | B | |
| 53 | C | C |
| 54 | D | C |
| 55 | A | B |
| 56 | D | |
| 57 | D | |
| 58 | A | B |
| 59 | A | D |
| 60 | C | D |
| 61 | C | |
| 62 | C | |
| 63 | C | C |
| 64 | D | |
| 65 | D | |
| 66 | D | |
| 67 | D | |
| 68 | C | |
| 69 | D | |
| 70 | D | |
| 71 | D | D |
| 72 | C | D |
| 73 | C | B |
| 74 | D | D |
| 75 | D | |
| 76 | D | D |
| 77 | D | |
| 78 | C | D |
| 79 | C | |
| 80 | A | B |
| 81 | D | |
| 82 | | |
| 83 | A | B |
| 84 | C | C |
| 85 | D | |
| 86 | D | |
| 87 | C | |
| 88 | B | |
| 89 | A | A |
| 90 | A | |
| 91 | A | B |
| 92 | C | |
| 93 | A | |
| 94 | C | |
| 95 | B | |
| 96 | A | B |
| 97 | B | |
| 98 | D | C |
| 99 | A | D |
| 100 | B | D |
| 101 | C | C |
| 102 | A | |
| 103 | D | C |
| 104 | B | B |
| 105 | A | B |
| 106 | A | D |
| 107 | A | B |
| 108 | D | B |
| 109 | B | B |
| 110 | B | C |
| 111 | A | B |
| 112 | B | D |
| 113 | A | C |
| 114 | A | D |
| 115 | B | A |
| 116 | B | B |
| 117 | B | B |
| 118 | D | |
| 119 | C | |
| 120 | B | D |
| 121 | C | D |
| 122 | A | D |
| 123 | C | |
| 124 | B | D |
| 125 | D | D |
| 126 | A | C |
| 127 | A | |
| 128 | A | |
| 129 | B | D |
| 130 | C | |
| 131 | C | |
| 132 | B | A |
| 133 | | A |
| 134 | D | C |
| 135 | C | A |
| 136 | B | B |
| 137 | B | D |
| 138 | B | C |
| 139 | B | |
| 140 | A | A |
| 141 | B | B |
| 142 | C | C |
| 143 | C | B |
| 144 | D | B |
| 145 | B | A |
| 146 | A | A |
| 147* | A | B |
| 148 | C | B |
| 149 | B | A |
| 150 | A | A |
| 151 | B | A |

*racemate

### Example 3. TEAD Mass Modification Assays

Compound stock (10 mM) was diluted to 2.5 mM with DMSO and His-Tev-Avi-TEAD1 was diluted to 5 μM with Reaction Buffer (50 mM Tris pH 8.0, 150 mM NaCl, and 1 mM TCEP). Compound (1 μL) is incubated with 49 μL protein at room temperature for 1 hour (final compound concentration was 50 μM). The sample (20 μL) was shipped on dry ice to MSBioworks for iMS analysis.

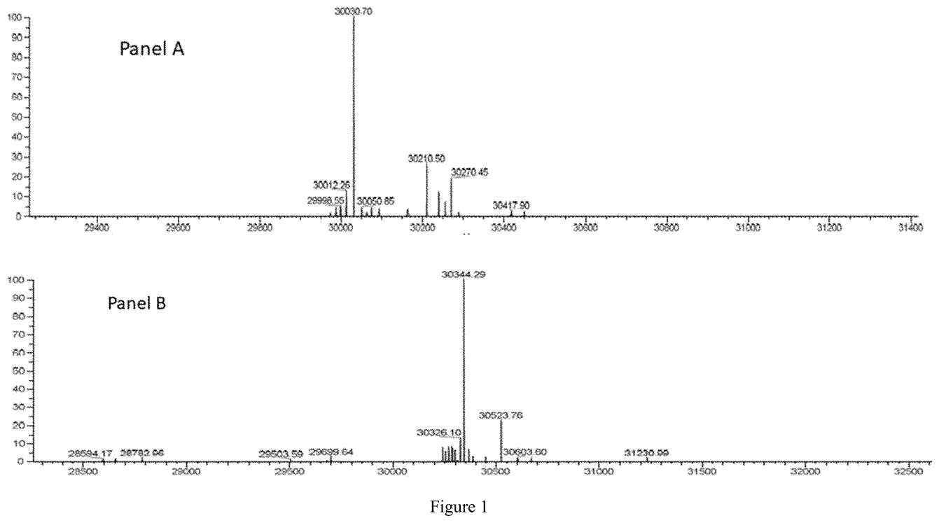

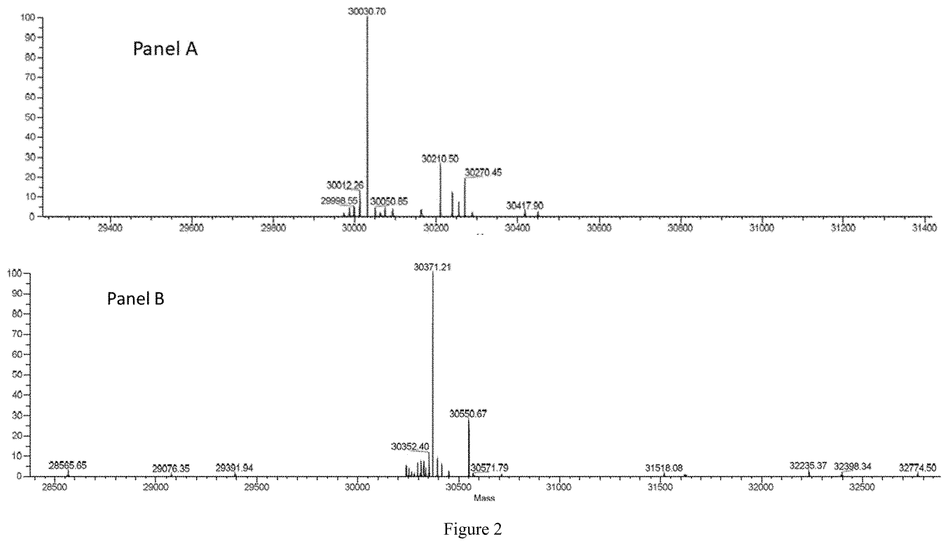

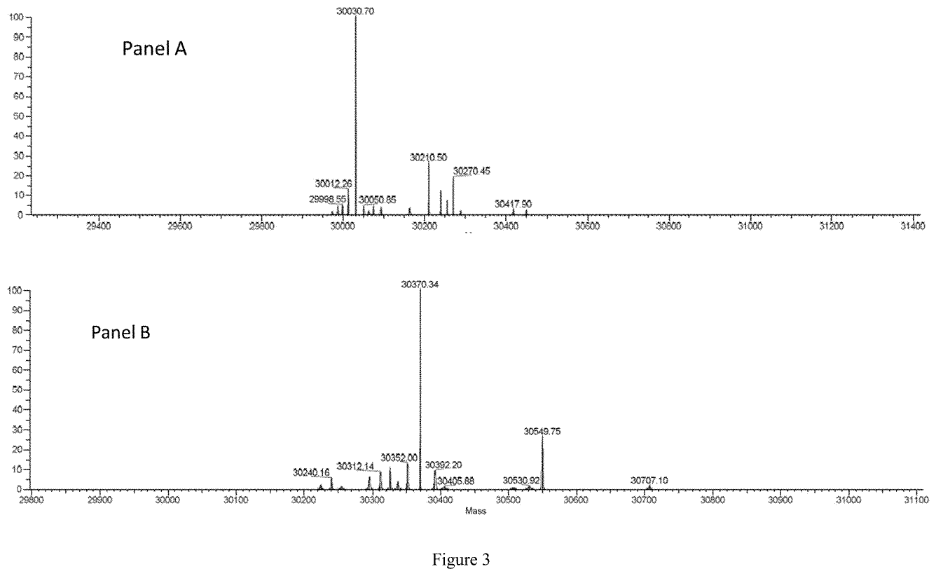

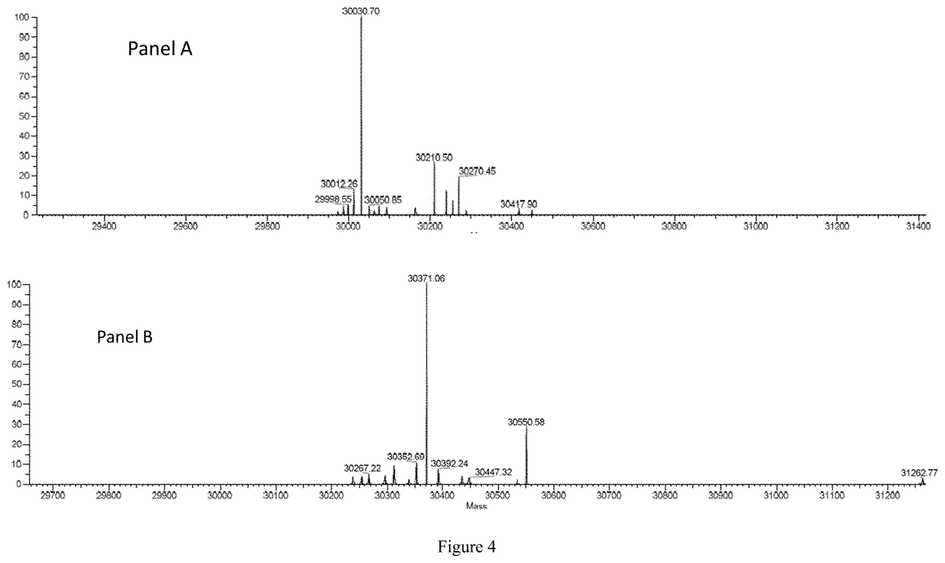

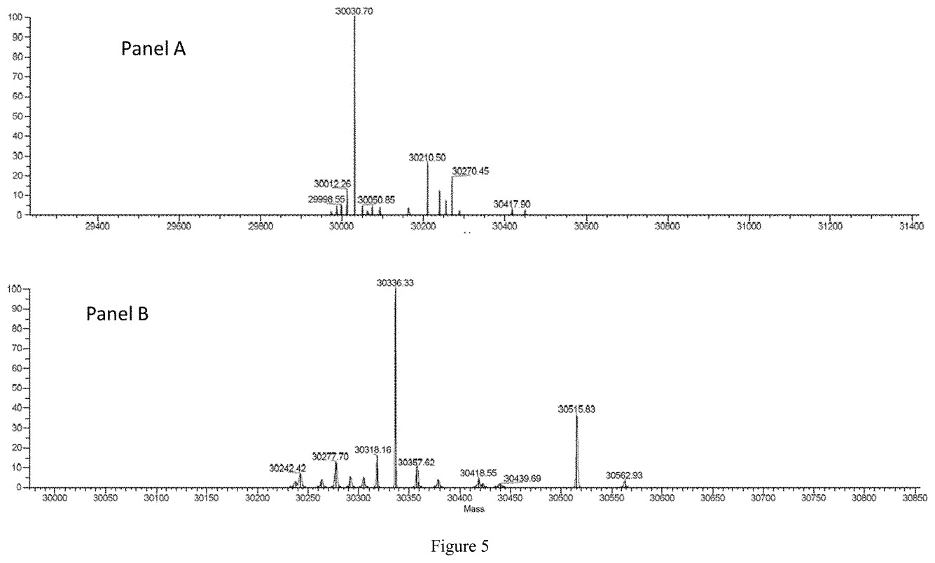

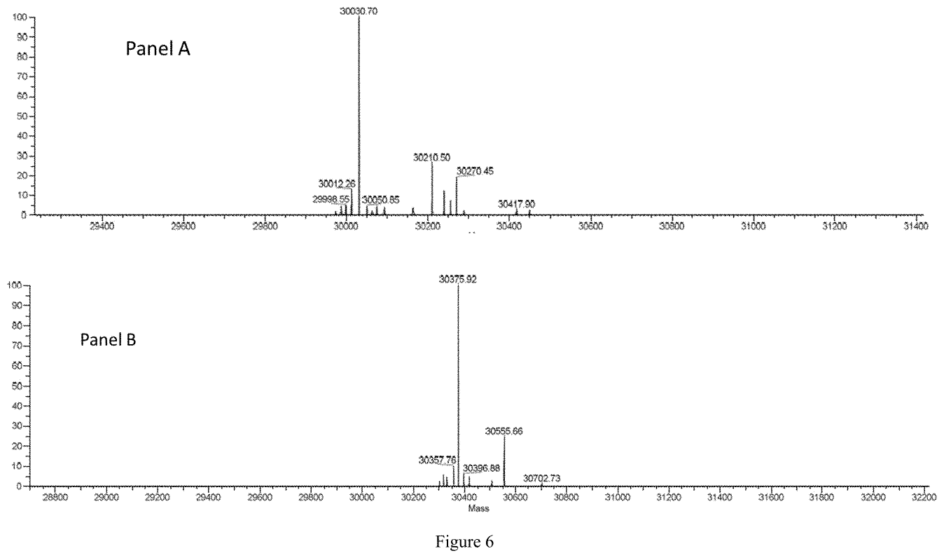

This assay confirms whether or not TEAD is covalently bound to a test compound (i.e., whether the protein's mass is modified). For example, to assess mass modification of TEAD1 with compound 58, the mass of the untreated protein (m/z 30,030.70; shown in FIG. 1, panel A) was compared to the mass of the protein treated with compound 58 (m/z 30,344.29; shown in FIG. 1, panel B). The mass shift indicates modification of TEAD1 by compound 58. Compounds 80 (see FIG. 2), 89 (see FIG. 3), 73 (see FIG. 4), 53 (see FIG. 5), and 59 (see FIG. 6) were also tested in this fashion.

### EXEMPLARY ENUMERATED EMBODIMENTS

1. A compound of Formula I:

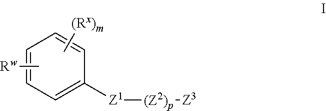

or a pharmaceutically acceptable salt thereof, wherein:
R^{w} is a warhead group;
each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
Z³ is an optionally substituted C₁₋₆ aliphatic or

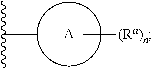

Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur;
each R^{a} is independently oxo, halogen, —CN, —NR₂, —OR, —SR, —C(O)R, —C(O)OR, NO₂, or an optionally substituted C₁₋₆ aliphatic group;
Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or
   when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or
   R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur;
each Z² is independently —CR^{z}—;
each R^{z} is independently selected from hydrogen or optionally substituted C₁₋₆ aliphatic;
m is 0, 1, 2, 3, or 4;
n is 0, 1, 2, 3, 4, or 5; and
p is 0, 1, 2, or 3.
   2. The compound according to embodiment 1, wherein the compound is of Formulae I-a, I-b, or I-c:

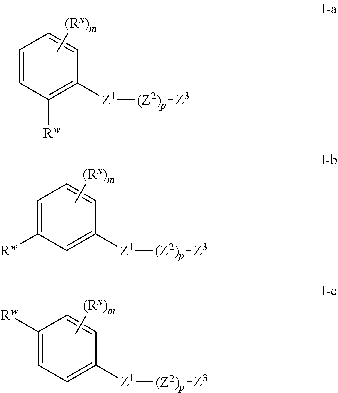

or a pharmaceutically acceptable salt thereof.
3. The compound according to embodiment 1 or 2, wherein the compound is of Formulae I-d, I-e, or I-f:

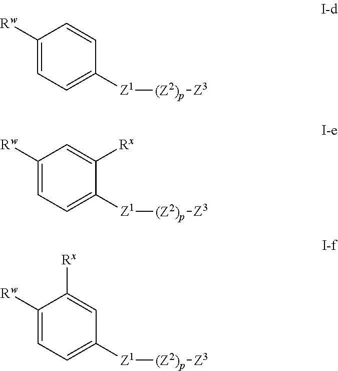

or a pharmaceutically acceptable salt thereof.
4. The compound according to embodiment 1, wherein the compound is of Formulae I-1, I-a1, I-b1, I-c1, I-d1, I-e1, or I-f1:

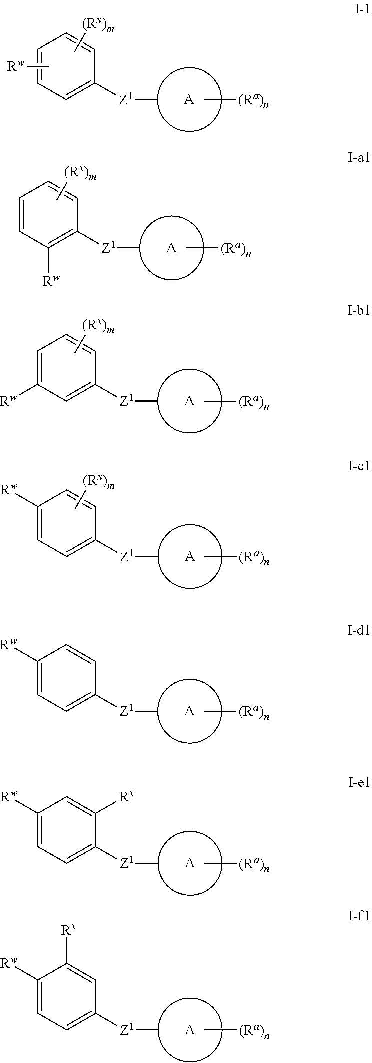

or a pharmaceutically acceptable salt thereof.
5. The compound according to embodiment 1 or 4, wherein the compound is of Formulae I-2, I-a2, I-b2, I-c2, I-d2, I-e2, or I-f2:

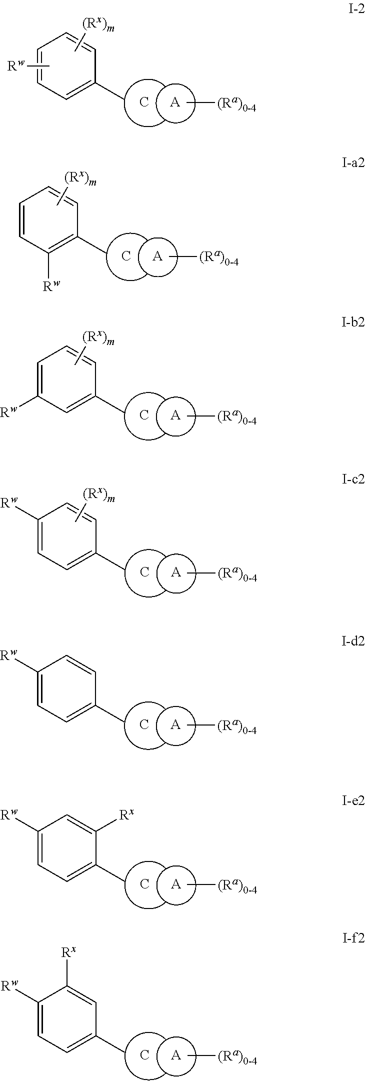

or a pharmaceutically acceptable salt thereof.
6. The compound according to embodiment 1 or 2, wherein the compound is of Formulae II, II-a, or II-b:

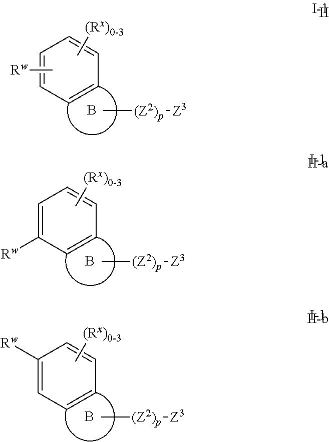

or a pharmaceutically acceptable salt thereof.
7. The compound according to any one of embodiments 1, 2, or 6, wherein the compound is of Formulae III, III-a, III-b, III-c, or III-d:

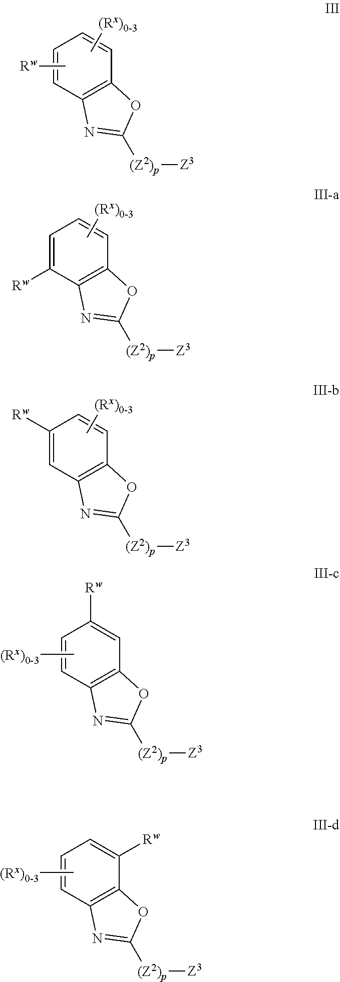

or a pharmaceutically acceptable salt thereof.
8. The compound according to any one of embodiments 1, 2, or 6, wherein the compound is of Formulae IV, IV-a, or IV-b:

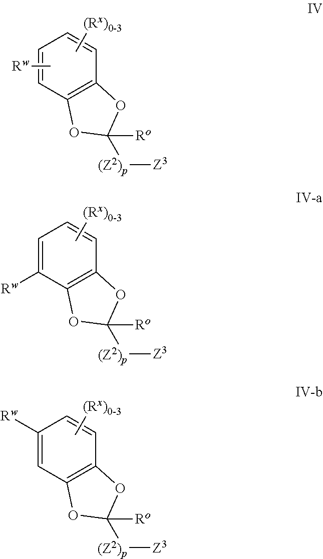

or a pharmaceutically acceptable salt thereof, wherein R^{∘} is hydrogen or C₁₋₆ aliphatic.
9. The compound according to any one of embodiments 1, 2, or 6, wherein the compound is of Formulae V, V-a, V-b, V-c, or V-d:

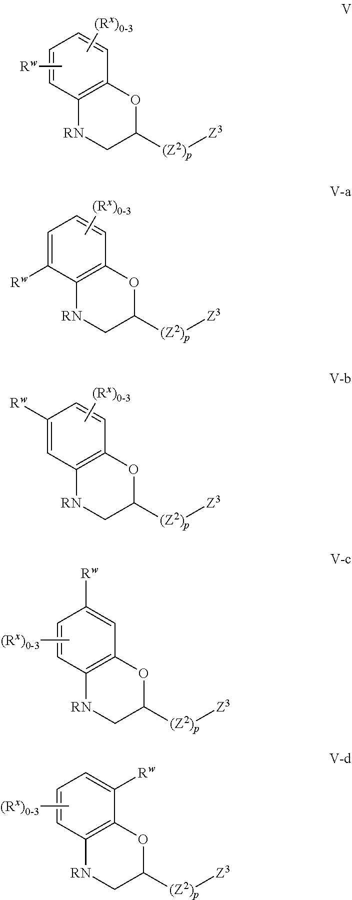

or a pharmaceutically acceptable salt thereof.
10. The compound according to any one of embodiments 1, 2, or 6, wherein the compound is of Formulae VI, VI-a, VI-b, VI-c, or VI-d:

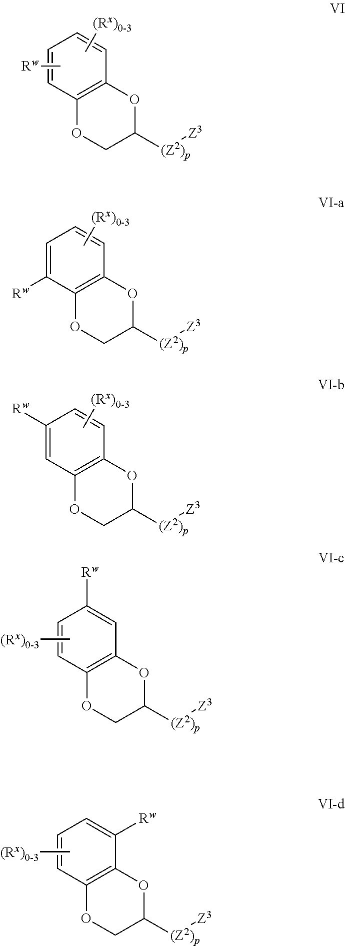

or a pharmaceutically acceptable salt thereof.
11. The compound according to any one of embodiments 1, 2, or 6, wherein the compound is of Formulae VII, VII-a, VII-b, VII-c, or VII-d:

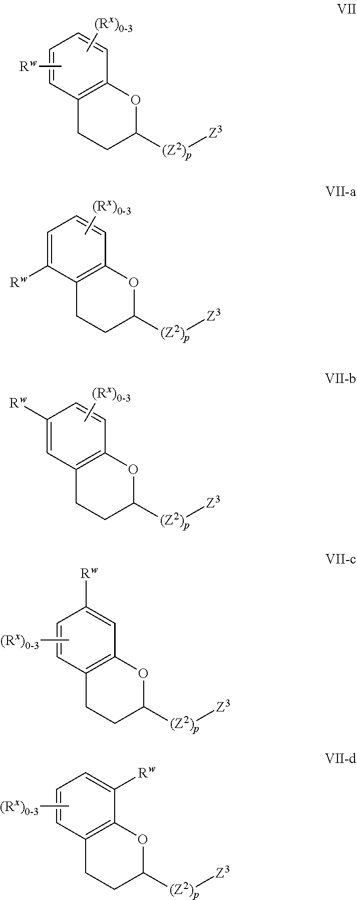

or a pharmaceutically acceptable salt thereof.
12. The compound according to any one of embodiments 1-11, wherein R^{x} is halogen, —CN, —NR₂, —OR, or an optionally substituted group selected from C₁₋₆ aliphatic or a 3- to 7-membered saturated or partially unsaturated carbocyclic ring.
13. The compound according to any one of embodiments 1-12, wherein R^{x} is selected from the group consisting of —CN, —F, —Cl, —CH₃, —CF₃, —OMe,

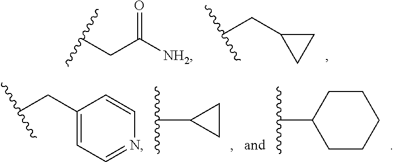

14. The compound according to any one of embodiments 1-3 or 6-13, wherein Z³ is an optionally substituted C₁₋₆ aliphatic.
15. The compound according to any one of embodiments 1-3 or 6-14, wherein Z³ is C₂₋₆ aliphatic optionally substituted with fluoro.
16. The compound according to any one of embodiments 1-3 or 6-14, wherein Z³ is selected from the group consisting of:

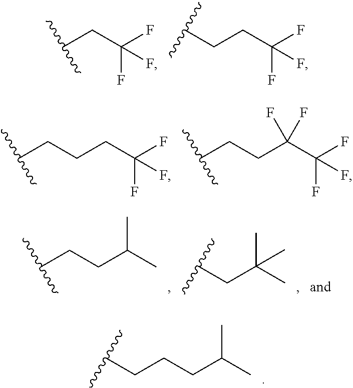

17. The compound according to any one of embodiments 1-3 or 6-16, wherein Z³ is

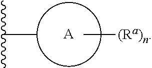

18. The compound according to any one of embodiments 1-13 or 17, wherein Ring A is cyclopropyl, cyclohexyl, phenyl, tetrahydropyranyl, dioxolyl, thiophenyl, thiazolyl, pyridinyl, pyrimidinyl, or benzothiophenyl.
19. The compound according to any one of embodiments 1-13 or 17-18, wherein Ring A is selected from the group consisting of:

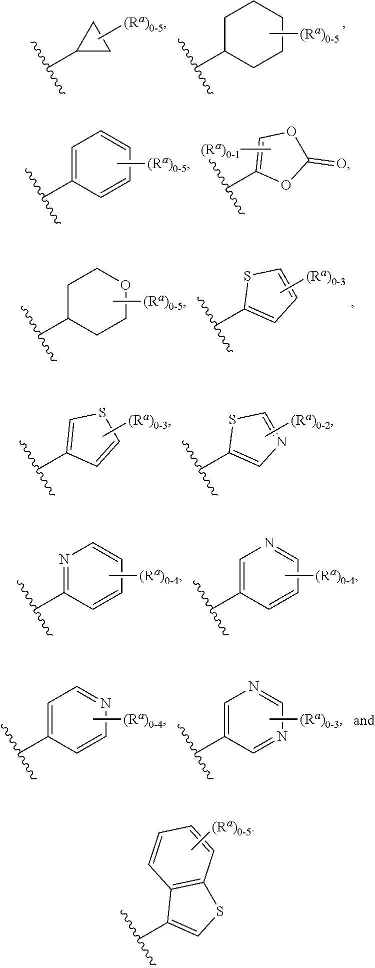

20. The compound according to any one of embodiments 1-13 or 17-19, wherein R^{a} is oxo, halogen, —CN, —NR, —OR, —C(O)OR, or an optionally substituted C₁₋₆ aliphatic group.
21. The compound according to any one of embodiments 1-13 or 17-20, wherein R^{a} is selected from the group consisting of oxo, fluoro, chloro, bromo, —CN, methyl, ethyl, ethynyl, —CF₃, —OCH₃, —C(O)OCH₃, and —C(O)OCH₂CH₃.
22. The compound according to any one of embodiments 1-4 or 12-21, wherein Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—.
23. The compound according to any one of embodiments 1-4 or 12-22, wherein Z¹ is selected from an optionally substituted bivalent straight C₂ hydrocarbon chain wherein one carbon atom of Z¹ is optionally replaced by a group selected from —O— or —N(R)—.
24. The compound according to any one of embodiments 1-4 or 12-23, wherein Z¹ is selected from the group consisting of:

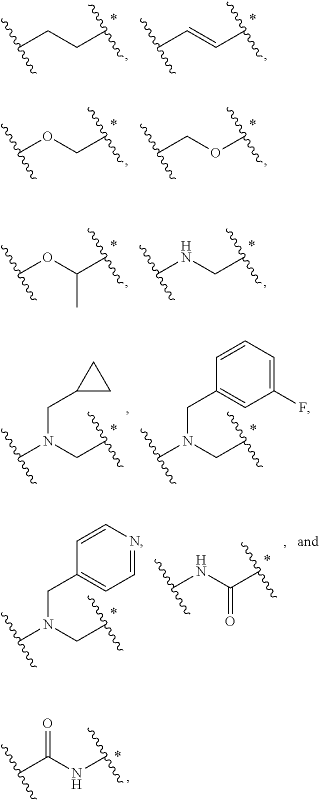

wherein * represents the point of attachment to Z².
25. The compound according to any one of embodiments 1-4 or 12-24, wherein Ring B is selected from an optionally substituted 5- to 6-membered partially unsaturated or aryl ring having 0-2 heteroatoms independently selected from nitrogen, oxygen, and sulfur.
26. The compound according to any one of embodiments 1-4, 6, 12-21, or 25, wherein Ring B is selected from an optionally substituted 5- to 6-membered partially unsaturated or aryl ring having 1-2 heteroatoms independently selected from nitrogen and oxygen.
27. The compound according to any one of embodiments 1-4, 6, 12-21, or 25, wherein Ring B is selected from:

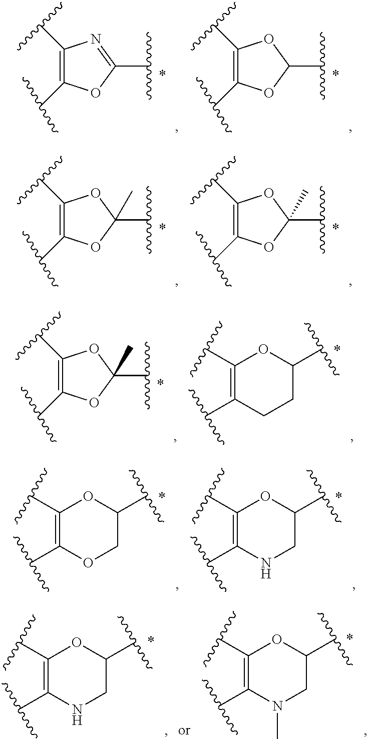

wherein * represents the point of attachment to Z².
28. The compound according to any one of embodiments 1-5, 12-13, or 17-24, wherein Ring C is an optionally substituted 5- to 6-membered saturated, partially unsaturated, or aryl ring having 0-2 heteroatoms independently selected from nitrogen, oxygen, and sulfur.
29. The compound according to any one of embodiments 1-5, 12-13, 17-24, or 28, wherein Ring C is an optionally substituted 6-membered saturated, partially unsaturated, or aryl ring having 0-2 heteroatoms independently selected from nitrogen or oxygen.
30. The compound according to any one of embodiments 1-5, 12-13, 17-24, or 28-29, wherein Ring C is selected from:

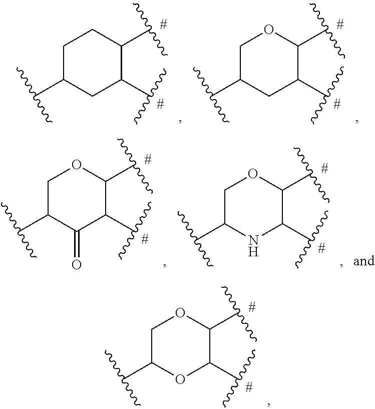 [SMILES: *C1CCC(*)C(*)C1 ; *C1COC(*)C(*)C1 ; *C1COC(*)C(*)C1=O ; *C1COC(*)C(*)N1 ; *C1COC(*)C(*)O1]

wherein each # represents a point of attachment to Ring A.
31. The compound according to any one of embodiments 1-2, 4-5, or 7-30, wherein m is 0, 1, or 2.
32. The compound according to any one of embodiments 1-13 or 17-31, wherein n is 0, 1, or 2.
33. The compound according to any one of embodiments 1-3, 6-27, or 31-32, wherein p is 0, 1, or 2.
34. The compound according to any one of embodiments 1-33, wherein R^{w} is a warhead group -L¹-X¹—Y¹, wherein:
L¹ is selected from a covalent bond or a straight or branched C₁-C₆ aliphatic, wherein one or two carbon atoms of L¹ are optionally and independently replaced by a group selected from —O— or —N(R)—;
X¹ is:

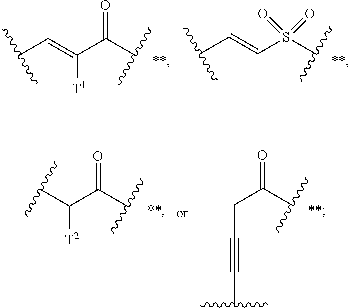

** represents the point of attachment to L¹;
T¹ is hydrogen, —CN, halogen, or optionally substituted C₁₋₆ aliphatic;
T² is halogen or —CN; and
Y¹ is hydrogen or optionally substituted C₁₋₆ aliphatic.
   35. The compound according to embodiment 34, wherein L¹ is a covalent bond or a straight or branched C₁-C₃ aliphatic, wherein one carbon atom of L¹ is optionally and independently replaced by a group selected from —O— or —N(R)—.
   36. The compound according to embodiment 34 or 35, wherein L¹ is a covalent bond, —N(H)—, or —O—.
   37. The compound according to any one of embodiments 34-36, wherein X¹ is

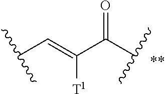

wherein ** represents the point of attachment to L¹.
38. The compound according to any one of embodiments 34-37, wherein T¹ is hydrogen, methyl, or fluoro.
39. The compound according to any one of embodiments 34-38, wherein Y¹ is hydrogen.
40. The compound according to any one of embodiments 1-39, wherein R^{w} is selected from the group consisting of:

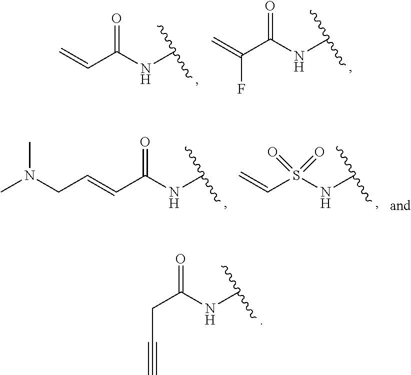

41. The compound according to any one of embodiments 1-40, wherein R^{w} is

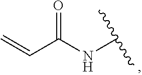 [SMILES: *NC(=O)C=C]

42. The compound according to embodiment 1, wherein the compound is compound is selected from the group consisting of:

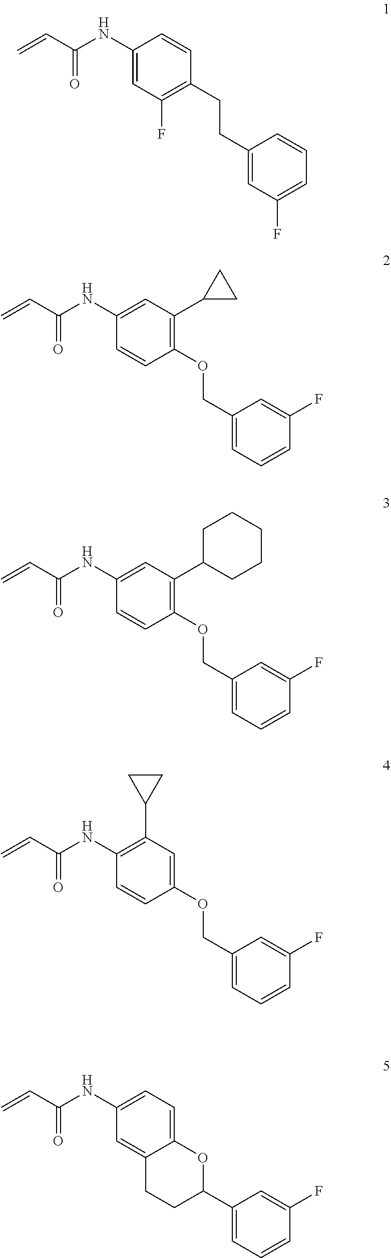 [SMILES: C=CC(=O)Nc1ccc(CCc2cccc(F)c2)c(F)c1 ; C=CC(=O)Nc1ccc(OCc2cccc(F)c2)c(C2CC2)c1 ; C=CC(=O)Nc1ccc(OCc2cccc(F)c2)c(C2CCCCC2)c1 ; C=CC(=O)Nc1ccc(OCc2cccc(F)c2)cc1C1CC1 ; C=CC(=O)Nc1ccc2c(c1)CCC(c1cccc(F)c1)O2]

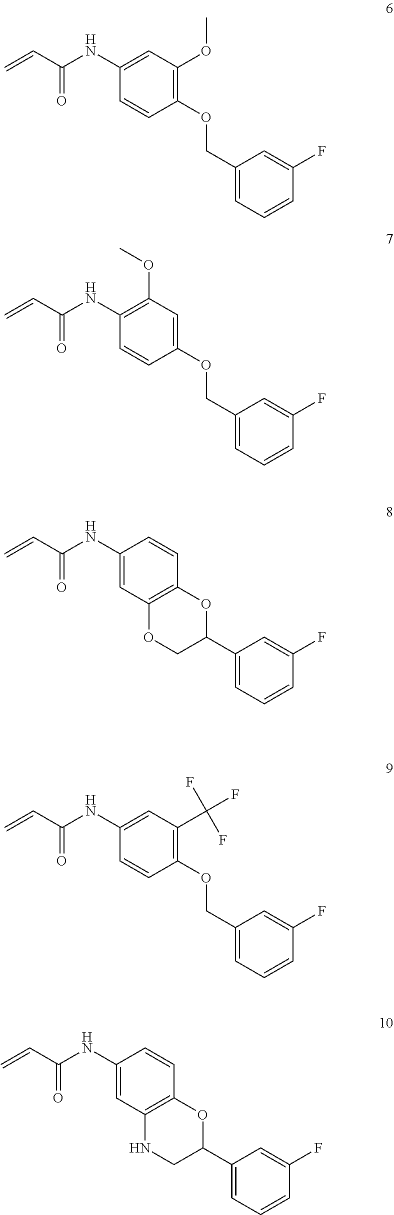 [SMILES: C=CC(=O)Nc1ccc(OCc2cccc(F)c2)c(OC)c1 ; C=CC(=O)Nc1ccc(OCc2cccc(F)c2)cc1OC ; C=CC(=O)Nc1ccc2c(c1)OCC(c1cccc(F)c1)O2 ; C=CC(=O)Nc1ccc(OCc2cccc(F)c2)c(C(F)(F)F)c1 ; C=CC(=O)Nc1ccc2c(c1)NCC(c1cccc(F)c1)O2]

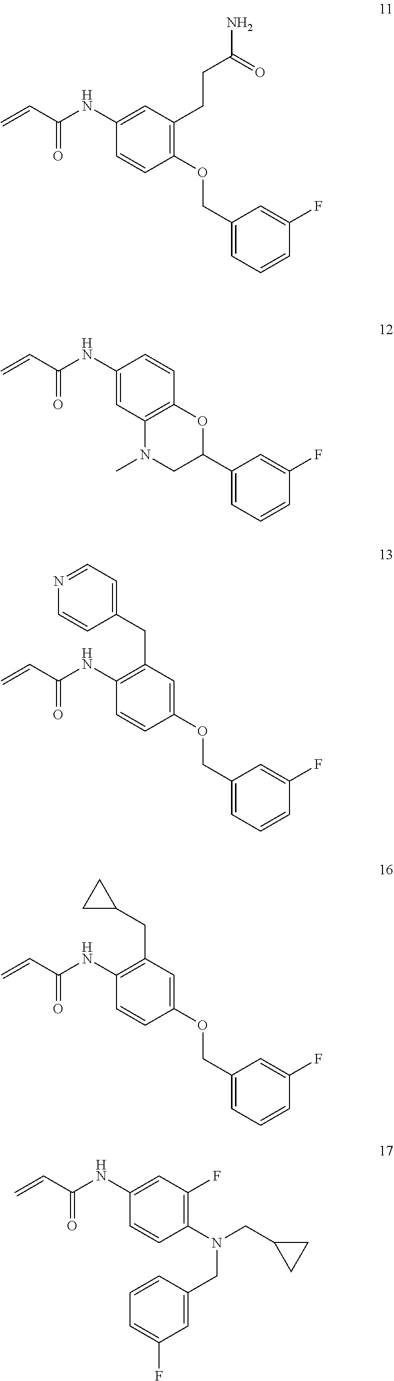 [SMILES: C=CC(=O)Nc1ccc(OCc2cccc(F)c2)c(CCC(N)=O)c1 ; C=CC(=O)Nc1ccc2c(c1)N(C)CC(c1cccc(F)c1)O2 ; C=CC(=O)Nc1ccc(OCc2cccc(F)c2)cc1Cc1ccncc1 ; C=CC(=O)Nc1ccc(OCc2cccc(F)c2)cc1CC1CC1 ; C=CC(=O)Nc1ccc(N(Cc2cccc(F)c2)CC2CC2)c(F)c1]

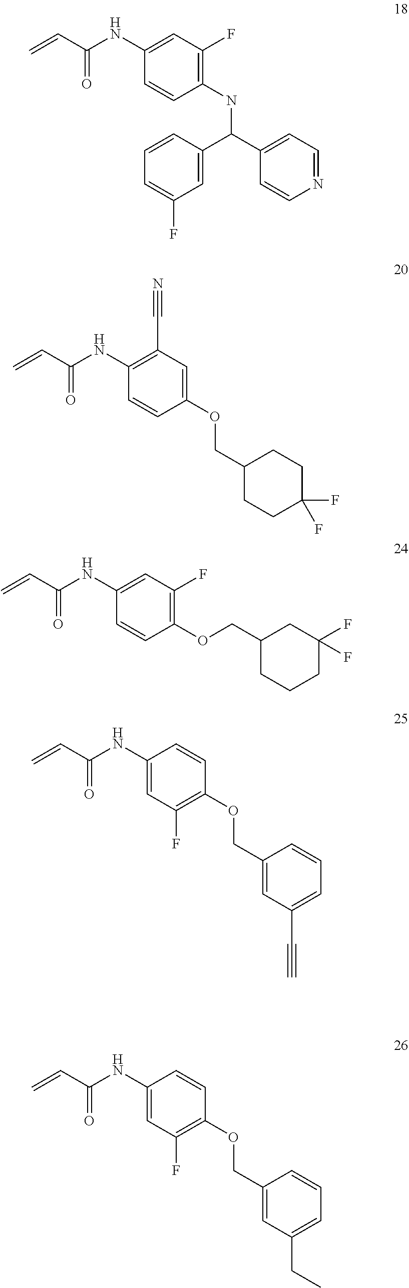 [SMILES: C=CC(=O)Nc1ccc(NC(c2ccncc2)c2cccc(F)c2)c(F)c1 ; C=CC(=O)Nc1ccc(OCC2CCC(F)(F)CC2)cc1C#N ; C=CC(=O)Nc1ccc(OCC2CCCC(F)(F)C2)c(F)c1 ; C#Cc1cccc(COc2ccc(NC(=O)C=C)cc2F)c1 ; C=CC(=O)Nc1ccc(OCc2cccc(CC)c2)c(F)c1]

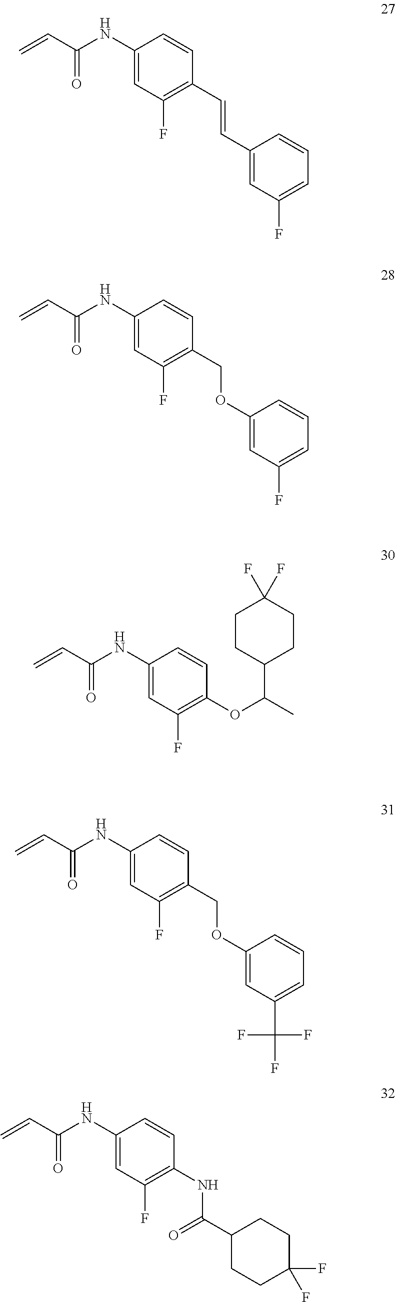 [SMILES: C=CC(=O)Nc1ccc(/C=C/c2cccc(F)c2)c(F)c1 ; C=CC(=O)Nc1ccc(COc2cccc(F)c2)c(F)c1 ; C=CC(=O)Nc1ccc(OC(C)C2CCC(F)(F)CC2)c(F)c1 ; C=CC(=O)Nc1ccc(COc2cccc(C(F)(F)F)c2)c(F)c1 ; C=CC(=O)Nc1ccc(NC(=O)C2CCC(F)(F)CC2)c(F)c1]

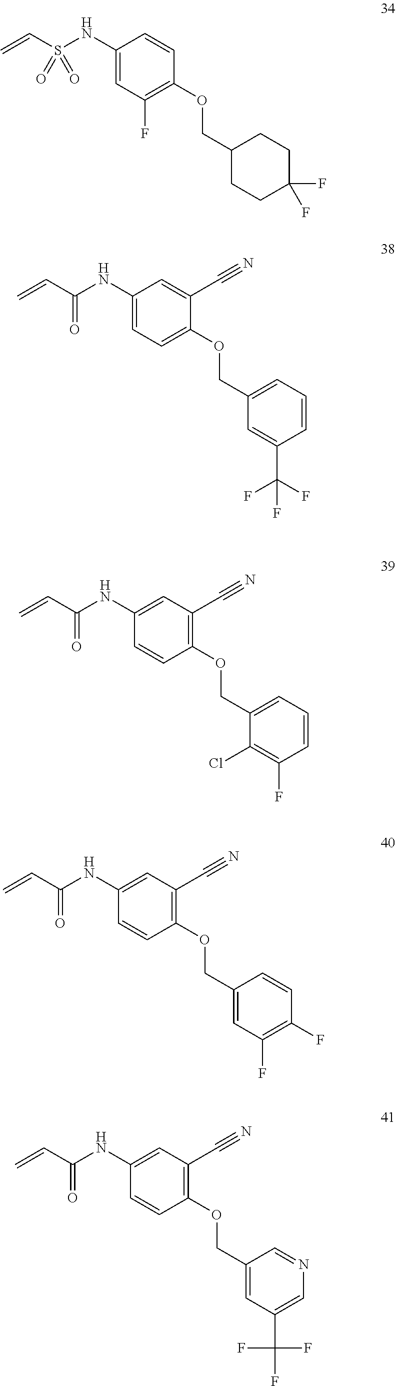 [SMILES: C=CS(=O)(=O)Nc1ccc(OCC2CCC(F)(F)CC2)c(F)c1 ; C=CC(=O)Nc1ccc(OCc2cccc(C(F)(F)F)c2)c(C#N)c1 ; C=CC(=O)Nc1ccc(OCc2cccc(F)c2Cl)c(C#N)c1 ; C=CC(=O)Nc1ccc(OCc2ccc(F)c(F)c2)c(C#N)c1 ; C=CC(=O)Nc1ccc(OCc2cncc(C(F)(F)F)c2)c(C#N)c1]

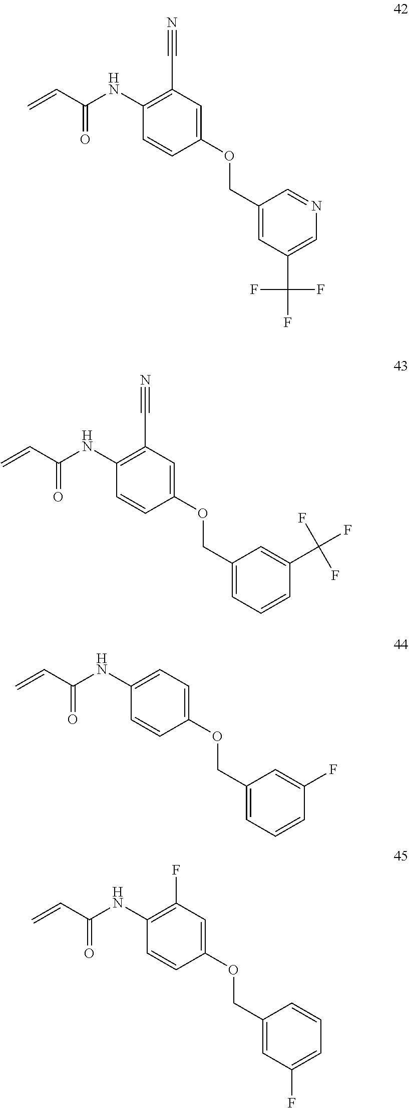 [SMILES: C=CC(=O)Nc1ccc(OCc2cncc(C(F)(F)F)c2)cc1C#N ; C=CC(=O)Nc1ccc(OCc2cccc(C(F)(F)F)c2)cc1C#N ; C=CC(=O)Nc1ccc(OCc2cccc(F)c2)cc1 ; C=CC(=O)Nc1ccc(OCc2cccc(F)c2)cc1F]

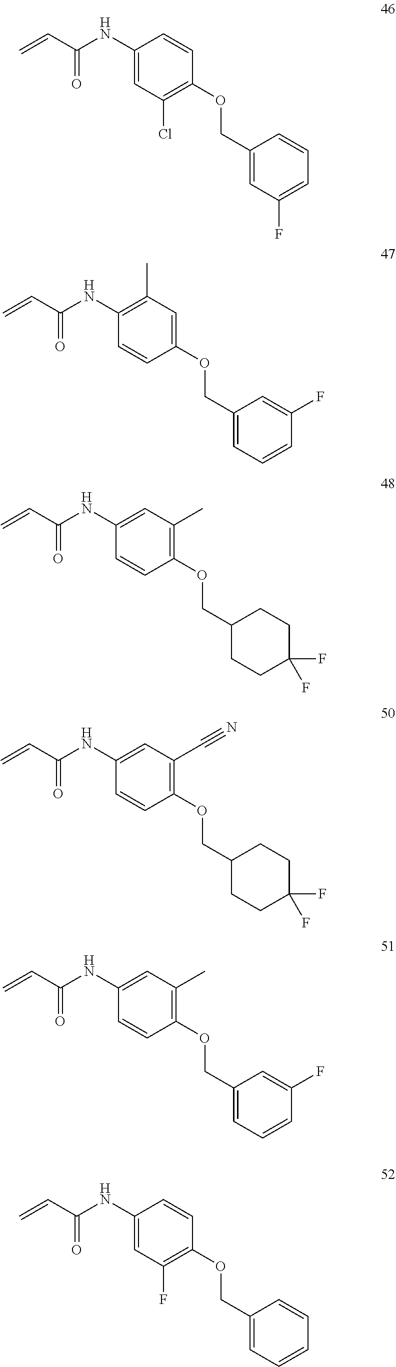 [SMILES: C=CC(=O)Nc1ccc(OCc2cccc(F)c2)c(Cl)c1 ; C=CC(=O)Nc1ccc(OCc2cccc(F)c2)cc1C ; C=CC(=O)Nc1ccc(OCC2CCC(F)(F)CC2)c(C)c1 ; C=CC(=O)Nc1ccc(OCC2CCC(F)(F)CC2)c(C#N)c1 ; C=CC(=O)Nc1ccc(OCc2cccc(F)c2)c(C)c1 ; C=CC(=O)Nc1ccc(OCc2ccccc2)c(F)c1]

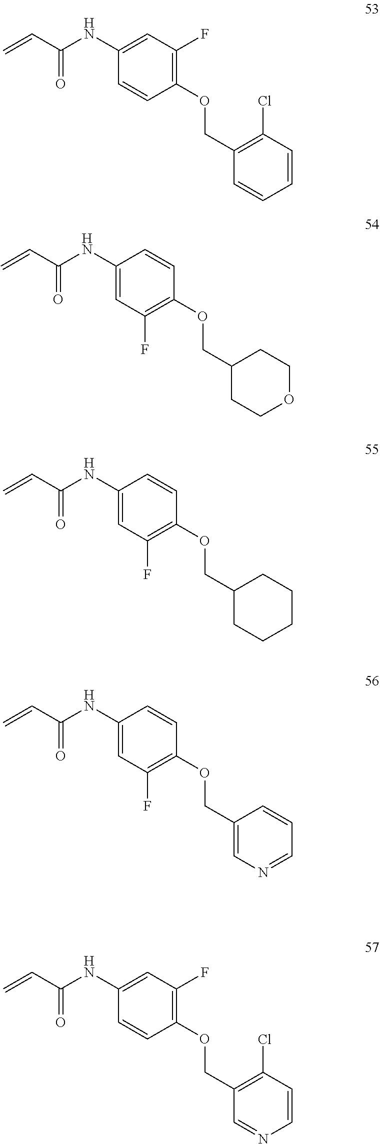 [SMILES: C=CC(=O)Nc1ccc(OCc2ccccc2Cl)c(F)c1 ; C=CC(=O)Nc1ccc(OCC2CCOCC2)c(F)c1 ; C=CC(=O)Nc1ccc(OCC2CCCCC2)c(F)c1 ; C=CC(=O)Nc1ccc(OCc2cccnc2)c(F)c1 ; C=CC(=O)Nc1ccc(OCc2cnccc2Cl)c(F)c1]

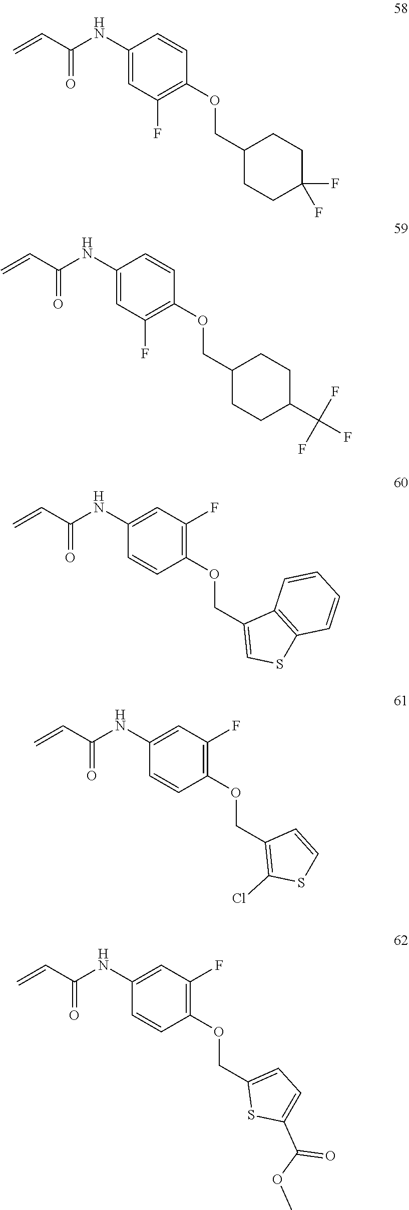 [SMILES: C=CC(=O)Nc1ccc(OCC2CCC(F)(F)CC2)c(F)c1 ; C=CC(=O)Nc1ccc(OCC2CCC(C(F)(F)F)CC2)c(F)c1 ; C=CC(=O)Nc1ccc(OCc2csc3ccccc23)c(F)c1 ; C=CC(=O)Nc1ccc(OCc2ccsc2Cl)c(F)c1 ; C=CC(=O)Nc1ccc(OCc2ccc(C(=O)OC)s2)c(F)c1]

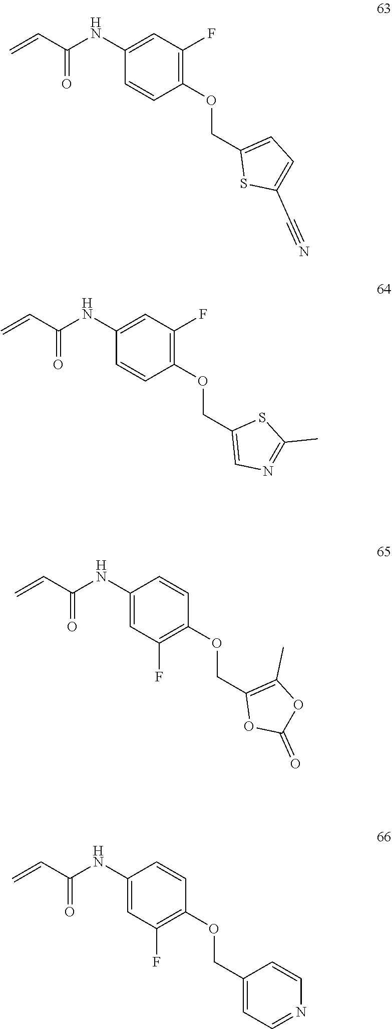

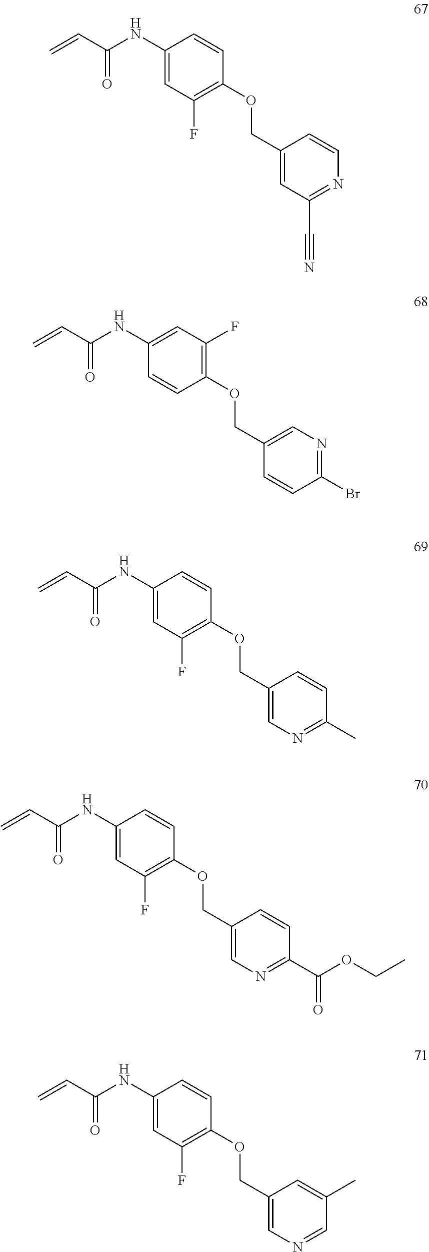

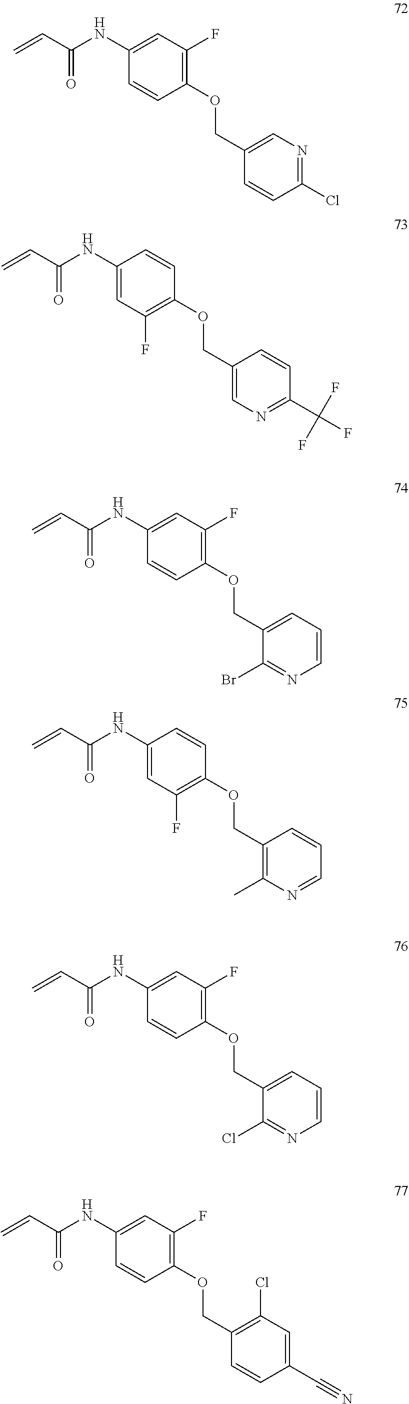 [SMILES: C=CC(=O)Nc1ccc(OCc2ccc(Cl)nc2)c(F)c1 ; C=CC(=O)Nc1ccc(OCc2ccc(C(F)(F)F)nc2)c(F)c1 ; C=CC(=O)Nc1ccc(OCc2cccnc2Br)c(F)c1 ; C=CC(=O)Nc1ccc(OCc2cccnc2C)c(F)c1 ; C=CC(=O)Nc1ccc(OCc2cccnc2Cl)c(F)c1 ; C=CC(=O)Nc1ccc(OCc2ccc(C#N)cc2Cl)c(F)c1]

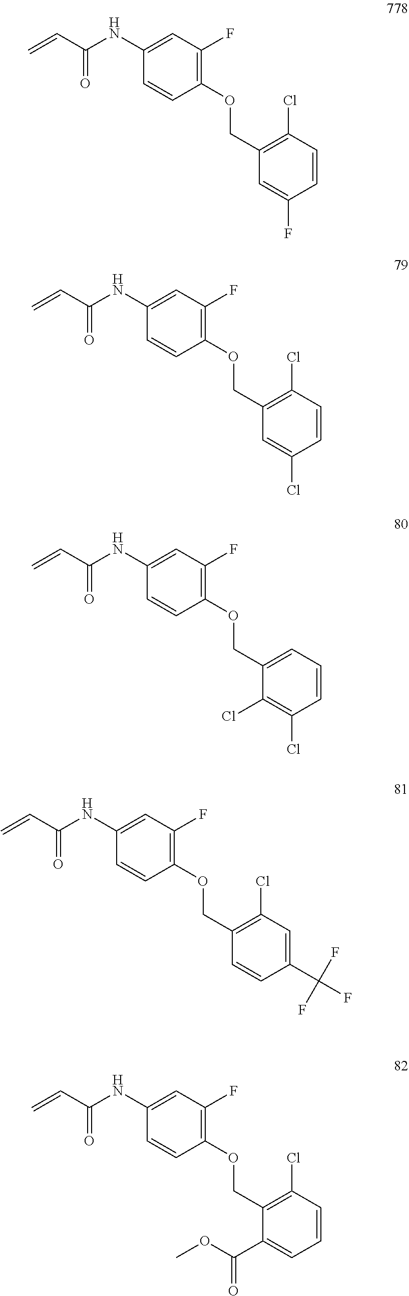 [SMILES: C=CC(=O)Nc1ccc(OCc2cc(F)ccc2Cl)c(F)c1 ; C=CC(=O)Nc1ccc(OCc2cc(Cl)ccc2Cl)c(F)c1 ; C=CC(=O)Nc1ccc(OCc2cccc(Cl)c2Cl)c(F)c1 ; C=CC(=O)Nc1ccc(OCc2ccc(C(F)(F)F)cc2Cl)c(F)c1 ; C=CC(=O)Nc1ccc(OCc2c(Cl)cccc2C(=O)OC)c(F)c1]

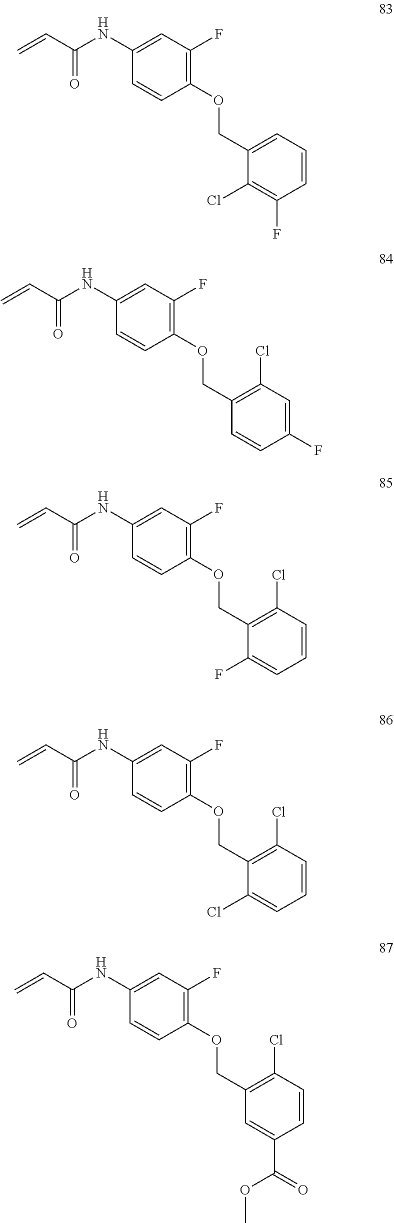 [SMILES: C=CC(=O)Nc1ccc(OCc2cccc(F)c2Cl)c(F)c1 ; C=CC(=O)Nc1ccc(OCc2ccc(F)cc2Cl)c(F)c1 ; C=CC(=O)Nc1ccc(OCc2c(F)cccc2Cl)c(F)c1 ; C=CC(=O)Nc1ccc(OCc2c(Cl)cccc2Cl)c(F)c1 ; C=CC(=O)Nc1ccc(OCc2cc(C(=O)OC)ccc2Cl)c(F)c1]

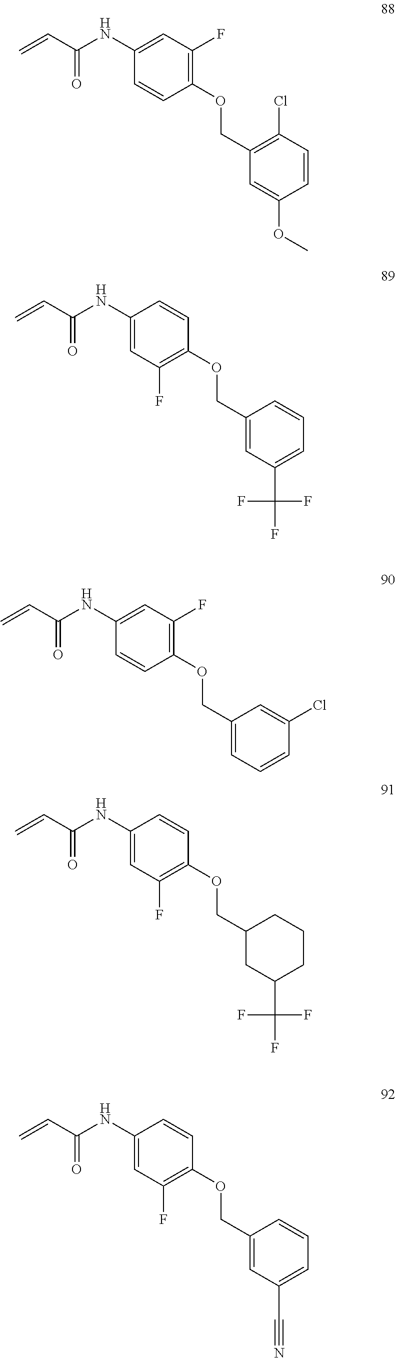

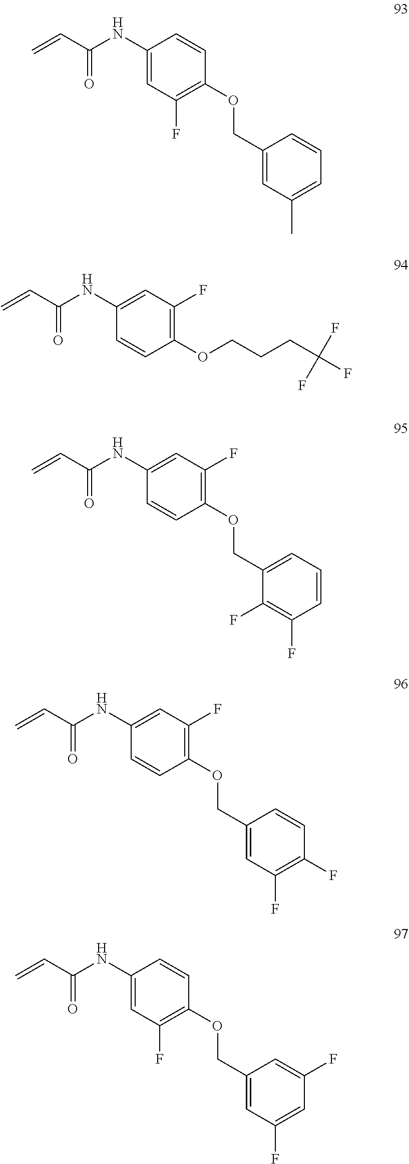 [SMILES: C=CC(=O)Nc1ccc(OCc2cccc(C)c2)c(F)c1 ; C=CC(=O)Nc1ccc(OCCCC(F)(F)F)c(F)c1 ; C=CC(=O)Nc1ccc(OCc2cccc(F)c2F)c(F)c1 ; C=CC(=O)Nc1ccc(OCc2ccc(F)c(F)c2)c(F)c1 ; C=CC(=O)Nc1ccc(OCc2cc(F)cc(F)c2)c(F)c1]

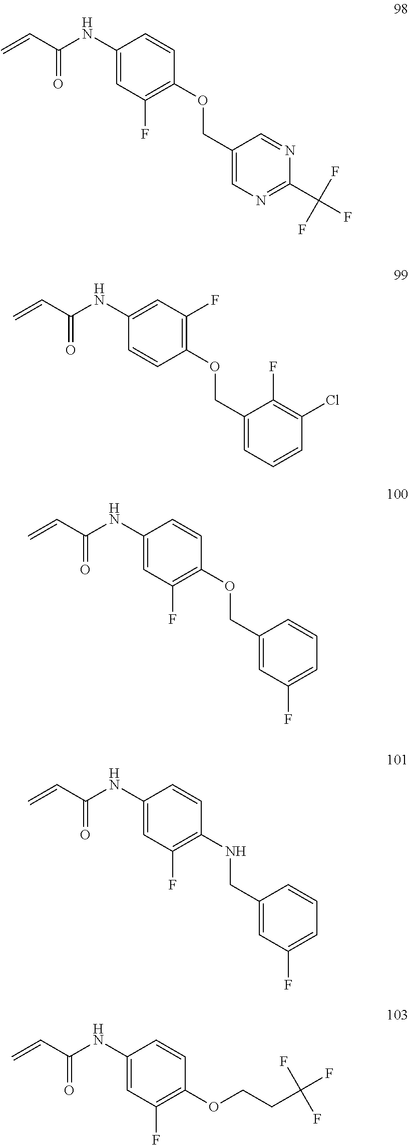 [SMILES: C=CC(=O)Nc1ccc(OCc2cnc(C(F)(F)F)nc2)c(F)c1 ; C=CC(=O)Nc1ccc(OCc2cccc(Cl)c2F)c(F)c1 ; C=CC(=O)Nc1ccc(OCc2cccc(F)c2)c(F)c1 ; C=CC(=O)Nc1ccc(NCc2cccc(F)c2)c(F)c1 ; C=CC(=O)Nc1ccc(OCCC(F)(F)F)c(F)c1]

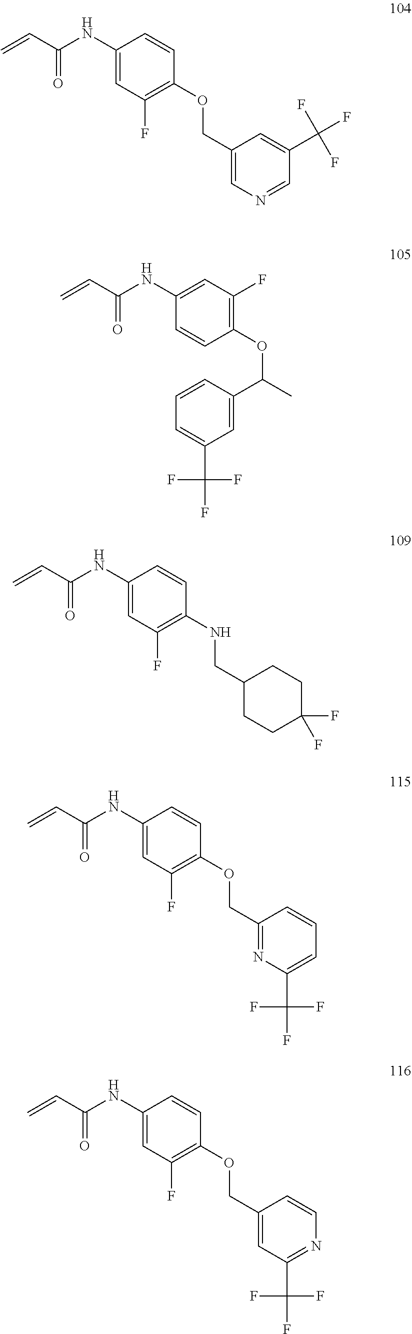

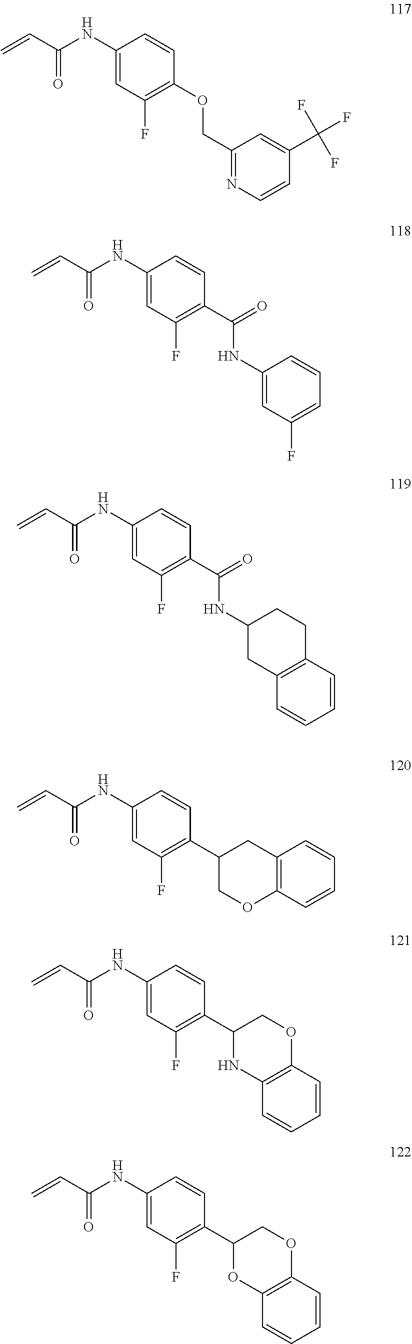 [SMILES: C=CC(=O)Nc1ccc(OCc2cc(C(F)(F)F)ccn2)c(F)c1 ; C=CC(=O)Nc1ccc(C(=O)Nc2cccc(F)c2)c(F)c1 ; C=CC(=O)Nc1ccc(C(=O)NC2CCc3ccccc3C2)c(F)c1 ; C=CC(=O)Nc1ccc(C2COc3ccccc3C2)c(F)c1 ; C=CC(=O)Nc1ccc(C2COc3ccccc3N2)c(F)c1 ; C=CC(=O)Nc1ccc(C2COc3ccccc3O2)c(F)c1]

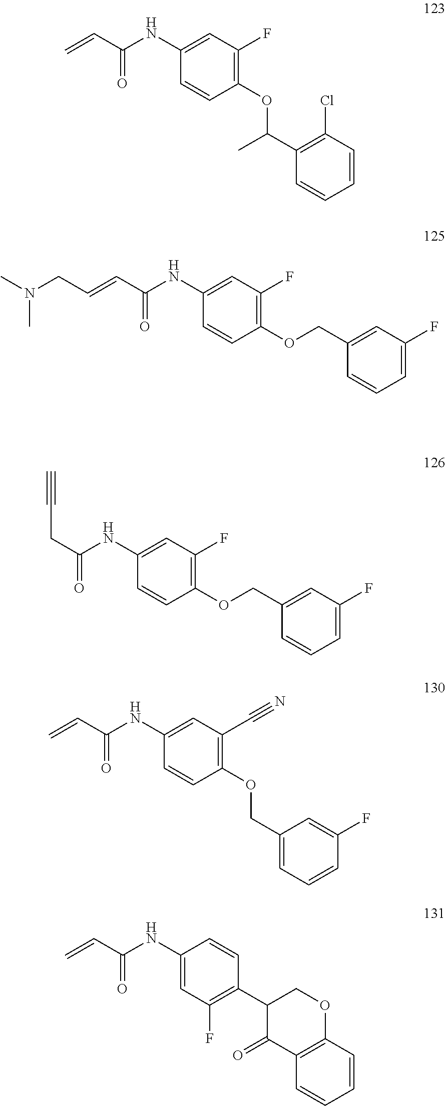 [SMILES: C=CC(=O)Nc1ccc(OC(C)c2ccccc2Cl)c(F)c1 ; CN(C)C/C=C/C(=O)Nc1ccc(OCc2cccc(F)c2)c(F)c1 ; C#CCC(=O)Nc1ccc(OCc2cccc(F)c2)c(F)c1 ; C=CC(=O)Nc1ccc(OCc2cccc(F)c2)c(C#N)c1 ; C=CC(=O)Nc1ccc(C2COc3ccccc3C2=O)c(F)c1]

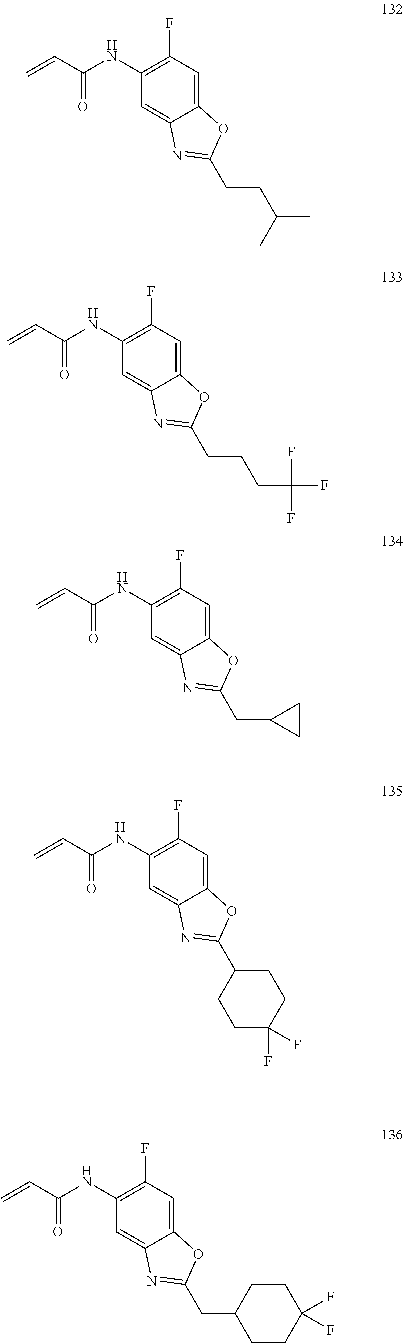

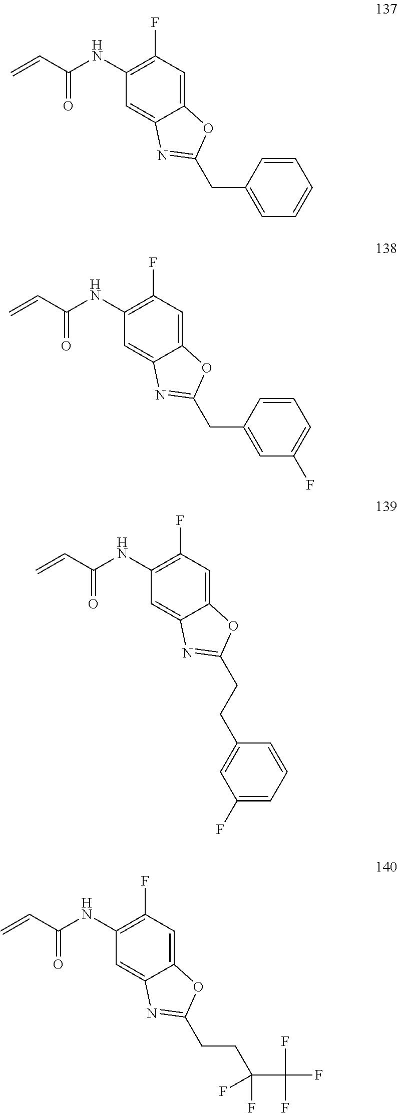 [SMILES: C=CC(=O)Nc1cc2nc(Cc3ccccc3)oc2cc1F ; C=CC(=O)Nc1cc2nc(Cc3cccc(F)c3)oc2cc1F ; C=CC(=O)Nc1cc2nc(CCc3cccc(F)c3)oc2cc1F ; C=CC(=O)Nc1cc2nc(CCC(F)(F)C(F)(F)F)oc2cc1F]

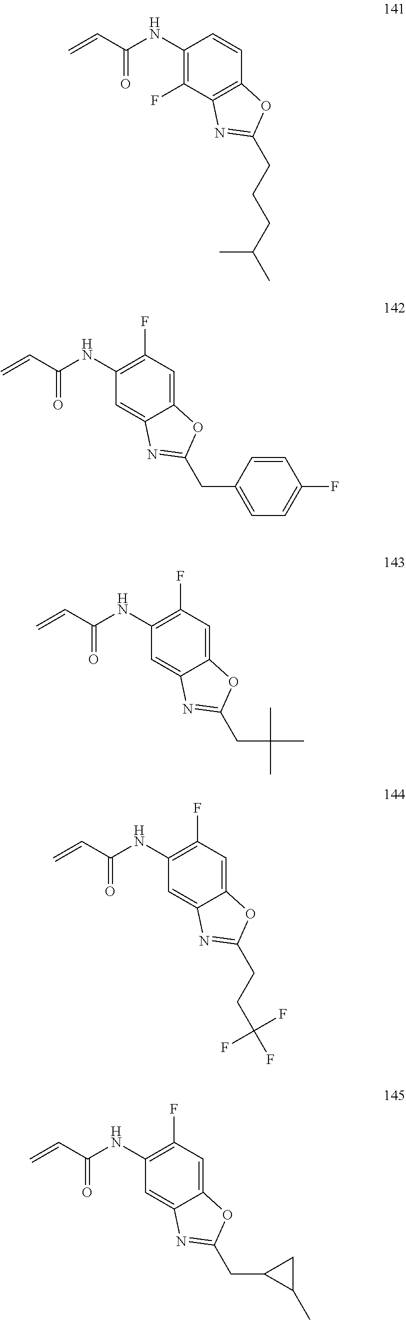

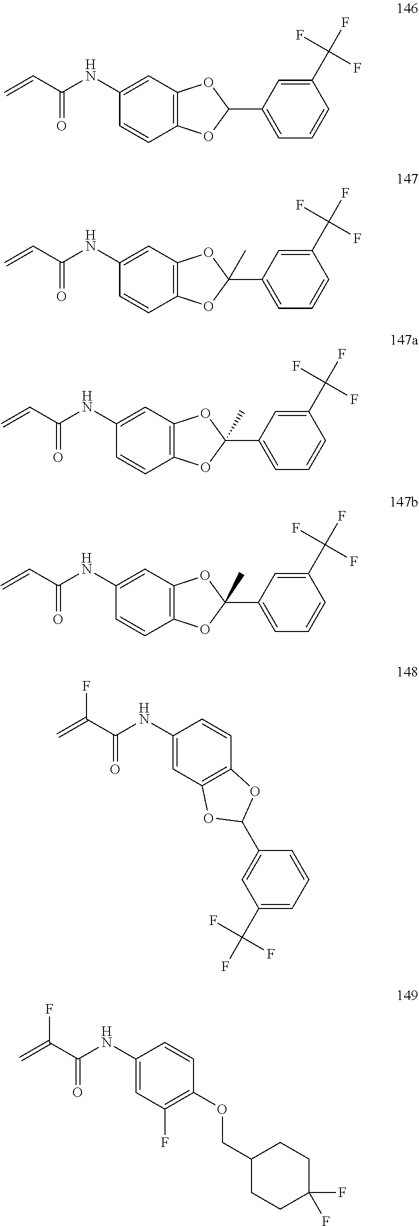 [SMILES: C=CC(=O)Nc1ccc2c(c1)OC(c1cccc(C(F)(F)F)c1)O2 ; C=CC(=O)Nc1ccc2c(c1)OC(C)(c1cccc(C(F)(F)F)c1)O2 ; C=CC(=O)Nc1ccc2c(c1)O[C@@](C)(c1cccc(C(F)(F)F)c1)O2 ; C=CC(=O)Nc1ccc2c(c1)O[C@](C)(c1cccc(C(F)(F)F)c1)O2 ; C=C(F)C(=O)Nc1ccc2c(c1)OC(c1cccc(C(F)(F)F)c1)O2 ; C=C(F)C(=O)Nc1ccc(OCC2CCC(F)(F)CC2)c(F)c1]

or a pharmaceutically acceptable salt thereof.
43. A compound selected from the group consisting of:

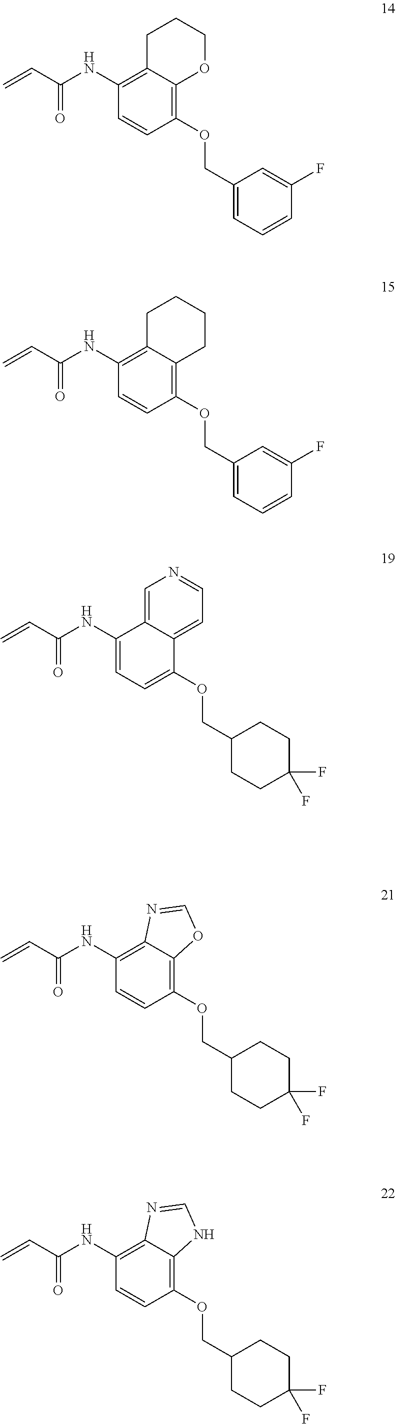 [SMILES: C=CC(=O)Nc1ccc(OCc2cccc(F)c2)c2c1CCCO2 ; C=CC(=O)Nc1ccc(OCc2cccc(F)c2)c2c1CCCC2 ; C=CC(=O)Nc1ccc(OCC2CCC(F)(F)CC2)c2ccncc12 ; C=CC(=O)Nc1ccc(OCC2CCC(F)(F)CC2)c2ocnc12 ; C=CC(=O)Nc1ccc(OCC2CCC(F)(F)CC2)c2[nH]cnc12]

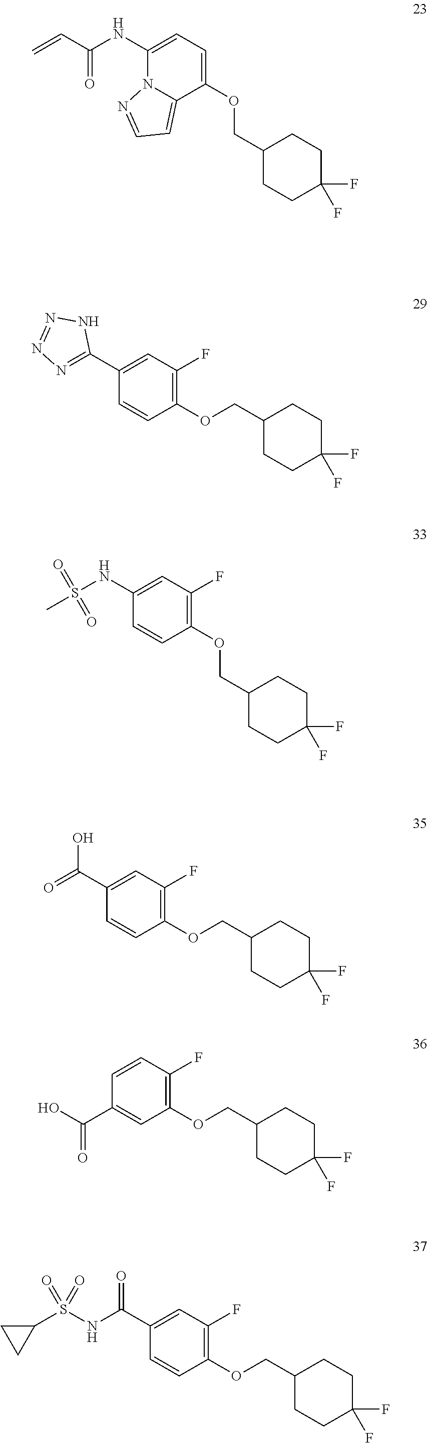 [SMILES: C=CC(=O)Nc1ccc(OCC2CCC(F)(F)CC2)c2ccnn12 ; Fc1cc(-c2nnn[nH]2)ccc1OCC1CCC(F)(F)CC1 ; CS(=O)(=O)Nc1ccc(OCC2CCC(F)(F)CC2)c(F)c1 ; O=C(O)c1ccc(OCC2CCC(F)(F)CC2)c(F)c1 ; O=C(O)c1ccc(F)c(OCC2CCC(F)(F)CC2)c1 ; O=C(NS(=O)(=O)C1CC1)c1ccc(OCC2CCC(F)(F)CC2)c(F)c1]

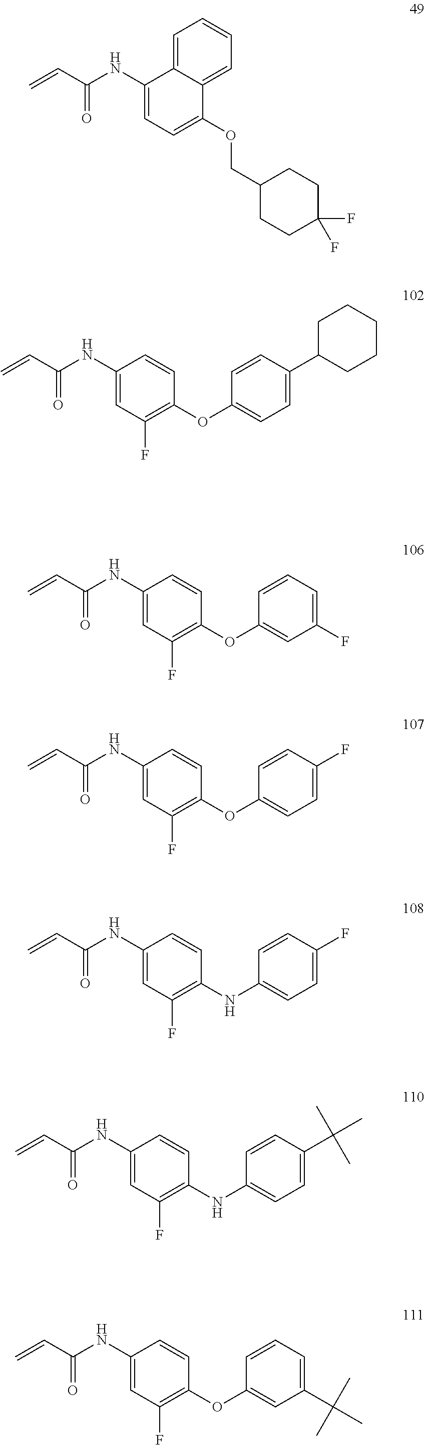

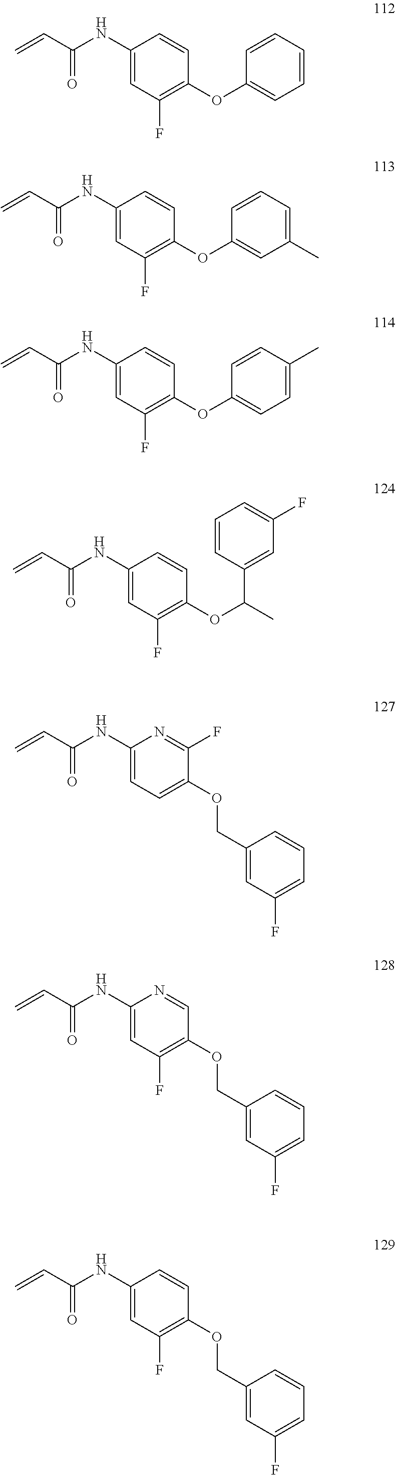

or a pharmaceutically acceptable salt thereof.
44. A pharmaceutical composition comprising a compound according to any one of embodiments 1-43, or a pharmaceutically acceptable salt thereof, and a pharmaceutically acceptable carrier, adjuvant, or vehicle.
45. A method of inhibiting activity of a TEAD transcription factor, or a mutant thereof, in a biological sample or in a patient, the method comprising a step of contacting the biological sample or administering to a patient a compound according to any one of embodiments 1-43, or a pharmaceutically acceptable salt thereof.
46. The method of embodiment 45, wherein the activity of a TEAD transcription factor, or a mutant thereof, is inhibited irreversibly.
47. A method of treating a disease or disorder associated with TEAD, the method comprising a step of administering to a patient in need thereof a compound according to any one of embodiments 1-43, or a pharmaceutically acceptable salt thereof.
48. The method according to embodiment 47, wherein the disease or disorder associated with TEAD is a proliferative disease.
49. The method according to embodiment 48, wherein the proliferative disease is a cancer.
50. The method according to embodiment 49, wherein the cancer is selected from a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

1. A compound of Formula I: 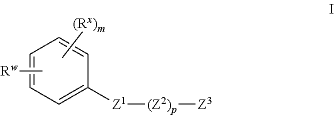
or a pharmaceutically acceptable salt thereof, wherein:
R^{w} is a warhead group -L¹-X¹—Y¹, wherein:
L¹ is selected from a covalent bond or a straight or branched C₁-C₆ aliphatic, wherein one or two carbon atoms of L¹ are optionally and independently replaced by a group selected from —O— or —N(R)—;
X is: 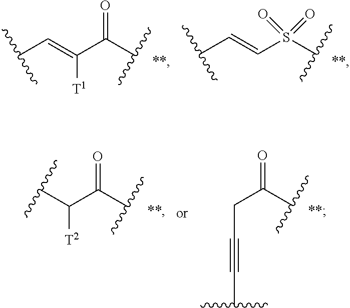
** represents the point of attachment to L¹;
T¹ is hydrogen, —CN, halogen, or optionally substituted C₁₋₆ aliphatic;
T² is halogen or —CN; and
Y¹ is hydrogen or optionally substituted C₁₋₆ aliphatic;
each R^{x} is independently halogen, —CN, —NR₂, —SR, —OR, —C(O)R, —C(O)OR, —NO₂, or an optionally substituted group selected from C₁₋₆ aliphatic, a 3- to 7-membered saturated or partially unsaturated carbocyclic ring, a 4- to 7-membered saturated or partially unsaturated heterocyclic ring having 1-3 heteroatoms independently selected from nitrogen, oxygen, and sulfur, phenyl, a 5- to 6-membered heteroaryl ring having 1-4 heteroatoms independently selected from nitrogen, oxygen, and sulfur, or an 8- to 10-membered bicyclic ring having 0-4 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
Z³ is an optionally substituted C₁₋₆ aliphatic or 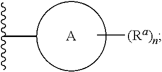
Ring A is selected from a 3- to 7-membered saturated or partially unsaturated carbocyclic ring, phenyl, a 4- to 7-membered saturated or partially unsaturated heterocyclic ring having 1-3 heteroatoms independently selected from nitrogen, oxygen, and sulfur, a 5- to 6-membered heteroaryl ring having 1-4 heteroatoms independently selected from nitrogen, oxygen, and sulfur, an 8- to 10-membered bicyclic heteroaryl ring having 1-4 heteroatoms independently selected from nitrogen, oxygen, and sulfur, or a 7-11 membered spirofused ring having 0-4 heteroatoms independently selected from nitrogen, oxygen, and sulfur;
each R^{a} is independently oxo, halogen, —CN, —NR₂, —OR, —SR, —C(O)R, —C(O)OR, NO₂, or an optionally substituted C₁₋₆ aliphatic group;
Z¹ is selected from an optionally substituted bivalent straight C₂₋₅ hydrocarbon chain wherein one or two carbon atoms of Z¹ are optionally and independently replaced by a group selected from —O—, —N(R)—, or —C(O)—; or
when R^{x} is attached to an atom adjacent to the atom where Z¹ is attached, R^{x} and Z¹, together with their intervening atoms, may form Ring B, wherein Ring B is selected from an optionally substituted 5- to 6-membered partially unsaturated or aryl ring having 0-2 heteroatoms independently selected from nitrogen, oxygen, and sulfur; or
R^{a} and Z¹, together with their intervening atoms, may form Ring C, wherein Ring C is an optionally substituted 5- to 6-membered saturated, partially unsaturated, or aryl ring having 0-2 heteroatoms independently selected from nitrogen, oxygen, and sulfur;
each Z² is independently —CR^{z}—;
each R^{z} is independently selected from hydrogen or optionally substituted C₁₋₆ aliphatic;
m is 0, 1, 2, 3, or 4;
n is 0, 1, 2, 3, 4, or 5; and
p is 0, 1, 2, or 3.

2. The compound according to claim 1, wherein the compound is of Formulae I-a, I-b, I-c, I-d, I-e, or I-f: 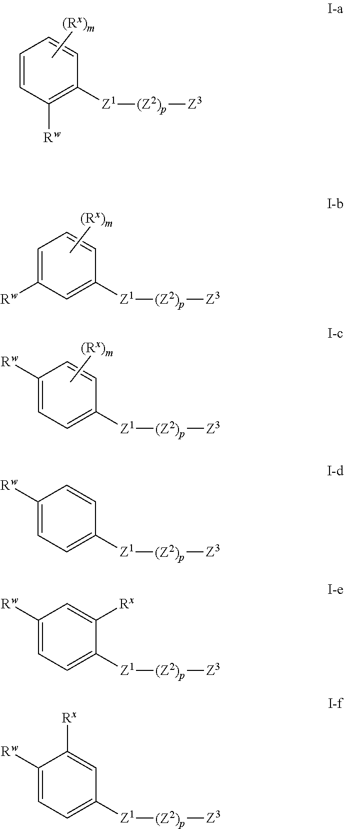
or a pharmaceutically acceptable salt thereof.

3. The compound according to claim 1, wherein the compound is of Formulae I-1, I-a1, I-b1, I-c1, I-d1, I-e1, I-f1, I-2, I-a2, I-b2, I-c2, I-d2, I-e2, or I-f2: 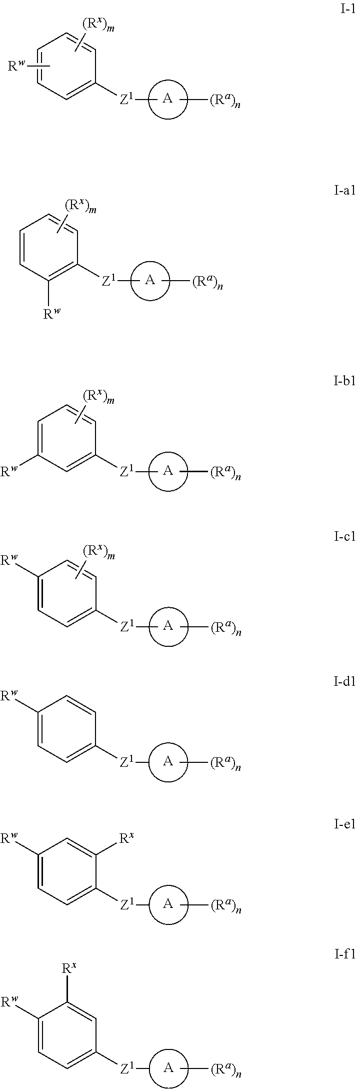 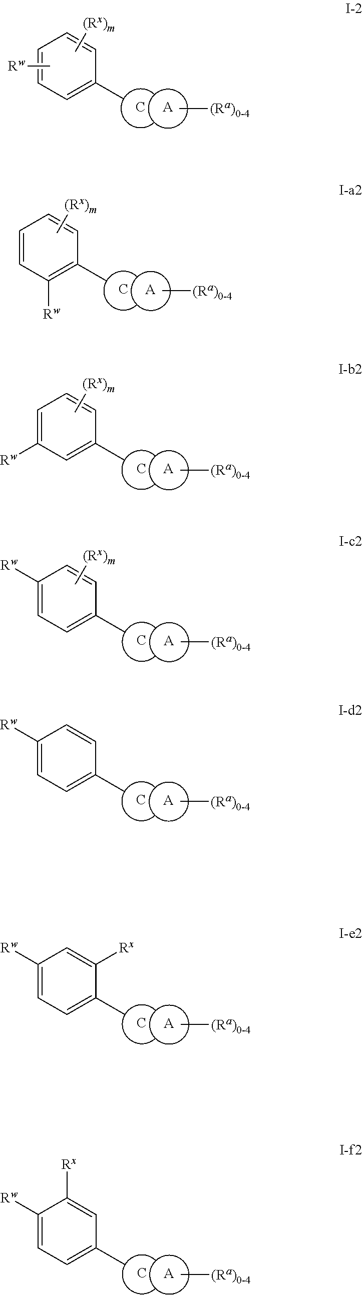
or a pharmaceutically acceptable salt thereof.

4. The compound according to claim 1, wherein the compound is of Formulae II, II-a, or II-b: 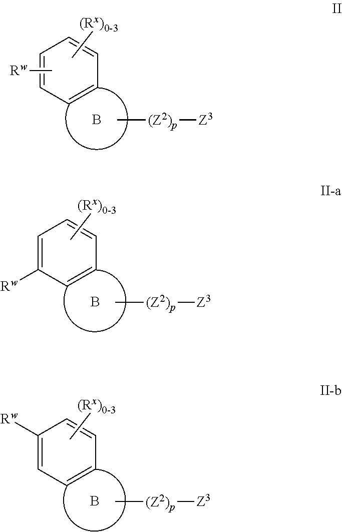
or a pharmaceutically acceptable salt thereof.

5. The compound according to claim 4, wherein the compound is of Formulae III, III-a, III-b, III-c, III-d, IV, IV-a, IV-b, V, V-a, V-b, V-c, V-d, VI, VI-a, VI-b, VI-c, VI-d, VII, VII-a, VII-b, VII-c, or VII-d: 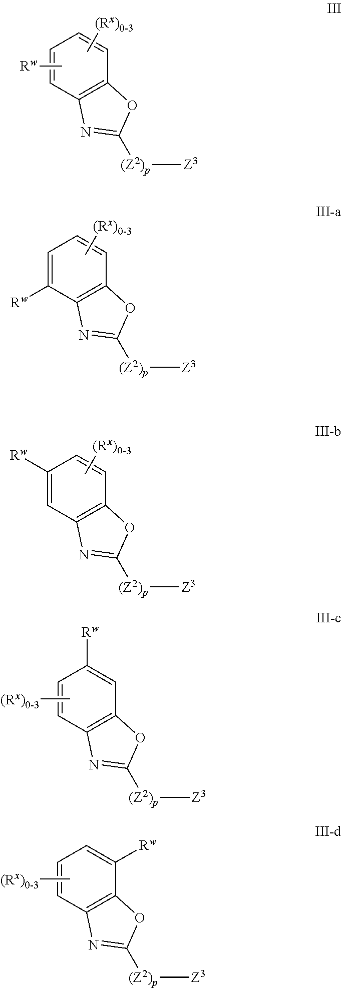 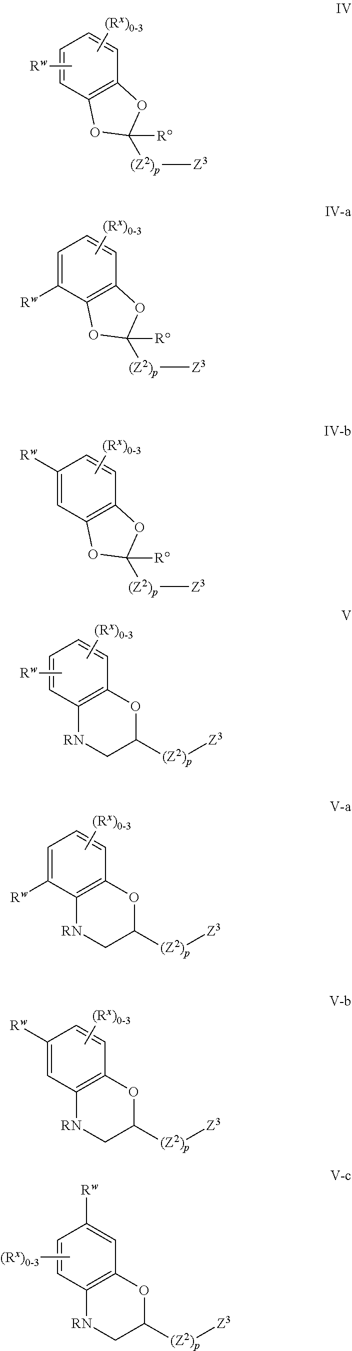 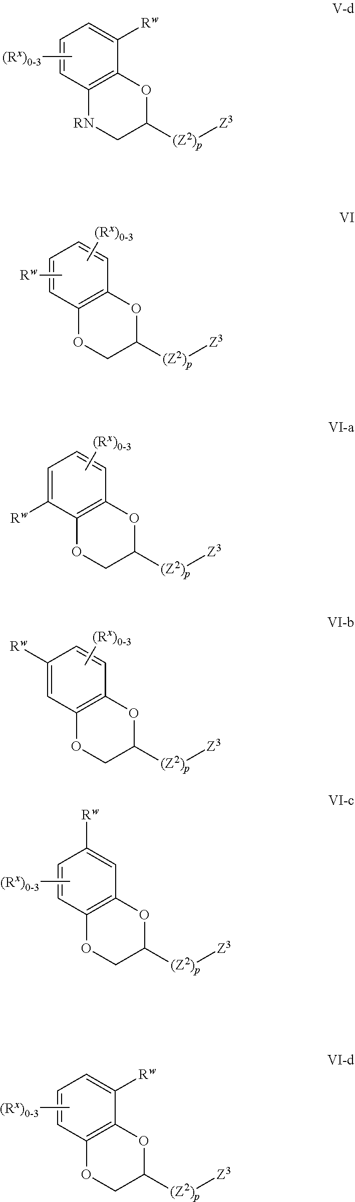 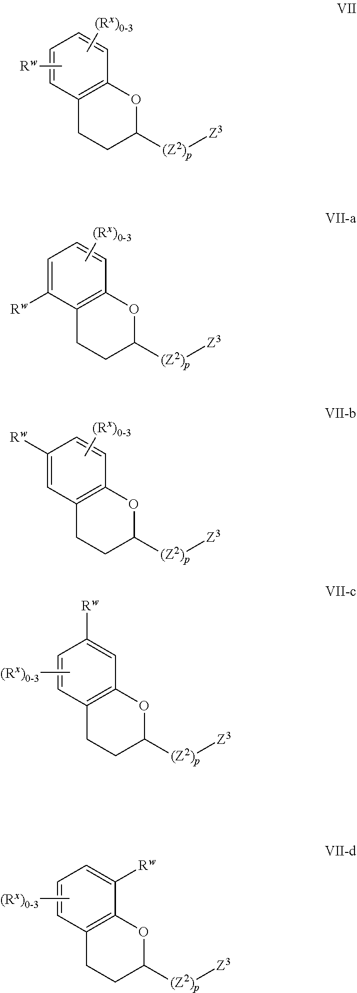
or a pharmaceutically acceptable salt thereof.

6. (canceled)

7. The compound according to claim 1, wherein R^{x} is selected from the group consisting of —CN, —F, —Cl, —CH₃, —C₅, —OMe,

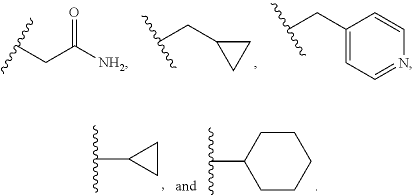

8. (canceled)

9. The compound according to claim 1, wherein Z³ is selected from the group consisting of:

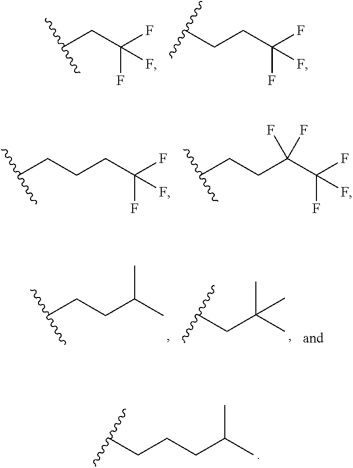

10. The compound according to claim 1, wherein Z³ is

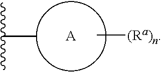

11. The compound according to claim 10, wherein Ring A is selected from the group consisting of:

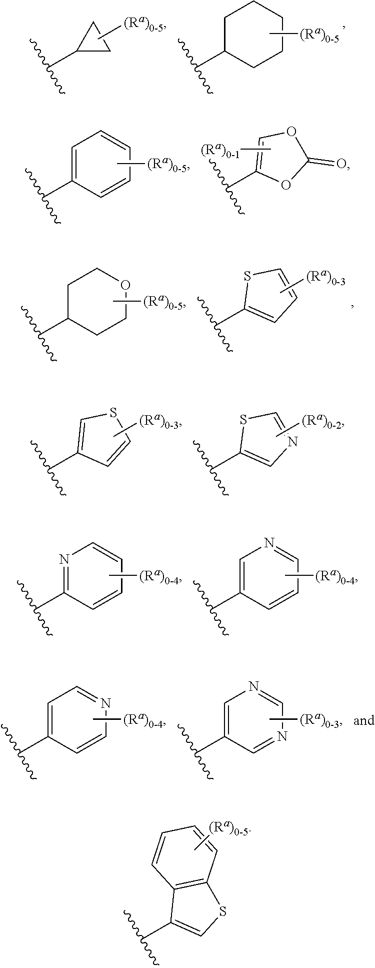

14. The compound according to claim 1, wherein Z¹ is selected from the group consisting of: 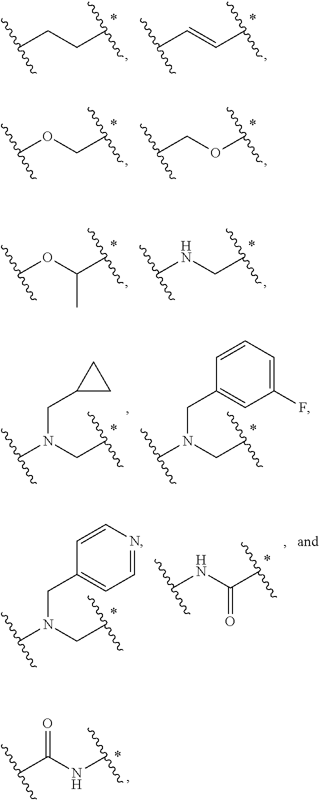
wherein * represents the point of attachment to Z².

15. The compound according to claim 1, wherein Ring B is selected from: 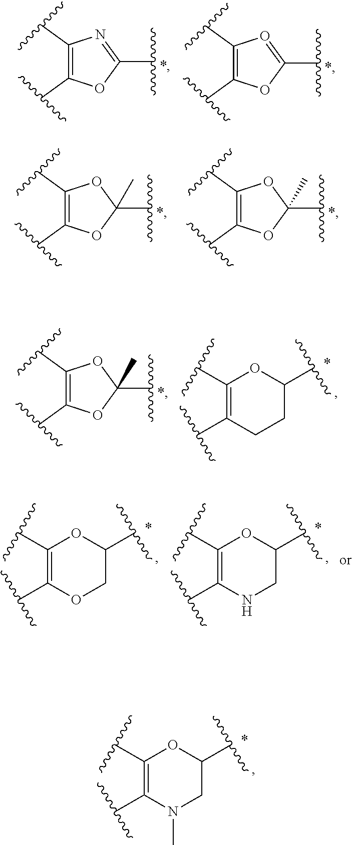
wherein * represents the point of attachment to Z².

16. The compound according to claim 1, wherein Ring C is selected from: 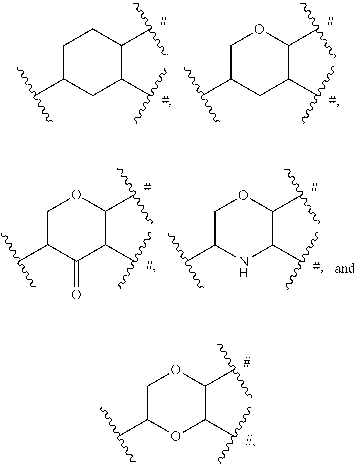 [SMILES: *C1CCC(*)C(*)C1 ; *C1COC(*)C(*)C1 ; *C1COC(*)C(*)C1=O ; *C1COC(*)C(*)N1 ; *C1COC(*)C(*)O1]
wherein each # represents a point of attachment to Ring A.

17. (canceled)

18. The compound according to claim 1, wherein L¹ is a covalent bond, —N(H)—, or —O—.

19-21. (canceled)

22. The compound according to claim 17, wherein R^{w} is selected from the group consisting of:

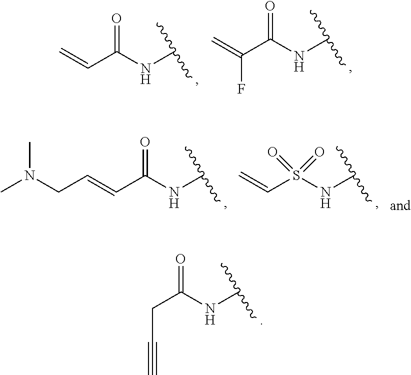

23. (canceled)

24. The compound according to claim 1, wherein the compound is compound is selected from the group consisting of: 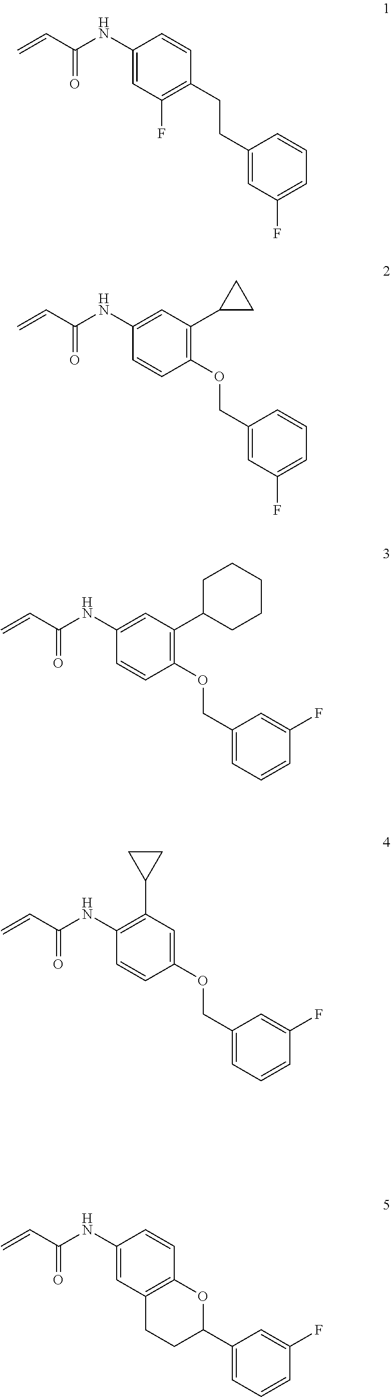 [SMILES: C=CC(=O)Nc1ccc(CCc2cccc(F)c2)c(F)c1 ; C=CC(=O)Nc1ccc(OCc2cccc(F)c2)c(C2CC2)c1 ; C=CC(=O)Nc1ccc(OCc2cccc(F)c2)c(C2CCCCC2)c1 ; C=CC(=O)Nc1ccc(OCc2cccc(F)c2)cc1C1CC1 ; C=CC(=O)Nc1ccc2c(c1)CCC(c1cccc(F)c1)O2] 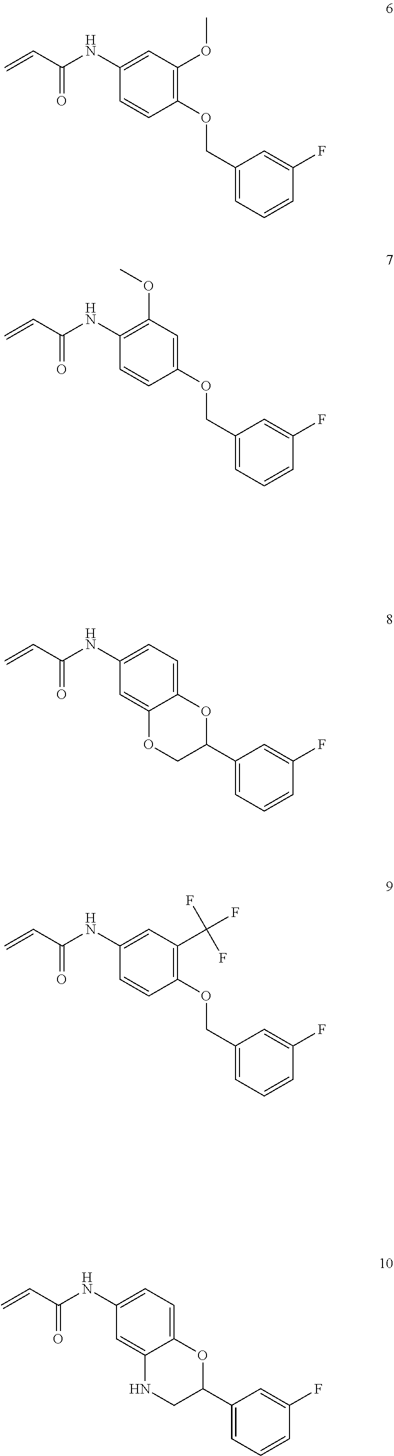 [SMILES: C=CC(=O)Nc1ccc(OCc2cccc(F)c2)c(OC)c1 ; C=CC(=O)Nc1ccc(OCc2cccc(F)c2)cc1OC ; C=CC(=O)Nc1ccc2c(c1)OCC(c1cccc(F)c1)O2 ; C=CC(=O)Nc1ccc(OCc2cccc(F)c2)c(C(F)(F)F)c1 ; C=CC(=O)Nc1ccc2c(c1)NCC(c1cccc(F)c1)O2] 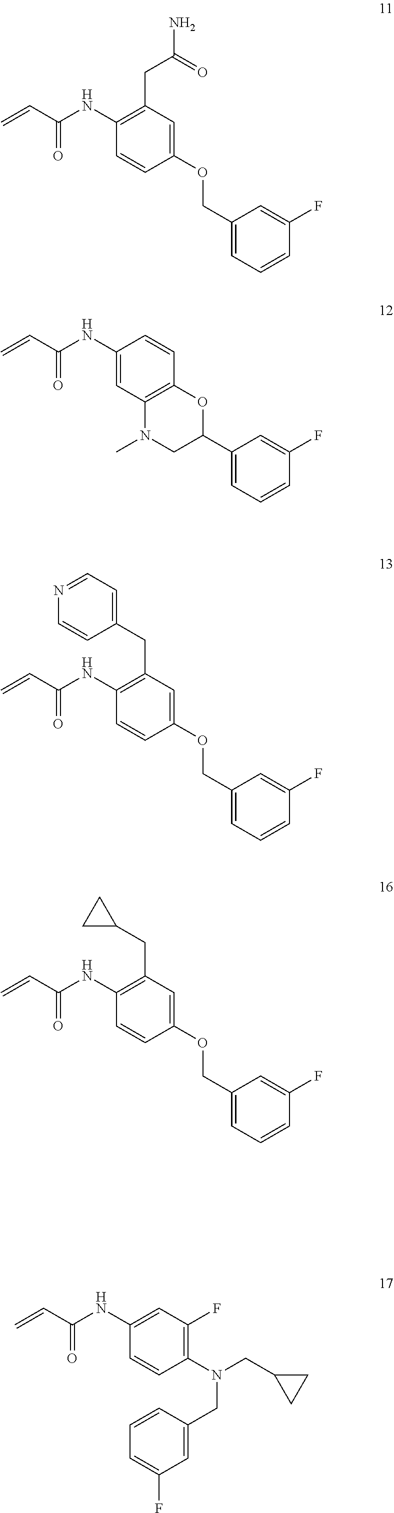 [SMILES: C=CC(=O)Nc1ccc(OCc2cccc(F)c2)cc1CC(N)=O ; C=CC(=O)Nc1ccc2c(c1)N(C)CC(c1cccc(F)c1)O2 ; C=CC(=O)Nc1ccc(OCc2cccc(F)c2)cc1Cc1ccncc1 ; C=CC(=O)Nc1ccc(OCc2cccc(F)c2)cc1CC1CC1 ; C=CC(=O)Nc1ccc(N(Cc2cccc(F)c2)CC2CC2)c(F)c1] 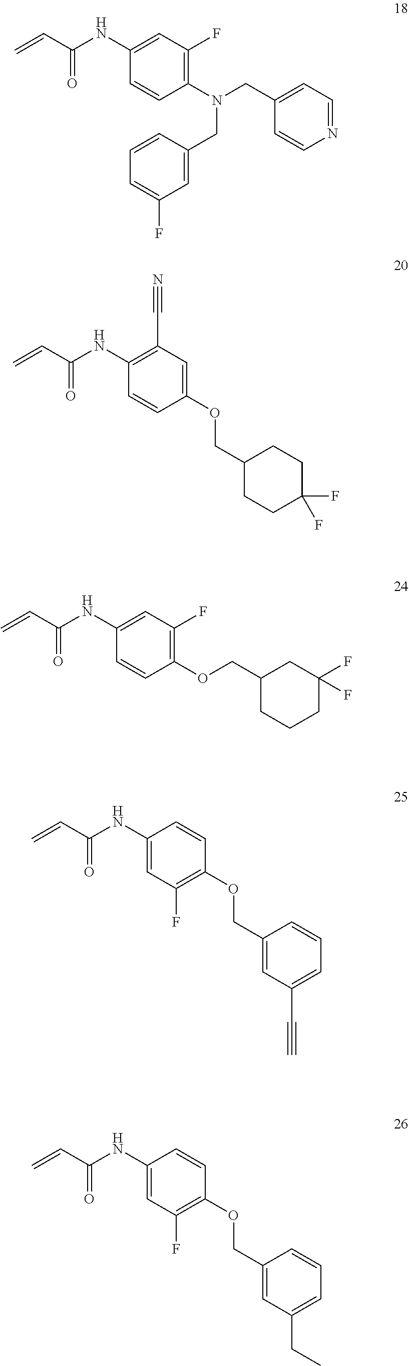 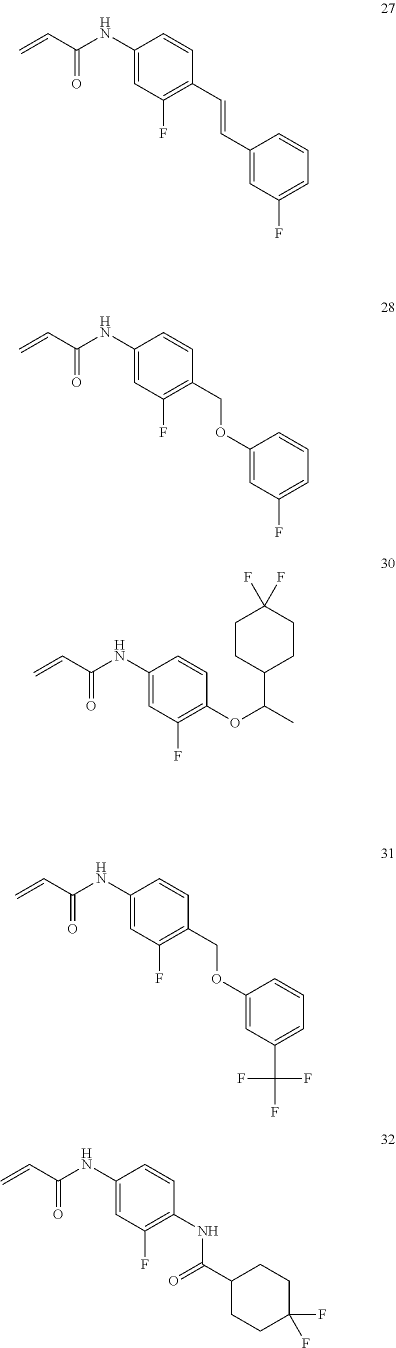 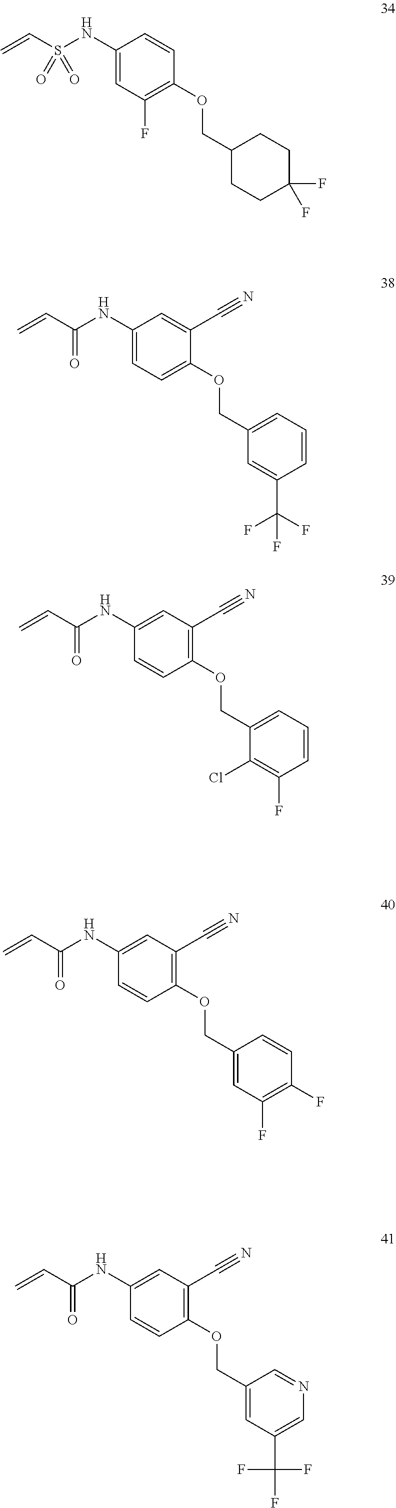 [SMILES: C=CS(=O)(=O)Nc1ccc(OCC2CCC(F)(F)CC2)c(F)c1 ; C=CC(=O)Nc1ccc(OCc2cccc(C(F)(F)F)c2)c(C#N)c1 ; C=CC(=O)Nc1ccc(OCc2cccc(F)c2Cl)c(C#N)c1 ; C=CC(=O)Nc1ccc(OCc2ccc(F)c(F)c2)c(C#N)c1 ; C=CC(=O)Nc1ccc(OCc2cncc(C(F)(F)F)c2)c(C#N)c1] 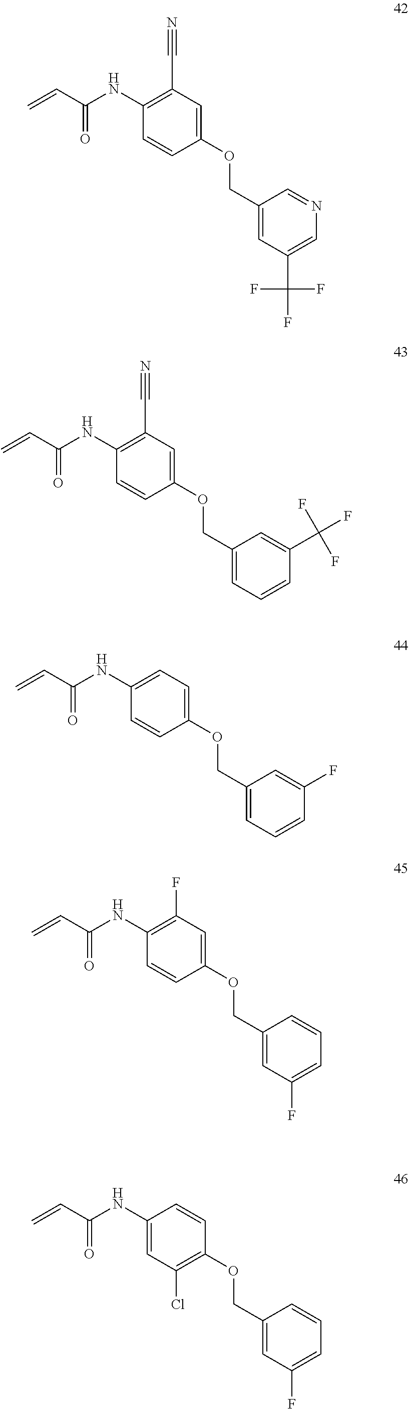 [SMILES: C=CC(=O)Nc1ccc(OCc2cncc(C(F)(F)F)c2)cc1C#N ; C=CC(=O)Nc1ccc(OCc2cccc(C(F)(F)F)c2)cc1C#N ; C=CC(=O)Nc1ccc(OCc2cccc(F)c2)cc1 ; C=CC(=O)Nc1ccc(OCc2cccc(F)c2)cc1F ; C=CC(=O)Nc1ccc(OCc2cccc(F)c2)c(Cl)c1] 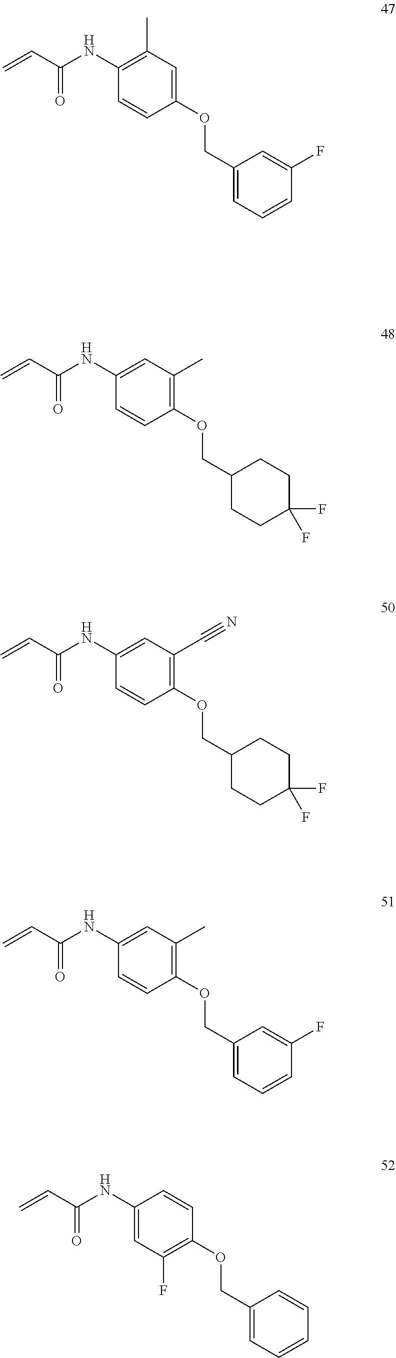 [SMILES: C=CC(=O)Nc1ccc(OCc2cccc(F)c2)cc1C ; C=CC(=O)Nc1ccc(OCC2CCC(F)(F)CC2)c(C)c1 ; C=CC(=O)Nc1ccc(OCC2CCC(F)(F)CC2)c(C#N)c1 ; C=CC(=O)Nc1ccc(OCc2cccc(F)c2)c(C)c1 ; C=CC(=O)Nc1ccc(OCc2ccccc2)c(F)c1] 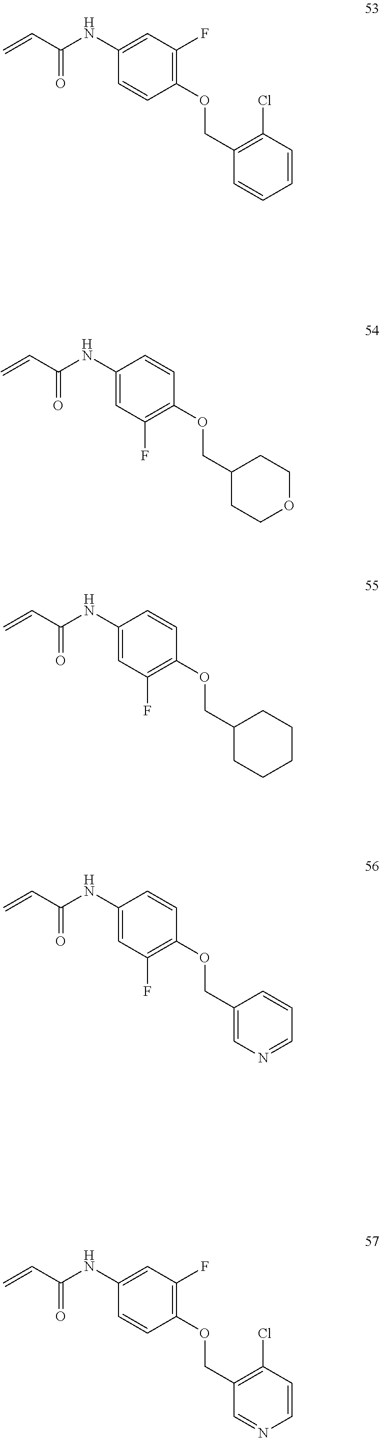 [SMILES: C=CC(=O)Nc1ccc(OCc2ccccc2Cl)c(F)c1 ; C=CC(=O)Nc1ccc(OCC2CCOCC2)c(F)c1 ; C=CC(=O)Nc1ccc(OCC2CCCCC2)c(F)c1 ; C=CC(=O)Nc1ccc(OCc2cccnc2)c(F)c1 ; C=CC(=O)Nc1ccc(OCc2cnccc2Cl)c(F)c1] 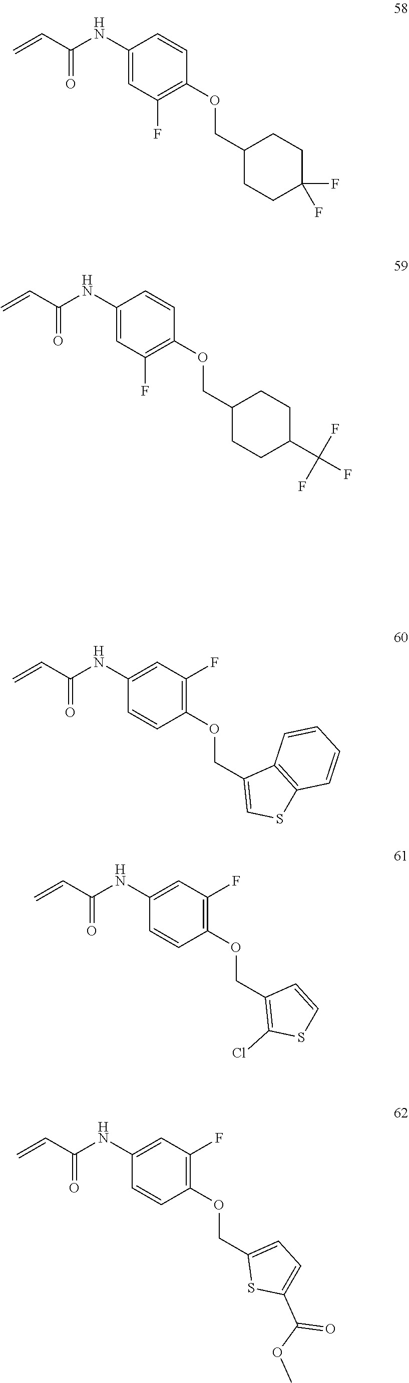 [SMILES: C=CC(=O)Nc1ccc(OCC2CCC(F)(F)CC2)c(F)c1 ; C=CC(=O)Nc1ccc(OCC2CCC(C(F)(F)F)CC2)c(F)c1 ; C=CC(=O)Nc1ccc(OCc2csc3ccccc23)c(F)c1 ; C=CC(=O)Nc1ccc(OCc2ccsc2Cl)c(F)c1 ; C=CC(=O)Nc1ccc(OCc2ccc(C(=O)OC)s2)c(F)c1] 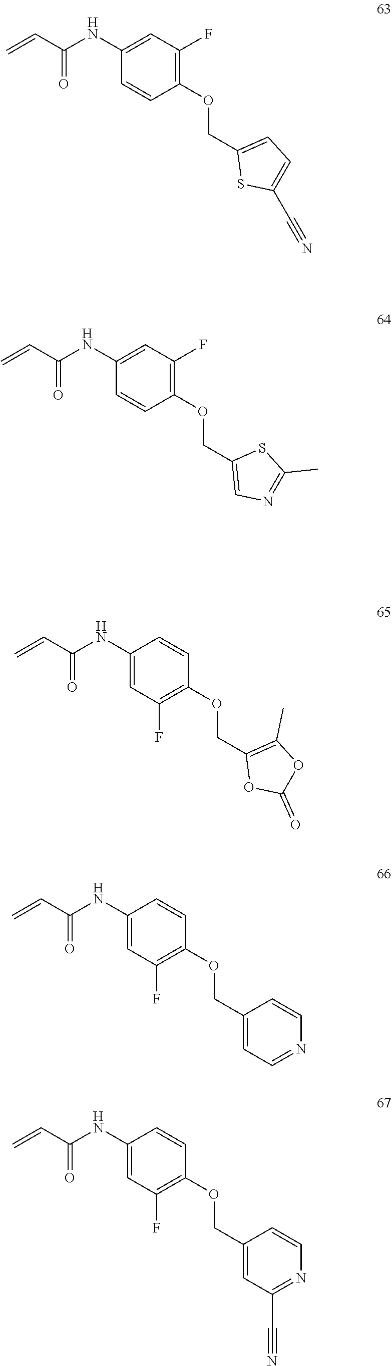 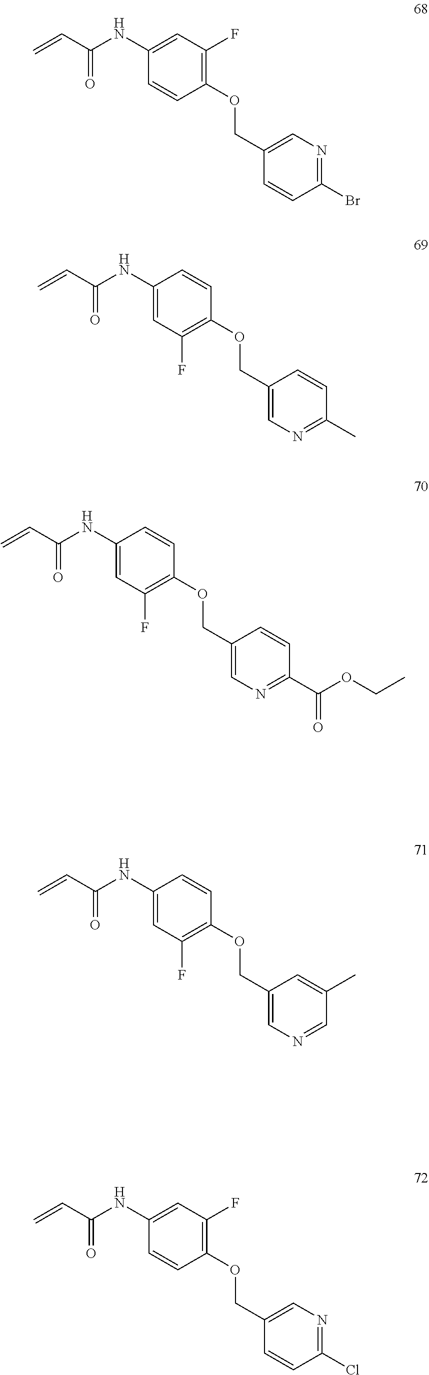 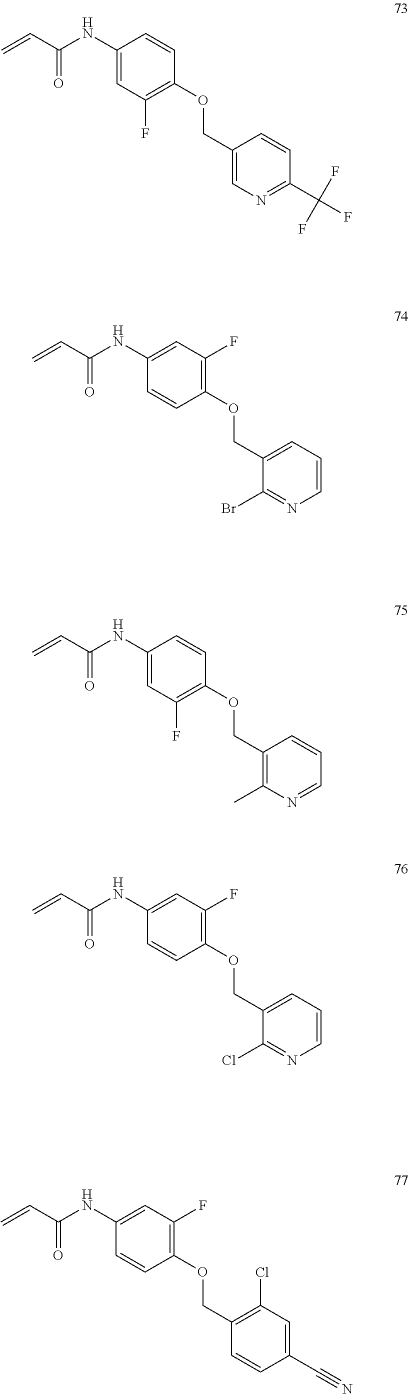 [SMILES: C=CC(=O)Nc1ccc(OCc2ccc(C(F)(F)F)nc2)c(F)c1 ; C=CC(=O)Nc1ccc(OCc2cccnc2Br)c(F)c1 ; C=CC(=O)Nc1ccc(OCc2cccnc2C)c(F)c1 ; C=CC(=O)Nc1ccc(OCc2cccnc2Cl)c(F)c1 ; C=CC(=O)Nc1ccc(OCc2ccc(C#N)cc2Cl)c(F)c1] 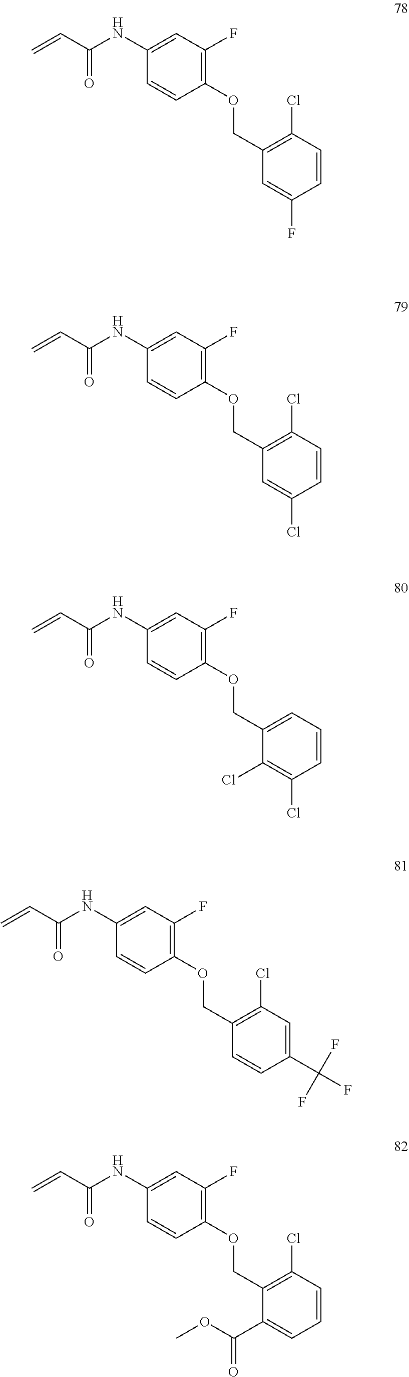 [SMILES: C=CC(=O)Nc1ccc(OCc2cc(F)ccc2Cl)c(F)c1 ; C=CC(=O)Nc1ccc(OCc2cc(Cl)ccc2Cl)c(F)c1 ; C=CC(=O)Nc1ccc(OCc2cccc(Cl)c2Cl)c(F)c1 ; C=CC(=O)Nc1ccc(OCc2ccc(C(F)(F)F)cc2Cl)c(F)c1 ; C=CC(=O)Nc1ccc(OCc2c(Cl)cccc2C(=O)OC)c(F)c1] 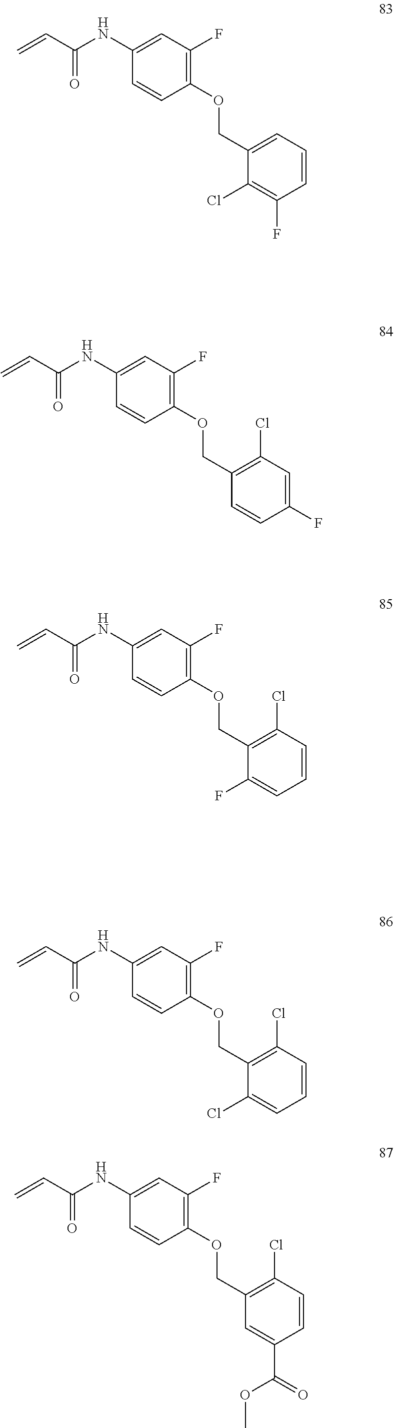 [SMILES: C=CC(=O)Nc1ccc(OCc2cccc(F)c2Cl)c(F)c1 ; C=CC(=O)Nc1ccc(OCc2ccc(F)cc2Cl)c(F)c1 ; C=CC(=O)Nc1ccc(OCc2c(F)cccc2Cl)c(F)c1 ; C=CC(=O)Nc1ccc(OCc2c(Cl)cccc2Cl)c(F)c1 ; C=CC(=O)Nc1ccc(OCc2cc(C(=O)OC)ccc2Cl)c(F)c1] 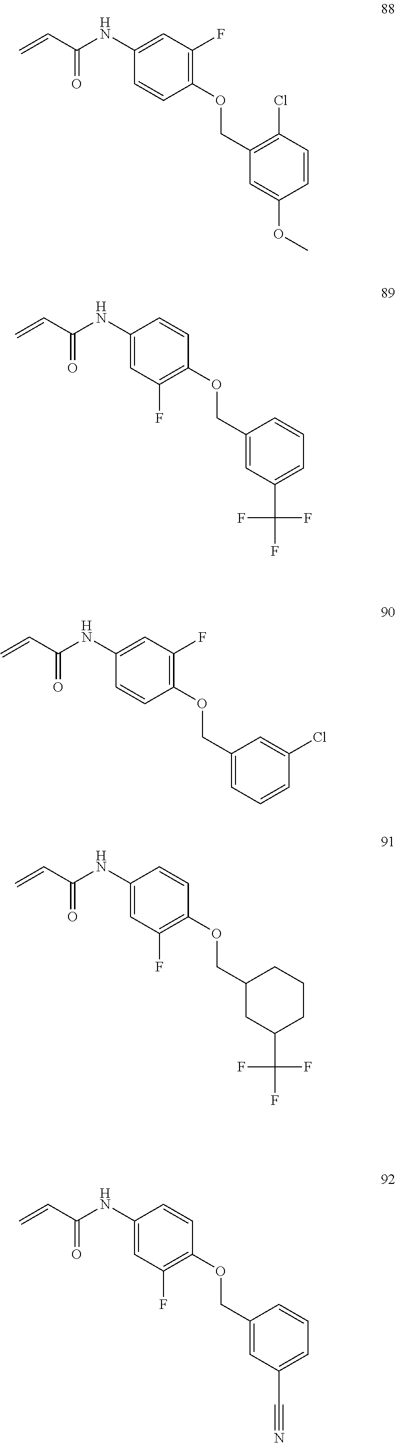 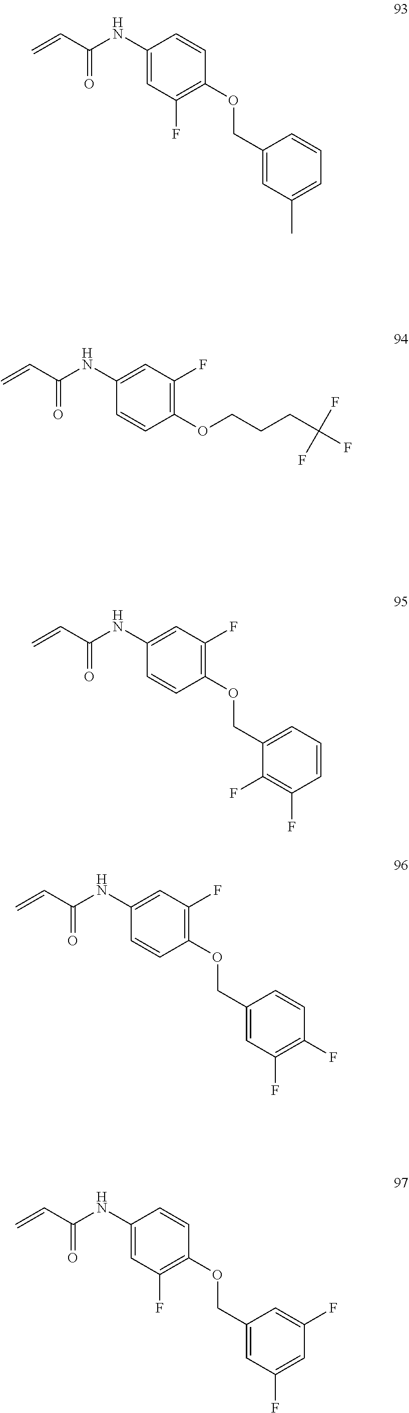 [SMILES: C=CC(=O)Nc1ccc(OCc2cccc(C)c2)c(F)c1 ; C=CC(=O)Nc1ccc(OCCCC(F)(F)F)c(F)c1 ; C=CC(=O)Nc1ccc(OCc2cccc(F)c2F)c(F)c1 ; C=CC(=O)Nc1ccc(OCc2ccc(F)c(F)c2)c(F)c1 ; C=CC(=O)Nc1ccc(OCc2cc(F)cc(F)c2)c(F)c1] 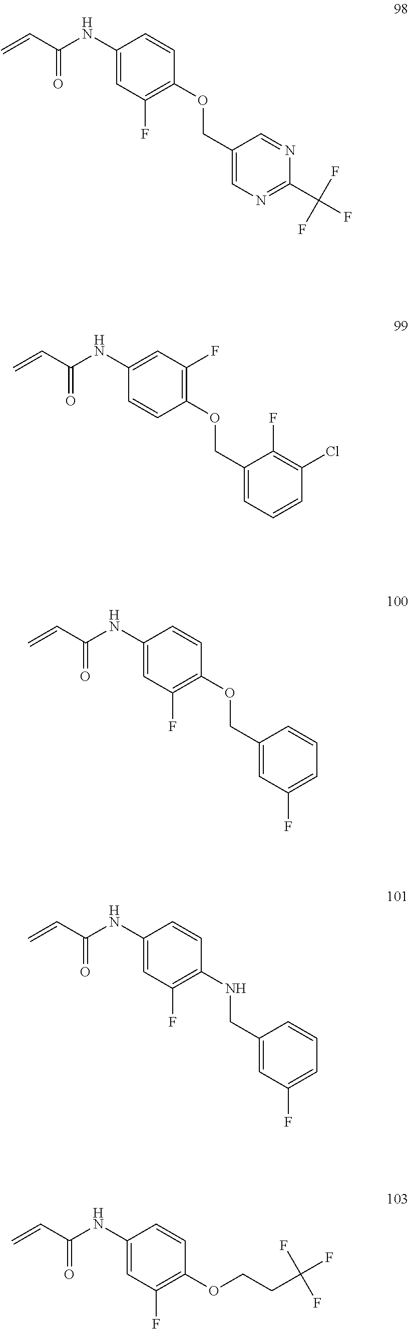 [SMILES: C=CC(=O)Nc1ccc(OCc2cnc(C(F)(F)F)nc2)c(F)c1 ; C=CC(=O)Nc1ccc(OCc2cccc(Cl)c2F)c(F)c1 ; C=CC(=O)Nc1ccc(OCc2cccc(F)c2)c(F)c1 ; C=CC(=O)Nc1ccc(NCc2cccc(F)c2)c(F)c1 ; C=CC(=O)Nc1ccc(OCCC(F)(F)F)c(F)c1] 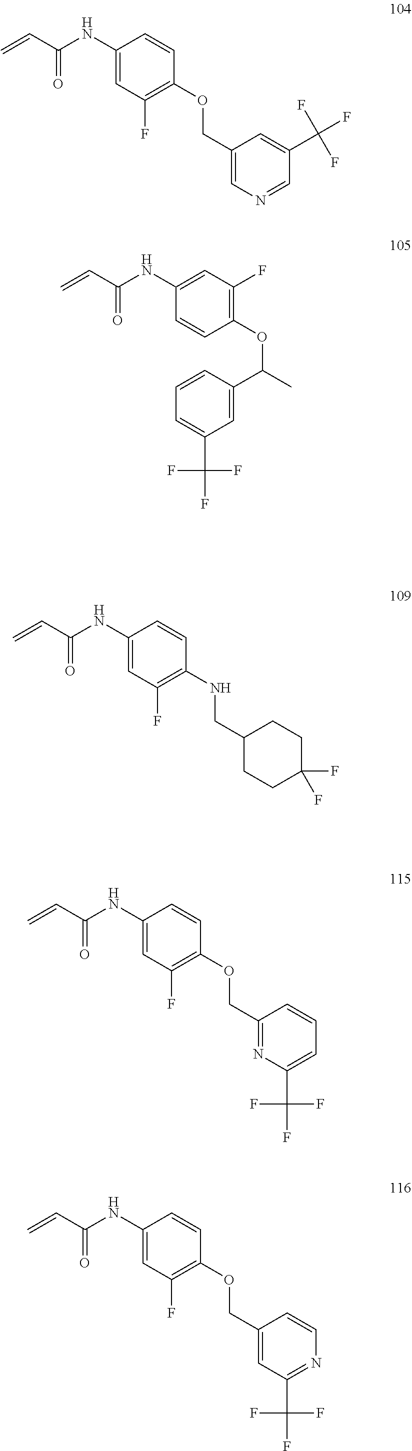 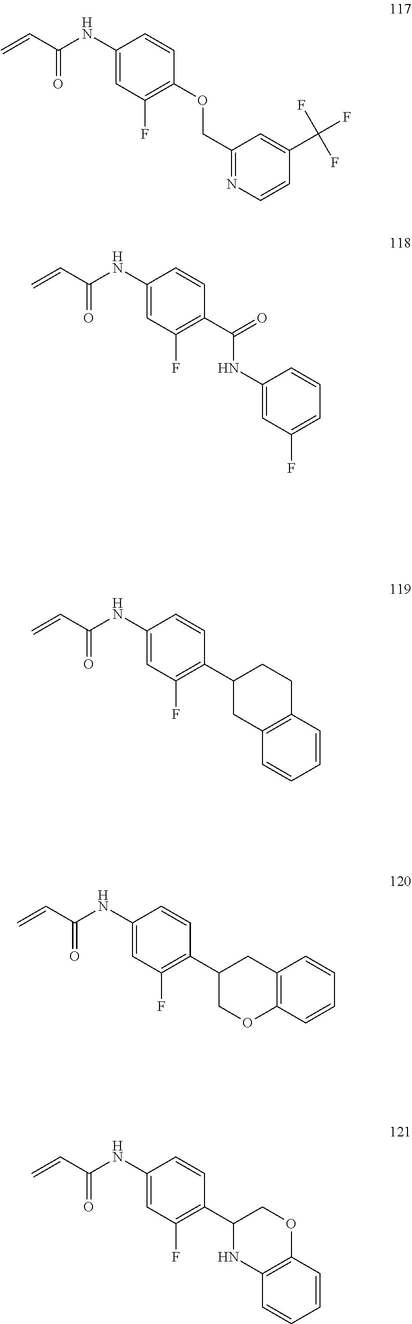 [SMILES: C=CC(=O)Nc1ccc(OCc2cc(C(F)(F)F)ccn2)c(F)c1 ; C=CC(=O)Nc1ccc(C(=O)Nc2cccc(F)c2)c(F)c1 ; C=CC(=O)Nc1ccc(C2CCc3ccccc3C2)c(F)c1 ; C=CC(=O)Nc1ccc(C2COc3ccccc3C2)c(F)c1 ; C=CC(=O)Nc1ccc(C2COc3ccccc3N2)c(F)c1] 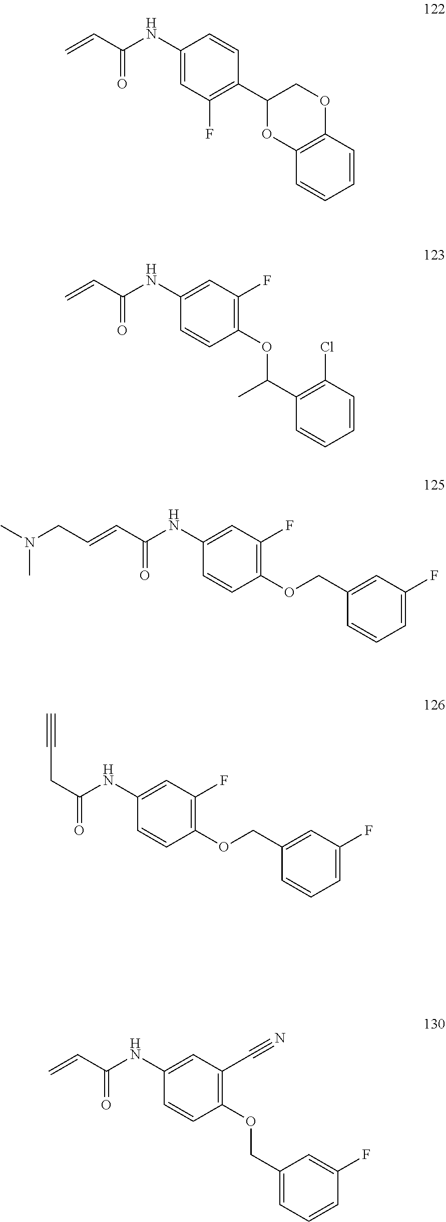 [SMILES: C=CC(=O)Nc1ccc(C2COc3ccccc3O2)c(F)c1 ; C=CC(=O)Nc1ccc(OC(C)c2ccccc2Cl)c(F)c1 ; CN(C)C/C=C/C(=O)Nc1ccc(OCc2cccc(F)c2)c(F)c1 ; C#CCC(=O)Nc1ccc(OCc2cccc(F)c2)c(F)c1 ; C=CC(=O)Nc1ccc(OCc2cccc(F)c2)c(C#N)c1] 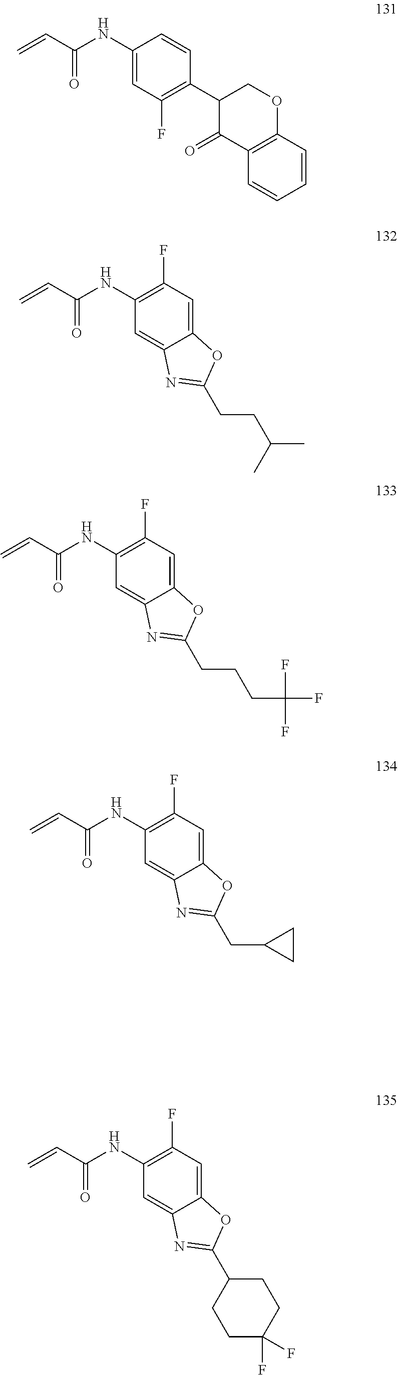 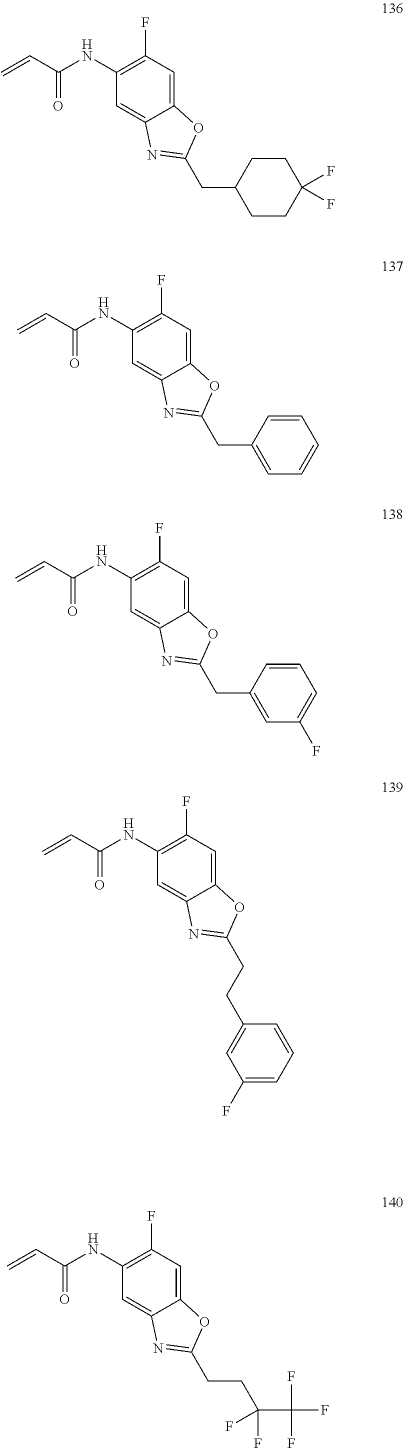 [SMILES: C=CC(=O)Nc1cc2nc(CC3CCC(F)(F)CC3)oc2cc1F ; C=CC(=O)Nc1cc2nc(Cc3ccccc3)oc2cc1F ; C=CC(=O)Nc1cc2nc(Cc3cccc(F)c3)oc2cc1F ; C=CC(=O)Nc1cc2nc(CCc3cccc(F)c3)oc2cc1F ; C=CC(=O)Nc1cc2nc(CCC(F)(F)C(F)(F)F)oc2cc1F] 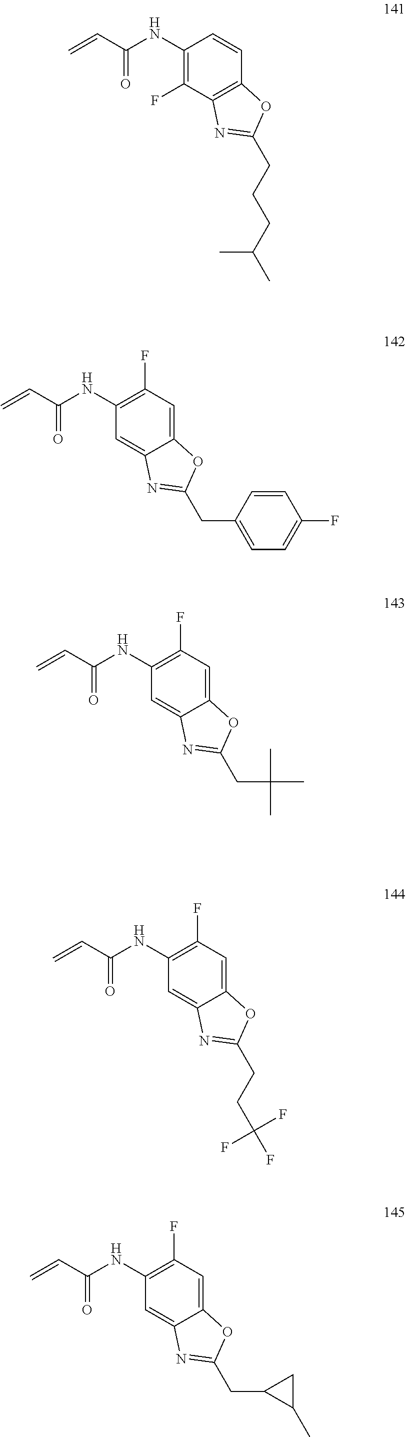 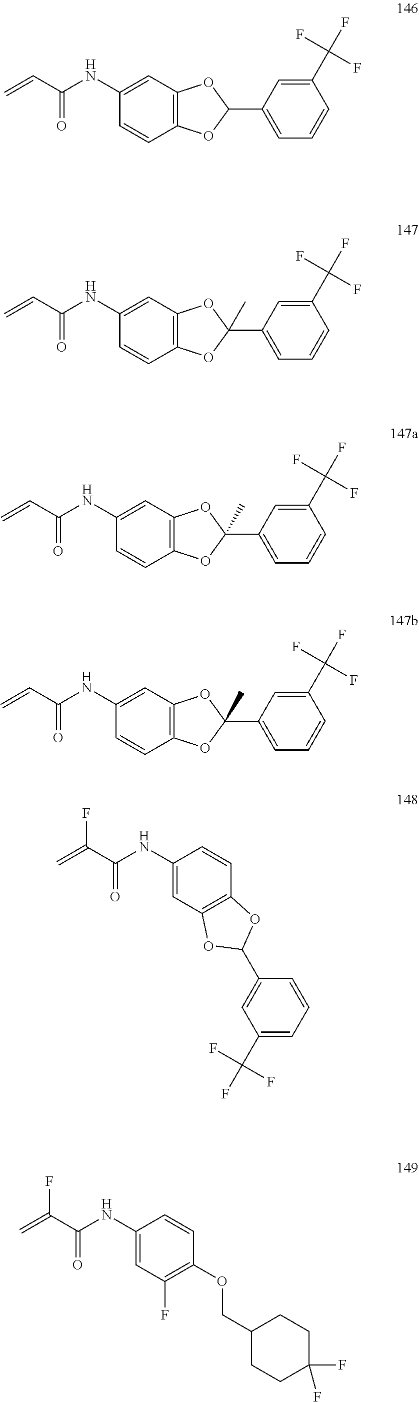 [SMILES: C=CC(=O)Nc1ccc2c(c1)OC(c1cccc(C(F)(F)F)c1)O2 ; C=CC(=O)Nc1ccc2c(c1)OC(C)(c1cccc(C(F)(F)F)c1)O2 ; C=CC(=O)Nc1ccc2c(c1)O[C@@](C)(c1cccc(C(F)(F)F)c1)O2 ; C=CC(=O)Nc1ccc2c(c1)O[C@](C)(c1cccc(C(F)(F)F)c1)O2 ; C=C(F)C(=O)Nc1ccc2c(c1)OC(c1cccc(C(F)(F)F)c1)O2 ; C=C(F)C(=O)Nc1ccc(OCC2CCC(F)(F)CC2)c(F)c1]
or a pharmaceutically acceptable salt thereof.

25. A compound selected from the group consisting of: 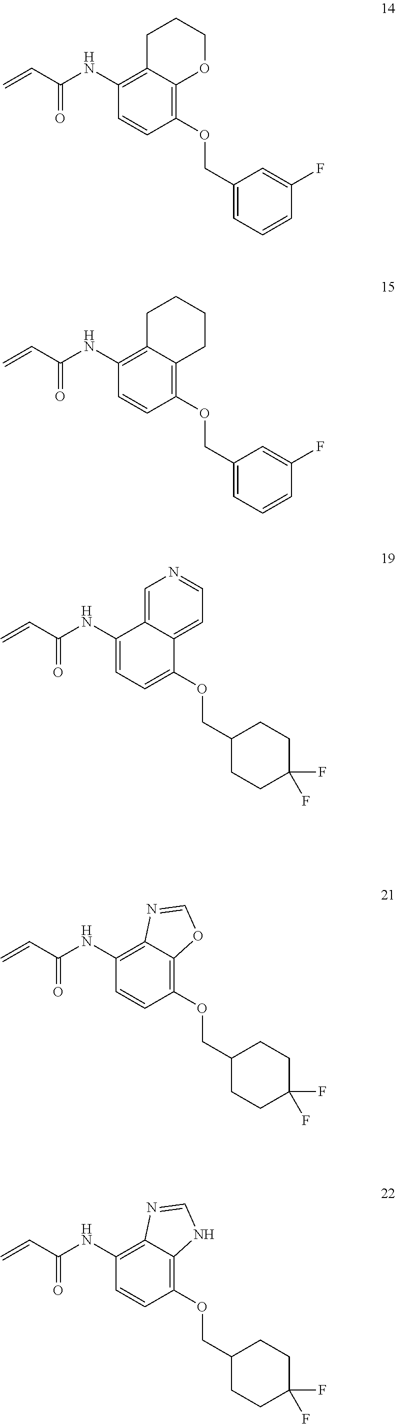 [SMILES: C=CC(=O)Nc1ccc(OCc2cccc(F)c2)c2c1CCCO2 ; C=CC(=O)Nc1ccc(OCc2cccc(F)c2)c2c1CCCC2 ; C=CC(=O)Nc1ccc(OCC2CCC(F)(F)CC2)c2ccncc12 ; C=CC(=O)Nc1ccc(OCC2CCC(F)(F)CC2)c2ocnc12 ; C=CC(=O)Nc1ccc(OCC2CCC(F)(F)CC2)c2[nH]cnc12] 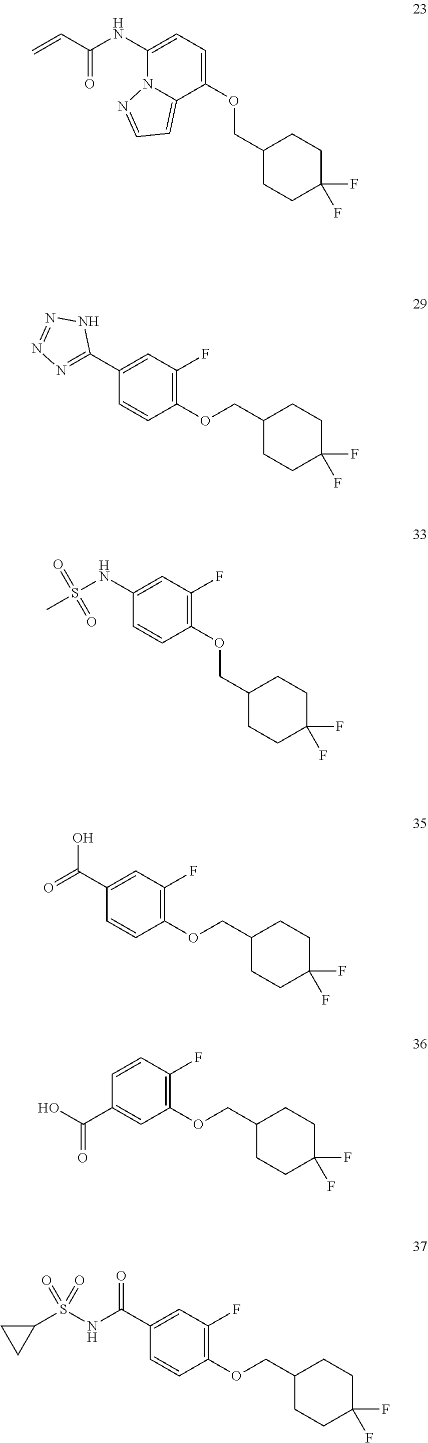 [SMILES: C=CC(=O)Nc1ccc(OCC2CCC(F)(F)CC2)c2ccnn12 ; Fc1cc(-c2nnn[nH]2)ccc1OCC1CCC(F)(F)CC1 ; CS(=O)(=O)Nc1ccc(OCC2CCC(F)(F)CC2)c(F)c1 ; O=C(O)c1ccc(OCC2CCC(F)(F)CC2)c(F)c1 ; O=C(O)c1ccc(F)c(OCC2CCC(F)(F)CC2)c1 ; O=C(NS(=O)(=O)C1CC1)c1ccc(OCC2CCC(F)(F)CC2)c(F)c1] 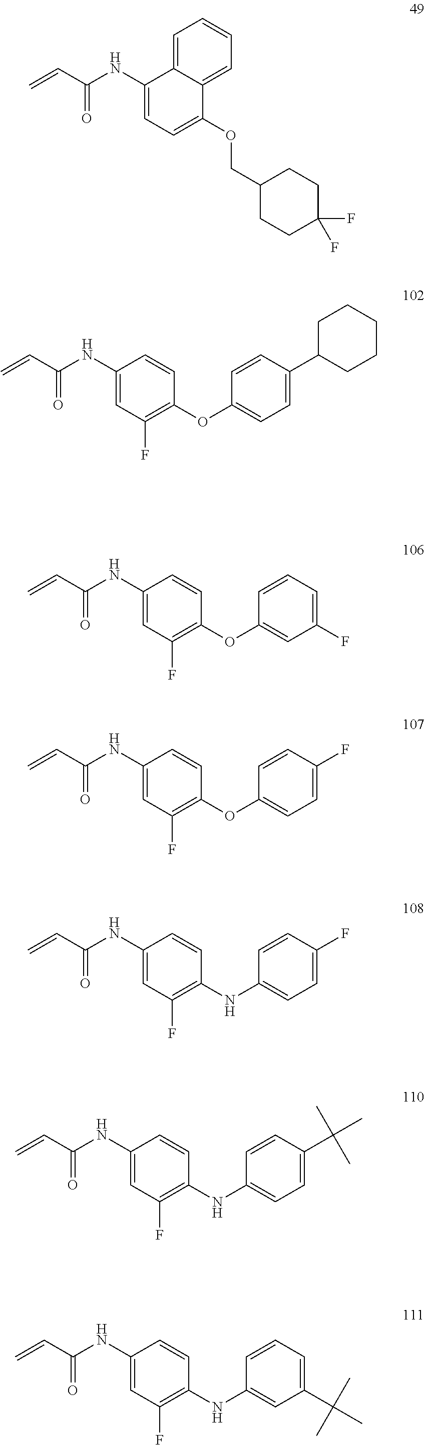 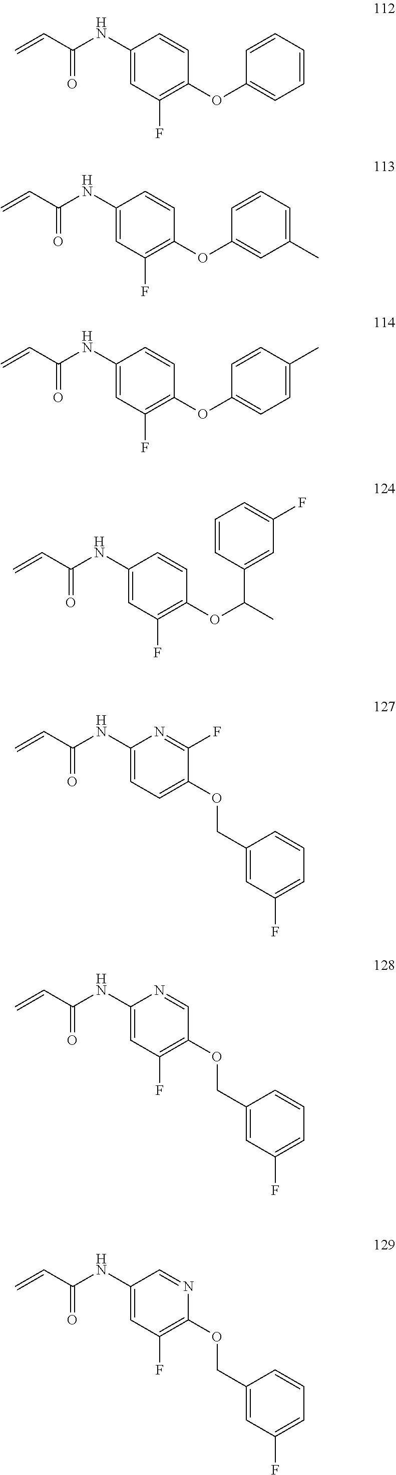 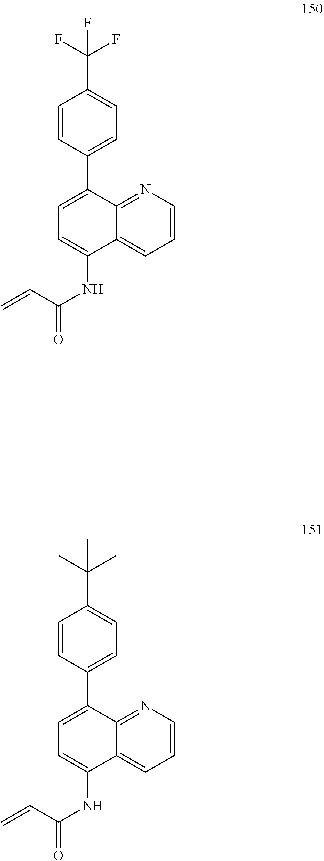 [SMILES: C=CC(=O)Nc1ccc(-c2ccc(C(F)(F)F)cc2)c2ncccc12 ; C=CC(=O)Nc1ccc(-c2ccc(C(C)(C)C)cc2)c2ncccc12]
or a pharmaceutically acceptable salt thereof.

26. A pharmaceutical composition comprising a compound according to am claim 1, or a pharmaceutically acceptable salt thereof, and a pharmaceutically acceptable carrier, adjuvant, or vehicle.

27. A method of inhibiting activity of a TEAD transcription factor, or a mutant thereof, in a biological sample or in a patient, the method comprising a step of contacting the biological sample or administering to a patient a compound according to claim 1, or a pharmaceutically acceptable salt thereof.

28. (canceled)

29. A method of treating a disease or disorder associated with TEAD, the method comprising a step of administering to a patient in need thereof a compound according to claim 1, or a pharmaceutically acceptable salt thereof.

30-32. (canceled)
